(19) United States
(12) Patent Application Publication
(10) Pub. No.: US 2023/0150970 A1
(43) Pub. Date: May 18, 2023

(54) AMINOPYRIMIDINE DERIVATIVES AND THEIR USE AS ARYL HYDROCARBON RECEPTOR MODULATORS

(71) Applicant: DONG-A ST CO., LTD., Seoul (KR)

(72) Inventors: Jung-Sang PARK, Osan-si (KR); Daewon CHA, Suwon-si (KR); Wonhyung LEE, Seoul (KR); Min Sung JOO, Seongnam-si (KR); Taeyoung YOON, Seoul (KR); Hyounmie DOH, Yongin-si (KR); Hyun Jung SUNG, Suwon-si (KR); Bo Ryeong LEE, Seoul (KR); Seunghyun SONG, Yongin-si (KR); Yoonjung KIM, Suwon-si (KR); Ji Hoon CHOI, Yongin-si (KR); Hyeon Seok JUNG, Yongin-si (KR)

(21) Appl. No.: 17/906,742

(22) PCT Filed: Mar. 29, 2021
(86) PCT No.: PCT/KR2021/003883

§ 371 Date: Sep. 19, 2022

Related U.S. Application Data
(60) Provisional application No. 63/000,584, filed on Mar. 27, 2020.

(51) Int. Cl. C07D 401/04; C07D 403/04; C07D 401/14; C07D 239/42; C07D 403/14; C07D 413/14; C07D 417/14; C07D 417/04

(52) U.S. Cl. CPC C07D 401/04; C07D 403/04; C07D 401/14; C07D 239/42; C07D 403/14; C07D 413/14; C07D 417/14; C07D 417/04

(57) ABSTRACT

The present invention relates to novel compounds effective as modulators Aryl hydrocarbon receptor (AhR), pharmaceutical composition comprising the compounds for the modulation of AhR, or prevention or treatment of a disease, disorder, or condition associated with AhR activity, as an active ingredient, and thus, can be useful as a medication for the prevention or treatment of a disease, disorder, or condition associated with AhR activity, in particular, cancer, cancerous condition, tumor, fibrotic disease, condition with dysregulated immune responses, etc.

### CROSS-REFERENCE TO RELATED APPLICATIONS

The present application claims priority from U.S. Provisional Application No. 63/000,584, filed Mar. 27, 2020, which are incorporated herein by reference in their entirety.

### TECHNICAL FIELD

The present invention relates to novel pyridopyrimidinone derivatives that can modulate the activities of aryl hydrocarbon receptor (AhR). The compounds of formula (I) of the present invention can also be used for inhibiting the growth of cancer cells, tumor cell metastasis and invasion and for the treatment of diseases related with dysregulated immune responses associated with AhR signaling (a sole agent or in combination with other active ingredients).

### BACKGROUND ART

Aryl hydrocarbon receptor (AhR) is a ligand-activated transcription factor and is well-known as an important intracellular chemosensor responsive to both natural and man-made environmental compounds. As is well known, the AhR is a member of the periodic circadian protein (PER)—AhR nuclear translocator (ARNT)—single-minded protein (SIM) superfamily of transcription factors in which the PER-ARNT-SIM (PAS) domain senses ligands. (Burbach et al, PNAS Sep. 1, 1992 89 (17) 8185-8189) The AhR, activated by several binding ligands translocates to the nucleus and dimerizes with its partner protein, the ARNT. This heterodimeric complex interacts with the xenobiotic response elements (XREs) and it control the expression of AhR related genes directly or indirectly. One of the endogenous ligands to be well-characterized is kynurenine, generated by TDO (Opitz et al, Nature, 2011 Oct. 5; 478(7368):197-203) or IDO (Mezrich, J Immunol. 2010 Sep. 15; 185(6):3190-8). Recent studies found that high concentrations of kynurenine in the plasma of diverse cancer patients and a high serum Kyn/Trp ratio correlates with poor prognosis after PD-1 blockade in several cancer types, including lung cancer, melanoma, and renal cell carcinomas. (Haoxin Li et al, Nat Commun. 2019 Sep. 25; 10(1):4346)

It has been well-known lately that AhR regulates the functions of a plethora of cells of both the innate and adaptive immune system. Activated AhR attenuates the induction of cytokines that promote the polarization of pathogenic T cell subsets and reduces MHC class II expression. In addition, AhR activation by agonist or modulator, inhibits the differentiation of helper Th17 cell and stabilizes regulatory T cell. Invigorated AhR also induces the generation of its ligands via a positive feedforward loop involving indolamine 2,3-dioxygenase 1 (IDO1). (Nguyen et al., PNAS, 2010, 107(46):19961-19966, Mascanfroni, I. D. et al. Nat. Med., 2015, 21:638-646) As an immune escape mechanism, Tumor-repopulating cells (TRCs) drive PD-1 upregulation in CD8+ T cells through a Kyn-AhR pathway. (Yuying Liu et al, Cancer cell, 2018 Mar. 12; 33(3):480-494.e7).

Moreover, several studies have shown that AhR signaling plays important roles in diverse disease such as autoimmunity, infection, and cancer. AhR signaling may be related to autoimmune diseases, including rheumatoid arthritis (RA), systemic lupus erythematosus (SLE), multiple sclerosis (MS). (Xiao-Song Wang et al, Inflammopharmacology, 2020 February; 28(1):63-81) Constitutive AhR activation reduces the type I IFN (IFN-I) antiviral response (Yamada et al, Nat immunol, 2016 June; 17(6):687-94). The AhR activation is induced by multiple viruses to evade the host immune response, a strategy exploited in mouse models to limit the replication of Zika virus, SARS-COV-2 infection. (Federico Giovannoni et al, Cell Research, 2021 December, 31:1-2) The AhR may affect the proliferation, tissue invasion, metastasis, and angiogenesis of cancer cells (Jae Eun Cheong et al, Trends in Pharmacological Sciences, 2018 March; 39(3):307-325). In addition, many cancer types can escape from immune recognition via an AhR pathway. Developing AhR-targeted therapeutics could be the potential opportunities to overcome immune related diseases.

### DISCLOSURE

### Technical Problem

Therefore, it is an object of the present invention to provide novel compounds, or an enantiomer, diastereomer, racemate, solvate, hydrate or pharmaceutically acceptable salt thereof as modulators of AhR.

It is an object of the present invention to provide a pharmaceutical composition for the modulation of AhR activity, comprising the compounds as modulators of AhR.

It is an object of the present invention to provide a pharmaceutical composition for the prevention or treatment of disease, disorder, or condition associated with AhR activity such as a cancer or an autoimmune disease, comprising the compounds as modulators of AhR.

It is an object of the present invention to provide a method for modulating AhR activity by administering the compounds as modulators of AhR.

It is an object of the present invention to provide a method for preventing or treating prostaglandin related diseases by administering the compounds as modulators of AhR.

It is an object of the present invention to provide a use of the prostaglandin analog for the modulation of AhR activity, or the prevention or treatment of disease, disorder, or condition associated with AhR.

### Technical Solution

### SUMMARY OF THE INVENTION

The present invention provides novel compounds, or an enantiomer, diastereomer, or pharmaceutically acceptable salt thereof are effective as modulators or antagonists of AhR. The compounds are represented by formula (I)

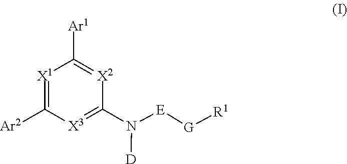

wherein:

X¹, X² and X³ are each independently CR², N or NR³;

Ar¹ and Ar² are each independently selected from substituted or unsubstituted mono- or bicyclic C₆₋₁₀ aryl, substituted or unsubstituted mono- or bicyclic C₅₋₁₀ heteroaryl and substituted or unsubstituted mono- or bicyclic C₃₋₁₀ heterocycloalkyl;

D is H, halo, cyano, hydroxy, amino, substituted or unsubstituted C₁₋₅ alkyl, mono- or bicyclic C₃₋₁₀ cycloalkyl, C₁₋₅ alkylhydroxy, C₁₋₅-Song Wang et al, alkenylhydroxy, C₁₋₅ alkynylhydroxy, C₁₋₅ alkylamine, C₁₋₅ alkenylamine, C₁₋₅ alkynylamine, mono- or bicyclic C₃₋₁₀ heterocycloalkyl, mono- or bicyclic C₃₋₁₀ heteroaryl,

E is absent (direct bond), amino, substituted or unsubstituted C₁₋₅ alkyl, mono- or bicyclic C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, mono- or bicyclic C₃₋₁₀ heterocycloalkyl, mono- or bicyclic C₃₋₁₀ heteroaryl,

or D and E, together with the atoms to which they are attached, are combined to form substituted or unsubstituted mono- or bicyclic C₃₋₁₀ heterocycloalkyl ring;

G is absent (direct bond), H, halo, cyano, hydroxy, amino, nitro, ether (—O—), thioether (—S—), sulfinyl (—SO—), sulfonyl(—SO₂—), sulfonylamido(—SO₂NR⁴—), aminosulfonyl(—NR⁴SO₂—), carbonyl(—(CO)—), amido(—(CO)NR⁴—), reverse amido(—NR⁴(CO)—), ester (—(CO)O—), substituted or unsubstituted mono- or bicyclic C₃₋₁₀cycloalkyl, substituted or unsubstituted mono- or bicyclic C₃₋₁₀ heterocycloalkyl, substituted or unsubstituted mono- or bicyclic C₆₋₁₀ aryl and substituted or unsubstituted mono- or bicyclic C₅₋₁₀ heteroaryl;

R¹ is absent, H, halo, cyano, hydroxy, amino, N(R⁵)₂, OR⁵, substituted or unsubstituted C₁₋₅ alkyl, C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, substituted or unsubstituted mono- or bicyclic C₃₋₁₀ heterocycloalkyl and substituted or unsubstituted mono- or bicyclic C₅₋₁₀ heteroaryl;

R² is H, halo, cyano, hydroxy and C₁₋₃ alkyl;

R³ is H, halo, cyano, hydroxyl and amino; and

R⁴ is H, substituted or unsubstituted C₁₋₅ alkyl, substituted or unsubstituted C₁₋₅ alkoxy and substituted or unsubstituted C₁₋₅ alkyl carboxylic acid; and

R⁵ is H, substituted or unsubstituted C₁₋₅ alkyl, substituted or unsubstituted C₁₋₅ alkoxy and substituted or unsubstituted C₁₋₅ alkyl carboxylic acid; In some embodiments of these aspects and all such aspects described herein, the AhR modulator of Formula (I) is an AhR modulator or AhR antagonist.

In some aspects, described herein are methods of modulating AhR activity, more specifically constitutive AhR activity in a subject in need thereof. Such methods comprise administering to a subject having constitutive AhR activity a therapeutically effective amount of an AhR modulator, such as an AhR antagonist of Formula (I), described herein. In some embodiments of these aspects and all such aspects described herein, the methods further comprise the step of selecting the subject having constitutive AhR activity.

Compounds of formula (I) of the present invention demonstrate a valuable pharmacological spectrum of action, which could not have been predicted. Compounds of the present invention have surprisingly been found to effectively inhibit AhR and it is possible therefore that said compounds be used for the treatment or prophylaxis of a disease or condition mediated by aryl hydrocarbon receptor (AhR), preferably cancer, cancerous conditions, tumor, fibrotic disorders, or conditions with dysregulated immune responses or other disorders associated with aberrant AhR signaling, in humans and animals.

Examples of said diseases related with dysregulated immune response associated with AhR signaling are sepsis (SIRS), multiple organ failure (MODS, MOF), inflammatory disorders of the kidney, chronic intestinal inflammations (IBD, Crohn's disease, UC), pancreatitis, peritonitis, inflammatory skin disorders and inflammatory eye disorders, autoimmune diseases, such as rheumatoid diseases including rheumatoid arthritis (RA), systemic lupus erythematosus (SLE), multiple sclerosis (MS), etc.

Examples of said fibrotic disorders are fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

In other aspects, described herein are methods of treating a cancer or a cancerous condition by modulating AhR activity. Such methods comprise administering to a subject having a cancer or cancerous condition a therapeutically effective amount of any of the pharmaceutical compositions comprising an AhR modulator, such as an AhR antagonist of Formula (I), described herein.

In some aspects, described herein are methods of inhibiting tumor cell invasiveness in a subject having a cancer, a cancerous condition, or a tumor. Such methods comprise administering to a subject having a cancer or a tumor a therapeutically effective amount of any of the pharmaceutical compositions comprising an AhR modulator, such as an AhR antagonist of Formula (I), described herein.

In some embodiments of these aspects and all such aspects described herein, the methods further comprise the step of selecting the subject having a cancer, a cancerous condition, or a tumor.

Said cancer, cancerous condition, or tumor particularly suitable for treatment with an AHR inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds or of the present invention can be used in particular in therapy and prevention, i.e. prophylaxis, of tumour growth and metastases, especially in solid tumours of all indications and stages with or without pre-treatment of the tumour growth the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia. In some such embodiments, the cancer is a hepatocellular cancer.

Some embodiments of these methods can further comprise administration or treatment with one or more additional anti-cancer therapies. In some such embodiments, the additional anti-cancer therapy comprises surgery, radiation therapy, biotherapy, immunotherapy, chemotherapy, or any combination thereof.

Some embodiments of these methods can further comprise administration or treatment with one or more anti-cancer therapeutic agents. In some such embodiments, the anti-cancer therapeutic agent is a chemotherapeutic agent, a growth inhibitor agent, an anti-angiogenesis agent, a cytotoxic agent, an anti-hormonal agent, a prodrug, or a cytokine.

In a further embodiment of the present invention, the compounds of formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of formula (I) of the present invention prior to the treatment of the cell to cause or induce cell death. In one aspect, after the cell is treated with one or more compounds of formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of formula (I) of the present invention to sensitize the cell to cell death, the cell is treated with at least one DNA damaging agent to kill the cell. DNA damaging agents useful in the present invention include, but are not limited to, chemotherapeutic agents (e.g. cisplatin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of formula (I) of the present invention is administered to a cell immediately after radiation or other induction of DNA damage in the cell has begun.

In another aspect, the cell is in vitro. In another embodiment, the cell is in vivo. The compounds of the present invention can be administered as the sole pharmaceutical agent or in combination with one or more other pharmaceutically active ingredients where the combination causes no unacceptable adverse effects.

The present invention also covers such pharmaceutical combinations. For example, the compounds of the present invention can be combined with: 131 1-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (1231), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors, e.g. aPD-1/-L1 axis antagonists.

PD-1, along with its ligands PD-L1 and PD-L2, function as negative regulators of T cell activation. AHR suppresses immune cell function while increasing cancer cell proliferation and motility. PD-L1 is overexpressed in many cancers and overexpression of PD-1 often occurs concomitantly in tumor infiltrating T cells. Thus results in attenuation of T cell activation and evasion of immune surveillance, which contributes to impaired antitumor immune responses. (Keir M E et al. (2008) Annu. Rev. Immunol. 26:677).

Simultaneously targeting both the PD-1/-L1 axis and AHR enhances antitumor immune responses more than in an additive manner, leading to a reduction of tumor growth that is unexpected.

Thus, compositions comprising a PD-1/-L1 axis antagonist and an AHR antagonist are surprisingly effective in enhancing an immune response and in the treatment of cancer.

In addition, the inventive compounds can also be used as a therapeutic in a variety of other disorders wherein AHR is involved.

Examples of other disorders associated with aberrant AhR signaling inflammation are vaccination for infection & cancer, viral infections, obesity and diet-induced obesity, adiposity, metabolic disorders, hepatic steatosis and uterine fibroids (uterine leiomyoma or uterine myoma) in women, chronic renal disorders, acute and chronic renal insufficiency, diabetic, inflammatory or hypertensive nephropaties, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer, and Creutzfeld-Jakob.

Also provided herein, in other aspects, are pharmaceutical compositions comprising an AhR modulator, such as an AhR antagonist of Formula (I), and pharmaceutically acceptable excipients.

In some aspects, pharmaceutical compositions comprising an AhR modulator, such as an AhR antagonist of Formula (I), are provided for use in for modulating constitutive AhR activity in a subject in need thereof.

In some aspects, pharmaceutical compositions comprising an AhR modulator, such as an AhR antagonist of Formula (I), are provided for use in treating a cancer or a cancerous condition by modulating AhR activity.

In some aspects, pharmaceutical compositions comprising an AhR modulator, such as an AhR antagonist of Formula (I), are provided for use in inhibiting proliferation, tissue invasion, metastasis and angiogenesis of cancer cells in a subject having a cancer, a cancerous condition, or a tumor.

In some embodiments of these aspects and all such aspects described herein, the use further comprises the step of selecting the subject having a cancer, a cancerous condition, or a tumor. In some such embodiments, the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia. In some such embodiments, the cancer is a hepatocellular cancer.

In some embodiments of these aspects and all such aspects described herein, the use further comprises one or more additional anti-cancer therapies. In some such embodiments, the additional anti-cancer therapy comprises surgery, radiation therapy, biotherapy, immunotherapy, or chemotherapy.

In some embodiments of these aspects and all such aspects described herein, the use further comprises one or more anti-cancer therapeutic agents. In some such embodiments, the anti-cancer therapeutic agent is a chemotherapeutic agent, a growth inhibitor agent, an anti-angiogenesis agent, a cytotoxic agent, an anti-hormonal agent, a prodrug, or a cytokine.

### Advantageous Effects

The novel compounds of Formula (I) according to the present invention effectively modulate AhR activity, and therefore they are useful as a therapeutic or prophylactic drug for various disease, disorder, or condition associated with AhR activity such as cancer, cancerous condition, tumor, fibrotic disease, conditions with dysregulated immune responses including autoimmune disease such as rheumatoid arthritis, systemic lupus erythematosus (SLE), multiple sclerosis (MS), or other disorders associated with aberrant AhR signaling etc.

### BEST MODE

Hereinafter, the present invention will be described in more detail.

Unless otherwise defined, all technical terms used herein have the same meaning as commonly understood by one of ordinary skill in the art to which this invention belongs. Also, although the invention has been described in conjunction with specific methods and samples, their analogs or equivalents should be within the scope of the present invention. Furthermore, the numerical values set forth herein are considered to include the meaning of “about” unless explicitly stated. All publications and other references mentioned herein are hereby incorporated by reference in their entirety.

The definition of residues used herein is described in detail. Unless otherwise indicated, each residue has the following definition and is used in the sense as commonly understood by one of ordinary skill in the art.

As used herein, the term “halo” “halogen”, “halide (s)” includes fluoro, chloro, bromo and iodo.

As used herein, the “alkyl” refers to an aliphatic hydrocarbon radical, and includes both linear and branched hydrocarbon radicals. For example, C₁₋₆ alkyl is an aliphatic hydrocarbon having 1 to 6 carbon atoms and includes methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl. Unless otherwise defined, the alkyl refers to C₁₋₆ alkyl, preferably C₁₋₄ alkyl, more preferably C₁₋₃ alkyl.

As used herein, the “alkenyl” refers to an aliphatic hydrocarbon radical comprising at least one carbon-carbon double bond, and includes both linear and branched hydrocarbon radicals. The unlimited example of the “alkenyl” is vinyl, allyl, but-1-enyl or but-2-enyl.

As used herein, the “alkynyl” refers to an aliphatic hydrocarbon radical comprising at least one carbon-carbon triple bond, and includes both linear and branched hydrocarbon radicals. The unlimited example of the “alkynyl” is ethynyl, propargyl, but-1-ynyl or but-2-ynyl.

As used herein, the “haloalkyl” refers to an alkyl group substituted with one or more halogen atom, and the alkyl group is defined as above. The “halo” refers to F, Cl, Br, or I, and the term is compatibly used with the term “halogen”. Unless otherwise defined, the haloalkyl refers tofluoromethyl, difluoromethyl, chloromethyl, trifluoromethyl or 2,2,2-trifluoromethyl.

As used herein, the term “alkoxy” refers to —O-alkyl or alkyl-O— group, and the alkyl group is defined as shown above. For example, it includes methoxy, ethoxy, n-propoxy, n-butoxy and t-butoxy.

As used herein, the “alkoxyalkyl” refers to alkyl-O-alkyl group, and the alkyl group is defined as above. The unlimited example is methoxymethyl, ethoxymethyl, methoxyethyl or isopropoxymethyl.

As used herein, the term “hydroxy” or “hydroxyl” alone or in combination with other terms means —OH.

As used herein, “cyano” refers to —CN, “cyanoalkyl” refers to alkyl substituted with —CN, wherein the alkyl group is as defined above.

As used herein, “amino” refers to —NH₂; and “nitro” refers to —NO₂.

As used herein, “carboxy” refers to —C(O)—OH group.

As used herein, “ester” refers to a group of —C(O)—OR, where R is alkyl may be C₁₋₁₀, preferably C₁₋₈, C₁₋₆ or C₁₋₄ alkyl. Such ester groups may or may not be substituted with one or more suitable substituents.

As used herein, the term “cycloalkyl” refers to a cyclic alkyl which may be substituted or unsubstituted, and for example, the C₃₋₂₀ cycloalkyl represents a monovalent saturated hydrocarbon ring system having 3 to 20 carbon atoms. Examples of the cycloalkyl include, but are not limited to, cyclopropyl, cyclobutyl, cyclopentyl, cyclohexyl, cycloheptyl, cyclooctyl and the like. Preferably, unless otherwise defined, the cycloalkyl may be C₃₋₈ cycloalkyl, or C₃₋₆ cycloalkyl.

As used herein, the term “aryl” refers to a monovalent aromatic hydrocarbon having, for example, 6 to 20 carbon atoms (C₆₋₂₀) that is derived by the removal of one hydrogen atom from a single carbon atom of a parent aromatic ring system. The aryl may include a bicyclic radical containing an aromatic ring fused to a saturated or partially unsaturated ring. Exemplary aryl groups may include radicals derived from benzene (phenyl), substituted phenyl, biphenyl, naphthyl, toluyl, naphthalenyl, anthracenyl, indenyl, indanyl, and the like. Unless otherwise defined, the aryl refers to C₆₋₁₂ aryl, preferably C₆₋₁₀ aryl.

As used herein, the “heteroaryl” refers to a monovalent or divalent substituent derived from a monoheterocyclic or polyheterocyclic aromatic hydrocarbon having 1 to 10 carbon ring members containing one or more, preferably one to three, heteroatoms selected among N, O, and S. Examples of the heteroaryl include, but are not limited to, thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl,1,2,4-oxadiazolyl,1,3,4-oxadiazolyl, 1,2,4-thiadiazolyl, 1,3,4-thiadiazolyl, triazolyl, tetrazolyl, triazinyl, indolyl, and the like. Examples of the bicyclic heteroaryl includeindolyl, benzothiophenyl, benzofuranyl, benzimidazolyl, benzoxazolyl, benzisoxazolyl, benzthiazolyl, benzthiadiazolyl, quinolinyl, isoquinolinyl, furinyl, furopyridinyl, octahydropyranopyridine, benzodioxolyl and similar groups thereof, but are not limited thereto. Unless otherwise defined, the heteroaryl is C₃₋₁₀ heteroaryl, preferably C₃₋₇ heteroaryl, more preferably C₃₋₅ heteroaryl.

As used herein, the “heterocycloalkyl” refers to monocyclic, bicyclic, tricyclic or higher cyclic alkyl having 3 to 10 carbon ring members containing one or more, for example, one to four, heteroatoms selected among N, O, and S. In addition, the heterocycle according to the present invention may also be a fused or bridged heterocycloalkyl. Examples of non-aromatic rings include azetidinyl, oxetanyl, tetrahydrothienyl, tetrahydrofuranyl, pyrrolinyl, pyrrolidinyl, imidazolinyl, imidazolidinyl, oxazolinyl, oxazolidinyl, oxapiperazinyl, oxapiperidinyl, pyrazolinyl, pyrazolidinyl, thiazolinyl, thiazolidinyl, tetrahydrofuranyl, tetrahydrofuryl, tetrahydroisothiazolyl, tetrahydrooxazolyl, tetrahydroisoxazolyl, piperidinyl, piperazinyl, tetrahydropyranyl, dihydropyranyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, indolinyl, indolinylmethyl, thiomorpholinyl, azepanyl, diazepanyl, N-oxide, azaadamantanyl, diazamantanyl, and the like, but are not limited thereto.

Attachment of a heterocycloalkyl substituent can occur via a carbon atom or a heteroatom. A heterocycloalkyl group may be optionally substituted with one or more suitable groups via one or more aforementioned groups. Unless otherwise defined, heterocycloalkyl refers to heterocycloalkyl having 3 to 10 carbon ring members, preferably C₃₋₇ heterocycloalkyl, more preferably heterocycloalkyl having 3 to 5 carbon ring atoms.

Unless otherwise specified herein, the term “substituted” means that at least one hydrogen atom is substituted by one to three substituents selected from the group consisting of a halogen atom (e.g., F, Cl, Br, or I), a cyano group, a hydroxyl group, a thiol group, a nitro group, an amino group, an imino group, an azido group, an amidino group, a hydrazino group, a hydrazono group, an oxo group, a carbonyl group, a carbamyl group, an ester group, an ether group, a carboxyl group or a salt thereof, a sulfonic acid group or a salt thereof, phosphoric acid or a salt thereof, a C₁₋₆ alkyl group, a halo C₁₋₆ alkyl group, a hydroxy C₁₋₆ alkyl group, a C₂₋₆ alkenyl group, a halo C₂₋₆ alkenyl group, a C₂₋₆ alkynyl group, a halo C₂₋₆ alkynyl group, a C₁₋₆ alkoxy group, a halo C₁₋₆ alkoxy group, a hydroxy C₁₋₆ alkoxy group, a C₁₋₂₀ alkylthio group, a C₁₋₆ alkylsulfonyl group, a C₁₋₆ alkylcarbonyl group, a C₁₋₆ alkoxycarbonyl group, a C₃₋₂₀ carbocyclic group (e.g., a C₃₋₉ cycloalkyl group, a halo C₃₋₉ cycloalkyl group, a C₃₋₉ cycloalkenyl group, a halo C₃₋₉ cycloalkenyl group, a C₁₋₉ heterocycloalkyl group, a halo C₁₋₉ heterocycloalkyl group, a C₂₋₉ heterocycloalkenyl group, a halo C₂₋₉ heterocycloalkenyl group) and a C₁₋₂₀ heterocyclic group (e.g., a C₆₋₂₀ aryl group, a C₆₋₂₀ aryloxy group, a C₆₋₂₀ arylthio group, a C₂₋₂₀ heteroaryl group, a C₂₋₂₀ heteroaryloxygroup, a C₂₋₂₀ heteroarylthio group).

Based on the studies conducted and the results obtained so far, it is believed that the following compounds of Formula (I), including isomers, mixtures of isomer as well as pharmaceutically acceptable salts and solvates thereof, are particularly interesting.

Aryl Hydrocarbon Receptor

The Aryl Hydrocarbon Receptor (“AhR”) is a ligand-dependent member of the family of basic-helix-loop-helix transcription factors that has been found to be activated by numerous structurally diverse synthetic and naturally occurring compounds, such as polycyclic aromatic hydrocarbons, indoles, and flavonoids. In the absence of bound ligand, the AhR is present in a latent conformation in the cytoplasmic compartment of the cell associated with two molecules of the molecular chaperone heat shock protein 90 (“hsp90”), an immunophilin-like protein, XAP2, and the hsp90 interacting protein, p23.

The term “aryl hydrocarbon receptor” or “AhR” as used herein refers to the 848 amino acid polypeptide, as described by, e.g., NP_001612, together with any naturally occurring allelic, splice variants, and processed forms thereof. Typically, AhR refers to human AhR. The term AhR is also used to refer to truncated forms or fragments of the AhR polypeptide, comprising, for example, specific AhR domains. Reference to any such forms of the AhR can be identified in the application, e.g., by “AhR (122-224).”

AhR Modulators

The inventors of the present invention have discovered that the novel AhR modulator compounds described herein, such as the small molecules of Formula (I), modulate constitutive AhR activity, by functioning as AhR antagonists. Further, they have discovered that such AhR modulator compounds can inhibit cancer cell growth, as well as tumor invasion, metastasis and angiogenesis. Accordingly, described herein are novel modulators of the AhR and constitutive AhR signaling for use in therapeutic compositions for, and methods of, treating and inhibiting cancer growth and tumor cell invasion, and immune related diseases such as autoimmune diseases.

The AhR mediates a variety of functional responses, including, but not limited to de novo transcription of target genes or AhR battery genes having the DRE or XRE responsive element 5′-TNGCGTG-3′. Alternative pathways of AhR signaling have also been described, such as binding to retinoblastoma protein, estrogen receptor (ER), the transcription factor E2F1 and to the NFκB pathway subunits RelA and RelB. The AhR can also act as a ubiquitin ligase. Accordingly, signaling via the AhR comprises multiple pathways, including constitutive and non-constitutive AhR signaling pathways or signaling activity, as those terms are defined herein.

As used herein, “constitutive AhR signaling” refers to one or more signaling pathways mediated or regulated by the AhR that are activated or driven by one or more endogenous AhR ligands, or one or more environmental ligands, such as toxins or pollutants, that cause constitutive or long-term translocation of the AhR to the nucleus, and activation or modulation of one or more AhR battery genes involved in unregulated cell growth and proliferation, tumor cell invasiveness, or a combination thereof.

As used herein, “non-constitutive AhR signaling” refers to one or more signaling pathways mediated or induced by the AhR that does not cause constitutive or long-term translocation of the AhR to the nucleus, nor activation or modulation of one or more AhR battery genes involved in unregulated cell growth, tumor cell invasiveness, or a combination thereof. In some embodiments, non-constitutive AhR signaling does not cause upregulation of expression of CYP1A1, CYPIB1, or a combination thereof.

Accordingly, an “AhR modulator,” as the term is used herein refers to an agent, such as a compound of Formula (I), that modulates or causes or facilitates a qualitative or quantitative change, alteration, or modification in one or more processes, mechanisms, effects, responses, functions, activities or pathways mediated by the AhR receptor. Such changes mediated by an AhR modulator, such as an antagonist of the AhR described herein, can refer to a decrease in, inhibition of, or diversion of, constitutive activity of the AhR. The term “expression,” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “modulate” in reference to an Ahr modulator is used consistently with its use in the art, e.g., meaning to cause or facilitate a qualitative or quantitative change, alteration, or modification in one or more biological processes, mechanisms, effects, responses, functions, activities, pathways, or other phenomena of interest. Accordingly, as used herein, modulate refers to a qualitative or quantitative change, alteration, or modification in one or more processes, mechanisms, effects, responses, functions, activities or pathways mediated by the AhR receptor.

The term “agent” as used herein in reference to an AhR modulator means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In certain embodiments, as described herein, agents are small molecules having a chemical moiety. For example, chemical moieties include unsubstituted or substituted alkyl, aromatic, or heterocyclyl moieties. Compounds can be known to have a desired activity and/or property, e.g., modulate AhR activity, or can be selected from a library of diverse compounds, using, for example, the screening methods described herein.

In some embodiments, an AhR modulator selectively binds to the AhR. As used herein, “selectively binds” or “specifically binds” refers to the ability of an AhR antagonist, described herein to bind to a target, such as the AhR, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if an antagonist described herein binds to the AhR with a K_{D} of 10⁻⁵ M or lower, but not to other molecules, or a related homologue, then the agent is said to specifically bind the AhR. Specific binding can be influenced by, for example, the affinity and avidity of the antagonist and the concentration of the antagonist used. The person of ordinary skill in the art can determine appropriate conditions under which the antagonists described herein selectively bind using any suitable methods, such as titration of an AhR antagonist in a suitable cell binding assay, such as those described herein.

In some aspects of the compositions and methods described herein, AhR modulators are AhR antagonists having the chemical structures of Formula (I), described herein.

As used herein, the AhR is an “AhR antagonist.” An AhR antagonist refers to an AhR inhibitor that does not provoke a biological response itself upon specifically binding to the AhR, but blocks or dampens agonist-mediated or ligand-mediated responses, i.e., an AhR antagonist can bind but does not activate the AhR, and the binding disrupts the interaction, displaces an AhR agonist, and/or inhibits the function of an AhR agonist. Thus, as used herein, an AhR antagonist does not function as an inducer of AhR activity when bound to the AhR, i.e., they function as pure AhR inhibitors. In some embodiments, an AhR antagonist selectively binds to the AhR.

In some embodiments of these aspects, the AhR antagonists described herein, such as the compounds of Formula (I) block constitutive AhR effector functions that mediate growth and progression of established tumors. In other embodiments, the small molecule AhR antagonists of Formula (I), described herein act as chemopreventatives by blocking AhR-mediated CYP1A1 induction and mutagen production on exposure to environmental ligands.

In some embodiments of these aspects, the AhR antagonists of Formula (I), described herein inhibit the early contributions of constitutively active AhR in driving malignant transformation. In some embodiments, the compounds of Formula (I) described herein inhibit constitutive AhR signaling-mediated cancer or tumor cell growth. In some embodiments, the compounds of Formula (I), described herein inhibit constitutive AhR signaling-mediated tumor invasion in driving malignant transformation.

Accordingly, provided for use in the various aspects described herein are AhR antagonist of Formula (I):

An aspect of the present invention relates to novel compounds that can modulate human aryl hydrocarbon receptor (AhR). These compounds bind specifically to AhR.

In some embodiments, the compound has the structure of formula (I), or an enantiomer, diastereomer, racemate, solvate, hydrate, or pharmaceutically acceptable salt thereof:

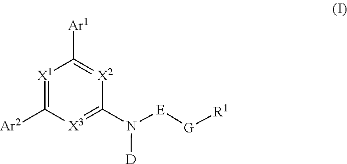

wherein:

X¹, X² and X³ are each independently CR², N or NR³;

Ar¹ and Ar² are each independently selected from substituted or unsubstituted mono- or bicyclic C₆₋₁₀ aryl, substituted or unsubstituted mono- or bicyclic C₅₋₁₀ heteroaryl and substituted or unsubstituted mono- or bicyclic C₃₋₁₀ heterocycloalkyl;

D is H, halo, cyano, hydroxy, amino, substituted or unsubstituted C₁₋₅ alkyl, mono- or bicyclic C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, mono- or bicyclic C₃₋₁₀ heterocycloalkyl, mono- or bicyclic C₃₋₁₀ heteroaryl,

E is absent (direct bond), amino, substituted or unsubstituted C₁₋₅ alkyl, mono- or bicyclic C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, mono- or bicyclic C₃₋₁₀ heterocycloalkyl, mono- or bicyclic C₃₋₁₀ heteroaryl,

or D and E, together with the atoms to which they are attached, are combined to form substituted or unsubstituted mono- or bicyclic C₃₋₁₀ heterocycloalkyl ring;

G is absent (direct bond), H, halo, cyano, hydroxy, amino, nitro, ether (—O—), thioether (—S—), sulfinyl(—SO—), sulfonyl (—SO₂—), sulfonylamido(—SO₂NR⁴—), aminosulfonyl(—NR⁴SO₂—), carbonyl(—(CO)—), amido(—(CO)NR⁴—), reverse amido(—NR⁴(CO)—), ester (—(CO)O—), substituted or unsubstituted mono- or bicyclic C₃₋₁₀cycloalkyl, substituted or unsubstituted mono- or bicyclic C₃₋₁₀ heterocycloalkyl, substituted or unsubstituted mono- or bicyclic C₆₋₁₀ aryl and substituted or unsubstituted mono- or bicyclic C₅₋₁₀ heteroaryl;

R¹ is absent, H, halo, cyano, hydroxy, amino, N(R⁵)₂, OR⁵, substituted or unsubstituted C₁₋₅ alkyl, C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, substituted or unsubstituted mono- or bicyclic C₃₋₁₀ heterocycloalkyl and substituted or unsubstituted mono- or bicyclic C₅₋₁₀ heteroaryl;

R² is H, halo, cyano, hydroxy and C₁₋₃ alkyl;

R³ is H, halo, cyano, hydroxyl and amino; and

R⁴ is H, substituted or unsubstituted C₁₋₅ alkyl, substituted or unsubstituted C₁₋₅ alkoxy and substituted or unsubstituted C₁₋₅ alkyl carboxylic acid; and

R⁵ is H, substituted or unsubstituted C₁₋₅ alkyl, substituted or unsubstituted C₁₋₅ alkoxy and substituted or unsubstituted C₁₋₅ alkyl carboxylic acid;

In a preferred embodiment, the Ar¹ may be substituted or unsubstituted monocyclic C₅₋₇ heteroaryl comprising one or more hetero atoms selected from the group consisting of N, O and S. More preferably, the Ar¹ may be monocyclic C₅₋₆ heteroaryl comprising one or two hetero atoms selected from the group consisting of N, O and S, which may be unsubstituted or substituted with C₁₋₃ alkyl. Far more preferably, the Ar¹ may be pyrazole or pyridine which may be unsubstituted or substituted with methyl.

In a preferred embodiment, the Ar² may be mono- or bicyclic C₆₋₁₀ aryl comprising one or more hetero atoms selected from the group consisting of N, O and S, which is unsubstituted or substituted with halo. More preferably, the Ar² may be phenyl which may be unsubstituted or substituted with chloro.

In a preferred embodiment, the D may be H or C₁₋₃ alkyl.

In a preferred embodiment, the E may absent (direct bond), amino, substituted or unsubstituted C₁₋₄ alkyl, mono- or bicyclic C₃₋₈ cycloalkyl, C₁₋₄ alkylhydroxy, C₁₋₄ alkenylhydroxy, C₁₋₄ alkynylhydroxy, C₁₋₄ alkylamine, C₁₋₄ alkenylamine, C₁₋₄ alkynylamine, mono- or bicyclic C₃₋₈ heterocycloalkyl, mono- or bicyclic C₃₋₈ heteroaryl, wherein the mono- or bicyclic C₃₋₈ heterocycloalkyl and mono- or bicyclic C₃₋₈ heteroaryl comprises one or more, preferably one or two heteroatoms selected from the group consisting of N, O and S.

In a preferred embodiment, the D and E, together with the atoms to which they are attached, may be combined to form substituted or unsubstituted mono- or bicyclic C₃₋₁₀ heterocycloalkyl ring one or more hetero atoms selected from the group consisting of N, O and S. More preferably, said mono- or bicyclic C₃₋₁₀ heterocycloalkyl ring may be unsubstituted or substituted pyrrolidine, piperidine, morpholine, thiomorpholine, piperazine, or octahydropyranopyridine.

In a preferred embodiment, G is absent (direct bond), H, halo, cyano, hydroxy, amino, nitro, ether (—O—), thioether (—S—), sulfinyl (—SO—), sulfonyl (—SO₂—), sulfonylamido (—SO₂NR⁴—), aminosulfonyl(—NR⁴SO₂—), carbonyl(—(CO)—), amido(—(CO)NR⁴—), reverse amido(—NR⁴(CO)—), ester (—(CO)O—), substituted or unsubstituted mono- or bicyclic C₃₋₈ cycloalkyl, substituted or unsubstituted mono- or bicyclic C₃₋₈ heterocycloalkyl, substituted or unsubstituted mono- or bicyclic C₆₋₁₀ aryl and substituted or unsubstituted mono- or bicyclic C₅₋₈ heteroaryl, wherein the mono- or bicyclic C₃₋₈ heterocycloalkyl and mono- or bicyclic C₅₋₈ heteroaryl comprises one or more, preferably one or two heteroatoms selected from the group consisting of N, O and S.

In a preferred embodiment, R¹ is absent, H, halo, cyano, hydroxy, amino, N(R⁵)₂, OR⁵, substituted or unsubstituted C₁₋₄ alkyl, C₃₋₈ cycloalkyl, C₁₋₄ alkylhydroxy, C₁₋₄ alkenylhydroxy, C₁₋₄ alkynylhydroxy, C₁₋₄ alkylamine, C₁₋₄ alkenylamine, C₁₋₄ alkynylamine, substituted or unsubstituted mono- or bicyclic C₃₋₈ heterocycloalkyl and substituted or unsubstituted mono- or bicyclic C₅₋₈ heteroaryl, phosphate, substituted or unsubstituted C₁₋₃ alkyl phosphate, wherein the mono- or bicyclic C₃₋₈ heterocycloalkyl and mono- or bicyclic C₅₋₈ heteroaryl comprises one or more, preferably one or two heteroatoms selected from the group consisting of N, O and S.

Further, in a more specific embodiment, the compound of the Formula I may be one selected from the group consisting of Compounds 1 to 276, as shown below:
1. (R)-2-((6-(4-chlorophenyl)-2-(pyridin-3-yl)pyrimidin-4-yl)amino)butan-1-ol
2. (S)-2-((6-(4-chlorophenyl)-2-(pyridin-3-yl)pyrimidin-4-yl)amino)propan-1-ol
3. (S)-2-((4-(4-chlorophenyl)-6-(pyridin-3-yl)pyrimidin-2-yl)amino)propan-1-ol
4. 2-((6-(4-chlorophenyl)-2-(pyridin-3-yl)pyrimidin-4-yl)amino)-2-methylpropan-1-ol
5. 2-((4-(4-chlorophenyl)-6-(pyridin-3-yl)pyrimidin-2-yl)amino)-2-methylpropan-1-ol
6. 2-((6-(4-chlorophenyl)-2-(pyridin-3-yl)pyrimidin-4-yl)amino)ethan-1-ol
7. 3-((6-(4-chlorophenyl)-2-(pyridin-3-yl)pyrimidin-4-yl)amino)propan-1-ol
8. (S)-1-((6-(4-chlorophenyl)-2-(pyridin-3-yl)pyrimidin-4-yl)amino)propan-2-ol
9. (R)-1-((6-(4-chlorophenyl)-2-(pyridin-3-yl)pyrimidin-4-yl)amino)propan-2-ol
10. 3-((6-(4-chlorophenyl)-2-(pyridin-3-yl)pyrimidin-4-yl)amino)propane-1,2-diol
11. (R)-2-((6-(4-chlorophenyl)-2-(pyridin-3-yl)pyrimidin-4-yl)amino)propan-1-ol
12. 2-((6-(4-chlorophenyl)-2-(pyridin-3-yl)pyrimidin-4-yl)amino)-3-methylbutan-1-ol
13. (S)-2-((6-(4-chlorophenyl)-2-(pyridin-3-yl)pyrimidin-4-yl)amino)-3-methylbutan-1-ol
14. (R)-2-((6-(4-chlorophenyl)-2-(pyridin-3-yl)pyrimidin-4-yl)amino)-3-methylbutan-1-ol
15. 2-((6-(4-chlorophenyl)-2-(pyridin-3-yl)pyrimidin-4-yl)amino)butan-1-ol
16. 2-((6-(4-chlorophenyl)-2-(pyridin-3-yl)pyrimidin-4-yl)amino)propane-1,3-diol
17. (R)-2-((6-(4-chlorophenyl)-2-(pyridin-3-yl)pyrimidin-4-yl)amino)-1-phenylethan-1-ol
18. (S)-2-((6-(4-chlorophenyl)-2-(pyridin-3-yl)pyrimidin-4-yl)amino)-1-phenylethan-1-ol
19. 6-(4-chlorophenyl)-2-(pyridin-3-yl)-N-((tetrahydro-2H-pyran-4-yl)methyl)pyrimidin-4-amine
20. N¹-(6-(4-chlorophenyl)-2-(pyridin-3-yl)pyrimidin-4-yl)-N³,N³-dimethylpropane-1,3-diamine
21. 6-(4-chlorophenyl)-N-ethyl-2-(pyridin-3-yl)pyrimidin-4-amine
22. 6-(4-chlorophenyl)-N-propyl-2-(pyridin-3-yl)pyrimidin-4-amine
23. N-butyl-6-(4-chlorophenyl)-2-(pyridin-3-yl)pyrimidin-4-amine
24. 1-((6-(4-chlorophenyl)-2-(pyridin-3-yl)pyrimidin-4-yl)amino)butan-2-ol
25. 6-(4-chlorophenyl)-N-(cyclopropylmethyl)-2-(pyridin-3-yl)pyrimidin-4-amine
26. 6-(4-chlorophenyl)-N-cyclopentyl-2-(pyridin-3-yl)pyrimidin-4-amine
27. 4-(4-chlorophenyl)-6-(4-methylpiperidin-1-yl)-2-(pyridin-3-yl)pyrimidine
28. N-(tert-butyl)-6-(4-chlorophenyl)-2-(pyridin-3-yl)pyrimidin-4-amine
29. (1R,2R)-2-((6-(4-chlorophenyl)-2-(pyridin-3-yl)pyrimidin-4-yl)amino)cyclopentan-1-ol
30. (1S,2R)-2-((6-(4-chlorophenyl)-2-(pyridin-3-yl)pyrimidin-4-yl)amino)cyclopentan-1-ol
31. 6-(4-chlorophenyl)-N-(pyridin-2-ylmethyl)-2-(pyridin-3-yl)pyrimidin-4-amine
32. 6-(4-chlorophenyl)-2-(pyridin-3-yl)-N-(pyridin-3-ylmethyl)pyrimidin-4-amine
33. 6-(4-chlorophenyl)-2-(pyridin-3-yl)-N-(pyridin-4-ylmethyl)pyrimidin-4-amine
34. trans-4-((6-(4-chlorophenyl)-2-(pyridin-3-yl)pyrimidin-4-yl)amino)cyclohexan-1-ol
35. trans-2-((6-(4-chlorophenyl)-2-(pyridin-3-yl)pyrimidin-4-yl)amino)cyclohexan-1-ol
36. (1-(6-(4-chlorophenyl)-2-(pyridin-3-yl)pyrimidin-4-yl)piperidin-2-yl)methanol
37. 2-(1-(6-(4-chlorophenyl)-2-(pyridin-3-yl)pyrimidin-4-yl)piperidin-2-yl)ethan-1-ol
38. (R)-1-(6-(4-chlorophenyl)-2-(pyridin-3-yl)pyrimidin-4-yl)piperidin-3-ol
39. 1-(6-(4-chlorophenyl)-2-(pyridin-3-yl)pyrimidin-4-yl)piperidin-3-ol
40. (1-(6-(4-chlorophenyl)-2-(pyridin-3-yl)pyrimidin-4-yl)piperidin-3-yl)methanol
41. 1-(6-(4-chlorophenyl)-2-(pyridin-3-yl)pyrimidin-4-yl)piperidin-4-ol
42. (1-(6-(4-chlorophenyl)-2-(pyridin-3-yl)pyrimidin-4-yl)piperidin-4-yl)methanol
43. 2-(1-(6-(4-chlorophenyl)-2-(pyridin-3-yl)pyrimidin-4-yl)piperidin-4-yl)ethan-1-ol
44. 3-(1-(6-(4-chlorophenyl)-2-(pyridin-3-yl)pyrimidin-4-yl)piperidin-4-yl)propan-1-ol
45. 4-(4-chlorophenyl)-6-(4-methoxypiperidin-1-yl)-2-(pyridin-3-yl)pyrimidine
46. 4-(4-chlorophenyl)-6-(piperidin-1-yl)-2-(pyridin-3-yl)pyrimidine
47. 4-(4-chlorophenyl)-6-(2-methylpiperidin-1-yl)-2-(pyridin-3-yl)pyrimidine
48. 4-(4-chlorophenyl)-6-(3-methylpiperidin-1-yl)-2-(pyridin-3-yl)pyrimidine
49. 4-(4-chlorophenyl)-6-(2,6-dimethylpiperidin-1-yl)-2-(pyridin-3-yl)pyrimidine
50. 4-(4-chlorophenyl)-6-(3,5-dimethylpiperidin-1-yl)-2-(pyridin-3-yl)pyrimidine
51. 4-(4-chlorophenyl)-6-(3,3-difluoropiperidin-1-yl)-2-(pyridin-3-yl)pyrimidine
52. 4-(4-chlorophenyl)-2-(pyridin-3-yl)-6-(3-(trifluoromethyl)piperidin-1-yl)pyrimidine
53. 4-(4-chlorophenyl)-6-(3-ethylpiperidin-1-yl)-2-(pyridin-3-yl)pyrimidine
54. 6-(4-chlorophenyl)-N-(piperidin-4-yl)-2-(pyridin-3-yl)pyrimidin-4-amine
55. 6-(4-chlorophenyl)-N-(piperidin-3-ylmethyl)-2-(pyridin-3-yl)pyrimidin-4-amine
56. 6-(4-chlorophenyl)-N-(piperidin-4-ylmethyl)-2-(pyridin-3-yl)pyrimidin-4-amine
57. 6-(4-chlorophenyl)-N-(1-methylpiperidin-4-yl)-2-(pyridin-3-yl)pyrimidin-4-amine
58. 6-(4-chlorophenyl)-N-(2-(piperidin-4-yl)ethyl)-2-(pyridin-3-yl)pyrimidin-4-amine
59. (1-(6-(4-chlorophenyl)-2-(pyridin-3-yl)pyrimidin-4-yl)piperidin-2-yl)methanamine
60. (R)-1-(6-(4-chlorophenyl)-2-(pyridin-3-yl)pyrimidin-4-yl)piperidin-3-amine
61. (S)-1-(6-(4-chlorophenyl)-2-(pyridin-3-yl)pyrimidin-4-yl)piperidin-3-amine
62. (1-(6-(4-chlorophenyl)-2-(pyridin-3-yl)pyrimidin-4-yl)piperidin-3-yl)methanamine
63. (S)-(1-(6-(4-chlorophenyl)-2-(pyridin-3-yl)pyrimidin-4-yl)piperidin-3-yl)methanamine
64. 1-(6-(4-chlorophenyl)-2-(pyridin-3-yl)pyrimidin-4-yl)piperidin-4-amine
65. (1-(6-(4-chlorophenyl)-2-(pyridin-3-yl)pyrimidin-4-yl)piperidin-4-yl)methanamine
66. (1R,2S)-2-((6-(4-chlorophenyl)-2-(pyridin-3-yl)pyrimidin-4-yl)amino)cyclopentan-1-ol
67. (1S,2S)-2-((6-(4-chlorophenyl)-2-(pyridin-3-yl)pyrimidin-4-yl)amino)cyclopentan-1-ol
68. trans-2-((6-(4-chlorophenyl)-2-(pyridin-3-yl)pyrimidin-4-yl)amino)cyclopentan-1-ol
69. (1R,2R)-2-((6-(4-chlorophenyl)-2-(pyridin-3-yl)pyrimidin-4-yl)amino)cyclohexan-1-ol
70. cis-4-(6-(4-chlorophenyl)-2-(pyridin-3-yl)pyrimidin-4-yl)-2,6-dimethylmorpholine
71. 4-(6-(4-chlorophenyl)-2-(pyridin-3-yl)pyrimidin-4-yl)morpholine
72. 6-(4-chlorophenyl)-N-(morpholin-2-ylmethyl)-2-(pyridin-3-yl)pyrimidin-4-amine
73. 4-(1-(6-(4-chlorophenyl)-2-(pyridin-3-yl)pyrimidin-4-yl)pyrrolidin-3-yl)morpholine
74. 4-(6-(4-chlorophenyl)-2-(pyridin-3-yl)pyrimidin-4-yl)thiomorpholine
75. 6-(4-chlorophenyl)-N-(3-morpholinopropyl)-2-(pyridin-3-yl)pyrimidin-4-amine
76. (R)-4-(4-chlorophenyl)-6-(2-methylpiperazin-1-yl)-2-(pyridin-3-yl)pyrimidine
77. (R)-(4-(6-(4-chlorophenyl)-2-(pyridin-3-yl)pyrimidin-4-yl)-3-methylpiperazin-1-yl)(phenyl)methanone
78. methyl (R)-4-(6-(4-chlorophenyl)-2-(pyridin-3-yl)pyrimidin-4-yl)piperazine-2-carboxylate
79. (R)-(4-(6-(4-chlorophenyl)-2-(pyridin-3-yl)pyrimidin-4-yl)piperazin-2-yl)methanol
80. 4-(4-chlorophenyl)-6-(4-(2,3-dichlorophenyl)piperazin-1-yl)-2-(pyridin-3-yl)pyrimidine
81. 4-(4-chlorophenyl)-6-(4-(2,5-dimethoxybenzyl)piperazin-1-yl)-2-(pyridin-3-yl)pyrimidine
82. 2-(4-(6-(4-chlorophenyl)-2-(pyridin-3-yl)pyrimidin-4-yl)piperazin-1-yl)ethan-1-ol
83. 4-(4-chlorophenyl)-6-(4-(2-methoxyphenyl)piperazin-1-yl)-2-(pyridin-3-yl)pyrimidine
84. 4-(4-chlorophenyl)-6-(4-(2-ethoxyphenyl)piperazin-1-yl)-2-(pyridin-3-yl)pyrimidine 85. 4-(4-chlorophenyl)-6-(4-(2-fluorophenyl)piperazin-1-yl)-2-(pyridin-3-yl)pyrimidine 86. (4-(6-(4-chlorophenyl)-2-(pyridin-3-yl)pyrimidin-4-yl)piperazin-1-yl)(furan-2-yl)methanone
87. 4-(4-chlorophenyl)-6-(4-phenethylpiperazin-1-yl)-2-(pyridin-3-yl)pyrimidine
88. 6-(4-chlorophenyl)-N-(2-(piperazin-1-yl)ethyl)-2-(pyridin-3-yl)pyrimidin-4-amine
89. 4-(4-chlorophenyl)-6-(4-(pyridin-2-yl)piperazin-1-yl)-2-(pyridin-3-yl)pyrimidine
90. 4-(4-chlorophenyl)-2-(pyridin-3-yl)-6-(4-(pyrimidin-2-yl)piperazin-1-yl)pyrimidine
91. 4-(2-(4-(6-(4-chlorophenyl)-2-(pyridin-3-yl)pyrimidin-4-yl)piperazin-1-yl)ethyl)morpholine
92. 4-(4-chlorophenyl)-6-(4-(4-methylpiperazin-1-yl)piperidin-1-yl)-2-(pyridin-3-yl)pyrimidine
93. trans-4-(4-chlorophenyl)-6-(4-cinnamylpiperazin-1-yl)-2-(pyridin-3-yl)pyrimidine
94. 4-(6-(4-chlorophenyl)-2-(pyridin-3-yl)pyrimidin-4-yl)piperazin-2-one
95. 4-(4-chlorophenyl)-6-(4-phenylpiperazin-1-yl)-2-(pyridin-3-yl)pyrimidine
96. 4-(4-chlorophenyl)-6-(4-propylpiperazin-1-yl)-2-(pyridin-3-yl)pyrimidine
97. 4-(4-(benzo[d][1,3]dioxol-5-ylmethyl)piperazin-1-yl)-6-(4-chlorophenyl)-2-(pyridin-3-yl)pyrimidine
98. (S)-(4-(6-(4-chlorophenyl)-2-(pyridin-3-yl)pyrimidin-4-yl)piperazin-2-yl)methanol
99. 4-(4-chlorophenyl)-6-(4-(4-fluorophenyl)piperazin-1-yl)-2-(pyridin-3-yl)pyrimidine
100. 6-(4-chlorophenyl)-N-(1,2,2,6,6-pentamethylpiperidin-4-yl)-2-(pyridin-3-yl)pyrimidin-4-amine
101. 6-(4-chlorophenyl)-N-(piperidin-3-yl)-2-(pyridin-3-yl)pyrimidin-4-amine
102. 4-(4-chlorophenyl)-6-(piperazin-1-yl)-2-(pyridin-3-yl)pyrimidine
103. Trans-4-(4-chlorophenyl)-6-(2,5-dimethylpiperazin-1-yl)-2-(pyridin-3-yl)pyrimidine
104. Cis-4-(4-chlorophenyl)-6-(3,5-dimethylpiperazin-1-yl)-2-(pyridin-3-yl)pyrimidine
105. 4-(4-chlorophenyl)-6-(4-methylpiperazin-1-yl)-2-(pyridin-3-yl)pyrimidine
106. 4-(4-chlorophenyl)-6-(4-ethylpiperazin-1-yl)-2-(pyridin-3-yl)pyrimidine
107. 4-(4-chlorophenyl)-6-(4-(methylsulfonyl)piperazin-1-yl)-2-(pyridin-3-yl)pyrimidine
108. 1-(4-(6-(4-chlorophenyl)-2-(pyridin-3-yl)pyrimidin-4-yl)piperazin-1-yl)ethan-1-one
109. 4-(4-chlorophenyl)-6-(3-ethylpiperazin-1-yl)-2-(pyridin-3-yl)pyrimidine
110. ethyl 4-(6-(4-chlorophenyl)-2-(pyridin-3-yl)pyrimidin-4-yl)piperazine-1-carboxylate
111. 4-(6-(4-chlorophenyl)-2-(pyridin-3-yl)pyrimidin-4-yl)piperazine-2-carboxylic acid
112. methyl 1-(6-(4-chlorophenyl)-2-(pyridin-3-yl)pyrimidin-4-yl)piperazine-2-carboxylate
113. (S)-4-(4-chlorophenyl)-6-(2-phenylpiperazin-1-yl)-2-(pyridin-3-yl)pyrimidine
114. 4-(4-chlorophenyl)-2-(pyridin-3-yl)-6-(4-(o-tolyl)piperazin-1-yl)pyrimidine
115. 4-(4-chlorophenyl)-2-(pyridin-3-yl)-6-(4-(p-tolyl)piperazin-1-yl)pyrimidine
116. 4-(4-chlorophenyl)-2-(pyridin-3-yl)-6-(4-(m-tolyl)piperazin-1-yl)pyrimidine
117. 4-(4-chlorophenyl)-2-(pyridin-3-yl)-6-(4-(3-(trifluoromethyl)phenyl)piperazin-1-yl)pyrimidine
118. 4-(4-chlorophenyl)-6-(4-(2,3-dimethylphenyl)piperazin-1-yl)-2-(pyridin-3-yl)pyrimidine
119. 4-(4-chlorophenyl)-6-(4-(3,4-dichlorophenyl)piperazin-1-yl)-2-(pyridin-3-yl)pyrimidine
120. 4-(4-chlorophenyl)-6-(4-(4-methoxyphenyl)piperazin-1-yl)-2-(pyridin-3-yl)pyrimidine
121. 4-(4-chlorophenyl)-6-(4-(4-nitrophenyl)piperazin-1-yl)-2-(pyridin-3-yl)pyrimidine
122. 4-(4-chlorophenyl)-6-(3-(4-methylpiperazin-1-yl)pyrrolidin-1-yl)-2-(pyridin-3-yl)pyrimidine
123. 4-(4-benzhydrylpiperazin-1-yl)-6-(4-chlorophenyl)-2-(pyridin-3-yl)pyrimidine
124. 4-(4-chlorophenyl)-6-(4-((4-chlorophenyl)(phenyl)methyl)piperazin-1-yl)-2-(pyridin-3-yl)pyrimidine
125. 1′-(6-(4-chlorophenyl)-2-(pyridin-3-yl)pyrimidin-4-yl)spiro[indene-1,4′-piperidine]
126. 6-(4-chlorophenyl)-2-(pyridin-3-yl)-N-(pyrrolidin-3-yl)pyrimidin-4-amine
127. (R)-6-(4-chlorophenyl)-2-(pyridin-3-yl)-N-(pyrrolidin-3-yl)pyrimidin-4-amine
128. (R)-6-(4-chlorophenyl)-2-(pyridin-3-yl)-N-(pyrrolidin-3-ylmethyl)pyrimidin-4-amine
129. 6-(4-chlorophenyl)-2-(pyridin-3-yl)-N-(2-(pyrrolidin-1-yl)ethyl)pyrimidin-4-amine
130. 6-(4-chlorophenyl)-2-(pyridin-3-yl)-N-(3-(pyrrolidin-1-yl)propyl)pyrimidin-4-amine
131. 6-(4-chlorophenyl)-N-(2-(1-methylpyrrolidin-2-yl)ethyl)-2-(pyridin-3-yl)pyrimidin-4-amine
132. N-(1-benzylpyrrolidin-3-yl)-6-(4-chlorophenyl)-2-(pyridin-3-yl)pyrimidin-4-amine
133. (3R,4S)-4-((6-(4-chlorophenyl)-2-(pyridin-3-yl)pyrimidin-4-yl)amino)pyrrolidin-3-ol
134. (3S,4R)-4-((6-(4-chlorophenyl)-2-(pyridin-3-yl)pyrimidin-4-yl)amino)pyrrolidin-3-ol
135. 4-(4-chlorophenyl)-2-(pyridin-3-yl)-6-(pyrrolidin-1-yl)pyrimidine
136. 4-(4-chlorophenyl)-6-(2-methylpyrrolidin-1-yl)-2-(pyridin-3-yl)pyrimidine
137. (S)-1-(6-(4-chlorophenyl)-2-(pyridin-3-yl)pyrimidin-4-yl)pyrrolidin-3-ol
138. (1-(6-(4-chlorophenyl)-2-(pyridin-3-yl)pyrimidin-4-yl)pyrrolidin-3-yl)methanol
139. (R)-4-(4-chlorophenyl)-6-(3-fluoropyrrolidin-1-yl)-2-(pyridin-3-yl)pyrimidine
140. 1-(6-(4-chlorophenyl)-2-(pyridin-3-yl)pyrimidin-4-yl)pyrrolidin-3-amine
141. 1-(6-(4-chlorophenyl)-2-(pyridin-3-yl)pyrimidin-4-yl)-N-methylpyrrolidin-3-amine
142. methyl (6-(4-chlorophenyl)-2-(pyridin-3-yl)pyrimidin-4-yl)prolinate
143. N-(1-(6-(4-chlorophenyl)-2-(pyridin-3-yl)pyrimidin-4-yl)pyrrolidin-3-yl)acetamide
144. (2R,3R)-3-((6-(4-chlorophenyl)-2-(pyridin-3-yl)pyrimidin-4-yl)amino)butan-2-ol
145. 3-((6-(4-chlorophenyl)-2-(pyridin-3-yl)pyrimidin-4-yl)amino)butan-2-ol
146. 1-(4-((6-(4-chlorophenyl)-2-(pyridin-3-yl)pyrimidin-4-yl)amino)piperidin-1-yl)ethan-1-one
147. (R)-(1-(6-(4-chlorophenyl)-2-(pyridin-3-yl)pyrimidin-4-yl)piperidin-3-yl)methanol
148. (S)-(1-(6-(4-chlorophenyl)-2-(pyridin-3-yl)pyrimidin-4-yl)piperidin-3-yl)methanol
149. 6-(4-chlorophenyl)-N-(2-(piperidin-1-yl)ethyl)-2-(pyridin-3-yl)pyrimidin-4-amine
150. 1-(6-(4-chlorophenyl)-2-(pyridin-3-yl)pyrimidin-4-yl)piperidine-4-carbonitrile
151. 1-(6-(4-chlorophenyl)-2-(pyridin-3-yl)pyrimidin-4-yl)-4-(3-(trifluoromethyl)phenyl)piperidin-4-ol
152. 4-(4-chlorophenyl)-2-(pyridin-3-yl)-6-(4-(pyrrolidin-1-yl)piperidin-1-yl)pyrimidine
153. 4-(4-chlorophenyl)-1-(6-(4-chlorophenyl)-2-(pyridin-3-yl)pyrimidin-4-yl)piperidin-4-ol
154. 1-(4-(((6-(4-chlorophenyl)-2-(pyridin-3-yl)pyrimidin-4-yl)amino)methyl)piperidin-1-yl)ethan-1-one
155. 1-(1-(6-(4-chlorophenyl)-2-(pyridin-3-yl)pyrimidin-4-yl)-4-phenylpiperidin-4-yl)ethan-1-one
156. 4-(1-(6-(4-chlorophenyl)-2-(pyridin-3-yl)pyrimidin-4-yl)piperidin-4-yl)morpholine 157. 4-(4-chlorophenyl)-6-(4-(3,5-dichlorophenyl)piperidin-1-yl)-2-(pyridin-3-yl)pyrimidine
158. 6-(4-chlorophenyl)-N-((1-cyclohexylpiperidin-3-yl)methyl)-2-(pyridin-3-yl)pyrimidin-4-amine
159. N-((1-benzylpiperidin-4-yl)methyl)-6-(4-chlorophenyl)-2-(pyridin-3-yl)pyrimidin-4-amine
160. ethyl 3-(1-(6-(4-chlorophenyl)-2-(pyridin-3-yl)pyrimidin-4-yl)piperidin-4-yl)-3-oxopropanoate
161. ethyl 2-(1-(6-(4-chlorophenyl)-2-(pyridin-3-yl)pyrimidin-4-yl)piperidin-4-yl)acetate
162. (1S,3R)-3-((6-(4-chlorophenyl)-2-(pyridin-3-yl)pyrimidin-4-yl)amino)cyclopentan-1-ol
163. (S)-1-(6-(4-chlorophenyl)-2-(pyridin-3-yl)pyrimidin-4-yl)piperidin-3-ol
164. 1-(6-(4-chlorophenyl)-2-(pyridin-3-yl)pyrimidin-4-yl)-N,N-dimethylpyrrolidin-3-amine
165. 2-(1-(6-(4-chlorophenyl)-2-(pyridin-3-yl)pyrimidin-4-yl)pyrrolidin-2-yl)-N,N-dimethylethan-1-amine
166. 1-(6-(4-chlorophenyl)-2-(pyridin-3-yl)pyrimidin-4-yl)piperidin-4-one
167. 6-(4-chlorophenyl)-N-methyl-N-(piperidin-4-yl)-2-(pyridin-3-yl)pyrimidin-4-amine
168. 6-(4-chlorophenyl)-N-(2-(1-methylpiperidin-2-yl)ethyl)-2-(pyridin-3-yl)pyrimidin-4-amine
169. 6-(4-chlorophenyl)-N-(1-(1-methylpiperidin-4-yl)ethyl)-2-(pyridin-3-yl)pyrimidin-4-amine
170. 6-(4-chlorophenyl)-N-((1-(2-methoxyethyl)piperidin-4-yl)methyl)-2-(pyridin-3-yl)pyrimidin-4-amine
171. methyl 2-(4-((6-(4-chlorophenyl)-2-(pyridin-3-yl)pyrimidin-4-yl)amino)piperidin-1-yl)acetate
172. 1-(1-(6-(4-chlorophenyl)-2-(pyridin-3-yl)pyrimidin-4-yl)piperidin-4-yl)ethyl 2,2,2-trifluoroacetate
173. 6-(4-chlorophenyl)-N-(1-methylpiperidin-3-yl)-2-(pyridin-3-yl)pyrimidin-4-amine
174. (1S,2R)-2-((6-(4-chlorophenyl)-2-(1-methyl-1H-pyrazol-4-yl)pyrimidin-4-yl)amino)cyclopentan-1-ol
175. (R)-1-(6-(4-chlorophenyl)-2-(1-methyl-1H-pyrazol-4-yl)pyrimidin-4-yl)piperidin-3-ol
176. 1-(6-(4-chlorophenyl)-2-(1-methyl-1H-pyrazol-4-yl)pyrimidin-4-yl)piperidin-4-ol
177. (1-(6-(4-chlorophenyl)-2-(1-methyl-1H-pyrazol-4-yl)pyrimidin-4-yl)piperidin-4-yl)methanol
178. 2-(1-(6-(4-chlorophenyl)-2-(1-methyl-1H-pyrazol-4-yl)pyrimidin-4-yl)piperidin-4-yl)ethan-1-ol
179. 3-(1-(6-(4-chlorophenyl)-2-(1-methyl-1H-pyrazol-4-yl)pyrimidin-4-yl)piperidin-4-yl)propan-1-ol
180. 4-(4-chlorophenyl)-2-(1-methyl-1H-pyrazol-4-yl)-6-(4-methylpiperidin-1-yl)pyrimidine
181. 4-(4-chlorophenyl)-2-(1-methyl-1H-pyrazol-4-yl)-6-(4-methylpiperazin-1-yl)pyrimidine
182. 2-(4-(6-(4-chlorophenyl)-2-(1-methyl-1H-pyrazol-4-yl)pyrimidin-4-yl)piperazin-1-yl)ethan-1-ol
183. (S)-1-(6-(4-chlorophenyl)-2-(1-methyl-1H-pyrazol-4-yl)pyrimidin-4-yl)pyrrolidin-3-ol
184. 1-(6-(4-chlorophenyl)-2-(1-methyl-1H-pyrazol-4-yl)pyrimidin-4-yl)piperidine-4-carbonitrile
185. (R)-(1-(6-(4-chlorophenyl)-2-(1-methyl-1H-pyrazol-4-yl)pyrimidin-4-yl)piperidin-3-yl)methanol
186. (R)-1-(6-(4-chlorophenyl)-2-(1-methyl-1H-pyrazol-4-yl)pyrimidin-4-yl)pyrrolidin-3-ol
187. (1S,3R)-3-((6-(4-chlorophenyl)-2-(1-methyl-1H-pyrazol-4-yl)pyrimidin-4-yl)amino)cyclopentan-1-ol
188. (R)-2-((6-(4-chlorophenyl)-2-(1-methyl-1H-pyrazol-4-yl)pyrimidin-4-yl)amino)butan-1-ol
189. Trans-4-((6-(4-chlorophenyl)-2-(1-methyl-1H-pyrazol-4-yl)pyrimidin-4-yl)amino)cyclohexan-1-ol
190. 7-(6-(4-chlorophenyl)-2-(pyridin-3-yl)pyrimidin-4-yl)octahydro-2H-pyrano[2,3-c]pyridine
191. 7-(6-(4-chlorophenyl)-2-(pyridin-3-yl)pyrimidin-4-yl)octahydro-2H-pyrano[2,3-c]pyridin-4-ol
192. (2R,3R)-3-((6-(4-chlorophenyl)-2-(pyridin-3-yl)pyrimidin-4-yl)amino)pentan-2-ol
193. 6-(4-chlorophenyl)-N-((1-methylpiperidin-4-yl)methyl)-2-(pyridin-3-yl)pyrimidin-4-amine
194. (R)-1-(6-(4-chlorophenyl)-2-(pyridin-3-yl)pyrimidin-4-yl)pyrrolidin-3-ol
195. (S)-6-(4-chlorophenyl)-N-(2-(methoxymethyl)pyrrolidin-1-yl)-2-(pyridin-3-yl)pyrimidin-4-amine
196. (S)-4-(4-chlorophenyl)-6-(3-fluoropyrrolidin-1-yl)-2-(pyridin-3-yl)pyrimidine
197. 4-(4-chlorophenyl)-2-(pyridin-3-yl)-6-(2-(trifluoromethyl)pyrrolidin-1-yl)pyrimidine
198. 4-(4-chlorophenyl)-6-(3,3-difluoropyrrolidin-1-yl)-2-(pyridin-3-yl)pyrimidine
199. 4-(1-(6-(4-chlorophenyl)-2-(pyridin-3-yl)pyrimidin-4-yl)pyrrolidin-3-yl)morpholine
200. 5-(((6-(4-chlorophenyl)-2-(pyridin-3-yl)pyrimidin-4-yl)amino)methyl)pyrrolidin-2-one
201. Trans-6-(4-chlorophenyl)-2-(pyridin-3-yl)-N-(4-(pyrrolidin-1-yl)tetrahydrofuran-3-yl)pyrimidin-4-amine
202. 6-(4-chlorophenyl)-N-((3S,4S)-4-methoxy-1-methylpyrrolidin-3-yl)-2-(pyridin-3-yl)pyrimidin-4-amine
203. (R)-6-(4-chlorophenyl)-N-(piperidin-3-yl)-2-(pyridin-3-yl)pyrimidin-4-amine
204. 6-(4-chlorophenyl)-N-(piperidin-3-yl)-2-(pyridin-3-yl)pyrimidin-4-amine
205. 6-(4-chlorophenyl)-N-((3R,4R)-3-fluoropiperidin-4-yl)-2-(pyridin-3-yl)pyrimidin-4-amine
206. (S)-6-(4-chlorophenyl)-2-(pyridin-3-yl)-N-(pyrrolidin-3-ylmethyl)pyrimidin-4-amine
207. methyl (2R,4R)-4-((6-(4-chlorophenyl)-2-(pyridin-3-yl)pyrimidin-4-yl)amino)pyrrolidine-2-carboxylate
208. (2R,4S)-4-((6-(4-chlorophenyl)-2-(pyridin-3-yl)pyrimidin-4-yl)amino)pyrrolidine-2-carboxylic acid
209. Trans-4-((6-(4-chlorophenyl)-2-(pyridin-3-yl)pyrimidin-4-yl)amino)-1-isopropylpyrrolidin-3-ol
210. (R)-4-(3-(chloromethyl)pyrrolidin-1-yl)-6-(4-chlorophenyl)-2-(pyridin-3-yl)pyrimidine
211. (S)-4-(3-(chloromethyl)pyrrolidin-1-yl)-6-(4-chlorophenyl)-2-(pyridin-3-yl)pyrimidine
212. 1-(6-(4-chlorophenyl)-2-(pyridin-3-yl)pyrimidin-4-yl)pyrrolidine-3-carbonitrile
213. (R)-1-((6-(4-chlorophenyl)-2-(pyridin-3-yl)pyrimidin-4-yl)amino)butan-2-ol
214. (1R,3S)-3-((6-(4-chlorophenyl)-2-(pyridin-3-yl)pyrimidin-4-yl)amino)cyclopentan-1-ol
215. Cis-(4-((6-(4-chlorophenyl)-2-(pyridin-3-yl)pyrimidin-4-yl)amino)cyclohexyl)methanol
216. Cis-4-((6-(4-chlorophenyl)-2-(pyridin-3-yl)pyrimidin-4-yl)amino)cyclohexan-1-ol
217. Trans-4-((6-(4-chlorophenyl)-2-(pyridin-3-yl)pyrimidin-4-yl)amino)cyclohexan-1-ol
218. 4-(4-chlorophenyl)-6-(4-(2-methoxyethyl)piperazin-1-yl)-2-(pyridin-3-yl)pyrimidine
219. (3S,4R)-1-(6-(4-chlorophenyl)-2-(pyridin-3-yl)pyrimidin-4-yl)-3-fluoropiperidin-4-ol
220. (3R,4S)-1-(6-(4-chlorophenyl)-2-(pyridin-3-yl)pyrimidin-4-yl)-3-fluoropiperidin-4-ol
221. (3R,4R)-1-(6-(4-chlorophenyl)-2-(pyridin-3-yl)pyrimidin-4-yl)-3-fluoropiperidin-4-ol
222. (3S,4S)-1-(6-(4-chlorophenyl)-2-(pyridin-3-yl)pyrimidin-4-yl)-3-fluoropiperidin-4-ol
223. 1-(4-(6-(4-chlorophenyl)-2-(pyridin-3-yl)pyrimidin-4-yl)piperazin-1-yl)-2-hydroxyethan-1-one
224. 2-(4-(6-(4-chlorophenyl)-2-(pyridin-3-yl)pyrimidin-4-yl)piperazin-1-yl)propan-1-ol
225. N-(1-(6-(4-chlorophenyl)-2-(pyridin-3-yl)pyrimidin-4-yl)piperidin-4-yl)-2-methoxyacetamide
226. (1-(2-(pyridin-3-yl)-6-(4-(trifluoromethyl)phenyl)pyrimidin-4-yl)piperidin-4-yl)methanol
227. (1-(2-(pyridin-3-yl)-6-(4-(trifluoromethoxy)phenyl)pyrimidin-4-yl)piperidin-4-yl)methanol
228. (1-(6-(4-methoxyphenyl)-2-(pyridin-3-yl)pyrimidin-4-yl)piperidin-4-yl)methanol
229. (1-(2-(pyridin-3-yl)-6-(p-tolyl)pyrimidin-4-yl)piperidin-4-yl)methanol
230. (3R,4R)-1-(6-(4-chlorophenyl)-2-(pyridin-3-yl)pyrimidin-4-yl)pyrrolidine-3,4-diol
231. (3R,4S)-1-(6-(4-chlorophenyl)-2-(pyridin-3-yl)pyrimidin-4-yl)pyrrolidine-3,4-diol
232. 1-(3-(6-(4-chlorophenyl)-2-(pyridin-3-yl)pyrimidin-4-yl)tetrahydropyrimidin-1 (2H)-yl)ethan-1-one
233. 1-(6-(4-chlorophenyl)-2-(pyridin-3-yl)pyrimidin-4-yl)-4-(hydroxymethyl)piperidin-3-ol
233. 1-(6-(4-chlorophenyl)-2-(pyridin-3-yl)pyrimidin-4-yl)-4-(hydroxymethyl)piperidin-3-ol
234. (3R,4R)-1-(6-(4-chlorophenyl)-2-(pyridin-3-yl)pyrimidin-4-yl)-4-fluoropyrrolidin-3-ol
235. (3R,4S)-1-(6-(4-chlorophenyl)-2-(pyridin-3-yl)pyrimidin-4-yl)-4-fluoropyrrolidin-3-ol
236. 1-(6-(4-chlorophenyl)-2-(pyridin-3-yl)pyrimidin-4-yl)-4-(hydroxymethyl)pyrrolidin-3-ol
237. N-(1-(6-(4-chlorophenyl)-2-(pyridin-3-yl)pyrimidin-4-yl)piperidin-4-yl)-2-hydroxypropanamide
238. N-(1-(6-(4-chlorophenyl)-2-(pyridin-3-yl)pyrimidin-4-yl)piperidin-4-yl)-2-hydroxyacetamide
239. 2-((4-(6-(4-chlorophenyl)-2-(pyridin-3-yl)pyrimidin-4-yl)piperazin-1-yl)sulfonyl)ethan-1-ol
240. (S)-(1-(6-(4-chlorophenyl)-2-(pyridin-3-yl)pyrimidin-4-yl)pyrrolidin-3-yl)methanol
241. N-((3R,4R)-1-(6-(4-chlorophenyl)-2-(pyridin-3-yl)pyrimidin-4-yl)-4-hydroxypyrrolidin-3-yl)acetamide
242. (3R,4R)-4-acetamido-1-(6-(4-chlorophenyl)-2-(pyridin-3-yl)pyrimidin-4-yl)pyrrolidin-3-yl acetate
243. N-((3R,4S)-1-(6-(4-chlorophenyl)-2-(pyridin-3-yl)pyrimidin-4-yl)-4-hydroxypyrrolidin-3-yl)acetamide
244. N-((3S,4R)-1-(6-(4-chlorophenyl)-2-(pyridin-3-yl)pyrimidin-4-yl)-4-hydroxypyrrolidin-3-yl)acetamide
245. N-((3S,4S)-1-(6-(4-chlorophenyl)-2-(pyridin-3-yl)pyrimidin-4-yl)-4-hydroxypyrrolidin-3-yl)acetamide
246. (1-(6-(4-chloro-3-fluorophenyl)-2-(pyridin-3-yl)pyrimidin-4-yl)piperidin-4-yl)methanol
247. (3S,4R)-1-(6-(4-chlorophenyl)-2-(pyridin-3-yl)pyrimidin-4-yl)-4-(hydroxymethyl)piperidin-3-ol
248. (3S,4R)-4-(hydroxymethyl)-1-(2-(pyridin-3-yl)-6-(4-(trifluoromethyl)phenyl)pyrimidin-4-yl)piperidin-3-ol
249. ((3S,4R)-1-(6-(4-chlorophenyl)-2-(pyridin-3-yl)pyrimidin-4-yl)-3-fluoropiperidin-4-yl)methanol
250. ((3S,4R)-3-fluoro-1-(2-(pyridin-3-yl)-6-(4-(trifluoromethyl)phenyl)pyrimidin-4-yl)piperidin-4-yl)methanol
251. ((3R,4S)-1-(6-(4-chlorophenyl)-2-(pyridin-3-yl)pyrimidin-4-yl)-3-fluoropiperidin-4-yl)methanol
252. ((3R,4S)-3-fluoro-1-(2-(pyridin-3-yl)-6-(4-(trifluoromethyl)phenyl)pyrimidin-4-yl)piperidin-4-yl)methanol
253. ((3R,4R)-1-(6-(4-chlorophenyl)-2-(pyridin-3-yl)pyrimidin-4-yl)-3-fluoropiperidin-4-yl)methanol
254. ((3R,4R)-3-fluoro-1-(2-(pyridin-3-yl)-6-(4-(trifluoromethyl)phenyl)pyrimidin-4-yl)piperidin-4-yl)methanol
255. (3S,4R)-1-(6-(4-chlorophenyl)-2-(pyridin-3-yl)pyrimidin-4-yl)-4-(hydroxymethyl)pyrrolidin-3-ol
256. (3S,4R)-4-(hydroxymethyl)-1-(2-(pyridin-3-yl)-6-(4-(trifluoromethyl)phenyl)pyrimidin-4-yl)pyrrolidin-3-ol
257. (3R,4R)-1-(6-(4-chlorophenyl)-2-(pyridin-3-yl)pyrimidin-4-yl)-4-(hydroxymethyl)pyrrolidin-3-ol
258. (3R,4R)-4-(hydroxymethyl)-1-(2-(pyridin-3-yl)-6-(4-(trifluoromethyl)phenyl)pyrimidin-4-yl)pyrrolidin-3-ol
259. (3R,4R)-1-(6-(4-chlorophenyl)-2-(pyridin-3-yl)pyrimidin-4-yl)-4-(hydroxymethyl)piperidin-3-ol
260. (3R,4R)-4-(hydroxymethyl)-1-(2-(pyridin-3-yl)-6-(4-(trifluoromethyl)phenyl)pyrimidin-4-yl)piperidin-3-ol
261. (3S,4S)-1-(6-(4-chlorophenyl)-2-(pyridin-3-yl)pyrimidin-4-yl)-4-(hydroxymethyl)piperidin-3-ol
262. (3S,4S)-4-(hydroxymethyl)-1-(2-(pyridin-3-yl)-6-(4-(trifluoromethyl)phenyl)pyrimidin-4-yl)piperidin-3-ol
263. (S)-1-(2-(4-methylpyridin-3-yl)-6-(4-(trifluoromethyl)phenyl)pyrimidin-4-yl)pyrrolidin-3-ol
264. (3R,4S)-1-(6-(4-chlorophenyl)-2-(pyridin-3-yl)pyrimidin-4-yl)-4-(hydroxymethyl)piperidin-3-ol
265. (3R,4S)-4-(hydroxymethyl)-1-(2-(pyridin-3-yl)-6-(4-(trifluoromethyl)phenyl)pyrimidin-4-yl)piperidin-3-ol
266. (3S,4S)-1-(6-(4-chlorophenyl)-2-(pyridin-3-yl)pyrimidin-4-yl)-4-(hydroxymethyl)pyrrolidin-3-ol
267. (3S,4S)-4-(hydroxymethyl)-1-(2-(pyridin-3-yl)-6-(4-(trifluoromethyl)phenyl)pyrimidin-4-yl)pyrrolidin-3-ol
268. (1-(6-(4-morpholinophenyl)-2-(pyridin-3-yl)pyrimidin-4-yl)piperidin-4-yl)methanol
269. (S)-1-(2-(2-methylpyridin-3-yl)-6-(4-(trifluoromethyl)phenyl)pyrimidin-4-yl)pyrrolidin-3-ol
270. (S)-3-(4-(3-hydroxypyrrolidin-1-yl)-6-(4-(trifluoromethyl)phenyl)pyrimidin-2-yl)pyridin-2-ol
271. 5-chloro-2-(6-(4-(hydroxymethyl)piperidin-1-yl)-2-(pyridin-3-yl)pyrimidin-4-yl)phenol
272. tert-butyl (S)-4-(6-(4-chlorophenyl)-2-(pyridin-3-yl)pyrimidin-4-yl)-2-(hydroxymethyl)piperazine-1-carboxylate
273. 2-chloro-5-(6-(4-(hydroxymethyl)piperidin-1-yl)-2-(pyridin-3-yl)pyrimidin-4-yl)phenol
274. N-(4-(6-(4-(hydroxymethyl)piperidin-1-yl)-2-(pyridin-3-yl)pyrimidin-4-yl)phenyl)methanesulfonamide
275. (1-(6-(4-(4-methylpiperazin-1-yl)phenyl)-2-(pyridin-3-yl)pyrimidin-4-yl)piperidin-4-yl)methanol
276. (1-(6-(3-fluoro-4-morpholinophenyl)-2-(pyridin-3-yl)pyrimidin-4-yl)piperidin-4-yl)methanol
277. (1-(2-(pyridin-3-yl)-6-(2,4,6-trifluorophenyl)pyrimidin-4-yl)piperidin-4-yl)methanol
278. (1-(2-(pyridin-3-yl)-6-(4-((tetrahydro-2H-pyran-2-yl)oxy)phenyl)pyrimidin-4-yl)piperidin-4-yl)methanol
279. (S)-(4-(2-(pyridin-3-yl)-6-(4-(trifluoromethyl)phenyl)pyrimidin-4-yl)morpholin-2-yl)methanol
280. (R)-(4-(2-(pyridin-3-yl)-6-(4-(trifluoromethyl)phenyl)pyrimidin-4-yl)morpholin-2-yl)methanol
281. ((3S,4S)-3-fluoro-1-(2-(pyridin-3-yl)-6-(4-(trifluoromethyl)phenyl)pyrimidin-4-yl)piperidin-4-yl)methanol
282. ((3S,4S)-1-(6-(4-chlorophenyl)-2-(pyridin-3-yl)pyrimidin-4-yl)-3-fluoropiperidin-4-yl)methanol
283. (3S,4S)-1-(2-(pyridin-3-yl)-6-(4-(trifluoromethyl)phenyl)pyrimidin-4-yl)pyrrolidine-3,4-diol
284. (3S,4S)-1-(6-(4-chlorophenyl)-2-(pyridin-3-yl)pyrimidin-4-yl)pyrrolidine-3,4-diol
285. 3-(4-(4-(hydroxymethyl)piperidin-1-yl)-6-(4-morpholinophenyl)pyrimidin-2-yl)pyridin-2-ol
286. (1-(2-(1-methyl-1H-pyrazol-4-yl)-6-(4-morpholinophenyl)pyrimidin-4-yl)piperidin-4-yl)methanol
287. (1-(6-(3-fluoro-4-morpholinophenyl)-2-(1-methyl-1H-pyrazol-4-yl)pyrimidin-4-yl)piperidin-4-yl)methanol
288. (1-(6-(1H-indazol-5-yl)-2-(pyridin-3-yl)pyrimidin-4-yl)piperidin-4-yl)methanol
289. (1-(6-(6-morpholinopyridin-3-yl)-2-(pyridin-3-yl)pyrimidin-4-yl)piperidin-4-yl)methanol
290. 5-(6-(4-(hydroxymethyl)piperidin-1-yl)-2-(pyridin-3-yl)pyrimidin-4-yl)indolin-2-one
291. 4-(4-(6-(4-(hydroxymethyl)piperidin-1-yl)-2-(pyridin-3-yl)pyrimidin-4-yl)phenyl)morpholin-3-one
292. 4-(6-(4-(hydroxymethyl)piperidin-1-yl)-2-(pyridin-3-yl)pyrimidin-4-yl)benzoic acid
293. 4 (1-(6-(1-methyl-1H-pyrazol-3-yl)-2-(pyridin-3-yl)pyrimidin-4-yl)piperidin-4-yl)methanol
294. (1-(6-(5-fluoropyridin-3-yl)-2-(pyridin-3-yl)pyrimidin-4-yl)piperidin-4-yl)methanol
295. (S)-3-(4-(3-hydroxypyrrolidin-1-yl)-6-(6-morpholinopyridin-3-yl)pyrimidin-2-yl)pyridin-2-ol
296. (S)-1-(6-(4-fluoro-3-nitrophenyl)-2-(pyridin-3-yl)pyrimidin-4-yl)pyrrolidin-3-ol
297. (S)-1-(6-(4-morpholino-3-nitrophenyl)-2-(pyridin-3-yl)pyrimidin-4-yl)pyrrolidin-3-ol
298. (S)-1-(6-(3-amino-4-morpholinophenyl)-2-(pyridin-3-yl)pyrimidin-4-yl)pyrrolidin-3-ol
299. (S)—N-(5-(6-(3-hydroxypyrrolidin-1-yl)-2-(pyridin-3-yl)pyrimidin-4-yl)-2-morpholinophenyl)acetamide
300. (S)-1-(6-(6-fluoropyridin-3-yl)-2-(pyridin-3-yl)pyrimidin-4-yl)pyrrolidin-3-ol
301. (S)-1-(6-(6-((2-(dimethylamino)ethyl)amino)pyridin-3-yl)-2-(pyridin-3-yl)pyrimidin-4-yl)pyrrolidin-3-ol
302. (3S)-1-(6-(6-(2,6-dimethylmorpholino)pyridin-3-yl)-2-(pyridin-3-yl)pyrimidin-4-yl)pyrrolidin-3-ol
303. 5-chloro-2-(6-(4-((2-hydroxyethyl)sulfonyl)piperazin-1-yl)-2-(pyridin-3-yl)pyrimidin-4-yl)phenol
304. (S)-3-(4-(4-chloro-2-hydroxyphenyl)-6-(3-hydroxypyrrolidin-1-yl)pyrimidin-2-yl)pyridin-2-ol
305. (S)-1-(6-(4-aminophenyl)-2-(pyridin-3-yl)pyrimidin-4-yl)pyrrolidin-3-ol
306. 4-(4-chlorophenyl)-2-(pyridin-3-yl)-6-(4-(vinylsulfonyl)piperazin-1-yl)pyrimidine
307. (1-(6-(2,4-dichlorophenyl)-2-(pyridin-3-yl)pyrimidin-4-yl)piperidin-4-yl)methanol
308. (S)-(4-(2-(pyridin-3-yl)-6-(4-(trifluoromethyl)phenyl)pyrimidin-4-yl)piperazin-2-yl)methanol
309. (R)-(4-(2-(pyridin-3-yl)-6-(4-(trifluoromethyl)phenyl)pyrimidin-4-yl)piperazin-2-yl)methanol
310. (R)-1-(2-(pyridin-3-yl)-6-(4-(trifluoromethyl)phenyl)pyrimidin-4-yl)pyrrolidine-3-carboxylic acid
311. (R)-1-(6-(4-chlorophenyl)-2-(pyridin-3-yl)pyrimidin-4-yl)pyrrolidine-3-carboxylic acid
312. (R)-1-(2-(1-methyl-1H-pyrazol-4-yl)-6-(4-(trifluoromethyl)phenyl)pyrimidin-4-yl)pyrrolidine-3-carboxylic acid
313. (R)-1-(6-(4-chlorophenyl)-2-(1-methyl-1H-pyrazol-4-yl)pyrimidin-4-yl)pyrrolidine-3-carboxylic acid
314. (R)-2-(2-(pyridin-3-yl)-6-(4-(trifluoromethyl)phenyl)pyrimidin-4-yl)isoxazolidin-4-ol
315. (S)-1-(6-(6-morpholinopyridin-3-yl)-2-(pyridin-3-yl)pyrimidin-4-yl)pyrrolidin-3-ol
316. (S)-1-(6-(4-chloro-2-hydroxyphenyl)-2-(pyridin-3-yl)pyrimidin-4-yl)pyrrolidin-3-ol
317. (S)-3-(6-(3-hydroxypyrrolidin-1-yl)-2-(pyridin-3-yl)pyrimidin-4-yl)pyridin-2-ol
318. (1-(6-(6-fluoropyridin-3-yl)-2-(pyridin-3-yl)pyrimidin-4-yl)piperidin-4-yl)methanol
319. 1-(6-(4-chlorophenyl)-2-(pyridin-3-yl)pyrimidin-4-yl)azetidin-3-ol
320. (1-(6-(4-chlorophenyl)-2-(pyridin-3-yl)pyrimidin-4-yl)azetidin-3-yl)methanol
321. 4-(4-chlorophenyl)-2-(1-methyl-1H-pyrazol-4-yl)-6-(4-(methylsulfonyl)piperazin-1-yl)pyrimidine
322. (S)-1-(6-(4-chlorophenyl)-2-(3-hydroxyphenyl)pyrimidin-4-yl)pyrrolidin-3-ol formate)
322. (S)-1-(6-(4-chlorophenyl)-2-(3-hydroxyphenyl)pyrimidin-4-yl)pyrrolidin-3-ol
323. (S)-1-(6-(4-chlorophenyl)-2-(5-fluoropyridin-3-yl)pyrimidin-4-yl)pyrrolidin-3-ol
324. (S)-1-(6-(4-chlorophenyl)-2-(pyridin-4-yl)pyrimidin-4-yl)pyrrolidin-3-ol
325. 4-(4-chlorophenyl)-2-(5-fluoropyridin-3-yl)-6-(4-(methylsulfonyl)piperazin-1-yl)pyrimidine
326. 4-(4-chlorophenyl)-6-(4-(methylsulfonyl)piperidin-1-yl)-2-(pyridin-3-yl)pyrimidine
327. (S)-1-(2-(5-fluoropyridin-3-yl)-6-(4-(trifluoromethyl)phenyl)pyrimidin-4-yl)pyrrolidin-3-ol
328. 4-(4-chlorophenyl)-6-(4-(cyclopropylsulfonyl)piperazin-1-yl)-2-(pyridin-3-yl)pyrimidine
329. (S)-1-(6-(4-chlorophenyl)-2-(pyridazin-4-yl)pyrimidin-4-yl)pyrrolidin-3-ol
330. 1-(6-(4-chlorophenyl)-2-(pyridin-3-yl)pyrimidin-4-yl)azepan-4-ol
331. 2-(6-(4-chlorophenyl)-2-(pyridin-3-yl)pyrimidin-4-yl)-5-(methylsulfonyl)-2,5-diazabicyclo[2.2.1]heptane
332. (S)-1-(6-(4-chlorophenyl)-2-(2-methyl-2H-tetrazol-5-yl)pyrimidin-4-yl)pyrrolidin-3-ol
333. (S)-1-(6-(4-chlorophenyl)-2-(2-methyl-2H-tetrazol-5-yl)pyrimidin-4-yl)pyrrolidin-3-ol
334. 4-(4-chlorophenyl)-2-(2-methyl-2H-tetrazol-5-yl)-6-(4-(methylsulfonyl)piperazin-1-yl)pyrimidine
335. 4-(4-chlorophenyl)-6-(4-((2-fluoroethyl)sulfonyl)piperazin-1-yl)-2-(pyridin-3-yl)pyrimidine
336. (S)-1-(6-(4-chlorophenyl)-2-(isoxazol-4-yl)pyrimidin-4-yl)pyrrolidin-3-ol
337. (1-(6-(4-chlorophenyl)-2-(pyridin-3-yl)pyrimidin-4-yl)-4-(methylsulfonyl)piperidin-4-yl)methanol
338. 1-(6-(4-chlorophenyl)-2-(pyridin-3-yl)pyrimidin-4-yl)azepan-3-ol
339. 4-(4-chlorophenyl)-6-(4-((difluoromethyl)sulfonyl)piperazin-1-yl)-2-(pyridin-3-yl)pyrimidine
340. (S)-1-(2-(pyridin-3-yl)-6-(4-(trifluoromethyl)phenyl)pyrimidin-4-yl)pyrrolidin-3-ol
341. 4-(4-(methylsulfonyl)piperazin-1-yl)-2-(pyridin-3-yl)-6-(4-(trifluoromethyl)phenyl)pyrimidine
342. (S)-1-(6-(4-chlorophenyl)-2-(5,6-difluoropyridin-3-yl)pyrimidin-4-yl)pyrrolidin-3-ol
343. (3S,4R)-1-(2-(pyridin-3-yl)-6-(4-(trifluoromethyl)phenyl)pyrimidin-4-yl)pyrrolidine-3,4-diol
344. (S)-1-(6-(4-chlorophenyl)-[2,5′-bipyrimidin]-4-yl)pyrrolidin-3-ol
345. (S)-1-(6-(4-chlorophenyl)-2-(6-fluoropyridin-3-yl)pyrimidin-4-yl)pyrrolidin-3-ol
346. (S)-1-(6-(4-chlorophenyl)-2-(2-fluoropyridin-3-yl)pyrimidin-4-yl)pyrrolidin-3-ol
347. (S)-1-(6-(4-chlorophenyl)-2-(pyridin-2-yl)pyrimidin-4-yl)pyrrolidin-3-ol
348. 2-((4-(6-(4-chlorophenyl)-2-(1-methyl-1H-pyrazol-4-yl)pyrimidin-4-yl)piperazin-1-yl)sulfonyl)ethanol
349. 2-((4-(2-(pyridin-3-yl)-6-(4-(trifluoromethyl)phenyl)pyrimidin-4-yl)piperazin-1-yl)sulfonyl)ethan-1-ol
350. (S)-1-(2-(isothiazol-4-yl)-6-(4-(trifluoromethyl)phenyl)pyrimidin-4-yl)pyrrolidin-3-ol
351. (4-(methylsulfonyl)-1-(2-(pyridin-3-yl)-6-(4-(trifluoromethyl)phenyl)pyrimidin-4-yl)piperidin-4-yl)methanol
352. 1-(4-(6-(4-chlorophenyl)-2-(pyridin-3-yl)pyrimidin-4-yl)piperazin-1-yl)-3-hydroxypropan-1-one
353. 2-((4-(2-(1-methyl-1H-pyrazol-4-yl)-6-(4-(trifluoromethyl)phenyl)pyrimidin-4-yl)piperazin-1-yl)sulfonyl)ethanol
354. 1-(6-(4-chlorophenyl)-2-(pyridin-3-yl)pyrimidin-4-yl)-3-(dimethylamino)piperidin-4-ol
355. 1-(6-(4-chlorophenyl)-2-(pyridin-3-yl)pyrimidin-4-yl)-5-(dimethylamino)piperidin-3-ol
356. (1-(6-(4-chlorophenyl)-2-(1-methyl-1H-pyrazol-4-yl)pyrimidin-4-yl)-4-(methylsulfonyl)piperidin-4-yl)methanol
357. (1-(2-(1-methyl-1H-pyrazol-4-yl)-6-(4-(trifluoromethyl)phenyl)pyrimidin-4-yl)-4-(methylsulfonyl)piperidin-4-yl)methanol
358. (1-(6-(4-chlorophenyl)-2-(pyridin-3-yl)pyrimidin-4-yl)-3-(dimethylamino)piperidin-4-yl)methanol
359. 2-((4-(6-(4-chlorophenyl)-2-(pyridin-3-yl)pyrimidin-4-yl)-3-methylpiperazin-1-yl)sulfonyl)ethan-1-ol
360. 2-((1-(6-(4-chlorophenyl)-2-(pyridin-3-yl)pyrimidin-4-yl)piperidin-4-yl)amino)ethanol
361. 1-(4-(6-(4-chlorophenyl)-2-(pyridin-3-yl)pyrimidin-4-yl)piperazin-1-yl)-4-hydroxybutan-1-one
362. 3-((4-(6-(4-chlorophenyl)-2-(pyridin-3-yl)pyrimidin-4-yl)piperazin-1-yl)sulfonyl)propan-1-ol
363. 1-(4-(6-(4-chlorophenyl)-2-(pyridin-3-yl)pyrimidin-4-yl)piperazin-1-yl)-3,4-dihydroxybutan-1-one
364. 1-(4-(6-(4-chlorophenyl)-2-(pyridin-3-yl)pyrimidin-4-yl)piperazin-1-yl)-2,3-dihydroxybutan-1-one
365. 4-(6-(4-chlorophenyl)-2-(pyridin-3-yl)pyrimidin-4-yl)-6-methylpiperazin-2-one
366. (S)-3-(4-(3-hydroxypyrrolidin-1-yl)-6-(6-morpholinopyridin-3-yl)pyrimidin-2-yl)pyridin-2-ol
367. (S)-4-(4-(6-(3-hydroxypyrrolidin-1-yl)-2-(pyridin-3-yl)pyrimidin-4-yl)phenyl)morpholin-3-one
368. 2-((4-(6-(4-chlorophenyl)-2-(isothiazol-4-yl)pyrimidin-4-yl)piperazin-1-yl)sulfonyl)ethanol
369. 3-((4-(6-(4-chlorophenyl)-2-(pyridin-3-yl)pyrimidin-4-yl)piperazin-1-yl)sulfonyl)propane-1,2-diol
369. 3-((4-(6-(4-chlorophenyl)-2-(pyridin-3-yl)pyrimidin-4-yl)piperazin-1-yl)sulfonyl)propane-1,2-diol
370. 2-((4-(2-(isothiazol-4-yl)-6-(4-(trifluoromethyl)phenyl)pyrimidin-4-yl)piperazin-1-yl)sulfonyl)ethanol
371. (S)-2-(4-(6-(4-chlorophenyl)-2-(pyridin-3-yl)pyrimidin-4-yl)piperazin-2-yl)ethanol
372. (S)-4-(5-(6-(3-hydroxypyrrolidin-1-yl)-2-(pyridin-3-yl)pyrimidin-4-yl)pyridin-2-yl)morpholin-3-one
373. (S)-3-(4-(3-fluoro-4-morpholinophenyl)-6-(3-hydroxypyrrolidin-1-yl)pyrimidin-2-yl)pyridin-2-ol
373. (S)-3-(4-(3-fluoro-4-morpholinophenyl)-6-(3-hydroxypyrrolidin-1-yl)pyrimidin-2-yl)pyridin-2-ol
374. (S)-1-(6-(4-((2-(dimethylamino)ethyl)amino)phenyl)-2-(pyridin-3-yl)pyrimidin-4-yl)pyrrolidin-3-ol
375. (S)-1-(6-(4-(2-(dimethylamino)ethoxy)phenyl)-2-(pyridin-3-yl)pyrimidin-4-yl)pyrrolidin-3-ol, and
376. (S)-1-(6-(4-((2-hydroxyethyl)amino)phenyl)-2-(pyridin-3-yl)pyrimidin-4-yl)pyrrolidin-3-ol.

Single stereochemical isomers, enantiomers, diastereomers, and pharmaceutically acceptable salts of the above exemplified compounds are also within the scope of the present disclosure. Pharmaceutically acceptable salts may be, for example, derived from suitable inorganic and organic acids and bases.

Acid addition salts can be prepared by reacting the purified compound in its free-based form, if possible, with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid.

Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium and N⁺(C₁₋₄alkyl)₄ salts.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

In addition, the compounds represented by Formula I according to the present invention include, but are not limited thereto, not only pharmaceutically acceptable salts thereof, but also all solvates or hydrates and all possible stereoisomers that can be prepared therefrom. All stereoisomers of the present compounds (e.g., those which may exist due to asymmetric carbons on various substituents), including enantiomeric forms and diastereomeric forms, are contemplated within the scope of this invention. Individual stereoisomers of the compounds of the present invention may, for example, be substantially free of other isomers (e.g., as a pure or substantially pure optical isomer having a specified activity), or may be admixed, for example, as racemates or with all other, or other selected, stereoisomers. The chiral centers of the compounds of the present invention may have the S or R configuration as defined by the IUPAC 1974 Recommendations. The racemic forms can be analyzed by physical methods, such as fractional crystallization, separation or crystallization of diastereomeric derivatives or separation by chiral column chromatography. The individual optical isomers can be obtained from the racemates by any suitable method, including without limitation, salt formation with an optically active acid followed by crystallization.

The solvate and stereoisomer of the compound represented by Formula I may be prepared from the compound represented by Formula I using methods known in the art.

Furthermore, the compounds represented by Formula I according to the present invention may be prepared either in a crystalline form or in a non-crystalline form, When the compound is prepared in a crystalline form, it may be optionally hydrated or solvated. In the present invention, the compound of Formula I may not only include a stoichiometric hydrate, but also include a compound containing various amounts of water. The solvate of the compound of Formula I according to the present invention includes both stoichiometric solvates and non-stoichiometric solvates.

The compounds of the present invention may be synthesized by methods known in the art or by methods illustrated in Examples 1-376 below.

Pharmaceutical Compositions, Methods and Use

In a specific embodiment, the pharmaceutical composition and the method provided herein comprises the compound of Formula (I).

The subject may be a mammal including human or a mammalian cell; for example, a mammal (e.g., human) suffering from the disease, disorder, or condition associated with AhR activity as described above or a mammalian cell isolated therefrom.

The compound as an active ingredient or the pharmaceutical composition may be administered orally or parenterally. For example, the parenteral administration may be performed by any one of intravenous injection, subcutaneous injection, intramuscular injection, intraperitoneal injection, endothelial administration, topical administration, intranasal administration, intrapulmonary administration, intrarectal administration, and the like.

The effective amount may refer to pharmaceutically and/or therapeutically effective amount, and may be prescribed depending on factors such as a type of preparation (formulation), administration route, the patient's age, body weight, gender, and/or pathologic conditions, and the like.

A pharmaceutically acceptable salt of the compound of Formula (I) may include addition salts formed by inorganic acids such as hydrochloride, sulfate, phosphate, hydrobromide, hydroiodide, nitrate, pyrosulfate, or metaphosphate, addition salts formed by organic acids such as citrate, oxalate, benzoate, acetate, trifluoroacetate, propionate, succinate, fumarate, lactate, maleate, tartrate, glutarate, or sulfonate, or metal salts such as lithium salt, sodium salt, potassium salt, magnesium salt and calcium salt, but is not limited thereto.

The pharmaceutical composition according to the present invention can be formulated into a suitable form together with a commonly used pharmaceutically acceptable carrier. The “pharmaceutically acceptable” refers to being physiologically acceptable, and not usually causing an allergic reaction or a similar reaction such as gastrointestinal disorders and dizziness when administered to humans. Further, the pharmaceutical composition of the present invention may be used after being formulated into an oral preparation, such as powders, granules, tablets, capsules, suspensions, emulsions, syrups, and aerosols, etc., and a parental preparation, such as epidermal formulations, suppositories, or sterile injection solutions, in accordance with a conventional method.

Examples of carriers, excipients and diluents that can be included in the composition, may include lactose, dextrose, sucrose, sorbitol, mannitol, xylitol, erythritol, maltitol, starch, arabic gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil, but are not limited thereto. When formulated into a preparation, a diluting agent or an excipient, such as commonly-used fillers, stabilizing agents, binding agents, disintegrating agents, and surfactants can be used. Solid preparations for oral administration include tablets, pills, powders, granules, capsules, and the like, and these solid preparations may be prepared by mixing the compound of the present invention with at least one excipient, for example, starch, microcrystalline cellulose, sucrose, lactose, low-substituted hydroxypropyl cellulose, hypromellose or the like. In addition to the simple excipient, a lubricant such as magnesium stearate and talc are also used. Liquid preparations for oral administration include a suspension, a liquid for internal use, an emulsion, a syrup, etc. In addition to a commonly used simple diluent such as water and liquid paraffin, various excipients such as a humectant, a sweetener, an aromatic, a preservative, etc. may also be contained. Formulations for parenteral administration include a sterilized aqueous solution, a non-aqueous solution, a suspension, an emulsion, a lyophilized formulation and a suppository. The non-aqueous solution or suspension may contain propylene glycol, polyethylene glycol, a vegetable oil such as olive oil, an injectable ester such as ethyl oleate, etc. As a base of the suppository, witepsol, macrogol, tween 61, cocoa butter, laurin butter, glycerogelatin, etc. may be used. In order to formulate the formulation for parenteral administration, the compound of Formula I or a pharmaceutically acceptable salt thereof may be mixed in water together with sterilized and/or contain adjuvants such as preservatives, stabilizers, auxiliary agents such as wettable powder or emulsifying accelerators, salt for controlling osmotic pressure and/or buffers and the like, and other therapeutically useful substances, to prepare a solution or suspension, which is then manufactured in the form of an ampoule or vial unit administration.

The pharmaceutical composition including the compound of Formula I disclosed herein as an active ingredient may be administered to mammals such as mice, livestock, and humans by various routes for the modulation of AhR activity, or the prevention or treatment of a disease, disorder, or condition associated with AhR activity.

In some embodiment, the disease, disorder, or condition associated with AhR activity. may be a cancer, cancerous condition, tumor, fibrotic diseases, immune related disease or other disease related with AhR signaling.

In some embodiment, the diseases related with dysregulated immune response associated with AhR signaling are selected from the group consisting of sepsis (SIRS), multiple organ failure (MODS, MOF), inflammatory disorders of the kidney, chronic intestinal inflammations (IBD, Crohn's disease, UC), pancreatitis, peritonitis, inflammatory skin disorders and inflammatory eye disorders, autoimmune diseases, such as rheumatoid diseases including rheumatoid arthritis (RA), systemic lupus erythematosus (SLE), and multiple sclerosis (MS).

In some embodiment, the fibrotic disorders are selected from the group consisting of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

In some embodiments of the cancer, cancerous condition, or tumor particularly suitable for treatment with an AHR antagonist of the present invention are liquid and solid tumours, such as a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

In some embodiments, the pharmaceutical composition of the preset invention can be used together with one or more additional anti-cancer therapies. In some such embodiments, the additional anti-cancer therapy comprises surgery, radiation therapy, biotherapy, immunotherapy, chemotherapy, or any combination thereof.

In some embodiments, the pharmaceutical composition of the preset invention can be used together with anti-cancer therapeutic agents. In some such embodiments, the anti-cancer therapeutic agent is a chemotherapeutic agent, a growth inhibitor agent, an anti-angiogenesis agent, a cytotoxic agent, an anti-hormonal agent, a prodrug, or a cytokine.

Examples of other disorders associated with aberrant AhR signaling inflammation are vaccination for infection & cancer, viral infections, obesity and diet-induced obesity, adiposity, metabolic disorders, hepatic steatosis and uterine fibroids (uterine leiomyoma or uterine myoma) in women, chronic renal disorders, acute and chronic renal insufficiency, diabetic, inflammatory or hypertensive nephropaties, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer, and Creutzfeld-Jakob.

Also provided herein, in other aspects, are pharmaceutical compositions comprising an AhR modulator, such as an AhR antagonist of Formula (I), and pharmaceutically acceptable excipients.

In some aspects, pharmaceutical compositions comprising an AhR modulator, such as an AhR antagonist of Formula (I), are provided for use in for modulating constitutive AhR activity in a subject in need thereof.

In some aspects, pharmaceutical compositions comprising an AhR modulator, such as an AhR antagonist of Formula (I), are provided for use in treating a cancer or a cancerous condition by modulating AhR activity.

In some aspects, pharmaceutical compositions comprising an AhR modulator, such as an AhR antagonist of Formula (I), are provided for use in inhibiting proliferation, tissue invasion, metastasis and angiogenesis of cancer cells in a subject having a cancer, a cancerous condition, or a tumor.

In some embodiment, the pharmaceutical composition of the present invention may be for use in inhibiting proliferation, tissue invasion, metastasis and angiogenesis of cancer cells in a subject having a cancer, a cancerous condition, or a tumor.

Pharmaceutical formulations described herein are administrable to a subject in a variety of by multiple administration routes, including but not limited to, oral, parenteral (e.g., intravenous, subcutaneous, intramuscular, rectal, enfometrial or cerebrovascular injection), intranasal, buccal, topical or transdermal administration routes.

In some embodiments, the compounds of Chemical Formula I are administered orally.

Another aspect of the present invention relates to a method of stimulating the immune system in a patient in need thereof, e.g., in a patient suffering from cancer or an infection (e.g., a viral, bacterial, or parasitic infection). The method includes administering to the patient a therapeutically effective amount of one or a combination of the compounds described herein. In some embodiments, the patient has an increased count of white blood cells, T and/or B lymphocytes, macrophases, dendritic cells, neutrophils, natural killer (NK) cells, and/or platelets after the administering step. In some embodiments, the compound decreases IL-21 level in the patient. The patient may have cancer, or may be immune-compromised.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival. “A therapeutically effective amount” is an amount of the medication that can achieve the desired curative, palliative, or prophylactic effect for the treated condition.

In some embodiments, the effective dose range of a compound is determined by measuring the patient's blood concentration of the compound under a specified dosing regimen to establish a concentration-time profile, consulting with an established correlation between the concentration-time profiles and effects on cancer inhibition or eradication obtained during a trial, and balancing the therapeutic effects achievable with possible toxicity to the patient, with further consideration of the health condition or physical durability of the patient. The dosing frequency of the compound may be determined similarly. The dosing may be continued until the patiunlessent is free from the cancer.

In some embodiments, an effective amount for tumor therapy may be measured by its ability to stabilize disease progression and/or ameliorate symptoms in a patient, and preferably to reverse disease progression, e.g., by reducing tumor size. In some embodiments, a maintenance dosing may be provided after the patient is free of cancer to ensure its complete elimination or eradication, or prevention of residual disease. The duration of the maintenance dosing can be determined based on clinical trial data.

In some embodiments, a compound may be administered in combination with one or more other cancer therapeutic agents that also target AhR or target molecules other than AhR. Compounds can be formulated either separately from, or together with, the other cancer therapeutic agents. Compounds can be administered either at the same schedule as, or at a different schedule from, the other cancer therapeutic agents. The proportion of a compound relative to other cancer therapeutic agents may be determined by clinical trials. Combining the compounds with the other cancer therapeutic agents may further enhance the efficacy of one another. For example, a compound of the present invention can be administered with an immune checkpoint inhibitor, such as an inhibitor of PD-1, PD-L1 or PD-L2 (e.g., pembrolizumab, nivolumab, or atezolizumab), or administered with CAR-T therapy (e.g., axicabtagene ciloleucel), to achieve additive or synergistic anti-cancer effect.

Dosage regimens may be adjusted to provide the optimum desired response. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values.

It is contemplated that a suitable dose of a compound of the present invention may be in the range of 0.001-200 mg/kg per day, and preferably from about 0.01 mg/kg to about 20 mg/kg body weight per day, such as about 0.5-50 mg/kg, e.g., about 1-20 mg/kg. The compound may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg, such as up to at the most 30 mg/kg, e.g., up to at the most 20 mg/kg, such as up to at the most 15 mg/kg. Administration will normally be repeated at suitable intervals, e.g., twice a day, thrice a day, once a day, once every week, once every two weeks, or once every three weeks, and for as long as deemed appropriate by the responsible doctor, who may optionally increase or decrease the dosage as necessary.

General Synthetic Methods

The compounds of Formula (I) of the present invention can be prepared in accordance with one or more of schemes discussed below.

These methods can be used either directly or with obvious variations to trained chemists to prepare key intermediates and certain compounds of this invention.

Suitable synthetic sequences are readily selected per specific structures of this invention, but within the art known to individuals practicing organic synthesis, such as methods summarized in available chemistry data bases, as in CAS Scifinder and Elesevier Reaxys. Based on these general methods, the enablement for making the compounds of this invention is straightforward and can be practiced within a common professional knowledge. Some general synthetic methods to prepare the compounds of this invention are illustrated below in Schemes 1-5 (general procedure A-E).

One general approach to the compounds of this invention is illustrated in general Scheme 1.

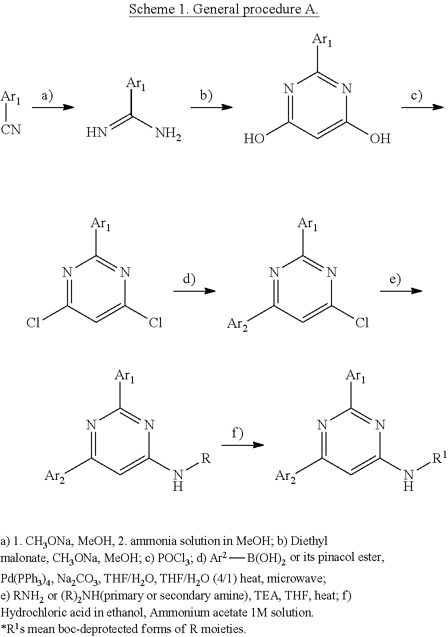

Another general approach to the compounds of this invention is illustrated in general Scheme 2.

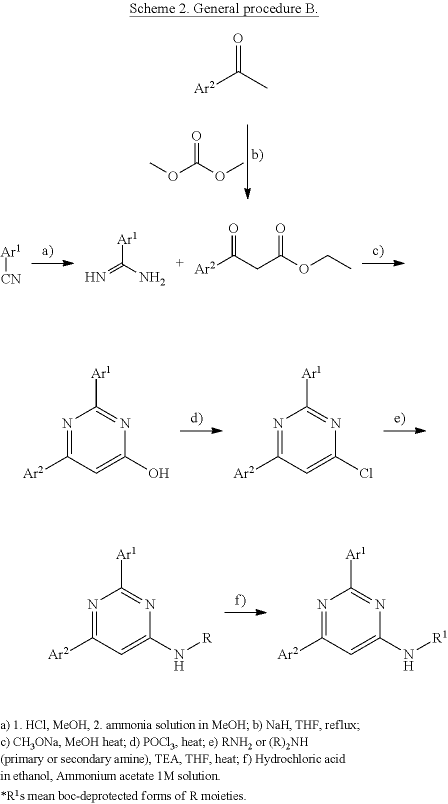

Another general approach to the compounds of this invention is illustrated in general Scheme 3.

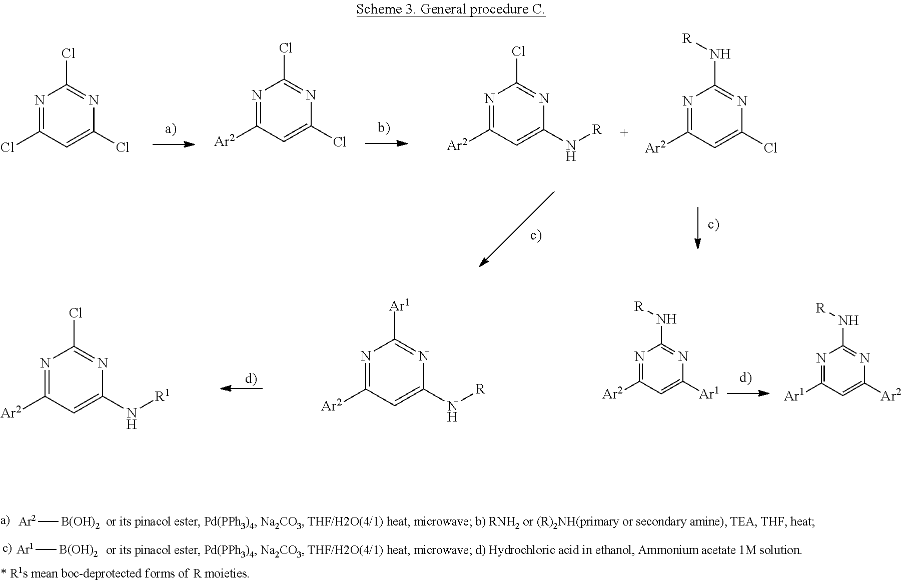

Another general approach to the compounds of this invention is illustrated in general Scheme 4.

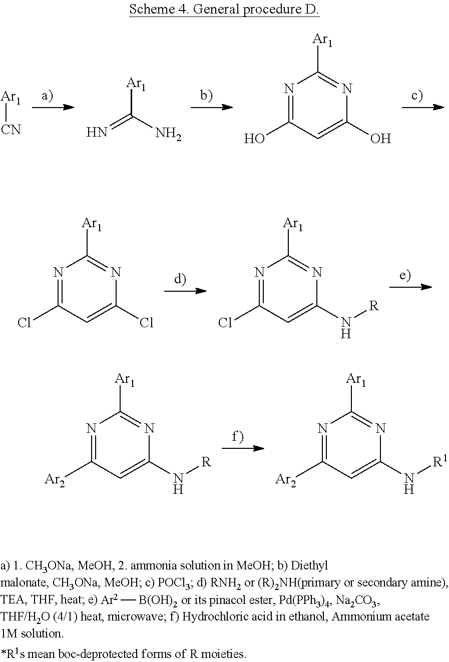

Another general approach to the compounds of this invention is illustrated in general Scheme 5.

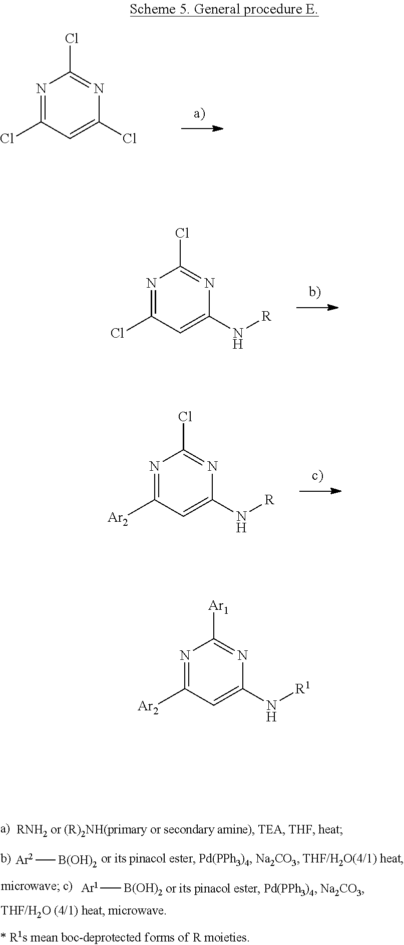

### MODE FOR INVENTION

### EXAMPLES

Embodiments of the present invention are described in the following examples, which are meant to illustrate and not limit the scope of this invention. Common abbreviations well known to those with ordinary skills in the synthetic art used throughout.

All chemical reagents were commercially available. Flash column chromatography means silica gel chromatography unless specified otherwise, which was performed on Teledyne Combiflash-RF200 System. ¹H NMR spectra (6, ppm) are recorded on 400 MHz or 600 MHz instrument. Mass spectroscopy data for a positive ionization method are provided. Preparative HPLC was performed on Agilent technologies G1361A and Gilson Preparative HPLC System.

### Example 1. (R)-2-((6-(4-chlorophenyl)-2-(pyridin-3-yl)pyrimidin-4-yl)amino)butan-1-ol

Scheme for the preparation of the Compound of Example 1:

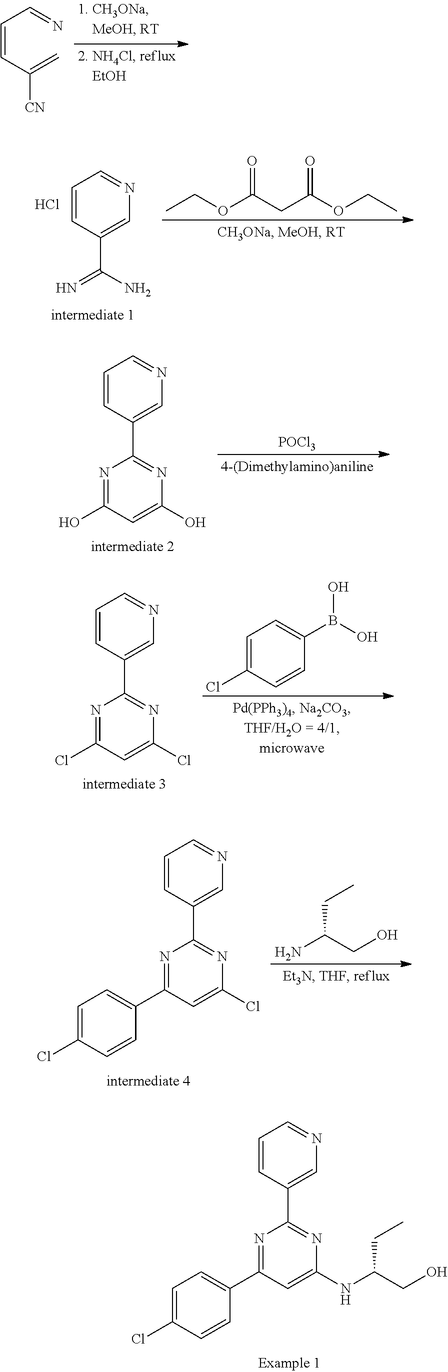

### Intermediate 1. Nicotinimidamide Hydrochloride

To a suspension of 3-Cyanopyridine (5 g, 48.03 mmol) in 50 mL of MeOH was added Sodium methoxide 30 wt % in MeOH (4 mL) and the mixture was stirred at room temperature for 24 hr. After adding NH₄Cl (16.5 g, 0.31 mol), the mixture was heated at reflux for 6 h and then cooled. The solvent was removed in vacuo and then EtOH (60 mL) was added and the mixture was heated to reflux for 30 min. After the reaction mixture was cooled to room temperature, solids were filtered off and the filtrate was concentrated in vacuo. The suspension of reaction mixture in 3 mL of EtOH was filtered and the solid product was dried to afford 4.9 g of the title compound.

¹H NMR (400 MHz, DMSO-d₆) δ [ppm]=9.43 (bs, 4H), 8.98 (d, J=1.6 Hz, 1H), 8.74 (dd, J=4.8 Hz, J=1.2 Hz, 1H), 8.20-8.23 (m, 1H), 7.64-7.67 (m, 1H); MS (ESI, m/z): 122.1 [M+H]⁺

### Intermediate 2. 2-Pyridin-3-yl-pyrimidine-4,6-diol

To a solution of 3-pyridyl amidine hydrochloride (4.8 g, 30.46 mmol) in MeOH (120 mL) was added diethyl malonate (4.63 mL, 30.46 mmol) followed by a solution of sodium methoxide 30 wt % in MeOH (20 mL) at 0° C. The resulting mixture was stirred for 24 h at room temperature. The solvents were removed in vacuo. The resulting residue was used without further purification.

MS (ESI, m/z): 190.0 [M+H]⁺

### Intermediate 3. 4,6-Dichloro-2-pyridin-3-yl-pyrimidine

To a solution of 2-pyridin-3-yl-pyrimidine-4,6-diol (crude 5.0 g of previous step) in POCl₃ (10 mL) was added dimethylamino aniline (4.77 g, 35.03 mmol) and the reaction mixture was heated at 120° C. for 4 h. The residue was cooled to room temperature, extracted with 500 mL of EtOAc and concentrated in vacuo. The crude product was purified by silicagel column chromatography to give 4.45 g of the title compound.

¹H NMR (400 MHz, DMSO-d₆) δ [ppm]=7.36 (s, 1H), 7.49 (dd, J=4.8 Hz, J=1.6 Hz, 1H), 8.80-8.72 (m, 2H), 9.64 (br, 1H); MS (ESI, m/z): 226.0 [M+H]+

### Intermediate 4. 4-chloro-6-(4-chlorophenyl)-2-(pyridin-3-yl)pyrimidine

To a mixture of 4,6-Dichloro-2-pyridin-3-yl-pyrimidine (1.03 g, 4.6 mmol), (4-chlorophenyl)boronic acid (0.66 g, 4.2 mmol) and sodium carbonate (1.01 g, 9.5 mmol) in 50 mL of tetrahydrofuran/H₂O (4/1) was added Pd(PPh₃)₄ (203 mg, 0.18 mmol). The mixture is heated under microwave at 80° C. for 20 minutes, cooled to room temperature and extracted three times with EtOAc (50 mL). The organic layer was dried over anhydrous MgSO₄ and concentrated under reduced pressure. The crude product was purified by silicagel column chromatography to give 1.02 g of the title compound.

¹H NMR (600 MHz, CDCl₃) δ [ppm]=7.52 (d, 2H), 7.74 (s, 1H), 7.79 (dd, 1H), 8.06 (d, 2H), 9.02 (d, 1H), 9.12 (d, 1H), 10.16 (s, 1H); MS (ESI, m/z): 302.0 [M+H]⁺

### Example 1. (R)-2-((6-(4-chlorophenyl)-2-(pyridin-3-yl)pyrimidin-4-yl)amino)butan-1-ol

To a solution of 4-chloro-6-(4-chlorophenyl)-2-(pyridin-3-yl)pyrimidine (45 mg, 0.15 mmol) in tetrahydrofuran (6 mL) was added triethylamine (0.3 mL, 2.15 mmol) followed by (R)-2-aminobutan-1-ol (27 mg, 0.30 mmol) at room temperature. The reaction mixture in sealed tube was heated at 120° C. for 4 h. and cooled to room temperature. The residue was filtered, evaporated in vacuo and isolated by Preparative HPLC to give 50 mg of the title compound.

¹H NMR (400 MHz, CD₃OD) δ [ppm]=1.05 (t, 3H), 1.59-1.64 (m, 1H), 1.83-1.86 (m, 1H), 3.70 (d, 2H), 4.39 (br, 1H), 6.96 (s, 1H), 7.50 (d, 2H), 8.10-8.13 (m, 3H), 8.90 (d, 1H), 9.44 (d, 1H), 9.69 (d, 1H); MS (ESI, m/z): 355.1 [M+H]⁺

### Example 2 and 3. (S)-2-((4-(4-chlorophenyl)-6-(pyridin-3-yl)pyrimidin-2-yl)amino)propan-1-ol and (S)-2-((6-(4-chlorophenyl)-2-(pyridin-3-yl)pyrimidin-4-yl)amino)propan-1-ol

Scheme for the preparation of the Compound of Example 2 and 3:

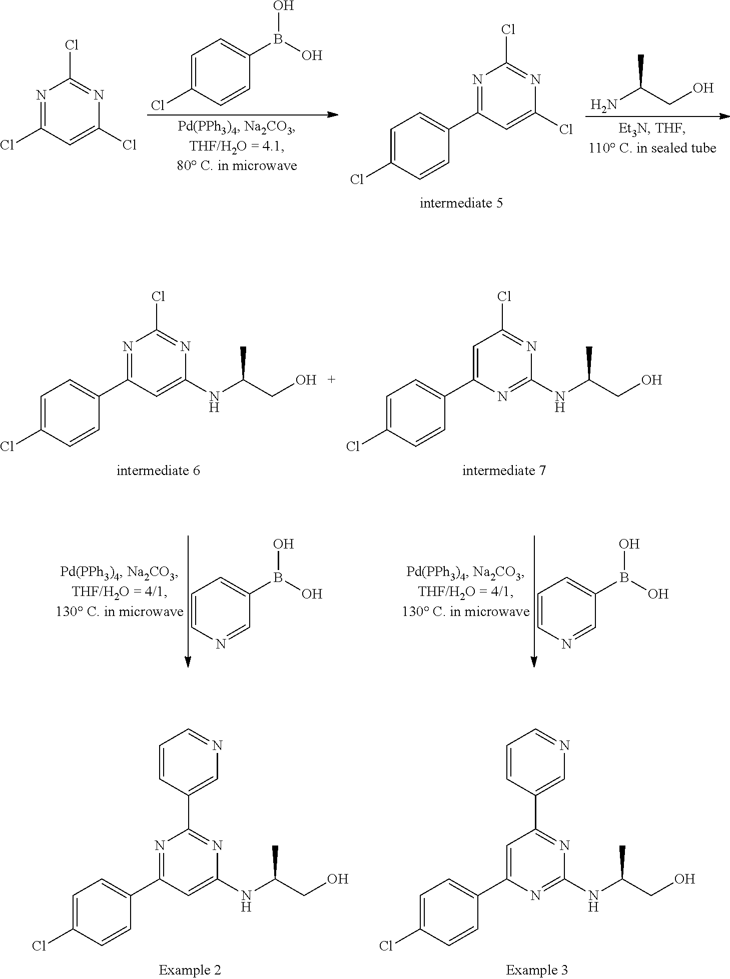

### Intermediate 5. 2,4-dichloro-6-(4-chlorophenyl)pyrimidine

To a solution of 2,4,6-trichloropyrimidine (813.5 mg, 4.43 mmol), (4-chlorophenyl)boronic acid (329.2 mg, 2.11 mmol) and sodium carbonate (314.6 mg, 2.97 mmol) in 20 mL of tetrahydrofuran/H₂O (4/1) was added Pd(PPh₃)₄ (253.7 mg, 0.22 mmol). The mixture is heated to 80° C. for overnight, cooled to room temperature and extracted three times with EtOAc (150 mL) two times. The organic layer was washed with brine, dried over anhydrous MgSO₄ and concentrated under reduced pressure. The crude product was purified by flash chromatography (silica gel, hexane/ethyl acetate, gradient) to give 510.0 mg of the title compound.

¹H NMR (600 MHz, CDCl₃) δ [ppm]=7.52 (d, 2H), 7.67 (s, 1H), 8.05 (d, 2H); MS (ESI, m/z): 258.0 [M+H]⁺

### Intermediate 6 and 7. (S)-2-((2-chloro-6-(4-chlorophenyl)pyrimidin-4-yl)amino)propan-1-ol and (S)-2-((4-chloro-6-(4-chlorophenyl)pyrimidin-2-yl)amino)propan-1-ol

To a solution of 2,4-dichloro-6-(4-chlorophenyl)pyrimidine (intermediate 5, 76.2 mg, 0.294 mmol) in tetrahydrofuran (6 mL) was added triethylamine (0.1 mL, 0.712 mmol) followed by (S)-2-aminopropan-1-ol (28.95 mg, 0.385 mmol) at room temperature. The reaction mixture was stirred at RT for overnight. The reaction mixture was filtered, evaporated in vacuo and isolated by flash chromatography (silica gel, hexane/ethyl acetate, gradient) to give 54.0 mg and 30.1 mg of the intermediate 6 and 7 respectively.

### Intermediate 6

¹H NMR (600 MHz, CDCl₃) δ [ppm]=1.28 (d, 3H), 3.61-3.65 (m, 1H), 3.78-3.81 (m, 1H), 4.15 (br, 1H), 5.48 (br, 1H), 6.59 (s, 1H), 7.41 (d, 2H), 7.86 (d, 2H); MS (ESI, m/z): 298.0 [M+H]⁺

### Intermediate 7

¹H NMR (600 MHz, CDCl₃) δ [ppm]=1.28 (d, 3H), 3.61-3.66 (m, 1H), 3.79-3.83 (m, 1H), 4.27 (br, 1H), 5.58 (br, 1H), 6.93 (s, 1H), 7.42 (d, 2H), 7.88 (d, 2H); MS (ESI, m/z): 298.0 [M+H]⁺

### Example 2. (S)-2-((6-(4-chlorophenyl)-2-(pyridin-3-yl)pyrimidin-4-yl)amino)propan-1-ol

To a solution (S)-2-((2-chloro-6-(4-chlorophenyl)pyrimidin-4-yl)amino)propan-1-ol (60 mg, 0.201 mmol), 3-pyridylboronic acid (80 mg, 0.651 mmol), sodium carbonate (90 mg, 0.849 mmol) in 10 mL of tetrahydrofuran/H₂O (4/1) was added Pd(PPh₃)₄ (80 mg, 0.069 mmol). The mixture is heated under microwave at 130° C. for 15 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, hexane/ethyl acetate, gradient) to give 32 mg of the title compound (Scheme 3. General procedure C.).

¹H NMR (600 MHz, CD₃OD) δ [ppm]=1.25 (d, 3H), 3.56-3.59 (m, 1H), 3.63-3.66 (m, 1H), 4.35 (br, 1H), 6.73 (s, 1H), 7.36 (d, 2H), 7.45-7.49 (m, 1H), 7.98 (d, 2H), 8.56 (br, 1H), 8.71 (d, 1H), 9.48 (br, 1H); MS (ESI, m/z): 341.1 [M+H]⁺

### Example 3. (S)-2-((4-(4-chlorophenyl)-6-(pyridin-3-yl)pyrimidin-2-yl)amino)propan-1-ol

To a solution (S)-2-((4-chloro-6-(4-chlorophenyl)pyrimidin-2-yl)amino)propan-1-ol (25 mg, 0.084 mmol), 3-pyridylboronic acid (31 mg, 0.252 mmol), sodium carbonate (35 mg, 0.336 mmol) in 5 mL of tetrahydrofuran/H₂O (4/1) was added Pd(PPh₃)₄ (34 mg, 0.029 mmol). The mixture is heated under microwave at 130° C. for 15 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, hexane/ethyl acetate, gradient) to give 17.6 mg of the title compound (Scheme 3. General procedure C.).

¹H NMR (600 MHz, CD₃OD) δ [ppm]=1.34 (d, 3H), 3.64-3.66 (m, 1H), 3.73-3.76 (m, 1H), 4.33-4.36 (m, 1H), 7.49 (d, 2H), 7.57-7.59 (m, 1H), 7.60 (s, 1H), 8.17 (d, 2H), 8.58 (d, 1H), 8.67 (s, 1H), 9.34 (s, 1H); MS (ESI, m/z): 341.1 [M+H]⁺

### Example 4. 2-((6-(4-chlorophenyl)-2-(pyridin-3-yl)pyrimidin-4-yl)amino)-2-methylpropan-1-ol

Using 2-amino-2-methylpropan-1-ol, the title compound was obtained as described for the example 2 (Scheme 3. General procedure C.).

¹H NMR (600 MHz, CD₃OD) δ [ppm]=1.53 (s, 6H), 3.91 (s, 2H), 7.01 (s, 1H), 7.53 (d, 2H), 8.11 (d, 2H), 8.05-8.15 (m, 1H), 8.90 (br, 1H), 9.33 (d, 1H), 9.68 (br, 1H); MS (ESI, m/z): 355.1 [M+H]⁺

### Example 5. 2-((4-(4-chlorophenyl)-6-(pyridin-3-yl)pyrimidin-2-yl)amino)-2-methylpropan-1-ol

Using 2-amino-2-methylpropan-1-ol, the title compound was obtained as described for the example 3 (Scheme 3. General procedure C.).

¹H NMR (600 MHz, CDCl₃) δ [ppm]=1.55 (s, 6H), 3.84 (s, 2H), 7.12 (d, 1H), 7.40 (s, 1H), 7.44-7.48 (m, 1H), 7.54 (d, 2H), 7.98 (d, 2H), 8.03 (d, 1H), 8.82 (br, 1H); MS (ESI, m/z): 355.1 [M+H]⁺

### Example 6. 2-((6-(4-chlorophenyl)-2-(pyridin-3-yl)pyrimidin-4-yl)amino)ethan-1-ol

Using 2-aminoethan-1-ol, the title compound was obtained as described for the example 1 (Scheme 1. General procedure A.).

¹H NMR (600 MHz, CD₃OD) δ [ppm]=3.80 (t, 2H), 4.10 (t, 2H), 6.84 (s, 1H), 7.45 (d, 2H), 7.50-7.52 (m, 1H), 8.07 (d, 2H), 8.59 (d, 1H), 8.79 (d, 1H), 9.52 (s, 1H); MS (ESI, m/z): 327.1 [M+H]⁺

### Example 7. 3-((6-(4-chlorophenyl)-2-(pyridin-3-yl)pyrimidin-4-yl)amino)propan-1-ol

Using 3-aminopropan-1-ol, the title compound was obtained as described for the example 1 (Scheme 1. General procedure A.).

¹H NMR (600 MHz, CD₃OD) δ [ppm]=1.91-1.94 (m, 2H), 3.72 (t, 2H), 4.10 (t, 2H), 6.89 (s, 1H), 7.82 (d, 2H), 7.56-7.58 (m, 1H), 8.15 (d, 2H), 8.63 (d, 1H), 8.87 (d, 1H), 9.58 (s, 1H); MS (ESI, m/z): 341.1 [M+H]⁺

### Example 8. (S)-1-((6-(4-chlorophenyl)-2-(pyridin-3-yl)pyrimidin-4-yl)amino)propan-2-ol

Using (S)-1-aminopropan-2-ol, the title compound was obtained as described for the example 1 (Scheme 1. General procedure A.).

¹H NMR (600 MHz, CDCl₃) δ [ppm]=1.31 (d, 3H), 3.41 (br, 1H), 3.70 (br, 1H), 4.12-4.15 (m, 1H), 5.63 (br, 1H), 6.65 (s, 1H), 7.43 (d, 2H), 7.39-7.45 (m, 1H), 8.00 (d, 2H), 8.66 (br, 1H), 8.72 (d, 1H), 9.64 (br, 1H); MS (ESI, m/z): 341.1 [M+H]⁺

### Example 9. (R)-1-((6-(4-chlorophenyl)-2-(pyridin-3-yl)pyrimidin-4-yl)amino)propan-2-ol

Using (R)-1-aminopropan-2-ol, the title compound was obtained as described for the example 1 (Scheme 1. General procedure A.).

¹H NMR (600 MHz, CDCl₃) δ [ppm]=1.31 (d, 3H), 3.41 (br, 1H), 3.70 (br, 1H), 4.12-4.15 (m, 1H), 5.63 (br, 1H), 6.65 (s, 1H), 7.43 (d, 2H), 7.39-7.45 (m, 1H), 8.00 (d, 2H), 8.66 (br, 1H), 8.72 (d, 1H), 9.64 (br, 1H); MS (ESI, m/z): 341.1 [M+H]⁺

### Example 10. 3-((6-(4-chlorophenyl)-2-(pyridin-3-yl)pyrimidin-4-yl)amino)propane-1,2-diol

Using 3-aminopropane-1,2-diol, the title compound was obtained as described for the example 1 (Scheme 1. General procedure A.).

MS (ESI, m/z): 357.1 [M+H]⁺

### Example 11. (R)-2-((6-(4-chlorophenyl)-2-(pyridin-3-yl)pyrimidin-4-yl)amino)propan-1-ol

Using (R)-2-aminopropan-1-ol and separation method of PREP. HPLC, the title compound was obtained as described for the example 1 (Scheme 1. General procedure A.)

¹H NMR (600 MHz, CD₃OD) δ [ppm]=1.31 (d, 3H), 3.61-3.64 (m, 1H), 3.67-3.70 (m, 1H), 4.50 (br, 1H), 6.95 (s, 1H), 7.52 (d, 2H), 8.01-8.19 (m, 1H), 8.12 (d, 2H), 8.85 (br, 1H), 9.38 (d, 1H), 9.71 (br, 1H); MS (ESI, m/z): 341.1 [M+H]⁺

### Example 12. 2-((6-(4-chlorophenyl)-2-(pyridin-3-yl)pyrimidin-4-yl)amino)-3-methylbutan-1-ol

Using 2-amino-3-methylbutan-1-ol and separation method of PREP. HPLC, the title compound was obtained as described for the example 1 (Scheme 1. General procedure A.).

¹H NMR (600 MHz, CD₃OD) δ [ppm]=1.06 (d, 3H), 1.09 (d, 3H), 2.10 (br, 1H), 3.73-3.76 (m, 1H), 3.80-3.83 (m, 1H), 4.41 (br, 1H), 7.02 (s, 1H), 7.54 (d, 2H), 8.06 (br, 1H), 8.14 (d, 2H), 8.88 (br, 1H), 9.38 (d, 1H), 9.69 (br, 1H); MS (ESI, m/z): 369.2 [M+H]⁺

### Example 13. (S)-2-((6-(4-chlorophenyl)-2-(pyridin-3-yl)pyrimidin-4-yl)amino)-3-methylbutan-1-ol

Using (S)-2-amino-3-methylbutan-1-ol and separation method of PREP. HPLC, the title compound was obtained as described for the example 1 (Scheme 1. General procedure A.).

¹H NMR (600 MHz, CD₃OD) δ [ppm]=1.06 (d, 3H), 1.09 (d, 3H), 2.10 (br, 1H), 3.73-3.76 (m, 1H), 3.80-3.83 (m, 1H), 4.41 (br, 1H), 7.02 (s, 1H), 7.54 (d, 2H), 8.06 (br, 1H), 8.14 (d, 2H), 8.88 (br, 1H), 9.38 (d, 1H), 9.69 (br, 1H); MS (ESI, m/z): 369.2 [M+H]⁺

### Example 14. (R)-2-((6-(4-chlorophenyl)-2-(pyridin-3-yl)pyrimidin-4-yl)amino)-3-methylbutan-1-ol

Using (R)-2-amino-3-methylbutan-1-ol and separation method of PREP. HPLC, the title compound was obtained as described for the example 1 (Scheme 1. General procedure A.).

¹H NMR (600 MHz, CD₃OD) δ [ppm]=1.06 (d, 3H), 1.09 (d, 3H), 2.10 (br, 1H), 3.73-3.76 (m, 1H), 3.80-3.83 (m, 1H), 4.41 (br, 1H), 7.02 (s, 1H), 7.54 (d, 2H), 8.06 (br, 1H), 8.14 (d, 2H), 8.88 (br, 1H), 9.38 (d, 1H), 9.69 (br, 1H); MS (ESI, m/z): 369.2 [M+H]⁺

### Example 15. 2-((6-(4-chlorophenyl)-2-(pyridin-3-yl)pyrimidin-4-yl)amino)butan-1-ol

Using 2-aminobutan-1-ol and separation method of PREP. HPLC, the title compound was obtained as described for the example 1 (Scheme 1. General procedure A.).

¹H NMR (400 MHz, CD₃OD) δ [ppm]=1.05 (t, 3H), 1.59-1.64 (m, 1H), 1.83-1.86 (m, 1H), 3.70 (d, 2H), 4.39 (br, 1H), 6.96 (s, 1H), 7.50 (d, 2H), 8.10-8.13 (m, 3H), 8.90 (d, 1H), 9.44 (d, 1H), 9.69 (d, 1H); MS (ESI, m/z): 355.1 [M+H]⁺

### Example 16. 2-((6-(4-chlorophenyl)-2-(pyridin-3-yl)pyrimidin-4-yl)amino)propane-1,3-diol

Using 2-aminopropane-1,3-diol and separation method of PREP. HPLC, the title compound was obtained as described for the example 1 (Scheme 1. General procedure A.).

¹H NMR (600 MHz, CD₃OD) δ [ppm]=3.81 (d, 4H), 4.54 (br, 1H), 7.02 (s, 1H), 7.51 (d, 2H), 8.06 (br, 1H), 8.13 (d, 2H), 8.87 (br, 1H), 9.43 (d, 1H), 9.70 (br, 1H); MS (ESI, m/z): 357.1 [M+H]⁺

### Example 17. (R)-2-((6-(4-chlorophenyl)-2-(pyridin-3-yl)pyrimidin-4-yl)amino)-1-phenylethan-1-ol

Using (R)-2-amino-1-phenylethan-1-ol and separation method of PREP. HPLC, the title compound was obtained as described for the example 1 (Scheme 1. General procedure A.).

¹H NMR (600 MHz, CD₃OD) δ [ppm]=3.77 (br, 1H), 3.91 (br, 1H), 4.95 (br, 1H), 6.95 (s, 1H), 7.25 (t, 1H), 7.35 (t, 2H), 7.47 (d, 2H), 7.53 (d, 2H), 8.00 (br, 1H), 8.13 (d, 2H), 8.86 (br, 1H), 9.28 (d, 1H), 9.68 (br, 1H); MS (ESI, m/z): 403.1 [M+H]⁺

### Example 18. (S)-2-((6-(4-chlorophenyl)-2-(pyridin-3-yl)pyrimidin-4-yl)amino)-1-phenylethan-1-ol

Using (S)-2-amino-1-phenylethan-1-ol and separation method of PREP. HPLC, the title compound was obtained as described for the example 1 (Scheme 1. General procedure A.).

¹H NMR (600 MHz, CD₃OD) δ [ppm]=3.77 (br, 1H), 3.91 (br, 1H), 4.95 (br, 1H), 6.95 (s, 1H), 7.25 (t, 1H), 7.35 (t, 2H), 7.47 (d, 2H), 7.53 (d, 2H), 8.00 (br, 1H), 8.13 (d, 2H), 8.86 (br, 1H), 9.28 (d, 1H), 9.68 (br, 1H); MS (ESI, m/z): 403.1 [M+H]⁺

### Example 19. 6-(4-chlorophenyl)-2-(pyridin-3-yl)-N-((tetrahydro-2H-pyran-4-yl)methyl)pyrimidin-4-amine

Using (tetrahydro-2H-pyran-4-yl)methanamine and separation method of PREP. HPLC, the title compound was obtained as described for the example 1 (Scheme 1. General procedure A.).

¹H NMR (600 MHz, CD₃OD) δ [ppm]=1.38-1.45 (m, 2H), 1.77 (d, 2H), 1.95-2.02 (m, 1H), 3.44 (t, 2H), 3.54 (br, 2H), 3.98 (d, 2H), 6.95 (s, 1H), 7.54 (d, 2H), 8.06 (br, 1H), 8.14 (d, 2H), 8.87 (br, 1H), 9.36 (d, 1H), 9.73 (br, 1H); MS (ESI, m/z): 381.1 [M+H]⁺

### Example 20. N¹-(6-(4-chlorophenyl)-2-(pyridin-3-yl)pyrimidin-4-yl)-N³,N³-dimethylpropane-1,3-diamine

Using N¹,N¹-dimethylpropane-1,3-diamine and separation method of PREP. HPLC, the title compound was obtained as described for the example 1 (Scheme 1. General procedure A.).

¹H NMR (600 MHz, CD₃OD) δ [ppm]=2.14-2.21 (m, 2H), 2.93 (s, 6H), 3.04-3.06 (m, 1H), 3.23-3.26 (m, 1H), 3.74 (br, 2H), 7.02 (s, 1H), 7.56 (d, 2H), 8.08 (br, 1H), 8.19 (d, 2H), 8.90 (br, 1H), 9.44 (d, 1H), 9.77 (br, 1H); MS (ESI, m/z): 368.2 [M+H]⁺

### Example 21. 6-(4-chlorophenyl)-N-ethyl-2-(pyridin-3-yl)pyrimidin-4-amine

Using ethanamine and separation method of PREP. HPLC, the title compound was obtained as described for the example 1 (Scheme 1. General procedure A.).

¹H NMR: (400 MHz, CD₃OD) δ [ppm]=1.32 (t, 3H), 3.60 (br, 2H), 6.91 (s, 1H), 7.53 (d, 2H), 7.97 (br, 1H), 8.14 (d, 2H), 8.85 (br, 1H), 9.27 (d, 1H), 9.71 (br, 1H); MS (ESI, m/z): 311.1 [M+H]⁺

### Example 22. 6-(4-chlorophenyl)-N-propyl-2-(pyridin-3-yl)pyrimidin-4-amine

Using propan-1-amine and separation method of PREP. HPLC, the title compound was obtained as described for the example 1 (Scheme 1. General procedure A.).

¹H NMR (400 MHz, CD₃OD) δ [ppm]=1.05 (t, 3H), 1.74 (q, 2H), 3.56 (br, 2H), 6.93 (s, 1H), 7.53 (d, 2H), 8.01 (br, 1H), 8.13 (d, 2H), 8.84 (br, 1H), 9.32 (d, 1H), 9.69 (br, 1H); MS (ESI, m/z): 325.1 [M+H]⁺

### Example 23. N-butyl-6-(4-chlorophenyl)-2-(pyridin-3-yl)pyrimidin-4-amine

Using butan-1-amine and separation method of PREP. HPLC, the title compound was obtained as described for the example 1 (Scheme 1. General procedure A.).

¹H NMR (400 MHz, CD₃OD) δ [ppm]=1.01 (t, 3H), 1.45-1.54 (m, 2H), 1.66-1.73 (m, 2H), 3.58 (br, 2H), 6.89 (s, 1H), 7.52 (d, 2H), 7.90 (br, 1H), 8.12 (d, 2H), 8.84 (br, 1H), 9.18 (d, 1H), 9.69 (br, 1H); MS (ESI, m/z): 339.1 [M+H]⁺

### Example 24. 1-((6-(4-chlorophenyl)-2-(pyridin-3-yl)pyrimidin-4-yl)amino)butan-2-ol

Using 1-aminobutan-2-ol and separation method of PREP. HPLC, the title compound was obtained as described for the example 1 (Scheme 1. General procedure A.).

¹H NMR (400 MHz, CD₃OD) δ [ppm]=1.01 (t, 3H), 1.45-1.54 (m, 2H), 1.66-1.73 (m, 2H), 3.58 (br, 2H), 6.89 (s, 1H), 7.52 (d, 2H), 7.90 (br, 1H), 8.12 (d, 2H), 8.84 (br, 1H), 9.18 (d, 1H), 9.69 (br, 1H); MS (ESI, m/z): 355.1 [M+H]⁺

### Example 25. 6-(4-chlorophenyl)-N-(cyclopropylmethyl)-2-(pyridin-3-yl)pyrimidin-4-amine

Using cyclopropylmethanamine and separation method of PREP. HPLC, the title compound was obtained as described for the example 1 (Scheme 1. General procedure A.).

¹H NMR (400 MHz, CD₃OD) δ [ppm]=0.32-0.36 (m, 2H), 0.57-0.61 (m, 2H), 1.14-1.26 (m, 1H), 3.46 (br, 2H), 6.93 (s, 1H), 7.53 (d, 2H), 8.05 (br, 1H), 8.13 (d, 2H), 8.97 (br, 1H), 9.29 (d, 1H), 9.82 (br, 1H); MS (ESI, m/z): 337.1 [M+H]⁺

### Example 26. 6-(4-chlorophenyl)-N-cyclopentyl-2-(pyridin-3-yl)pyrimidin-4-amine

Using cyclopentanamine and separation method of PREP. HPLC, the title compound was obtained as described for the example 1 (Scheme 1. General procedure A.).

¹H NMR (400 MHz, CD₃OD) δ [ppm]=1.59-1.69 (m, 2H), 1.70-1.77 (m, 2H), 1.79-1.89 (m, 2H), 2.12-2.20 (m, 2H), 4.59 (br, 1H), 6.93 (s, 1H), 7.54 (d, 2H), 8.08 (br, 1H), 8.13 (d, 2H), 8.89 (br, 1H), 9.38 (d, 1H), 9.72 (br, 1H); MS (ESI, m/z): 351.1 [M+H]⁺

### Example 27. 4-(4-chlorophenyl)-6-(4-methylpiperidin-1-yl)-2-(pyridin-3-yl)pyrimidine

Using 4-methylpiperidine and separation method of PREP. HPLC, the title compound was obtained as described for the example 1 (Scheme 1. General procedure A.).

¹H NMR (400 MHz, CD₃OD) δ [ppm]=1.02 (d, 3H), 1.18-1.31 (m, 2H), 1.74-1.92 (m, 3H), 3.09 (t, 2H), 3.33-3.38 (m, 2H), 7.23 (s, 1H), 7.53 (d, 2H), 8.09 (br, 1H), 8.20 (d, 2H), 8.94 (br, 1H), 9.38 (d, 1H), 9.76 (br, 1H); MS (ESI, m/z): 365.2 [M+H]⁺

### Example 28. N-(tert-butyl)-6-(4-chlorophenyl)-2-(pyridin-3-yl)pyrimidin-4-amine

Using 2-methylpropan-2-amine and separation method of PREP. HPLC, the title compound was obtained as described for the example 1 (Scheme 1. General procedure A.).

¹H NMR (600 MHz, CD₃OD) δ [ppm]=4.64 (s, 9H), 7.63 (d, 2H), 7.66 (br, 1H), 8.10 (s, 1H), 8.36 (d, 2H), 8.75 (br, 1H), 8.94 (d, 1H), 9.67 (br, 1H); MS (ESI, m/z): 339.1 [M+H]⁺

### Example 29. (1R,2R)-2-((6-(4-chlorophenyl)-2-(pyridin-3-yl)pyrimidin-4-yl)amino)cyclopentan-1-ol

Using (1R,2R)-2-aminocyclopentan-1-ol and separation method of PREP. HPLC, the title compound was obtained as described for the example 1 (Scheme 1. General procedure A.).

¹H NMR (400 MHz, CD₃OD) δ [ppm]=1.61-1.75 (m, 2H), 1.79-1.96 (m, 2H), 1.98-2.07 (m, 1H), 2.27-2.35 (m, 1H), 4.16 (br, 1H), 4.44 (br, 1H), 6.97 (s, 1H), 7.53 (d, 2H), 8.07 (br, 1H), 8.14 (d, 2H), 8.88 (br, 1H), 9.45 (d, 1H), 9.73 (br, 1H); MS (ESI, m/z): 367.1 [M+H]⁺

### Example 30. (1S,2R)-2-((6-(4-chlorophenyl)-2-(pyridin-3-yl)pyrimidin-4-yl)amino)cyclopentan-1-ol

Using (1S,2R)-2-aminocyclopentan-1-ol and separation method of PREP. HPLC, the title compound was obtained as described for the example 1 (Scheme 1. General procedure A.).

¹H NMR (600 MHz, CD₃OD) δ [ppm]=1.67-1.82 (m, 3H), 1.91-1.97 (m, 1H), 2.00-2.07 (m, 1H), 2.13 (br, 1H), 4.37 (br, 1H), 4.49 (br, 1H), 7.04 (s, 1H), 7.53 (d, 2H), 7.70 (br, 3H), 8.86 (br, 1H), 9.35 (d, 1H), 9.67 (br, 1H); MS (ESI, m/z): 367.1 [M+H]⁺

### Example 31. 6-(4-chlorophenyl)-N-(pyridin-2-ylmethyl)-2-(pyridin-3-yl)pyrimidin-4-amine

Using pyridin-2-ylmethanamine and separation method of PREP. HPLC, the title compound was obtained as described for the example 1 (Scheme 1. General procedure A.).

¹H NMR (600 MHz, CD₃OD) δ [ppm]=5.16 (s, 2H), 7.24 (s, 1H), 7.54 (d, 2H), 7.88 (t, 1H), 8.07-8.11 (m, 2H), 8.20 (d, 2H), 8.49 (t, 1H), 8.76 (d, 1H), 8.88 (d, 1H), 9.32 (d, 1H), 9.57 (s, 1H); MS (ESI, m/z): 374.1 [M+H]⁺

### Example 32. 6-(4-chlorophenyl)-2-(pyridin-3-yl)-N-(pyridin-3-ylmethyl)pyrimidin-4-amine

Using pyridin-3-ylmethanamine and separation method of PREP. HPLC, the title compound was obtained as described for the example 1 (Scheme 1. General procedure A.).

¹H NMR (600 MHz, CD₃OD) δ [ppm]=5.04 (s, 2H), 7.15 (s, 1H), 7.53 (d, 2H), 8.06 (br, 1H), 8.14 (br, 1H), 8.19 (d, 2H), 8.67 (d, 1H), 8.79 (br, 1H), 8.97 (br, 2H), 9.43 (d, 1H), 9.71 (s, 1H); MS (ESI, m/z): 374.1 [M+H]⁺

### Example 33. 6-(4-chlorophenyl)-2-(pyridin-3-yl)-N-(pyridin-4-ylmethyl)pyrimidin-4-amine

Using pyridin-4-ylmethanamine and separation method of PREP. HPLC, the title compound was obtained as described for the example 1 (Scheme 1. General procedure A.).

¹H NMR (600 MHz, CD₃OD) δ [ppm]=5.12 (s, 2H), 7.23 (s, 1H), 7.55 (d, 2H), 8.09 (br, 1H), 8.13 (d, 2H), 8.21 (d, 2H), 8.79 (d, 2H), 8.88 (br, 1H), 9.34 (d, 1H), 9.59 (s, 1H); MS (ESI, m/z): 374.1 [M+H]⁺

### Example 34. trans-4-((6-(4-chlorophenyl)-2-(pyridin-3-yl)pyrimidin-4-yl)amino)cyclohexan-1-ol

Using trans-4-aminocyclohexan-1-ol and separation method of PREP. HPLC, the title compound was obtained as described for the example 1 (Scheme 1. General procedure A.).

¹H NMR (600 MHz, CD₃OD) δ [ppm]=1.39-1.46 (m, 2H), 1.48-1.54 (m, 2H), 2.02-2.07 (m, 2H), 2.14-2.19 (m, 2H), 3.62-3.67 (m, 1H), 4.14 (br, 1H), 6.90 (s, 1H), 7.53 (d, 2H), 7.99 (br, 1H), 8.13 (d, 2H), 8.83 (br, 1H), 9.28 (d, 1H), 9.66 (br, 1H); MS (ESI, m/z): 381.1 [M+H]⁺

### Example 35. trans-2-((6-(4-chlorophenyl)-2-(pyridin-3-yl)pyrimidin-4-yl)amino)cyclohexan-1-ol

Using trans-2-aminocyclohexan-1-ol and separation method of PREP. HPLC, the title compound was obtained as described for the example 1 (Scheme 1. General procedure A.).

¹H NMR (600 MHz, CD₃OD) δ [ppm]=1.34-1.52 (m, 4H), 1.77-1.84 (m, 2H), 2.08-2.16 (m, 2H), 3.55 (br, 1H), 4.19 (br, 1H), 6.95 (s, 1H), 7.53 (d, 2H), 8.08 (br, 1H), 8.12 (d, 2H), 8.89 (br, 1H), 9.37 (d, 1H), 9.67 (br, 1H); MS (ESI, m/z): 381.1 [M+H]⁺

### Example 36. (1-(6-(4-chlorophenyl)-2-(pyridin-3-yl)pyrimidin-4-yl)piperidin-2-yl)methanol

Using piperidin-2-ylmethanol and separation method of PREP. HPLC, the title compound was obtained as described for the example 1 (Scheme 1. General procedure A.).

¹H NMR (400 MHz, CD₃OD) δ [ppm]=1.54-1.66 (m, 1H), 1.67-1.81 (m, 3H), 1.84-1.92 (m, 1H), 1.98-2.05 (m, 1H), 3.11-3.21 (m, 1H), 3.79-3.91 (m, 2H), 4.21-4.40 (m, 1H), 4.70 (br, 1H), 7.26 (s, 1H), 7.53 (d, 2H), 8.12 (br, 1H), 8.19 (d, 2H), 8.92 (br, 1H), 9.45 (d, 1H), 9.73 (br, 1H); MS (ESI, m/z): 381.1 [M+H]⁺

### Example 37. 2-(1-(6-(4-chlorophenyl)-2-(pyridin-3-yl)pyrimidin-4-yl)piperidin-2-yl)ethan-1-ol

Using 2-(piperidin-2-yl)ethan-1-ol and separation method of PREP. HPLC, the title compound was obtained as described for the example 1 (Scheme 1. General procedure A.).

¹H NMR (400 MHz, CD₃OD) δ [ppm]=1.29-1.35 (m, 2H), 1.53-1.65 (m, 1H), 1.70-1.76 (m, 1H), 1.78-1.90 (m, 4H), 1.90-1.97 (m, 1H), 2.07-2.16 (m, 1H), 3.10-3.20 (m, 1H), 3.57-3.69 (m, 2H), 7.31 (s, 1H), 7.54 (d, 2H), 8.03 (br, 1H), 8.20 (d, 2H), 8.87 (br, 1H), 9.37 (d, 1H), 9.71 (br, 1H); MS (ESI, m/z): 395.2 [M+H]⁺

### Example 38. (R)-1-(6-(4-chlorophenyl)-2-(pyridin-3-yl)pyrimidin-4-yl)piperidin-3-ol

Using (R)-piperidin-3-ol and separation method of PREP. HPLC, the title compound was obtained as described for the example 1 (Scheme 1. General procedure A.).

¹H NMR (400 MHz, CD₃OD) δ [ppm]=1.56-1.73 (m, 2H), 1.90-1.99 (m, 1H), 2.02-2.09 (m, 1H), 4.47 (dd, 1H), 3.63 (br, 1H), 3.77-3.82 (m, 1H), 4.04 (br, 1H), 4.29 (br, 1H), 7.21 (s, 1H), 7.50 (d, 2H), 8.13 (br, 1H), 8.17 (d, 2H), 8.95 (br, 1H), 9.43 (d, 1H), 9.73 (br, 1H); MS

### Example 39. 1-(6-(4-chlorophenyl)-2-(pyridin-3-yl)pyrimidin-4-yl)piperidin-3-ol

Using piperidin-3-ol and separation method of PREP. HPLC, the title compound was obtained as described for the example 1 (Scheme 1. General procedure A.).

¹H NMR (400 MHz, CD₃OD) δ [ppm]=1.56-1.73 (m, 2H), 1.90-1.99 (m, 1H), 2.02-2.09 (m, 1H), 4.47 (dd, 1H), 3.63 (br, 1H), 3.77-3.82 (m, 1H), 4.04 (br, 1H), 4.29 (br, 1H), 7.21 (s, 1H), 7.50 (d, 2H), 8.13 (br, 1H), 8.17 (d, 2H), 8.95 (br, 1H), 9.43 (d, 1H), 9.73 (br, 1H); MS (ESI, m/z): 367.1 [M+H]⁺

### Example 40. (1-(6-(4-chlorophenyl)-2-(pyridin-3-yl)pyrimidin-4-yl)piperidin-3-yl)methanol

Using piperidin-3-ylmethanol and separation method of PREP. HPLC, the title compound was obtained as described for the example 1 (Scheme 1. General procedure A.).

¹H NMR (400 MHz, CD₃OD) δ [ppm]=1.35-1.46 (m, 1H), 1.56-1.68 (m, 1H), 1.78-1.95 (m, 4H), 3.02 (dd, 1H), 3.23 (dd, 1H), 3.63 (br, 1H), 3.38-3.46 (m, 1H), 3.47-3.61 (m, 2H), 7.24 (s, 1H), 7.52 (d, 2H), 8.10 (dd, 1H), 8.19 (d, 2H), 8.89 (br, 1H), 9.45 (d, 1H), 9.71 (br, 1H); MS (ESI, m/z): 381.1 [M+H]⁺

### Example 41. 1-(6-(4-chlorophenyl)-2-(pyridin-3-yl)pyrimidin-4-yl)piperidin-4-ol

Using piperidin-4-ol and separation method of PREP. HPLC, the title compound was obtained as described for the example 1 (Scheme 1. General procedure A.).

¹H NMR (400 MHz, CD₃OD) δ [ppm]=1.54-1.62 (m, 2H), 1.96-2.03 (m, 2H), 3.46-3.53 (m, 2H), 3.93-3.99 (m, 2H), 4.36 (br, 2H), 7.23 (s, 1H), 7.50 (d, 2H), 8.10 (br, 1H), 8.18 (d, 2H), 8.89 (br, 1H), 9.43 (d, 1H), 9.70 (br, 1H); MS (ESI, m/z): 367.1 [M+H]⁺

### Example 42. (1-(6-(4-chlorophenyl)-2-(pyridin-3-yl)pyrimidin-4-yl)piperidin-4-yl)methanol

Using piperidin-4-ylmethanol and separation method of PREP. HPLC, the title compound was obtained as described for the example 1 (Scheme 1. General procedure A.).

¹H NMR (400 MHz, CD₃OD) δ [ppm]=1.24-1.34 (m, 2H), 1.82-1.94 (m, 3H), 3.08 (t, 2H), 3.47 (d, 2H), 4.77 (br, 2H), 7.21 (s, 1H), 7.50 (d, 2H), 8.11 (br, 1H), 8.17 (d, 2H), 8.90 (br, 1H), 9.43 (d, 1H), 9.69 (br, 1H); MS (ESI, m/z): 381.1 [M+H]⁺

### Example 43. 2-(1-(6-(4-chlorophenyl)-2-(pyridin-3-yl)pyrimidin-4-yl)piperidin-4-yl)ethan-1-ol

Using 2-(piperidin-4-yl)ethan-1-ol and separation method of PREP. HPLC, the title compound was obtained as described for the example 1 (Scheme 1. General procedure A.).

¹H NMR (400 MHz, CD₃OD) δ [ppm]=1.22-1.32 (m, 2H), 1.51-1.56 (m, 2H), 1.81-1.93 (m, 3H), 3.07 (t, 2H), 3.67 (t, 2H), 4.72 (br, 2H), 7.20 (s, 1H), 7.51 (d, 2H), 8.10 (br, 1H), 8.17 (d, 2H), 8.89 (br, 1H), 9.41 (d, 1H), 9.68 (br, 1H); MS (ESI, m/z): 395.2 [M+H]⁺

### Example 44. 3-(1-(6-(4-chlorophenyl)-2-(pyridin-3-yl)pyrimidin-4-yl)piperidin-4-yl)propan-1-ol

Using 3-(piperidin-4-yl)propan-1-ol and separation method of PREP. HPLC, the title compound was obtained as described for the example 1 (Scheme 1. General procedure A.).

¹H NMR (400 MHz, CD₃OD) δ [ppm]=1.18-1.29 (m, 2H), 1.34-1.39 (m, 2H), 1.58-1.73 (m, 3H), 1.93 (d, 2H), 3.07 (t, 2H), 3.57 (t, 2H), 4.75 (br, 2H), 7.22 (s, 1H), 7.52 (d, 2H), 8.11 (br, 1H), 8.18 (d, 2H), 8.90 (br, 1H), 9.42 (d, 1H), 9.70 (br, 1H); MS (ESI, m/z): 409.2 [M+H]⁺

### Example 45. 4-(4-chlorophenyl)-6-(4-methoxypiperidin-1-yl)-2-(pyridin-3-yl)pyrimidine

Using 4-methoxypiperidine and separation method of PREP. HPLC, the title compound was obtained as described for the example 1 (Scheme 1. General procedure A.).

¹H NMR (400 MHz, CD₃OD) δ [ppm]=1.60-1.68 (m, 2H), 1.98-2.05 (m, 2H), 3.42 (s, 3H), 3.57-3.65 (m, 3H), 4.21 (br, 2H), 7.22 (s, 1H), 7.50 (d, 2H), 8.09 (br, 1H), 8.18 (d, 2H), 8.89 (br, 1H), 9.41 (d, 1H), 9.69 (br, 1H); MS (ESI, m/z): 381.1 [M+H]⁺

### Example 46. 4-(4-chlorophenyl)-6-(piperidin-1-yl)-2-(pyridin-3-yl)pyrimidine

Using piperidine and separation method of PREP. HPLC, the title compound was obtained as described for the example 1 (Scheme 1. General procedure A.).

¹H NMR (400 MHz, CD₃OD) δ [ppm]=1.71 (br, 4H), 1.78 (br, 2H), 3.90 (br, 4H), 7.21 (s, 1H), 7.53 (d, 2H), 8.03 (br, 1H), 8.19 (d, 2H), 8.88 (br, 1H), 9.33 (d, 1H), 9.69 (br, 1H); MS (ESI, m/z): 351.1 [M+H]⁺

### Example 47. 4-(4-chlorophenyl)-6-(2-methylpiperidin-1-yl)-2-(pyridin-3-yl)pyrimidine

Using 2-methylpiperidine and separation method of PREP. HPLC, the title compound was obtained as described for the example 1 (Scheme 1. General procedure A.).

¹H NMR (400 MHz, CD₃OD) δ [ppm]=1.33 (d, 3H), 1.53-1.63 (m, 1H), 1.70-1.91 (m, 5H), 3.14-3.21 (m, 1H), 4.60 (br, 1H), 5.06 (br, 1H), 7.20 (s, 1H), 7.53 (d, 2H), 8.13 (br, 1H), 8.19 (d, 2H), 8.96 (br, 1H), 9.39 (d, 1H), 9.75 (br, 1H); MS (ESI, m/z): 365.2 [M+H]⁺

### Example 48. 4-(4-chlorophenyl)-6-(3-methylpiperidin-1-yl)-2-(pyridin-3-yl)pyrimidine

Using 3-methylpiperidine and separation method of PREP. HPLC, the title compound was obtained as described for the example 1 (Scheme 1. General procedure A.).

¹H NMR (400 MHz, CD₃OD) δ [ppm]=1.04 (d, 3H), 1.28-1.35 (m, 1H), 1.56-1.76 (m, 2H), 1.83-1.96 (m, 2H), 2.81 (dd, 1H), 3.12 (dd, 1H), 4.59 (br, 2H), 7.23 (s, 1H), 7.53 (d, 2H), 8.11 (br, 1H), 8.19 (d, 2H), 8.91 (br, 1H), 9.42 (d, 1H), 9.70 (br, 1H), 9.75 (br, 1H); MS (ESI, m/z): 365.2 [M+H]⁺

### Example 49. 4-(4-chlorophenyl)-6-(2,6-dimethylpiperidin-1-yl)-2-(pyridin-3-yl)pyrimidine

Using 2,6-dimethylpiperidine and separation method of PREP. HPLC, the title compound was obtained as described for the example 1 (Scheme 1. General procedure A.).

MS (ESI, m/z): 379.2 [M+H]⁺

### Example 50. 4-(4-chlorophenyl)-6-(3,5-dimethylpiperidin-1-yl)-2-(pyridin-3-yl)pyrimidine

Using 3,5-dimethylpiperidine and separation method of PREP. HPLC, the title compound was obtained as described for the example 1 (Scheme 1. General procedure A.).

¹H NMR (400 MHz, CD₃OD) δ [ppm]=0.94 (q, 1H), 1.03 (d, 6H), 1.59-1.73 (m, 3H), 1.91 (d, 1H), 2.51 (t, 2H), 4.68 (br, 1H), 7.21 (s, 1H), 7.51 (d, 2H), 8.11 (br, 1H), 8.18 (d, 2H), 8.94 (br, 1H), 9.39 (d, 1H), 9.73 (br, 1H); MS (ESI, m/z): 379.2 [M+H]⁺

### Example 51. 4-(4-chlorophenyl)-6-(3,3-difluoropiperidin-1-yl)-2-(pyridin-3-yl)pyrimidine

Using 3,3-difluoropiperidine and separation method of PREP. HPLC, the title compound was obtained as described for the example 1 (Scheme 1. General procedure A.).

¹H NMR (400 MHz, CD₃OD) δ [ppm]=1.87-1.93 (m, 2H), 2.14-2.24 (m, 2H), 3.92 (br, 2H), 4.23 (t, 2H), 7.32 (s, 1H), 7.51 (d, 2H), 8.06 (br, 1H), 8.23 (d, 2H), 8.87 (br, 1H), 9.40 (d, 1H), 9.72 (br, 1H); MS (ESI, m/z): 387.1 [M+H]⁺

### Example 52. 4-(4-chlorophenyl)-2-(pyridin-3-yl)-6-(3-(trifluoromethyl)piperidin-1-yl)pyrimidine

Using 3-(trifluoromethyl)piperidine and separation method of PREP. HPLC, the title compound was obtained as described for the example 1 (Scheme 1. General procedure A.).

¹H NMR (400 MHz, CD₃OD) δ [ppm]=1.59-1.82 (m, 2H), 1.94-1.97 (m, 1H), 2.12-2.15 (m, 1H), 2.44-2.56 (m, 1H), 3.19-3.27 (m, 2H), 4.48 (br, 1H), 4.95 (br, 1H), 7.28 (s, 1H), 7.52 (d, 2H), 8.07 (br, 1H), 8.23 (d, 2H), 8.88 (br, 1H), 9.37 (d, 1H), 9.70 (br, 1H); MS (ESI, m/z): 419.1 [M+H]⁺

### Example 53. 4-(4-chlorophenyl)-6-(3-ethylpiperidin-1-yl)-2-(pyridin-3-yl)pyrimidine

Using 3-(trifluoromethyl)piperidine and separation method of PREP. HPLC, the title compound was obtained as described for the example 1 (Scheme 1. General procedure A.).

MS (ESI, m/z): 379.2 [M+H]⁺

### Example 54. 6-(4-chlorophenyl)-N-(piperidin-4-yl)-2-(pyridin-3-yl)pyrimidin-4-amine

To a solution of 4-chloro-6-(4-chlorophenyl)-2-(pyridin-3-yl)pyrimidine (15 mg, 0.05 mmol) in tetrahydrofuran (2 mL) was added triethylamine (0.06 mL, 0.43 mmol) followed by tert-butyl 4-aminopiperidine-1-carboxylate (20 mg, 0.10 mmol) at room temperature. The reaction mixture in sealed tube was heated at 120° C. for 4 h. and cooled to room temperature.

The residue was filtered, evaporated in vacuo. To a solution of the reaction mixture in ethanol (1 mL) was added 1M hydrochloric acid ethanol solution (2 mL). The reaction mixture was stirred at 50° C. for overnight, evaporated in vacuo and quenched with 1M ammonium acetate methanol solution (2 mL). The residue was filtered and purified by Preparative HPLC to give 7.6 mg of the title compound (Scheme 1. General procedure A.).

¹H NMR (400 MHz, CD₃OD) δ [ppm]=1.81-1.92 (m, 2H), 2.37 (d, 2H), 3.26 (d, 2H), 3.52 (d, 2H), 4.47 (br, 1H), 7.00 (s, 1H), 7.54 (d, 2H), 8.07 (br, 1H), 8.16 (d, 2H), 8.88 (br, 1H), 9.43 (d, 1H), 9.76 (br, 1H); MS (ESI, m/z): 366.1 [M+H]⁺

### Example 55. 6-(4-chlorophenyl)-N-(piperidin-3-ylmethyl)-2-(pyridin-3-yl)pyrimidin-4-amine

Using tert-butyl 3-(aminomethyl)piperidine-1-carboxylate, the title compound was obtained as described for the example 54 (Scheme 1. General procedure A.).

¹H NMR (400 MHz, CD₃OD) δ [ppm]=1.39-1.48 (m, 1H), 1.70-1.82 (m, 1H), 2.02 (t, 2H), 2.26 (br, 1H), 2.83 (t, 1H), 2.94 (t, 1H), 3.37 (d, 1H), 3.50 (d, 1H), 3.62 (br, 2H), 6.99 (s, 1H), 7.53 (d, 2H), 8.06 (br, 1H), 8.16 (d, 2H), 8.88 (br, 1H), 9.41 (d, 1H), 9.75 (br, 1H); MS (ESI, m/z): 380.2 [M+H]⁺

### Example 56. 6-(4-chlorophenyl)-N-(piperidin-4-ylmethyl)-2-(pyridin-3-yl)pyrimidin-4-amine

Using tert-butyl 4-(aminomethyl)piperidine-1-carboxylate, the title compound was obtained as described for the example 54 (Scheme 1. General procedure A.).

¹H NMR (400 MHz, CD₃OD) δ [ppm]=1.29-1.40 (m, 1H), 1.45-1.55 (m, 1H), 2.01 (t, 4H), 2.91 (d, 2H), 2.98-3.14 (m, 2H), 3.45 (d, 1H), 7.26 (s, 1H), 7.52 (d, 2H), 8.10 (br, 1H), 8.21 (d, 2H), 8.89 (br, 1H), 9.42 (d, 1H), 9.72 (br, 1H); MS (ESI, m/z): 380.2 [M+H]⁺

### Example 57. 6-(4-chlorophenyl)-N-(1-methylpiperidin-4-yl)-2-(pyridin-3-yl)pyrimidin-4-amine

Using 1-methylpiperidin-4-amine and separation method of PREP. HPLC, the title compound was obtained as described for the example 1 (Scheme 1. General procedure A.).

¹H NMR (400 MHz, CD₃OD) δ [ppm]=1.83-2.04 (m, 2H), 2.18-2.29 (m, 2H), 2.89 (s, 3H), 3.10-3.24 (m, 2H), 3.42-3.54 (m, 2H), 3.63 (br, 1H), 6.97 (s, 1H), 7.53 (d, 2H), 8.06 (br, 1H), 8.15 (d, 2H), 8.88 (br, 1H), 9.42 (d, 1H), 9.75 (br, 1H); MS (ESI, m/z): 380.2 [M+H]⁺

### Example 58. 6-(4-chlorophenyl)-N-(2-(piperidin-4-yl)ethyl)-2-(pyridin-3-yl)pyrimidin-4-amine

Using tert-butyl 4-(2-aminoethyl)piperidine-1-carboxylate, the title compound was obtained as described for the example 54 (Scheme 1. General procedure A.).

¹H NMR (400 MHz, CD₃OD) δ [ppm]=1.42-1.52 (m, 2H), 1.70-1.86 (m, 3H), 2.07 (d, 2H), 2.98 (t, 2H), 3.40 (d, 2H), 3.68 (br, 2H), 6.94 (s, 1H), 7.54 (d, 2H), 8.00 (br, 1H), 8.14 (d, 2H), 8.86 (br, 1H), 9.32 (d, 1H), 9.69 (br, 1H); MS (ESI, m/z): 394.2 [M+H]⁺

### Example 59. (1-(6-(4-chlorophenyl)-2-(pyridin-3-yl)pyrimidin-4-yl)piperidin-2-yl)methanamine

Using tert-butyl (piperidin-2-ylmethyl)carbamate, the title compound was obtained as described for the example 54 (Scheme 1. General procedure A.).

¹H NMR (400 MHz, CD₃OD) δ [ppm]=1.63 (br, 2H), 1.76-1.91 (m, 5H), 3.24-3.33 (m, 2H), 3.61 (t, 2H), 4.47 (br, 1H), 5.50 (br, 1H), 7.37 (s, 1H), 7.55 (d, 2H), 8.07 (br, 1H), 8.27 (d, 2H), 8.89 (br, 1H), 9.47 (d, 1H), 9.81 (br, 1H); MS (ESI, m/z): 380.2 [M+H]⁺

### Example 60. (R)-1-(6-(4-chlorophenyl)-2-(pyridin-3-yl)pyrimidin-4-yl)piperidin-3-amine

Using tert-butyl (R)-piperidin-3-ylcarbamate, the title compound was obtained as described for the example 54 (Scheme 1. General procedure A.).

¹H NMR (400 MHz, CD₃OD) δ [ppm]=1.72-1.88 (m, 2H), 1.93-1.99 (m, 1H), 2.19-2.26 (m, 1H), 3.40-3.46 (m, 1H), 3.51-3.57 (m, 1H), 3.62-3.67 (m, 1H), 4.18 (d, 1H), 4.71 (d, 1H), 7.33 (s, 1H), 7.51 (d, 2H), 8.05 (br, 1H), 8.25 (d, 2H), 8.89 (br, 1H), 9.42 (d, 1H), 9.80 (br, 1H); MS (ESI, m/z): 366.1 [M+H]⁺

### Example 61. (S)-1-(6-(4-chlorophenyl)-2-(pyridin-3-yl)pyrimidin-4-yl)piperidin-3-amine

Using tert-butyl (S)-piperidin-3-ylcarbamate, the title compound was obtained as described for the example 54 (Scheme 1. General procedure A.).

¹H NMR (400 MHz, CD₃OD) δ [ppm]=1.72-1.88 (m, 2H), 1.93-1.99 (m, 1H), 2.19-2.26 (m, 1H), 3.40-3.46 (m, 1H), 3.51-3.57 (m, 1H), 3.62-3.67 (m, 1H), 4.18 (d, 1H), 4.71 (d, 1H), 7.33 (s, 1H), 7.51 (d, 2H), 8.05 (br, 1H), 8.25 (d, 2H), 8.89 (br, 1H), 9.42 (d, 1H), 9.80 (br, 1H); MS (ESI, m/z): 366.1 [M+H]⁺

### Example 62. (1-(6-(4-chlorophenyl)-2-(pyridin-3-yl)pyrimidin-4-yl)piperidin-3-yl)methanamine

Using tert-butyl (piperidin-3-ylmethyl)carbamate, the title compound was obtained as described for the example 54 (Scheme 1. General procedure A.).

¹H NMR (400 MHz, CD₃OD) δ [ppm]=1.44-1.53 (m, 1H), 1.61-1.71 (m, 1H), 1.91-2.07 (m, 3H), 2.92-3.09 (m, 3H), 3.15-3.24 (m, 1H), 4.57 (br, 1H), 4.77 (br, 1H), 7.30 (s, 1H), 7.53 (d, 2H), 8.09 (br, 1H), 8.23 (d, 2H), 8.90 (br, 1H), 9.46 (d, 1H), 9.79 (br, 1H); MS (ESI, m/z): 380.2 [M+H]⁺

### Example 63. (S)-(1-(6-(4-chlorophenyl)-2-(pyridin-3-yl)pyrimidin-4-yl)piperidin-3-yl)methanamine

Using tert-butyl (R)-(piperidin-3-ylmethyl)carbamate, the title compound was obtained as described for the example 54 (Scheme 1. General procedure A.).

¹H NMR (400 MHz, CD₃OD) δ [ppm]=1.44-1.53 (m, 1H), 1.61-1.71 (m, 1H), 1.91-2.07 (m, 3H), 2.92-3.09 (m, 3H), 3.15-3.24 (m, 1H), 4.57 (br, 1H), 4.77 (br, 1H), 7.30 (s, 1H), 7.53 (d, 2H), 8.09 (br, 1H), 8.23 (d, 2H), 8.90 (br, 1H), 9.46 (d, 1H), 9.79 (br, 1H); MS (ESI, m/z): 380.2 [M+H]⁺

### Example 64. 1-(6-(4-chlorophenyl)-2-(pyridin-3-yl)pyrimidin-4-yl)piperidin-4-amine

Using tert-butyl piperidin-4-ylcarbamate, the title compound was obtained as described for the example 54 (Scheme 1. General procedure A.).

¹H NMR (400 MHz, CD₃OD) δ [ppm]=1.60-1.71 (m, 2H), 2.18 (d, 2H), 3.18 (t, 2H), 3.47-3.56 (m, 1H), 4.83-4.94 (m, 2H), 7.33 (s, 1H), 7.54 (d, 2H), 7.98 (br, 1H), 8.25 (d, 2H), 8.84 (br, 1H), 9.32 (d, 1H), 9.73 (br, 1H); MS (ESI, m/z): 366.1 [M+H]⁺

### Example 65. (1-(6-(4-chlorophenyl)-2-(pyridin-3-yl)pyrimidin-4-yl)piperidin-4-yl)methanamine

Using tert-butyl (piperidin-4-ylmethyl)carbamate, the title compound was obtained as described for the example 54 (Scheme 1. General procedure A.).

¹H NMR (400 MHz, CD₃OD) δ [ppm]=1.31-1.42 (m, 2H), 1.98 (d, 2H), 2.01-2.10 (m, 1H), 2.91 (d, 2H), 3.13 (t, 2H), 4.81-4.95 (m, 2H), 7.29 (s, 1H), 7.54 (d, 2H), 8.00 (br, 1H), 8.23 (d, 2H), 8.85 (br, 1H), 9.34 (d, 1H), 9.71 (br, 1H); MS (ESI, m/z): 380.2 [M+H]⁺

### Example 66. (1R,2S)-2-((6-(4-chlorophenyl)-2-(pyridin-3-yl)pyrimidin-4-yl)amino)cyclopentan-1-ol

Using (1R,2S)-2-aminocyclopentan-1-ol and separation method of PREP. HPLC, the title compound was obtained as described for the example 1 (Scheme 1. General procedure A.).

¹H NMR (400 MHz, CD₃OD) δ [ppm]=1.59-1.74 (m, 2H), 1.79-1.94 (m, 2H), 1.98-2.07 (m, 1H), 2.27-2.35 (m, 1H), 4.16 (br, 1H), 4.43 (br, 1H), 6.96 (s, 1H), 7.53 (d, 2H), 8.02 (br, 1H), 8.14 (d, 2H), 8.86 (br, 1H), 9.38 (d, 1H), 9.72 (br, 1H); MS (ESI, m/z): 367.1 [M+H]⁺

### Example 67. (1S,2S)-2-((6-(4-chlorophenyl)-2-(pyridin-3-yl)pyrimidin-4-yl)amino)cyclopentan-1-ol

Using (1S,2S)-2-aminocyclopentan-1-ol and separation method of PREP. HPLC, the title compound was obtained as described for the example 1 (Scheme 1. General procedure A.).

¹H NMR (400 MHz, CD₃OD) δ [ppm]=1.59-1.74 (m, 2H), 1.79-1.94 (m, 2H), 1.98-2.07 (m, 1H), 2.27-2.35 (m, 1H), 4.16 (br, 1H), 4.43 (br, 1H), 6.96 (s, 1H), 7.53 (d, 2H), 8.02 (br, 1H), 8.14 (d, 2H), 8.86 (br, 1H), 9.38 (d, 1H), 9.72 (br, 1H); MS (ESI, m/z): 367.1 [M+H]⁺

### Example 68. trans-2-((6-(4-chlorophenyl)-2-(pyridin-3-yl)pyrimidin-4-yl)amino)cyclopentan-1-ol

Using trans-2-aminocyclopentan-1-ol and separation method of PREP. HPLC, the title compound was obtained as described for the example 1 (Scheme 1. General procedure A.).

¹H NMR (400 MHz, CD₃OD) δ [ppm]=1.59-1.74 (m, 2H), 1.79-1.94 (m, 2H), 1.98-2.07 (m, 1H), 2.27-2.35 (m, 1H), 4.16 (br, 1H), 4.43 (br, 1H), 6.96 (s, 1H), 7.53 (d, 2H), 8.02 (br, 1H), 8.14 (d, 2H), 8.86 (br, 1H), 9.38 (d, 1H), 9.72 (br, 1H); MS (ESI, m/z): 367.1 [M+H]⁺

### Example 69. (1R,2R)-2-((6-(4-chlorophenyl)-2-(pyridin-3-yl)pyrimidin-4-yl)amino)cyclohexan-1-ol

Using (1R,2R)-2-aminocyclohexan-1-ol and separation method of PREP. HPLC, the title compound was obtained as described for the example 1 (Scheme 1. General procedure A.).

¹H NMR (400 MHz, CD₃OD) δ [ppm]=1.28-1.51 (m, 4H), 1.76-1.84 (m, 2H), 2.08-2.16 (m, 2H), 3.52-3.57 (m, 1H), 4.16 (br, 1H), 6.95 (s, 1H), 7.52 (d, 2H), 8.03 (br, 1H), 8.12 (d, 2H), 8.86 (br, 1H), 9.32 (d, 1H), 9.66 (br, 1H); MS (ESI, m/z): 381.1 [M+H]⁺

### Example 70. cis-4-(6-(4-chlorophenyl)-2-(pyridin-3-yl)pyrimidin-4-yl)-2,6-dimethylmorpholine

Using cis-2,6-dimethylmorpholine and separation method of PREP. HPLC, the title compound was obtained as described for the example 1 (Scheme 1. General procedure A.).

¹H NMR (400 MHz, CD₃OD) δ [ppm]=1.29 (d, 6H), 2.69 (t, 2H), 3.67-3.74 (m, 2H), 4.56 (br, 2H), 7.23 (s, 1H), 7.51 (d, 2H), 8.05 (br, 1H), 8.22 (d, 2H), 8.88 (br, 1H), 9.38 (d, 1H), 9.72 (br, 1H); MS (ESI, m/z): 381.1 [M+H]⁺

### Example 71. 4-(6-(4-chlorophenyl)-2-(pyridin-3-yl)pyrimidin-4-yl)morpholine

Using morpholine and separation method of PREP. HPLC, the title compound was obtained as described for the example 1 (Scheme 1. General procedure A.).

¹H NMR (400 MHz, CD₃OD) δ [ppm]=3.81-3.88 (m, 8H), 7.22 (s, 1H), 7.51 (d, 2H), 8.12 (br, 1H), 8.21 (d, 2H), 8.88 (br, 1H), 9.41 (d, 1H), 9.83 (br, 1H); MS (ESI, m/z): 353.1 [M+H]⁺

### Example 72. 6-(4-chlorophenyl)-N-(morpholin-2-ylmethyl)-2-(pyridin-3-yl)pyrimidin-4-amine

Using tert-butyl 2-(aminomethyl)morpholine-4-carboxylate and separation method of PREP. HPLC, the title compound was obtained as described for the example 54 (Scheme 1. General procedure A.).

¹H NMR (400 MHz, CD₃OD) δ [ppm]=3.88-4.18 (m, 9H), 7.07 (s, 1H), 7.60 (d, 2H), 8.15 (br, 1H), 8.33 (d, 2H), 8.89 (br, 1H), 9.41 (d, 1H), 9.73 (br, 1H); MS (ESI, m/z): 382.9 [M+H]⁺

### Example 73. 4-(1-(6-(4-chlorophenyl)-2-(pyridin-3-yl)pyrimidin-4-yl)pyrrolidin-3-yl)morpholine

Using 4-(pyrrolidin-3-yl)morpholine and separation method of PREP. HPLC, the title compound was obtained as described for the example 1 (Scheme 1. General procedure A.).

¹H NMR (400 MHz, CD₃OD) δ [ppm]=2.43 (br, 1H), 2.68 (br, 1H), 3.50 (br, 4H), 3.69 (br, 1H), 3.86-4.40 (m, 8H), 7.05 (s, 1H), 7.53 (d, 2H), 8.14 (br, 1H), 8.25 (d, 2H), 8.92 (br, 1H), 9.52 (d, 1H), 9.79 (br, 1H); MS (ESI, m/z): 422.2 [M+H]⁺

### Example 74. 4-(6-(4-chlorophenyl)-2-(pyridin-3-yl)pyrimidin-4-yl)thiomorpholine

Using thiomorpholine and separation method of PREP. HPLC, the title compound was obtained as described for the example 1 (Scheme 1. General procedure A.)

¹H NMR (400 MHz, CD₃OD) δ [ppm]=2.74-7.76 (m, 4H), 4.24 (br, 4H), 7.25 (s, 1H), 7.52 (d, 2H), 8.12 (br, 1H), 8.22 (d, 2H), 8.91 (br, 1H), 9.45 (d, 1H), 9.73 (br, 1H); MS (ESI, m/z): 422.2 [M+H]⁺

### Example 75. 6-(4-chlorophenyl)-N-(3-morpholinopropyl)-2-(pyridin-3-yl)pyrimidin-4-amine

Using 3-morpholinopropan-1-amine and separation method of PREP. HPLC, the title compound was obtained as described for the example 1 (Scheme 1. General procedure A.).

¹H NMR (400 MHz, CD₃OD) δ [ppm]=2.15-2.22 (m, 2H), 3.15 (br, 2H), 3.32-3.35 (m, 2H), 3.51 (br, 2H), 3.72 (br, 4H), 4.03 (br, 2H), 6.98 (s, 1H), 7.53 (d, 2H), 8.00 (br, 1H), 8.15 (d, 2H), 8.85 (br, 1H), 9.35 (d, 1H), 9.72 (br, 1H); MS (ESI, m/z): 410.2 [M+H]⁺

### Example 76. (R)-4-(4-chlorophenyl)-6-(2-methylpiperazin-1-yl)-2-(pyridin-3-yl)pyrimidine

Using tert-butyl (R)-3-methylpiperazine-1-carboxylate and separation method of PREP. HPLC, the title compound was obtained as described for the example 54 (Scheme 1. General procedure A.).

¹H NMR (400 MHz, CD₃OD) δ [ppm]=2.15-2.22 (m, 2H), 3.15 (br, 2H), 3.32-3.35 (m, 2H), 3.51 (br, 2H), 3.72 (br, 4H), 4.03 (br, 2H), 6.98 (s, 1H), 7.53 (d, 2H), 8.00 (br, 1H), 8.15 (d, 2H), 8.85 (br, 1H), 9.35 (d, 1H), 9.72 (br, 1H); MS (ESI, m/z): 366.1 [M+H]⁺

### Example 77. (R)-(4-(6-(4-chlorophenyl)-2-(pyridin-3-yl)pyrimidin-4-yl)-3-methylpiperazin-1-yl)(phenyl)methanone

Using (R)-(3-methylpiperazin-1-yl)(phenyl)methanone and separation method of PREP. HPLC, the title compound was obtained as described for the example 1 (Scheme 1. General procedure A.).

MS (ESI, m/z): 470.2 [M+H]⁺

### Example 78. methyl (R)-4-(6-(4-chlorophenyl)-2-(pyridin-3-yl)pyrimidin-4-yl)piperazine-2-carboxylate

Using 1-(tert-butyl) 2-methyl (R)-piperazine-1,2-dicarboxylate and separation method of PREP. HPLC, the title compound was obtained as described for the example 54 (Scheme 1. General procedure A.).

¹H NMR (400 MHz, CD₃OD) δ [ppm]=1.60 (br, 1H), 2.03 (br, 1H), 3.64 (s, 3H), 3.77 (br, 1H), 4.01 (br, 1H), 4.46 (br, 1H), 4.55 (br, 1H), 5.35 (br, 1H), 7.43 (s, 1H), 7.56 (d, 2H), 7.74 (br, 1H), 8.29 (d, 2H), 8.74 (br, 1H), 9.06 (d, 1H), 9.67 (br, 1H); MS (ESI, m/z): 410.1 [M+H]⁺

### Example 79. (R)-(4-(6-(4-chlorophenyl)-2-(pyridin-3-yl)pyrimidin-4-yl)piperazin-2-yl)methanol

Using tert-butyl (R)-2-(hydroxymethyl)piperazine-1-carboxylate and separation method of PREP. HPLC, the title compound was obtained as described for the example 54 (Scheme 1. General procedure A.).

¹H NMR (400 MHz, CD₃OD) δ [ppm]=3.27-3.35 (m, 2H), 3.42-3.58 (m, 4H), 3.80-3.85 (m, 2H), 3.94-3.98 (m, 2H), 4.82-4.85 (m, 1H), 7.44 (s, 1H), 7.56 (d, 2H), 8.03 (br, 1H), 8.30 (d, 2H), 8.87 (br, 1H), 9.40 (d, 1H), 9.77 (br, 1H); MS (ESI, m/z): 410.1 [M+H]⁺

### Example 80. 4-(4-chlorophenyl)-6-(4-(2,3-dichlorophenyl)piperazin-1-yl)-2-(pyridin-3-yl)pyrimidine

Using 1-(2,3-dichlorophenyl)piperazine and separation method of PREP. HPLC, the title compound was obtained as described for the example 1 (Scheme 1. General procedure A.).

¹H NMR (400 MHz, CD₃OD) δ [ppm]=3.21 (br, 4H), 4.10 (br, 4H), 7.13-7.17 (m, 1H), 7.28 (d, 2H), 7.34 (s, 1H), 7.55 (d, 2H), 8.12 (br, 1H), 8.26 (d, 2H), 8.91 (br, 1H), 9.50 (d, 1H), 9.77 (br, 1H); MS (ESI, m/z): 496.1 [M+H]⁺

### Example 81. 4-(4-chlorophenyl)-6-(4-(2,5-dimethoxybenzyl)piperazin-1-yl)-2-(pyridin-3-yl)pyrimidine

Using 1-(2,5-dimethoxybenzyl)piperazine and separation method of PREP. HPLC, the title compound was obtained as described for the example 1 (Scheme 1. General procedure A.).

¹H NMR (400 MHz, CD₃OD) δ [ppm]=3.54 (br, 8H), 3.80 (s, 3H), 3.90 (s, 3H), 4.43 (s, 2H), 7.09 (s, 3H), 7.42 (s, 1H), 7.55 (d, 2H), 8.14 (br, 1H), 8.28 (d, 2H), 8.92 (br, 1H), 9.54 (d, 1H), 9.81 (br, 1H); MS (ESI, m/z): 502.2 [M+H]⁺

### Example 82. 2-(4-(6-(4-chlorophenyl)-2-(pyridin-3-yl)pyrimidin-4-yl)piperazin-1-yl)ethan-1-ol

Using 2-(piperazin-1-yl)ethan-1-ol and separation method of PREP. HPLC, the title compound was obtained as described for the example 1 (Scheme 1. General procedure A.).

¹H NMR (400 MHz, CD₃OD) δ [ppm]=3.38 (t, 2H), 3.52 (br, 8H), 3.96 (t, 2H), 7.42 (s, 1H), 7.55 (d, 2H), 8.00 (br, 1H), 8.28 (d, 2H), 8.86 (br, 1H), 9.37 (d, 1H), 9.76 (br, 1H); MS (ESI, m/z): 396.2 [M+H]⁺

### Example 83. 4-(4-chlorophenyl)-6-(4-(2-methoxyphenyl)piperazin-1-yl)-2-(pyridin-3-yl)pyrimidine

Using 1-(2-methoxyphenyl)piperazine and separation method of PREP. HPLC, the title compound was obtained as described for the example 1 (Scheme 1. General procedure A.).

¹H NMR (400 MHz, CD₃OD) δ [ppm]=3.50 (br, 4H), 3.98 (s, 3H), 4.22 (br, 4H), 7.04 (dd, 1H), 7.15 (d, 1H), 7.26-7.32 (m, 2H), 7.37 (s, 1H), 7.53 (d, 2H), 8.12 (br, 1H), 8.26 (d, 2H), 8.91 (br, 1H), 9.50 (d, 1H), 9.77 (br, 1H); MS (ESI, m/z): 458.2 [M+H]⁺

### Example 84. 4-(4-chlorophenyl)-6-(4-(2-ethoxyphenyl)piperazin-1-yl)-2-(pyridin-3-yl)pyrimidine

Using 1-(2-ethoxyphenyl)piperazine and separation method of PREP. HPLC, the title compound was obtained as described for the example 1 (Scheme 1. General procedure A.).

¹H NMR (400 MHz, CD₃OD) δ [ppm]=1.48 (t, 3H), 3.47 (br, 4H), 4.17-4.22 (m, 6H), 7.01 (dd, 1H), 7.10 (d, 1H), 7.19-7.25 (m, 2H), 7.38 (s, 1H), 7.55 (d, 2H), 8.11 (br, 1H), 8.26 (d, 2H), 8.90 (br, 1H), 9.49 (d, 1H), 9.78 (br, 1H); MS (ESI, m/z): 472.2 [M+H]⁺

### Example 85. 4-(4-chlorophenyl)-6-(4-(2-fluorophenyl)piperazin-1-yl)-2-(pyridin-3-yl)pyrimidine

Using 1-(2-fluorophenyl)piperazine and separation method of PREP. HPLC, the title compound was obtained as described for the example 1 (Scheme 1. General procedure A.).

¹H NMR (400 MHz, CD₃OD) δ [ppm]=3.22 (br, 4H), 4.07 (br, 4H), 6.99-7.13 (m, 4H), 7.31 (s, 1H), 7.52 (d, 2H), 8.12 (br, 1H), 8.23 (d, 2H), 8.90 (br, 1H), 9.46 (d, 1H), 9.75 (br, 1H); MS (ESI, m/z): 446.2 [M+H]⁺

### Example 86. (4-(6-(4-chlorophenyl)-2-(pyridin-3-yl)pyrimidin-4-yl)piperazin-1-yl)(furan-2-yl)methanone

Using furan-2-yl(piperazin-1-yl)methanone and separation method of PREP. HPLC, the title compound was obtained as described for the example 1 (Scheme 1. General procedure A.).

¹H NMR (400 MHz, CD₃OD) δ [ppm]=4.02 (br, 8H), 6.64 (dd, 1H), 7.14 (d, 1H), 7.29 (s, 1H), 7.53 (d, 2H), 7.74 (d, 1H), 8.06 (dd, 1H), 8.25 (d, 2H), 8.87 (br, 1H), 9.43 (d, 1H), 9.74 (br, 1H); MS (ESI, m/z): 446.1 [M+H]⁺

### Example 87. 4-(4-chlorophenyl)-6-(4-phenethylpiperazin-1-yl)-2-(pyridin-3-yl)pyrimidine

Using 1-phenethylpiperazine and separation method of PREP. HPLC, the title compound was obtained as described for the example 1 (Scheme 1. General procedure A.).

¹H NMR (400 MHz, CD₃OD) δ [ppm]=3.14-3.18 (m, 2H), 3.45-3.50 (m, 2H), 3.61 (br, 8H), 7.26-7.38 (m, 5H), 7.44 (s, 1H), 7.54 (d, 2H), 8.16 (br, 1H), 8.28 (d, 2H), 8.94 (br, 1H), 9.54 (d, 1H), 9.81 (br, 1H); MS (ESI, m/z): 456.2 [M+H]⁺

### Example 88. 6-(4-chlorophenyl)-N-(2-(piperazin-1-yl)ethyl)-2-(pyridin-3-yl)pyrimidin-4-amine

Using tert-butyl 4-(2-aminoethyl)piperazine-1-carboxylate and separation method of PREP. HPLC, the title compound was obtained as described for the example 54 (Scheme 1. General procedure A.).

¹H NMR (400 MHz, CD₃OD) δ [ppm]=2.70 (t, 2H), 2.75 (br, 4H), 3.08 (t, 2H), 4.18 (br, 4H), 7.37 (s, 1H), 7.52 (d, 2H), 8.11 (br, 1H), 8.25 (d, 2H), 8.91 (br, 1H), 9.48 (d, 1H), 9.78 (br, 1H); MS (ESI, m/z): 395.2 [M+H]⁺

### Example 89. 4-(4-chlorophenyl)-6-(4-(pyridin-2-yl)piperazin-1-yl)-2-(pyridin-3-yl)pyrimidine

Using 1-(pyridin-2-yl)piperazine and separation method of PREP. HPLC, the title compound was obtained as described for the example 1 (Scheme 1. General procedure A.).

¹H NMR (400 MHz, CD₃OD) δ [ppm]=3.98 (br, 4H), 4.20 (br, 4H), 7.06 (t, 1H), 7.30 (s, 1H), 7.44 (d, 1H), 7.54 (d, 2H), 8.04 (d, 1H), 8.08-8.16 (m, 2H), 8.27 (d, 2H), 8.93 (br, 1H), 9.53 (d, 1H), 9.80 (br, 1H); MS (ESI, m/z): 429.2 [M+H]⁺

### Example 90. 4-(4-chlorophenyl)-2-(pyridin-3-yl)-6-(4-(pyrimidin-2-yl)piperazin-1-yl)pyrimidine

Using 1-(pyridin-2-yl)piperazine and separation method of PREP. HPLC, the title compound was obtained as described for the example 1 (Scheme 1. General procedure A.).

¹H NMR (400 MHz, CD₃OD) δ [ppm]=4.04 (br, 8H), 6.72 (t, 1H), 7.36 (s, 1H), 7.55 (d, 2H), 8.04 (d, 1H), 8.19 (br, 1H), 8.28 (d, 2H), 8.43 (d, 2H), 8.93 (br, 1H), 9.59 (d, 1H), 9.81 (br, 1H); MS (ESI, m/z): 429.2 [M+H]⁺

### Example 91. 4-(2-(4-(6-(4-chlorophenyl)-2-(pyridin-3-yl)pyrimidin-4-yl)piperazin-1-yl)ethyl)morpholine

Using 4-(2-(piperazin-1-yl)ethyl)morpholine and separation method of PREP. HPLC, the title compound was obtained as described for the example 1 (Scheme 1. General procedure A.).

¹H NMR (400 MHz, CD₃OD) δ [ppm]=3.27 (br, 4H), 3.35 (br, 4H), 3.41 (t, 2H), 3.55 (t, 2H), 3.95 (br, 4H), 4.18 (br, 4H), 7.39 (s, 1H), 7.54 (d, 2H), 8.16 (br, 1H), 8.26 (d, 2H), 8.93 (br, 1H), 9.54 (d, 1H), 9.79 (br, 1H); MS (ESI, m/z): 429.2 [M+H]⁺

### Example 92. 4-(4-chlorophenyl)-6-(4-(4-methylpiperazin-1-yl)piperidin-1-yl)-2-(pyridin-3-yl)pyrimidine

Using 1-methyl-4-(piperidin-4-yl)piperazine and separation method of PREP. HPLC, the title compound was obtained as described for the example 1 (Scheme 1. General procedure A.).

¹H NMR (400 MHz, CD₃OD) δ [ppm]=1.69-1.79 (m, 2H), 2.53 (d, 2H), 2.93 (s, 3H), 3.14 (t, 2H), 3.44 (br, 4H), 3.52 (br, 4H), 3.63 (br, 2H), 4.29 (br, 1H), 7.34 (s, 1H), 7.54 (d, 2H), 8.14 (br, 1H), 8.24 (d, 2H), 8.92 (br, 1H), 9.51 (d, 1H), 9.76 (br, 1H); MS (ESI, m/z): 449.2 [M+H]⁺

### Example 93. trans-4-(4-chlorophenyl)-6-(4-cinnamylpiperazin-1-yl)-2-(pyridin-3-yl)pyrimidine

Using trans-1-cinnamylpiperazine and separation method of PREP. HPLC, the title compound was obtained as described for the example 1 (Scheme 1. General procedure A.).

¹H NMR (400 MHz, CD₃OD) δ [ppm]=3.58 (br, 8H), 4.04 (d, 2H), 6.35-6.43 (m, 1H), 6.96 (d, 1H), 7.32-7.40 (m, 3H), 7.44 (s, 1H), 7.50-7.55 (m, 4H), 8.16 (br, 1H), 8.28 (d, 2H), 8.93 (br, 1H), 9.56 (d, 1H), 9.82 (br, 1H); MS (ESI, m/z): 468.2 [M+H]⁺

### Example 94. 4-(6-(4-chlorophenyl)-2-(pyridin-3-yl)pyrimidin-4-yl)piperazin-2-one

Using piperazin-2-one and separation method of PREP. HPLC, the title compound was obtained as described for the example 1 (Scheme 1. General procedure A.).

¹H NMR (400 MHz, CD₃OD) δ [ppm]=3.53 (t, 2H), 4.10 (br, 2H), 4.46 (s, 2H), 7.29 (s, 1H), 7.54 (d, 2H), 8.11 (br, 1H), 8.28 (d, 2H), 8.89 (br, 1H), 9.49 (d, 1H), 9.77 (br, 1H); MS (ESI, m/z): 366.1 [M+H]⁺

### Example 95. 4-(4-chlorophenyl)-6-(4-phenylpiperazin-1-yl)-2-(pyridin-3-yl)pyrimidine

Using 1-phenylpiperazine and separation method of PREP. HPLC, the title compound was obtained as described for the example 1 (Scheme 1. General procedure A.).

¹H NMR (400 MHz, CD₃OD) δ [ppm]=3.43 (t, 4H), 4.13 (br, 4H), 7.02 (t, 1H), 7.14 (d, 2H), 7.34 (t, 3H), 7.53 (d, 2H), 8.14-8.17 (m, 1H), 8.25 (d, 2H), 8.92 (br, 1H), 9.54 (d, 1H), 9.77 (br, 1H); MS (ESI, m/z): 428.2 [M+H]⁺

### Example 96. 4-(4-chlorophenyl)-6-(4-propylpiperazin-1-yl)-2-(pyridin-3-yl)pyrimidine

Using 1-propylpiperazine and separation method of PREP. HPLC, the title compound was obtained as described for the example 1 (Scheme 1. General procedure A.).

¹H NMR (400 MHz, CD₃OD) δ [ppm]=1.06 (t, 3H), 1.80-1.90 (m, 2H), 3.17-3.21 (m, 2H), 3.54 (br, 8H), 7.43 (s, 1H), 7.55 (d, 2H), 8.03 (t, 1H), 8.28 (d, 2H), 8.87 (br, 1H), 9.41 (d, 1H), 9.77 (br, 1H); MS (ESI, m/z): 394.2 [M+H]⁺

### Example 97. 4-(4-(benzo[d][1,3]dioxol-5-ylmethyl)piperazin-1-yl)-6-(4-chlorophenyl)-2-(pyridin-3-yl)pyrimidine

Using 1-(benzo[d][1,3]dioxol-5-ylmethyl)piperazine and separation method of PREP. HPLC, the title compound was obtained as described for the example 1 (Scheme 1. General procedure A.).

¹H NMR (400 MHz, CD₃OD) δ [ppm]=3.44 (br, 8H), 4.34 (s, 2H), 6.04 (s, 2H), 6.94 (d, 1H), 7.03 (d, 1H), 7.05 (s, 1H), 7.42 (s, 1H), 7.54 (d, 2H), 8.11-8.14 (m, 1H), 8.28 (d, 2H), 8.92 (br, 1H), 9.51 (d, 1H), 9.80 (br, 1H); MS (ESI, m/z): 486.2 [M+H]⁺

### Example 98. (S)-(4-(6-(4-chlorophenyl)-2-(pyridin-3-yl)pyrimidin-4-yl)piperazin-2-yl)methanol

Using tert-butyl 4-(2-aminoethyl)piperazine-1-carboxylate, the title compound was obtained as described for the example 54 (Scheme 1. General procedure A.).

¹H NMR (400 MHz, CD₃OD) δ [ppm]=3.55 (br, 1H), 3.45-3.59 (m, 4H), 3.62-3.67 (m, 1H), 3.72-3.77 (m, 1H), 3.82-3.87 (m, 1H), 3.95-4.00 (m, 1H), 7.50 (s, 1H), 7.56 (d, 2H), 8.25-8.28 (m, 1H), 8.33 (d, 2H), 8.99 (d, 1H), 9.69 (d, 1H), 9.88 (s, 1H); MS (ESI, m/z): 382.1 [M+H]⁺

### Example 99. 4-(4-chlorophenyl)-6-(4-(4-fluorophenyl)piperazin-1-yl)-2-(pyridin-3-yl)pyrimidine

Using 1-(4-fluorophenyl)piperazine and separation method of PREP. HPLC, the title compound was obtained as described for the example 1 (Scheme 1. General procedure A.).

¹H NMR (400 MHz, CD₃OD) δ [ppm]=4.09 (br, 8H), 7.02-7.07 (m, 2H), 7.10-7.13 (m, 2H), 7.32 (s, 1H), 7.52 (d, 2H), 8.14-8.17 (m, 1H), 8.23 (d, 2H), 8.92 (d, 1H), 9.53 (d, 1H), 9.76 (s, 1H); MS (ESI, m/z): 446.2 [M+H]⁺

### Example 100. 6-(4-chlorophenyl)-N-(1,2,2,6,6-pentamethylpiperidin-4-yl)-2-(pyridin-3-yl)pyrimidin-4-amine

Using 1,2,2,6,6-pentamethylpiperidin-4-amine and separation method of PREP. HPLC, the title compound was obtained as described for the example 1 (Scheme 1. General procedure A.).

¹H NMR (400 MHz, CD₃OD) δ [ppm]=1.55 (s, 6H), 1.68 (s, 6H), 1.86 (t, 1H), 2.43 (d, 4H), 2.92 (s, 3H), 7.00 (s, 1H), 7.54 (d, 2H), 8.06-8.09 (m, 1H), 8.16 (d, 2H), 8.89 (d, 1H), 9.41 (d, 1H), 9.73 (s, 1H); MS (ESI, m/z): 436.2 [M+H]⁺

### Example 101. 6-(4-chlorophenyl)-N-(piperidin-3-yl)-2-(pyridin-3-yl)pyrimidin-4-amine

Using tert-butyl 3-aminopiperidine-1-carboxylate and separation method of PREP. HPLC, the title compound was obtained as described for the example 54 (Scheme 1. General procedure A.).

¹H NMR (400 MHz, CD₃OD) δ [ppm]=1.74-1.83 (m, 1H), 1.92-2.03 (m, 1H), 2.12-2.23 (m, 2H), 2.96-3.11 (m, 2H), 3.40 (d, 1H), 3.71 (d, 1H), 4.55 (br, 1H), 7.03 (s, 1H), 7.53 (d, 2H), 8.10 (br, 1H), 8.16 (d, 2H), 8.91 (br, 1H), 9.49 (d, 1H), 9.83 (br, 1H); MS (ESI, m/z): 366.1 [M+H]⁺

### Example 102. 4-(4-chlorophenyl)-6-(piperazin-1-yl)-2-(pyridin-3-yl)pyrimidine

Using tert-butyl piperazine-1-carboxylate and separation method of PREP. HPLC, the title compound was obtained as described for the example 54 (Scheme 1. General procedure A.).

¹H NMR (400 MHz, CD₃OD) δ [ppm]=3.41 (br, 4H), 4.19 (br, 4H), 7.41 (s, 1H), 7.55 (d, 2H), 8.04 (t, 1H), 8.28 (d, 2H), 8.87 (d, 1H), 9.41 (d, 1H), 9.77 (s, 1H); MS (ESI, m/z): 352.1 [M+H]⁺

### Example 103. Trans-4-(4-chlorophenyl)-6-(2,5-dimethylpiperazin-1-yl)-2-(pyridin-3-yl)pyrimidine

Using tert-butyl trans-2,5-dimethylpiperazine-1-carboxylate and separation method of PREP. HPLC, the title compound was obtained as described for the example 54 (Scheme 1. General procedure A.).

¹H NMR (400 MHz, CD₃OD) δ [ppm]=1.47 (d, 3H), 1.48 (d, 3H), 3.62-3.73 (m, 3H), 3.91 (br, 1H), 4.67 (d, 1H), 5.17 (br, 1H), 7.35 (s, 1H), 7.56 (d, 2H), 8.02-8.05 (m, 1H), 8.28 (d, 2H), 8.87 (d, 1H), 9.41 (d, 1H), 9.76 (s, 1H); MS (ESI, m/z): 380.2 [M+H]⁺

### Example 104. Cis-4-(4-chlorophenyl)-6-(3,5-dimethylpiperazin-1-yl)-2-(pyridin-3-yl)pyrimidine

Using tert-butyl cis-2,6-dimethylpiperazine-1-carboxylate and separation method of PREP. HPLC, the title compound was obtained as described for the example 54 (Scheme 1. General procedure A.).

¹H NMR (400 MHz, CD₃OD) δ [ppm]=1.48 (d, 6H), 3.03-3.09 (m, 2H), 3.46-3.51 (m, 2H), 5.04 (br, 2H), 7.47 (s, 1H), 7.55 (d, 2H), 8.08-8.12 (m, 1H), 8.30 (d, 2H), 8.91 (d, 1H), 9.50 (d, 1H), 9.81 (s, 1H); MS (ESI, m/z): 380.2 [M+H]⁺

### Example 105. 4-(4-chlorophenyl)-6-(4-methylpiperazin-1-yl)-2-(pyridin-3-yl)pyrimidine

Using 1-methylpiperazine and separation method of PREP. HPLC, the title compound was obtained as described for the example 1 (Scheme 1. General procedure A.).

¹H NMR (400 MHz, CD₃OD) δ [ppm]=3.00 (s, 3H), 3.49 (br, 4H), 3.55 (br, 4H), 7.43 (s, 1H), 7.56 (d, 2H), 7.97-8.00 (m, 1H), 8.29 (d, 2H), 8.86 (d, 1H), 9.36 (d, 1H), 9.76 (s, 1H); MS (ESI, m/z): 366.1 [M+H]⁺

### Example 106. 4-(4-chlorophenyl)-6-(4-ethylpiperazin-1-yl)-2-(pyridin-3-yl)pyrimidine

Using 1-ethylpiperazine and separation method of PREP. HPLC, the title compound was obtained as described for the example 1 (Scheme 1. General procedure A.).

¹H NMR (400 MHz, CD₃OD) δ [ppm]=1.43 (t, 3H), 3.27 (q, 2H), 3.54 (br, 4H), 3.56 (br, 4H), 7.43 (s, 1H), 7.56 (d, 2H), 7.97-8.01 (m, 1H), 8.29 (d, 2H), 8.86 (d, 1H), 9.36 (d, 1H), 9.76 (s, 1H); MS (ESI, m/z): 380.2 [M+H]⁺

### Example 107. 4-(4-chlorophenyl)-6-(4-(methylsulfonyl)piperazin-1-yl)-2-(pyridin-3-yl)pyrimidine

Using 1-(methylsulfonyl)piperazine and separation method of PREP. HPLC, the title compound was obtained as described for the example 1 (Scheme 1. General procedure A.).

¹H NMR (400 MHz, CD₃OD) δ [ppm]=2.90 (s, 3H), 3.40 (br, 4H), 4.06 (br, 4H), 7.35 (s, 1H), 7.55 (d, 2H), 8.02 (br, 1H), 8.27 (d, 2H), 8.85 (br, 1H), 9.38 (d, 1H), 9.76 (br, 1H); MS (ESI, m/z): 430.1 [M+H]⁺

### Example 108. 1-(4-(6-(4-chlorophenyl)-2-(pyridin-3-yl)pyrimidin-4-yl)piperazin-1-yl)ethan-1-one

Using 1-(piperazin-1-yl)ethan-1-one and separation method of PREP. HPLC, the title compound was obtained as described for the example 1 (Scheme 1. General procedure A.).

¹H NMR (400 MHz, CD₃OD) δ [ppm]=2.19 (s, 3H), 3.73-3.78 (m, 4H), 3.93 (br, 2H), 3.99 (br, 2H), 7.28 (s, 1H), 7.54 (d, 2H), 7.96-7.99 (m, 1H), 8.25 (d, 2H), 8.83 (d, 1H), 9.34 (d, 1H), 9.71 (s, 1H); MS (ESI, m/z): 394.1 [M+H]⁺

### Example 109. 4-(4-chlorophenyl)-6-(3-ethylpiperazin-1-yl)-2-(pyridin-3-yl)pyrimidine

Using tert-butyl 2-ethylpiperazine-1-carboxylate and separation method of PREP. HPLC, the title compound was obtained as described for the example 54 (Scheme 1. General procedure A.).

¹H NMR (400 MHz, CD₃OD) δ [ppm]=1.19 (t, 3H), 1.73-1.90 (m, 3H), 3.25-3.35 (m, 4H), 3.49-3.59 (m, 2H), 7.45 (s, 1H), 7.56 (d, 2H), 8.07-8.10 (m, 1H), 8.30 (d, 2H), 8.90 (d, 1H), 9.46 (d, 1H), 9.78 (s, 1H); MS (ESI, m/z): 380.2 [M+H]⁺

### Example 110. ethyl 4-(6-(4-chlorophenyl)-2-(pyridin-3-yl)pyrimidin-4-yl)piperazine-1-carboxylate

Using ethyl piperazine-1-carboxylate and separation method of PREP. HPLC, the title compound was obtained as described for the example 1 (Scheme 1. General procedure A.).

¹H NMR (400 MHz, CD₃OD) δ [ppm]=1.31 (t, 3H), 3.66 (br, 4H), 3.94 (br, 4H), 4.19 (q, 2H), 7.29 (s, 1H), 7.53 (d, 2H), 8.02-8.06 (m, 1H), 8.24 (d, 2H), 8.86 (d, 1H), 9.41 (d, 1H), 9.73 (s, 1H); MS (ESI, m/z): 424.2 [M+H]⁺

### Example 111. 4-(6-(4-chlorophenyl)-2-(pyridin-3-yl)pyrimidin-4-yl)piperazine-2-carboxylic acid

Using 1-(tert-butoxycarbonyl)piperazine-2-carboxylic acid and separation method of PREP. HPLC, the title compound was obtained as described for the example 54 (Scheme 1. General procedure A.).

MS (ESI, m/z): 396.1 [M+H]⁺

### Example 112. methyl 1-(6-(4-chlorophenyl)-2-(pyridin-3-yl)pyrimidin-4-yl)piperazine-2-carboxylate

Using 1-(tert-butyl) 3-methyl piperazine-1,3-dicarboxylate and separation method of PREP. HPLC, the title compound was obtained as described for the example 54 (Scheme 1. General procedure A.).

¹H NMR (400 MHz, CD₃OD) δ [ppm]=1.49 (s, 3H), 3.47-3.57 (m, 3H), 3.63 (br, 1H), 3.83 (br, 1H), 4.07 (br, 1H), 4.10 (br, 1H), 7.43 (s, 1H), 7.55 (d, 2H), 7.80-7.84 (m, 1H), 8.28 (d, 2H), 8.77 (d, 1H), 9.16 (d, 1H), 9.68 (s, 1H); MS (ESI, m/z): 410.1 [M+H]⁺

### Example 113. (S)-4-(4-chlorophenyl)-6-(2-phenylpiperazin-1-yl)-2-(pyridin-3-yl)pyrimidine

Using tert-butyl (S)-3-phenylpiperazine-1-carboxylate and separation method of PREP. HPLC, the title compound was obtained as described for the example 54 (Scheme 1. General procedure A.).

¹H NMR (400 MHz, CD₃OD) δ [ppm]=3.47-3.55 (m, 2H), 3.58-3.71 (m, 2H), 4.55-4.60 (m, 2H), 5.09-5.12 (m, 1H), 7.47 (s, 1H), 7.53-7.63 (m, 7H), 7.79-7.82 (m, 1H), 8.29 (d, 2H), 8.76 (d, 1H), 9.15 (d, 1H), 9.67 (s, 1H); MS (ESI, m/z): 428.2 [M+H]⁺

### Example 114. 4-(4-chlorophenyl)-2-(pyridin-3-yl)-6-(4-(o-tolyl)piperazin-1-yl)pyrimidine

Using 1-(o-tolyl)piperazine and separation method of PREP. HPLC, the title compound was obtained as described for the example 1 (Scheme 1. General procedure A.).

¹H NMR (400 MHz, CD₃OD) δ [ppm]=2.40 (s, 3H), 3.06 (t, 4H), 4.07 (br, 4H), 7.01 (t, 1H), 7.08 (d, 1H), 7.31 (s, 1H), 7.53 (d, 2H), 7.59 (d, 1H), 8.11-8.14 (m, 1H), 8.24 (d, 2H), 8.42 (t, 1H), 8.91 (d, 1H), 9.50 (d, 1H), 9.75 (s, 1H); MS (ESI, m/z): 442.2 [M+H]⁺

### Example 115. 4-(4-chlorophenyl)-2-(pyridin-3-yl)-6-(4-(p-tolyl)piperazin-1-yl)pyrimidine

Using 1-(p-tolyl)piperazine and separation method of PREP. HPLC, the title compound was obtained as described for the example 1 (Scheme 1. General procedure A.).

¹H NMR (400 MHz, CD₃OD) δ [ppm]=2.31 (s, 3H), 3.44 (t, 4H), 4.17 (br, 4H), 7.14 (d, 2H), 7.20 (d, 2H), 7.37 (s, 1H), 7.54 (d, 2H), 8.11-8.14 (m, 1H), 8.26 (d, 2H), 8.91 (d, 1H), 9.52 (d, 1H), 9.78 (s, 1H); MS (ESI, m/z): 442.2 [M+H]⁺

### Example 116. 4-(4-chlorophenyl)-2-(pyridin-3-yl)-6-(4-(m-tolyl)piperazin-1-yl)pyrimidine

Using 1-(m-tolyl)piperazine and separation method of PREP. HPLC, the title compound was obtained as described for the example 1 (Scheme 1. General procedure A.).

¹H NMR (400 MHz, CD₃OD) δ [ppm]=2.34 (s, 3H), 3.43 (t, 4H), 4.13 (br, 4H), 6.87 (d, 1H), 6.98 (d, 1H), 7.01 (s, 1H), 7.22 (t, 1H), 7.35 (s, 1H), 7.54 (d, 2H), 8.12-8.15 (m, 1H), 8.26 (d, 2H), 8.91 (d, 1H), 9.52 (d, 1H), 9.77 (s, 1H); MS (ESI, m/z): 442.2 [M+H]⁺

### Example 117. 4-(4-chlorophenyl)-2-(pyridin-3-yl)-6-(4-(3-(trifluoromethyl)phenyl)piperazin-1-yl)pyrimidine

Using 1-(3-(trifluoromethyl)phenyl)piperazine and separation method of PREP. HPLC, the title compound was obtained as described for the example 1 (Scheme 1. General procedure A.).

¹H NMR (400 MHz, CD₃OD) δ [ppm]=3.42 (t, 4H), 4.08 (br, 4H), 7.13 (d, 1H), 7.25 (s, 1H), 7.26 (d, 1H), 7.31 (s, 1H), 7.44 (t, 1H), 7.53 (d, 2H), 8.10-8.14 (m, 1H), 8.24 (d, 2H), 8.90 (d, 1H), 9.50 (d, 1H), 9.75 (s, 1H); MS (ESI, m/z): 496.1 [M+H]⁺

### Example 118. 4-(4-chlorophenyl)-6-(4-(2,3-dimethylphenyl)piperazin-1-yl)-2-(pyridin-3-yl)pyrimidine

Using 1-(2,3-dimethylphenyl)piperazine and separation method of PREP. HPLC, the title compound was obtained as described for the example 1 (Scheme 1. General procedure A.).

¹H NMR (400 MHz, CD₃OD) δ [ppm]=2.29 (s, 3H), 2.33 (s, 3H), 3.03 (br, 4H), 4.08 (br, 4H), 6.94 (t, 2H), 7.06 (t, 1H), 7.32 (s, 1H), 7.54 (d, 2H), 8.10-8.13 (m, 1H), 8.25 (d, 2H), 8.90 (d, 1H), 9.49 (d, 1H), 9.75 (s, 1H); MS (ESI, m/z): 456.2 [M+H]⁺

### Example 119. 4-(4-chlorophenyl)-6-(4-(3,4-dichlorophenyl)piperazin-1-yl)-2-(pyridin-3-yl)pyrimidine

Using 1-(3,4-dichlorophenyl)piperazine and separation method of PREP. HPLC, the title compound was obtained as described for the example 1 (Scheme 1. General procedure A.).

¹H NMR (400 MHz, CD₃OD) δ [ppm]=3.39 (t, 4H), 4.09 (br, 4H), 6.96 (d, 1H), 6.99 (d, 1H), 7.35 (d, 2H), 7.55 (d, 2H), 8.10-8.14 (m, 1H), 8.27 (d, 2H), 8.90 (d, 1H), 9.51 (d, 1H), 9.77 (s, 1H); MS (ESI, m/z): 496.1 [M+H]⁺

### Example 120. 4-(4-chlorophenyl)-6-(4-(4-methoxyphenyl)piperazin-1-yl)-2-(pyridin-3-yl)pyrimidine

Using 1-(4-methoxyphenyl)piperazine and separation method of PREP. HPLC, the title compound was obtained as described for the example 1 (Scheme 1. General procedure A.).

¹H NMR (400 MHz, CD₃OD) δ [ppm]=3.57 (t, 4H), 3.83 (s, 3H), 4.27 (br, 4H), 7.04 (d, 2H), 7.39 (d, 2H), 7.44 (s, 1H), 7.57 (d, 2H), 8.13-8.16 (m, 1H), 8.30 (d, 2H), 8.93 (d, 1H), 9.54 (d, 1H), 9.81 (s, 1H); MS (ESI, m/z): 458.2 [M+H]⁺

### Example 121. 4-(4-chlorophenyl)-6-(4-(4-nitrophenyl)piperazin-1-yl)-2-(pyridin-3-yl)pyrimidine

Using 1-(4-nitrophenyl)piperazine and separation method of PREP. HPLC, the title compound was obtained as described for the example 1 (Scheme 1. General procedure A.).

¹H NMR (400 MHz, CD₃OD) δ [ppm]=3.73 (t, 4H), 4.13 (br, 4H), 7.05 (d, 2H), 7.32 (s, 1H), 7.56 (d, 2H), 8.03-8.07 (m, 1H), 8.17 (d, 2H), 8.28 (d, 2H), 8.87 (d, 1H), 9.42 (d, 1H), 9.76 (s, 1H); MS (ESI, m/z): 473.1 [M+H]⁺

### Example 122. 4-(4-chlorophenyl)-6-(3-(4-methylpiperazin-1-yl)pyrrolidin-1-yl)-2-(pyridin-3-yl)pyrimidine

Using 1-methyl-4-(pyrrolidin-3-yl)piperazine and separation method of PREP. HPLC, the title compound was obtained as described for the example 1 (Scheme 1. General procedure A.).

¹H NMR (400 MHz, CD₃OD) δ [ppm]=2.02 (br, 2H), 2.41 (br, 2H), 2.93 (s, 3H), 2.97 (br, 2H), 3.28 (br, 2H), 3.38 (br, 4H), 3.62 (br, 2H), 4.16 (br, 1H), 6.99 (s, 1H), 7.54 (d, 2H), 8.10-8.13 (m, 1H), 8.23 (d, 2H), 8.91 (d, 1H), 9.49 (d, 1H), 9.77 (s, 1H); MS (ESI, m/z): 435.2 [M+H]⁺

### Example 123. 4-(4-benzhydrylpiperazin-1-yl)-6-(4-chlorophenyl)-2-(pyridin-3-yl)pyrimidine

Using 1-benzhydrylpiperazine and separation method of PREP. HPLC, the title compound was obtained as described for the example 1 (Scheme 1. General procedure A.).

¹H NMR (400 MHz, CD₃OD) δ [ppm]=3.35 (br, 4H), 4.22 (br, 4H), 5.41 (s, 1H), 7.31 (t, 2H), 7.37 (s, 1H), 7.43-7.55 (m, 8H), 7.69 (d, 2H), 8.00-8.04 (m, 1H), 8.26 (d, 2H), 8.86 (d, 1H), 9.40 (d, 1H), 9.74 (s, 1H); MS (ESI, m/z): 518.2 [M+H]⁺

### Example 124. 4-(4-chlorophenyl)-6-(4-((4-chlorophenyl)(phenyl)methyl)piperazin-1-yl)-2-(pyridin-3-yl)pyrimidine

Using 1-((4-chlorophenyl)(phenyl)methyl)piperazine and separation method of PREP. HPLC, the title compound was obtained as described for the example 1 (Scheme 1. General procedure A.).

¹H NMR (400 MHz, CD₃OD) δ [ppm]=3.29 (br, 4H), 4.21 (br, 4H), 5.36 (s, 1H), 7.30-7.34 (m, 1H), 7.36 (s, 1H), 7.42-7.54 (m, 7H), 7.67-7.71 (m, 3H), 8.07-8.11 (m, 1H), 8.25 (d, 2H), 8.90 (br, 1H), 9.47 (d, 1H), 9.76 (s, 1H); MS (ESI, m/z): 552.2 [M+H]⁺

### Example 125. 1′-(6-(4-chlorophenyl)-2-(pyridin-3-yl)pyrimidin-4-yl)spiro[indene-1,4′-piperidine]

Using spiro[indene-1,4′-piperidine] and separation method of PREP. HPLC, the title compound was obtained as described for the example 1 (Scheme 1. General procedure A.).

¹H NMR (400 MHz, CD₃OD) δ [ppm]=2.17-2.24 (m, 4H), 3.53-3.59 (m, 4H), 6.89 (d, 1H), 7.12 (d, 1H), 7.17 (t, 1H), 7.23 (t, 1H), 7.35 (d, 2H), 7.37 (s, 1H), 7.55 (d, 2H), 8.09-8.13 (m, 1H), 8.25 (d, 2H), 8.90 (d, 1H), 9.49 (d, 1H), 9.76 (s, 1H); MS (ESI, m/z): 451.2 [M+H]⁺

### Example 126. 6-(4-chlorophenyl)-2-(pyridin-3-yl)-N-(pyrrolidin-3-yl)pyrimidin-4-amine

Using tert-butyl 3-aminopyrrolidine-1-carboxylate and separation method of PREP. HPLC, the title compound was obtained as described for the example 54 (Scheme 1. General procedure A.).

¹H NMR (400 MHz, CD₃OD) δ [ppm]=2.16 (br, 1H), 2.50 (br, 1H), 3.18-3.23 (m, 1H), 3.38-3.46 (m, 1H), 3.52-3.59 (m, 1H), 3.70-3.75 (m, 1H), 4.04-4.11 (m, 1H), 7.00 (s, 1H), 7.53 (d, 2H), 7.70-7.74 (m, 1H), 8.16 (d, 2H), 8.72 (d, 1H), 9.06 (d, 1H), 9.63 (s, 1H); MS (ESI, m/z): 352.1 [M+H]⁺

### Example 127. (R)-6-(4-chlorophenyl)-2-(pyridin-3-yl)-N-(pyrrolidin-3-yl)pyrimidin-4-amine

Using tert-butyl (R)-3-aminopyrrolidine-1-carboxylate and separation method of PREP. HPLC, the title compound was obtained as described for the example 54 (Scheme 1. General procedure A.).

¹H NMR (400 MHz, CD₃OD) δ [ppm]=2.11-2.24 (m, 1H), 2.46-2.56 (m, 1H), 3.37-3.46 (m, 1H), 3.48-3.61 (m, 2H), 3.71-3.81 (m, 1H), 4.05-4.12 (m, 1H), 7.06 (s, 1H), 7.53 (d, 2H), 8.02-8.06 (m, 1H), 8.18 (d, 2H), 8.88 (d, 1H), 9.41 (d, 1H), 9.75 (s, 1H); MS (ESI, m/z): 352.1 [M+H]⁺

### Example 128. (R)-6-(4-chlorophenyl)-2-(pyridin-3-yl)-N-(pyrrolidin-3-ylmethyl)pyrimidin-4-amine

Using tert-butyl (S)-3-(aminomethyl)pyrrolidine-1-carboxylate and separation method of PREP. HPLC, the title compound was obtained as described for the example 54 (Scheme 1. General procedure A.).

¹H NMR (400 MHz, CD₃OD) δ [ppm]=1.85-1.94 (m, 1H), 2.22-2.32 (m, 1H), 2.79-2.87 (m, 1H), 3.00-3.15 (m, 2H), 3.40-3.46 (m, 1H), 3.47-3.57 (m, 1H), 3.73 (br, 2H), 7.00 (s, 1H), 7.53 (d, 2H), 8.05-8.08 (m, 1H), 8.16 (d, 2H), 8.88 (d, 1H), 9.43 (d, 1H), 9.75 (s, 1H); MS (ESI, m/z): 366.1 [M+H]⁺

### Example 129. 6-(4-chlorophenyl)-2-(pyridin-3-yl)-N-(2-(pyrrolidin-1-yl)ethyl)pyrimidin-4-amine

Using 2-(pyrrolidin-1-yl)ethan-1-amine and separation method of PREP. HPLC, the title compound was obtained as described for the example 1 (Scheme 1. General procedure A.).

¹H NMR (400 MHz, CD₃OD) δ [ppm]=3.19 (br, 4H), 3.40 (d, 2H), 3.53 (d, 2H), 3.76 (br, 4H), 7.06 (s, 1H), 7.54 (d, 2H), 8.02-8.05 (m, 1H), 8.18 (d, 2H), 8.88 (d, 1H), 9.41 (d, 1H), 9.76 (s, 1H); MS (ESI, m/z): 380.2 [M+H]⁺

### Example 130. 6-(4-chlorophenyl)-2-(pyridin-3-yl)-N-(3-(pyrrolidin-1-yl)propyl)pyrimidin-4-amine

Using 3-(pyrrolidin-1-yl)propan-1-amine and separation method of PREP. HPLC, the title compound was obtained as described for the example 1 (Scheme 1. General procedure A.).

¹H NMR (400 MHz, CD₃OD) δ [ppm]=2.00-2.18 (br, 6H), 3.03-3.13 (br, 6H), 3.69 (br, 2H), 6.98 (s, 1H), 7.52 (d, 2H), 7.95-7.98 (m, 1H), 8.15 (d, 2H), 8.84 (d, 1H), 9.31 (d, 1H), 9.71 (s, 1H); MS (ESI, m/z): 394.2 [M+H]⁺

### Example 131. 6-(4-chlorophenyl)-N-(2-(1-methylpyrrolidin-2-yl)ethyl)-2-(pyridin-3-yl)pyrimidin-4-amine

Using 2-(1-methylpyrrolidin-2-yl)ethan-1-amine and separation method of PREP. HPLC, the title compound was obtained as described for the example 1 (Scheme 1. General procedure A.).

¹H NMR (400 MHz, CD₃OD) δ [ppm]=1.89-2.00 (m, 2H), 2.02-2.18 (m, 2H), 2.35-2.43 (m, 1H), 2.93 (s, 3H), 3.03-3.07 (m, 2H), 3.11-3.20 (m, 1H), 3.34-3.44 (m, 1H), 3.72 (br, 2H), 6.96 (s, 1H), 7.52 (d, 2H), 7.87-7.90 (m, 1H), 8.15 (d, 2H), 8.80 (d, 1H), 9.21 (d, 1H), 9.67 (s, 1H); MS (ESI, m/z): 394.2 [M+H]⁺

### Example 132. N-(1-benzylpyrrolidin-3-yl)-6-(4-chlorophenyl)-2-(pyridin-3-yl)pyrimidin-4-amine

Using 1-benzylpyrrolidin-3-amine and separation method of PREP. HPLC, the title compound was obtained as described for the example 1 (Scheme 1. General procedure A.).

¹H NMR (400 MHz, CD₃OD) δ [ppm]=2.21 (br, 1H), 2.62 (br, 1H), 3.39-4.17 (br, 5H), 4.48 (s, 2H), 7.03 (s, 1H), 7.47-7.49 (m, 3H), 7.52-7.56 (m, 4H), 8.10-8.14 (m, 1H), 8.16 (d, 2H), 8.87 (d, 1H), 9.36 (d, 1H), 9.73 (s, 1H); MS (ESI, m/z): 442.2 [M+H]⁺

### Example 133. (3R,4S)-4-((6-(4-chlorophenyl)-2-(pyridin-3-yl)pyrimidin-4-yl)amino)pyrrolidin-3-ol

Using tert-butyl (3S,4R)-3-amino-4-hydroxypyrrolidine-1-carboxylate and separation method of PREP. HPLC, the title compound was obtained as described for the example 54 (Scheme 1. General procedure A.).

¹H NMR (400 MHz, CD₃OD) δ [ppm]=1.06 (br, 2H), 2.18 (br, 1H), 3.80 (br, 1H), 4.17 (br, 2H), 7.09 (s, 1H), 7.53 (d, 2H), 7.65-7.70 (m, 1H), 8.17 (d, 2H), 8.69 (d, 1H), 9.01 (d, 1H), 9.62 (s, 1H); MS (ESI, m/z): 368.1 [M+H]⁺

### Example 134. (3S,4R)-4-((6-(4-chlorophenyl)-2-(pyridin-3-yl)pyrimidin-4-yl)amino)pyrrolidin-3-ol

Using tert-butyl (3R,4S)-3-amino-4-hydroxypyrrolidine-1-carboxylate and separation method of PREP. HPLC, the title compound was obtained as described for the example 54 (Scheme 1. General procedure A.).

¹H NMR (400 MHz, CD₃OD) δ [ppm]=1.06 (br, 2H), 2.18 (br, 1H), 3.80 (br, 1H), 4.17 (br, 2H), 7.09 (s, 1H), 7.53 (d, 2H), 7.65-7.70 (m, 1H), 8.17 (d, 2H), 8.69 (d, 1H), 9.01 (d, 1H), 9.62 (s, 1H); MS (ESI, m/z): 368.1 [M+H]⁺

### Example 135. 4-(4-chlorophenyl)-2-(pyridin-3-yl)-6-(pyrrolidin-1-yl)pyrimidine

Using pyrrolidine and separation method of PREP. HPLC, the title compound was obtained as described for the example 1 (Scheme 1. General procedure A.).

¹H NMR (400 MHz, CD₃OD) δ [ppm]=2.09 (br, 4H), 3.59 (br, 2H), 3.82 (br, 2H), 6.95 (s, 1H), 7.54 (d, 2H), 8.05-8.08 (m, 1H), 8.20 (d, 2H), 8.88 (d, 1H), 9.40 (d, 1H), 9.69 (s, 1H); MS (ESI, m/z): 337.1 [M+H]⁺

### Example 136. 4-(4-chlorophenyl)-6-(2-methylpyrrolidin-1-yl)-2-(pyridin-3-yl)pyrimidine

Using 2-methylpyrrolidine and separation method of PREP. HPLC, the title compound was obtained as described for the example 1 (Scheme 1. General procedure A.).

¹H NMR (400 MHz, CD₃OD) δ [ppm]=1.36 (br, 3H), 1.87 (br, 1H), 2.20 (br, 3H), 3.70 (br, 2H), 4.63 (br, 1H), 6.91 (s, 1H), 7.53 (d, 2H), 8.04-8.07 (m, 1H), 8.18 (d, 2H), 8.88 (d, 1H), 9.37 (d, 1H), 9.67 (s, 1H); MS (ESI, m/z): 351.1 [M+H]⁺

### Example 137. (S)-1-(6-(4-chlorophenyl)-2-(pyridin-3-yl)pyrimidin-4-yl)pyrrolidin-3-ol

Using (S)-pyrrolidin-3-ol and separation method of PREP. HPLC, the title compound was obtained as described for the example 1 (Scheme 1. General procedure A.).

¹H NMR (400 MHz, CD₃OD) δ [ppm]=2.17 (br, 2H), 3.70 (br, 2H), 3.86 (br, 2H), 4.59 (br, 1H), 6.95 (s, 1H), 7.52 (d, 2H), 8.07-8.11 (m, 1H), 8.19 (d, 2H), 8.89 (d, 1H), 9.43 (d, 1H), 9.69 (s, 1H); MS (ESI, m/z): 353.1 [M+H]⁺

### Example 138. (1-(6-(4-chlorophenyl)-2-(pyridin-3-yl)pyrimidin-4-yl)pyrrolidin-3-yl)methanol

Using pyrrolidin-3-ylmethanol and separation method of PREP. HPLC, the title compound was obtained as described for the example 1 (Scheme 1. General procedure A.).

¹H NMR (400 MHz, CD₃OD) δ [ppm]=1.92 (br, 1H), 2.21 (br, 1H), 2.60 (br, 1H), 3.52-3.59 (m, 2H), 3.60-3.71 (m, 3H), 3.97 (br, 1H), 6.91 (s, 1H), 7.52 (d, 2H), 8.04-8.08 (m, 1H), 8.18 (d, 2H), 8.88 (d, 1H), 9.39 (d, 1H), 9.68 (s, 1H); MS (ESI, m/z): 367.1 [M+H]⁺

### Example 139. (R)-4-(4-chlorophenyl)-6-(3-fluoropyrrolidin-1-yl)-2-(pyridin-3-yl)pyrimidine

Using (R)-3-fluoropyrrolidine and separation method of PREP. HPLC, the title compound was obtained as described for the example 1 (Scheme 1. General procedure A.).

¹H NMR (400 MHz, CD₃OD) δ [ppm]=2.25 (br, 1H), 2.45 (br, 1H), 3.71 (br, 1H), 3.79 (br, 2H), 3.88 (br, 1H), 4.17 (br, 1H), 7.00 (s, 1H), 7.53 (d, 2H), 8.07-8.10 (m, 1H), 8.23 (d, 2H), 8.88 (d, 1H), 9.46 (d, 1H), 9.72 (s, 1H); MS (ESI, m/z): 355.1 [M+H]⁺

### Example 140. 1-(6-(4-chlorophenyl)-2-(pyridin-3-yl)pyrimidin-4-yl)pyrrolidin-3-amine

Using tert-butyl pyrrolidin-3-ylcarbamate and separation method of PREP. HPLC, the title compound was obtained as described for the example 54 (Scheme 1. General procedure A.).

¹H NMR (400 MHz, CD₃OD) δ [ppm]=2.31 (br, 1H), 2.57 (br, 1H), 3.87 (br, 2H), 4.06 (br, 2H), 4.14 (br, 1H), 7.04 (s, 1H), 7.54 (d, 2H), 8.06-8.09 (m, 1H), 8.25 (d, 2H), 8.89 (d, 1H), 9.45 (d, 1H), 9.75 (s, 1H); MS (ESI, m/z): 352.1 [M+H]⁺

### Example 141. 1-(6-(4-chlorophenyl)-2-(pyridin-3-yl)pyrimidin-4-yl)-N-methylpyrrolidin-3-amine

Using tert-butyl methyl(pyrrolidin-3-yl)carbamate and separation method of PREP. HPLC, the title compound was obtained as described for the example 54 (Scheme 1. General procedure A.).

¹H NMR (400 MHz, CD₃OD) δ [ppm]=2.38 (br, 1H), 2.59 (br, 1H), 2.85 (s, 3H), 3.80 (br, 1H), 3.89 (br, 1H), 4.05 (br, 3H), 7.04 (s, 1H), 7.53 (d, 2H), 8.08-8.12 (m, 1H), 8.25 (d, 2H), 8.90 (d, 1H), 9.47 (d, 1H), 9.76 (s, 1H); MS (ESI, m/z): 366.1 [M+H]⁺

### Example 142. methyl (6-(4-chlorophenyl)-2-(pyridin-3-yl)pyrimidin-4-yl)prolinate

Using methyl prolinate and separation method of PREP. HPLC, the title compound was obtained as described for the example 1 (Scheme 1. General procedure A.).

¹H NMR (400 MHz, CD₃OD) δ [ppm]=2.13-2.29 (m, 3H), 2.39-2.49 (m, 1H), 3.63-3.80 (m, 5H), 4.76-4.79 (m, 1H), 7.06 (s, 1H), 7.52 (d, 2H), 8.05-8.08 (m, 1H), 8.23 (d, 2H), 8.88 (d, 1H), 9.32 (d, 1H), 9.59 (s, 1H); MS (ESI, m/z): 395.1 [M+H]⁺

### Example 143. N-(1-(6-(4-chlorophenyl)-2-(pyridin-3-yl)pyrimidin-4-yl)pyrrolidin-3-yl)acetamide

Using N-(pyrrolidin-3-yl)acetamide and separation method of PREP. HPLC, the title compound was obtained as described for the example 1 (Scheme 1. General procedure A.).

¹H NMR (400 MHz, CD₃OD) δ [ppm]=1.98 (s, 3H), 2.13 (br, 1H), 2.37 (br, 1H), 3.46 (br, 1H), 3.72 (br, 1H), 3.89 (br, 1H), 4.02 (br, 1H), 4.52 (br, 1H), 6.97 (s, 1H), 7.53 (d, 2H), 8.13-8.16 (m, 1H), 8.21 (d, 2H), 8.92 (d, 1H), 9.50 (d, 1H), 9.73 (s, 1H); MS (ESI, m/z): 394.1 [M+H]⁺

### Example 144. (2R,3R)-3-((6-(4-chlorophenyl)-2-(pyridin-3-yl)pyrimidin-4-yl)amino)butan-2-ol

Using (2R,3R)-3-aminobutan-2-ol and separation method of PREP. HPLC, the title compound was obtained as described for the example 1 (Scheme 1. General procedure A.).

¹H NMR (600 MHz, CD₃OD) a [ppm]=1.23 (d, 3H), 1.30 (d, 3H), 3.94 (br, 1H), 4.51 (br, 1H), 7.01 (s, 1H), 7.55 (d, 2H), 8.07-8.10 (m, 1H), 8.15 (d, 2H), 8.89 (d, 1H), 9.44 (d, 1H), 9.71 (s, 1H); MS (ESI, m/z): 355.1 [M+H]⁺

### Example 145. 3-((6-(4-chlorophenyl)-2-(pyridin-3-yl)pyrimidin-4-yl)amino)butan-2-ol

Using 3-aminobutan-2-ol and separation method of PREP. HPLC, the title compound was obtained as described for the example 1 (Scheme 1. General procedure A.).

¹H NMR (600 MHz, CD₃OD) a [ppm]=1.27 (d, 3H), 1.31 (d, 3H), 3.90-3.94 (m, 1H), 4.46 (br, 1H), 7.00 (s, 1H), 7.55 (d, 2H), 8.13-8.20 (m, 3H), 8.92 (d, 1H), 9.48 (d, 1H), 9.71 (s, 1H); MS (ESI, m/z): 355.1 [M+H]⁺

### Example 146. 1-(4-((6-(4-chlorophenyl)-2-(pyridin-3-yl)pyrimidin-4-yl)amino)piperidin-1-yl)ethan-1-one

Using 1-(4-aminopiperidin-1-yl)ethan-1-one and separation method of PREP. HPLC, the title compound was obtained as described for the example 1 (Scheme 1. General procedure A.).

¹H NMR (600 MHz, CD₃OD) δ [ppm]=1.49-1.65 (m, 4H), 2.05-2.22 (m, 4H), 2.17 (s, 3H), 4.49 (br, 1H), 6.99 (s, 1H), 7.57 (d, 2H), 8.14-8.19 (m, 3H), 8.93 (d, 1H), 9.52 (d, 1H), 9.77 (s, 1H); MS (ESI, m/z): 408.2 [M+H]⁺

### Example 147. (R)-(1-(6-(4-chlorophenyl)-2-(pyridin-3-yl)pyrimidin-4-yl)piperidin-3-yl)methanol

Using (R)-piperidin-3-ylmethanol and separation method of PREP. HPLC, the title compound was obtained as described for the example 1 (Scheme 1. General procedure A.).

¹H NMR (600 MHz, CD₃OD) δ [ppm]=1.39-1.45 (m, 1H), 1.59-1.66 (m, 1H), 1.79-1.84 (m, 1H), 1.86-1.92 (m, 2H), 3.03 (t, 1H), 3.24 (t, 1H), 3.35 (d, 1H), 3.50 (dd, 1H), 3.60 (dd, 1H), 4.58 (br, 1H), 7.27 (s, 1H), 7.54 (d, 2H), 8.13-8.15 (m, 1H), 8.21 (d, 2H), 8.92 (d, 1H), 9.50 (d, 1H), 9.73 (s, 1H); MS (ESI, m/z): 381.1 [M+H]⁺

### Example 148. (S)-(1-(6-(4-chlorophenyl)-2-(pyridin-3-yl)pyrimidin-4-yl)piperidin-3-yl)methanol

Using (S)-piperidin-3-ylmethanol and separation method of PREP. HPLC, the title compound was obtained as described for the example 1 (Scheme 1. General procedure A.).

¹H NMR (600 MHz, CD₃OD) δ [ppm]=1.39-1.45 (m, 1H), 1.59-1.66 (m, 1H), 1.79-1.84 (m, 1H), 1.86-1.92 (m, 2H), 3.03 (t, 1H), 3.24 (t, 1H), 3.35 (d, 1H), 3.50 (dd, 1H), 3.60 (dd, 1H), 4.58 (br, 1H), 7.27 (s, 1H), 7.54 (d, 2H), 8.13-8.15 (m, 1H), 8.21 (d, 2H), 8.92 (d, 1H), 9.50 (d, 1H), 9.73 (s, 1H); MS (ESI, m/z): 381.1 [M+H]⁺

### Example 149. 6-(4-chlorophenyl)-N-(2-(piperidin-1-yl)ethyl)-2-(pyridin-3-yl)pyrimidin-4-amine

Using 2-(piperidin-1-yl)ethan-1-amine and separation method of PREP. HPLC, the title compound was obtained as described for the example 1 (Scheme 1. General procedure A.).

¹H NMR (600 MHz, CD₃OD) δ [ppm]=1.51-1.57 (m, 1H), 1.75-1.86 (m, 3H), 1.97 (d, 2H), 3.05 (t, 2H), 3.48 (t, 2H), 3.72 (d, 2H), 4.05 (br, 2H), 7.08 (s, 1H), 7.57 (d, 2H), 8.10-8.12 (m, 1H), 8.21 (d, 2H), 8.92 (d, 1H), 9.48 (d, 1H), 9.80 (s, 1H); MS (ESI, m/z): 394.2 [M+H]⁺

### Example 150. 1-(6-(4-chlorophenyl)-2-(pyridin-3-yl)pyrimidin-4-yl)piperidine-4-carbonitrile

Using piperidine-4-carbonitrile and separation method of PREP. HPLC, the title compound was obtained as described for the example 1 (Scheme 1. General procedure A.).

¹H NMR (600 MHz, CD₃OD) δ [ppm]=1.89-1.95 (m, 2H), 2.08-2.13 (m, 2H), 3.16-3.20 (m, 1H), 3.76-3.80 (m, 2H), 4.24 (br, 2H), 7.32 (s, 1H), 7.54 (d, 2H), 8.11-8.13 (m, 1H), 8.25 (d, 2H), 8.90 (d, 1H), 9.50 (d, 1H), 9.75 (s, 1H); MS (ESI, m/z): 376.1 [M+H]⁺

### Example 151. 1-(6-(4-chlorophenyl)-2-(pyridin-3-yl)pyrimidin-4-yl)-4-(3-(trifluoromethyl)phenyl)piperidin-4-ol

Using 4-(3-(trifluoromethyl)phenyl)piperidin-4-ol and separation method of PREP. HPLC, the title compound was obtained as described for the example 1 (Scheme 1. General procedure A.).

¹H NMR (600 MHz, CD₃OD) δ [ppm]=1.92 (d, 2H), 2.14-2.19 (m, 2H), 3.59 (br, 2H), 4.70 (br, 2H), 7.33 (s, 1H), 7.53 (d, 2H), 7.53-7.59 (m, 2H), 7.76 (d, 1H), 7.88 (s, 1H), 8.13-8.15 (m, 1H), 8.22 (d, 2H), 8.92 (d, 1H), 9.50 (d, 1H), 9.74 (s, 1H); MS (ESI, m/z): 511.1 [M+H]⁺

### Example 152. 4-(4-chlorophenyl)-2-(pyridin-3-yl)-6-(4-(pyrrolidin-1-yl)piperidin-1-yl)pyrimidine

Using 4-(pyrrolidin-1-yl)piperidine and separation method of PREP. HPLC, the title compound was obtained as described for the example 1 (Scheme 1. General procedure A.).

¹H NMR (600 MHz, CD₃OD) δ [ppm]=1.71-1.78 (m, 2H), 2.03 (br, 2H), 2.18 (br, 2H), 2.34 (d, 2H), 3.13 (t, 2H), 3.22 (br, 2H), 3.31 (br, 2H), 3.52-3.56 (m, 1H), 3.70 (br, 2H), 7.36 (s, 1H), 7.54 (d, 2H), 8.16-8.18 (m, 1H), 8.25 (d, 2H), 8.94 (d, 1H), 9.55 (d, 1H), 9.78 (s, 1H); MS (ESI, m/z): 420.2 [M+H]⁺

### Example 153. 4-(4-chlorophenyl)-1-(6-(4-chlorophenyl)-2-(pyridin-3-yl)pyrimidin-4-yl)piperidin-4-ol

Using 4-(4-chlorophenyl)piperidin-4-ol and separation method of PREP. HPLC, the title compound was obtained as described for the example 1 (Scheme 1. General procedure A.).

¹H NMR (600 MHz, CD₃OD) δ [ppm]=1.90 (d, 2H), 2.08-2.13 (m, 2H), 3.58 (br, 2H), 4.66 (br, 2H), 7.32 (s, 1H), 7.33 (d, 2H), 7.51 (d, 2H), 7.53 (d, 2H), 8.13-8.15 (m, 1H), 8.22 (d, 2H), 8.92 (d, 1H), 9.50 (d, 1H), 9.74 (s, 1H); MS (ESI, m/z): 477.1 [M+H]⁺

### Example 154. 1-(4-(((6-(4-chlorophenyl)-2-(pyridin-3-yl)pyrimidin-4-yl)amino)methyl)piperidin-1-yl)ethan-1-one

Using 1-(4-(aminomethyl)piperidin-1-yl)ethan-1-one and separation method of PREP. HPLC, the title compound was obtained as described for the example 1 (Scheme 1. General procedure A.).

¹H NMR (600 MHz, CD₃OD) δ [ppm]=1.22-1.39 (m, 3H), 1.91 (d, 2H), 1.96 (s, 3H), 3.08-3.13 (m, 4H), 4.79 (br, 2H), 7.28 (s, 1H), 7.54 (d, 2H), 8.14-8.16 (m, 1H), 8.22 (d, 2H), 8.92 (d, 1H), 9.50 (d, 1H), 9.73 (s, 1H); MS (ESI, m/z): 422.2 [M+H]⁺

### Example 155. 1-(1-(6-(4-chlorophenyl)-2-(pyridin-3-yl)pyrimidin-4-yl)-4-phenylpiperidin-4-yl)ethan-1-one

Using 1-(4-phenylpiperidin-4-yl)ethan-1-one and separation method of PREP. HPLC, the title compound was obtained as described for the example 1 (Scheme 1. General procedure A.).

¹H NMR (600 MHz, CD₃OD) δ [ppm]=1.99 (s, 3H), 2.14-2.19 (m, 2H), 2.58 (d, 2H), 3.60 (t, 2H), 4.27 (br, 2H), 7.24 (s, 1H), 7.30-7.34 (m, 1H), 7.42 (d, 4H), 7.51 (d, 2H), 8.10-8.12 (m, 1H), 8.20 (d, 2H), 8.90 (d, 1H), 9.46 (d, 1H), 9.71 (s, 1H); MS (ESI, m/z): 469.2 [M+H]⁺

### Example 156. 4-(1-(6-(4-chlorophenyl)-2-(pyridin-3-yl)pyrimidin-4-yl)piperidin-4-yl)morpholine

Using 4-(piperidin-4-yl)morpholine and separation method of PREP. HPLC, the title compound was obtained as described for the example 1 (Scheme 1. General procedure A.).

¹H NMR (600 MHz, CD₃OD) δ [ppm]=1.75-1.82 (m, 2H), 2.35 (d, 2H), 3.14 (t, 2H), 3.24 (t, 2H), 3.55 (d, 2H), 3.60-3.66 (m, 2H), 3.80 (t, 2H), 4.10 (d, 2H), 5.00 (br, 2H), 7.37 (s, 1H), 7.54 (d, 2H), 8.17-8.19 (m, 1H), 8.26 (d, 2H), 8.94 (d, 1H), 9.56 (d, 1H), 9.79 (s, 1H); MS (ESI, m/z): 436.2 [M+H]⁺

### Example 157. 4-(4-chlorophenyl)-6-(4-(3,5-dichlorophenyl)piperidin-1-yl)-2-(pyridin-3-yl)pyrimidine

Using 4-(3,5-dichlorophenyl)piperidine and separation method of PREP. HPLC, the title compound was obtained as described for the example 1 (Scheme 1. General procedure A.).

¹H NMR (600 MHz, CD₃OD) δ [ppm]=1.72-1.79 (m, 2H), 2.04 (d, 2H), 2.98-3.04 (m, 1H), 3.20 (t, 2H), 4.95 (br, 2H), 7.27 (s, 2H), 7.29 (s, 1H), 7.35 (s, 1H), 7.55 (d, 2H), 8.15-8.18 (m, 1H), 8.25 (d, 2H), 8.93 (d, 1H), 9.54 (d, 1H), 9.76 (s, 1H); MS (ESI, m/z): 495.1 [M+H]⁺

### Example 158. 6-(4-chlorophenyl)-N-((1-cyclohexylpiperidin-3-yl)methyl)-2-(pyridin-3-yl)pyrimidin-4-amine

Using (1-cyclohexylpiperidin-3-yl)methanamine and separation method of PREP. HPLC, the title compound was obtained as described for the example 1 (Scheme 1. General procedure A.).

¹H NMR (600 MHz, CD₃OD) δ [ppm]=1.35-1.41 (m, 2H), 1.46-1.53 (m, 2H), 1.61-1.72 (m, 3H), 1.93 (d, 2H), 2.08 (d, 3H), 2.15 (d, 2H), 3.07 (t, 2H), 3.14-3.19 (m, 1H), 3.54 (d, 2H), 3.59 (br, 1H), 4.87-4.91 (m, 2H), 6.99 (s, 1H), 7.53 (d, 2H), 8.14-8.16 (m, 3H), 8.93 (d, 1H), 9.51 (d, 1H), 9.76 (s, 1H); MS (ESI, m/z): 462.2 [M+H]⁺

### Example 159. N-((1-benzylpiperidin-4-yl)methyl)-6-(4-chlorophenyl)-2-(pyridin-3-yl)pyrimidin-4-amine

Using (1-benzylpiperidin-4-yl)methanamine and separation method of PREP. HPLC, the title compound was obtained as described for the example 1 (Scheme 1. General procedure A.).

¹H NMR (600 MHz, CD₃OD) δ [ppm]=1.56-1.63 (m, 2H), 2.07 (br, 2H), 2.14 (d, 2H), 3.05 (t, 2H), 3.56 (d, 2H), 3.59 (br, 1H), 4.30 (s, 2H), 7.00 (s, 1H), 7.49 (s, 5H), 7.54 (d, 2H), 8.13-8.15 (m, 3H), 8.94 (d, 1H), 9.51 (d, 1H), 9.77 (s, 1H); MS (ESI, m/z): 470.2 [M+H]⁺

### Example 160. ethyl 3-(1-(6-(4-chlorophenyl)-2-(pyridin-3-yl)pyrimidin-4-yl)piperidin-4-yl)-3-oxopropanoate

Using ethyl 3-oxo-3-(piperidin-4-yl)propanoate and separation method of PREP. HPLC, the title compound was obtained as described for the example 1 (Scheme 1. General procedure A.).

¹H NMR (600 MHz, CD₃OD) δ [ppm]=1.24 (t, 3H), 1.60-1.71 (m, 4H), 2.18-2.24 (m, 1H), 3.20-3.29 (m, 2H), 3.70 (s, 2H), 4.69 (br, 4H), 7.26 (s, 1H), 7.53 (d, 2H), 8.06-8.08 (m, 1H), 8.22 (d, 2H), 8.88 (d, 1H), 9.42 (d, 1H), 9.71 (s, 1H); MS (ESI, m/z): 465.2 [M+H]⁺

### Example 161. ethyl 2-(1-(6-(4-chlorophenyl)-2-(pyridin-3-yl)pyrimidin-4-yl)piperidin-4-yl)acetate

Using ethyl 2-(piperidin-4-yl)acetate and separation method of PREP. HPLC, the title compound was obtained as described for the example 1 (Scheme 1. General procedure A.).

¹H NMR (600 MHz, CD₃OD) δ [ppm]=1.28 (t, 3H), 1.95 (d, 2H), 2.15-2.23 (m, 1H), 2.35 (d, 2H), 3.13 (t, 2H), 3.80 (br, 2H), 4.17 (q, 2H), 4.78 (br, 2H), 7.27 (s, 1H), 7.55 (d, 2H), 8.16-8.18 (m, 1H), 8.22 (d, 2H), 8.94 (d, 1H), 9.51 (d, 1H), 9.74 (s, 1H); MS (ESI, m/z): 437.2 [M+H]⁺

### Example 162. (1S,3R)-3-((6-(4-chlorophenyl)-2-(pyridin-3-yl)pyrimidin-4-yl)amino)cyclopentan-1-ol

Using (1S,3R)-3-aminocyclopentan-1-ol and separation method of PREP. HPLC, the title compound was obtained as described for the example 1 (Scheme 1. General procedure A.).

¹H NMR (600 MHz, CD₃OD) δ [ppm]=1.61-1.67 (m, 1H), 1.79-1.95 (m, 4H), 2.15-2.22 (m, 1H), 2.42-2.49 (m, 1H), 4.34-4.39 (m, 1H), 4.60 (br, 1H), 6.92 (s, 1H), 7.50 (d, 2H), 8.08-8.14 (m, 3H), 8.91 (d, 1H), 9.44 (d, 1H), 9.69 (s, 1H); MS (ESI, m/z): 367.1 [M+H]⁺

### Example 163. (S)-1-(6-(4-chlorophenyl)-2-(pyridin-3-yl)pyrimidin-4-yl)piperidin-3-ol

Using (S)-piperidin-3-ol and separation method of PREP. HPLC, the title compound was obtained as described for the example 1 (Scheme 1. General procedure A.).

¹H NMR (600 MHz, CD₃OD) δ [ppm]=1.59-1.66 (m, 1H), 1.67-1.72 (m, 1H), 1.93-1.99 (m, 1H), 2.04-2.08 (m, 1H), 3.51 (q, 1H), 3.65 (br, 1H), 3.80-3.82 (m, 1H), 4.06 (br, 1H), 4.30 (br, 1H), 7.26 (s, 1H), 7.53 (d, 2H), 8.14-8.16 (m, 1H), 8.20 (d, 2H), 8.92 (d, 1H), 9.50 (d, 1H), 9.73 (s, 1H); MS (ESI, m/z): 367.1 [M+H]⁺

### Example 164. 1-(6-(4-chlorophenyl)-2-(pyridin-3-yl)pyrimidin-4-yl)-N,N-dimethylpyrrolidin-3-amine

Using N,N-dimethylpyrrolidin-3-amine and separation method of PREP. HPLC, the title compound was obtained as described for the example 1 (Scheme 1. General procedure A.).

¹H NMR (600 MHz, CD₃OD) δ [ppm]=2.40 (br, 1H), 2.68 (br, 1H), 3.05 (s, 6H), 3.72 (br, 1H), 3.94 (br, 1H), 4.13 (br, 2H), 4.33 (br, 1H), 7.08 (s, 1H), 7.54 (d, 2H), 8.15-8.18 (m, 1H), 8.27 (d, 2H), 8.94 (d, 1H), 9.57 (d, 1H), 9.81 (s, 1H); MS (ESI, m/z): 380.2 [M+H]⁺

### Example 165. 2-(1-(6-(4-chlorophenyl)-2-(pyridin-3-yl)pyrimidin-4-yl)pyrrolidin-2-yl)-N,N-dimethylethan-1-amine

Using N,N-dimethyl-2-(pyrrolidin-2-yl)ethan-1-amine and separation method of PREP. HPLC, the title compound was obtained as described for the example 1 (Scheme 1. General procedure A.).

¹H NMR (600 MHz, CD₃OD) δ [ppm]=2.08-2.15 (m, 2H), 2.39-2.45 (m, 2H), 2.88 (s, 6H), 3.08-3.13 (m, 2H), 3.26-3.31 (m, 2H), 3.50-3.59 (m, 2H), 3.92-3.96 (m, 1H), 7.31 (s, 1H), 7.56 (d, 2H), 8.20-8.22 (m, 1H), 8.26 (d, 2H), 8.95 (d, 1H), 9.58 (d, 1H), 9.80 (s, 1H); MS (ESI, m/z): 408.2 [M+H]⁺

### Example 166. 1-(6-(4-chlorophenyl)-2-(pyridin-3-yl)pyrimidin-4-yl)piperidin-4-one

Using piperidin-4-one and separation method of PREP. HPLC, the title compound was obtained as described for the example 1 (Scheme 1. General procedure A.).

¹H NMR (600 MHz, CD₃OD) δ [ppm]=1.85-1.90 (m, 4H), 3.93 (br, 2H), 3.99 (br, 2H), 7.31 (s, 1H), 7.54 (d, 2H), 8.12-8.14 (m, 1H), 8.23 (d, 2H), 8.91 (d, 1H), 9.49 (d, 1H), 9.74 (s, 1H); MS (ESI, m/z): 365.1 [M+H]⁺

### Example 167. 6-(4-chlorophenyl)-N-methyl-N-(piperidin-4-yl)-2-(pyridin-3-yl)pyrimidin-4-amine

Using tert-butyl 4-(methylamino)piperidine-1-carboxylate and separation method of PREP. HPLC, the title compound was obtained as described for the example 54 (Scheme 1. General procedure A.).

¹H NMR (600 MHz, CD₃OD) δ [ppm]=2.05 (d, 2H), 2.14-2.21 (m, 2H), 3.17 (s, 3H), 3.34-3.36 (m, 2H), 3.59 (d, 2H), 5.26 (br, 1H), 7.23 (s, 1H), 7.55 (d, 2H), 8.16-8.18 (m, 1H), 8.27 (d, 2H), 8.94 (d, 1H), 9.59 (d, 1H), 9.85 (s, 1H); MS (ESI, m/z): 380.2 [M+H]⁺

### Example 168. 6-(4-chlorophenyl)-N-(2-(1-methylpiperidin-2-yl)ethyl)-2-(pyridin-3-yl)pyrimidin-4-amine

Using 2-(1-methylpiperidin-2-yl)ethan-1-amine and separation method of PREP. HPLC, the title compound was obtained as described for the example 1 (Scheme 1. General procedure A.).

¹H NMR (600 MHz, CD₃OD) δ [ppm]=1.90-1.96 (m, 4H), 2.30 (d, 1H), 2.41-2.46 (m, 1H), 2.92 (s, 3H), 3.05-3.10 (m, 1H), 3.52-3.57 (m, 2H), 3.73 (br, 2H), 3.77 (br, 2H), 7.01 (s, 1H), 7.54 (d, 2H), 8.13-8.15 (m, 1H), 8.17 (d, 2H), 8.93 (d, 1H), 9.49 (d, 1H), 9.76 (s, 1H); MS (ESI, m/z): 408.2 [M+H]⁺

### Example 169. 6-(4-chlorophenyl)-N-(1-(1-methylpiperidin-4-yl)ethyl)-2-(pyridin-3-yl)pyrimidin-4-amine

Using 1-(1-methylpiperidin-4-yl)ethan-1-amine and separation method of PREP. HPLC, the title compound was obtained as described for the example 1 (Scheme 1. General procedure A.).

¹H NMR (600 MHz, CD₃OD) δ [ppm]=1.32 (d, 3H), 1.39-1.45 (m, 2H), 1.62-1.67 (m, 1H), 1.94 (br, 2H), 2.11-2.18 (m, 1H), 3.85 (s, 3H), 2.97-3.13 (m, 2H), 3.48-3.63 (m, 2H), 7.29 (s, 1H), 7.54 (d, 2H), 7.94-7.97 (m, 1H), 8.23 (d, 2H), 8.83 (d, 1H), 9.30 (d, 1H), 9.70 (s, 1H); MS (ESI, m/z): 408.2 [M+H]⁺

### Example 170. 6-(4-chlorophenyl)-N-((1-(2-methoxyethyl)piperidin-4-yl)methyl)-2-(pyridin-3-yl)pyrimidin-4-amine

Using (1-(2-methoxyethyl)piperidin-4-yl)methanamine and separation method of PREP. HPLC, the title compound was obtained as described for the example 1 (Scheme 1. General procedure A.).

¹H NMR (600 MHz, CD₃OD) δ [ppm]=1.65 (br, 2H), 2.12 (br, 2H), 3.02 (br, 2H), 3.40 (s, 3H), 3.48 (br, 1H), 3.60 (br, 1H), 3.64-3.72 (m, 2H), 4.51 (br, 1H), 4.90-4.97 (m, 4H), 6.97 (s, 1H), 7.54 (d, 2H), 7.94-7.98 (m, 1H), 8.16 (d, 2H), 8.83 (d, 1H), 9.29 (d, 1H), 9.70 (s, 1H); MS (ESI, m/z): 438.2 [M+H]⁺

### Example 171. methyl 2-(4-((6-(4-chlorophenyl)-2-(pyridin-3-yl)pyrimidin-4-yl)amino)piperidin-1-yl)acetate

Using methyl 2-(4-aminopiperidin-1-yl)acetate and separation method of PREP. HPLC, the title compound was obtained as described for the example 1 (Scheme 1. General procedure A.).

¹H NMR (600 MHz, CD₃OD) δ [ppm]=1.97 (br, 4H), 2.43 (br, 4H), 3.89 (s, 3H), 4.23 (s, 2H), 4.47 (br, 1H), 6.97 (s, 1H), 7.54 (d, 2H), 7.81-7.84 (m, 1H), 8.16 (d, 2H), 8.77 (d, 1H), 9.16 (d, 1H), 9.66 (s, 1H); MS (ESI, m/z): 438.2 [M+H]⁺

### Example 172. 1-(1-(6-(4-chlorophenyl)-2-(pyridin-3-yl)pyrimidin-4-yl)piperidin-4-yl)ethyl 2,2,2-trifluoroacetate

Using 1-(piperidin-4-yl)ethyl 2,2,2-trifluoroacetate and separation method of PREP. HPLC, the title compound was obtained as described for the example 1 (Scheme 1. General procedure A.).

¹H NMR (600 MHz, CD₃OD) δ [ppm]=1.20 (d, 3H), 1.29-1.44 (m, 4H), 1.66 (br, 1H), 3.05 (br, 4H), 3.53-3.59 (m, 1H), 7.26 (s, 1H), 7.54 (d, 2H), 8.02-8.05 (m, 1H), 8.21 (d, 2H), 8.86 (d, 1H), 9.37 (d, 1H), 9.69 (s, 1H); MS (ESI, m/z): 491.1 [M+H]⁺

### Example 173. 6-(4-chlorophenyl)-N-(1-methylpiperidin-3-yl)-2-(pyridin-3-yl)pyrimidin-4-amine

Using 1-methylpiperidin-3-amine and separation method of PREP. HPLC, the title compound was obtained as described for the example 1 (Scheme 1. General procedure A.).

¹H NMR (600 MHz, CD₃OD) δ [ppm]=1.61-1.71 (m, 1H), 1.94-2.07 (m, 2H), 2.14-2.26 (m, 2H), 2.79 (t, 1H), 2.96 (s, 3H), 3.57-3.62 (m, 1H), 3.88-3.93 (m, 1H), 4.62 (br, 1H), 7.00 (s, 1H), 7.55 (d, 2H), 7.97-8.00 (m, 1H), 8.17 (d, 2H), 8.86 (d, 1H), 9.38 (d, 1H), 9.79 (s, 1H); MS (ESI, m/z): 380.2 [M+H]⁺

### Example 174. (1S,2R)-2-((6-(4-chlorophenyl)-2-(1-methyl-1H-pyrazol-4-yl)pyrimidin-4-yl)amino)cyclopentan-1-ol

Scheme for the preparation of the Compound of Example 174:

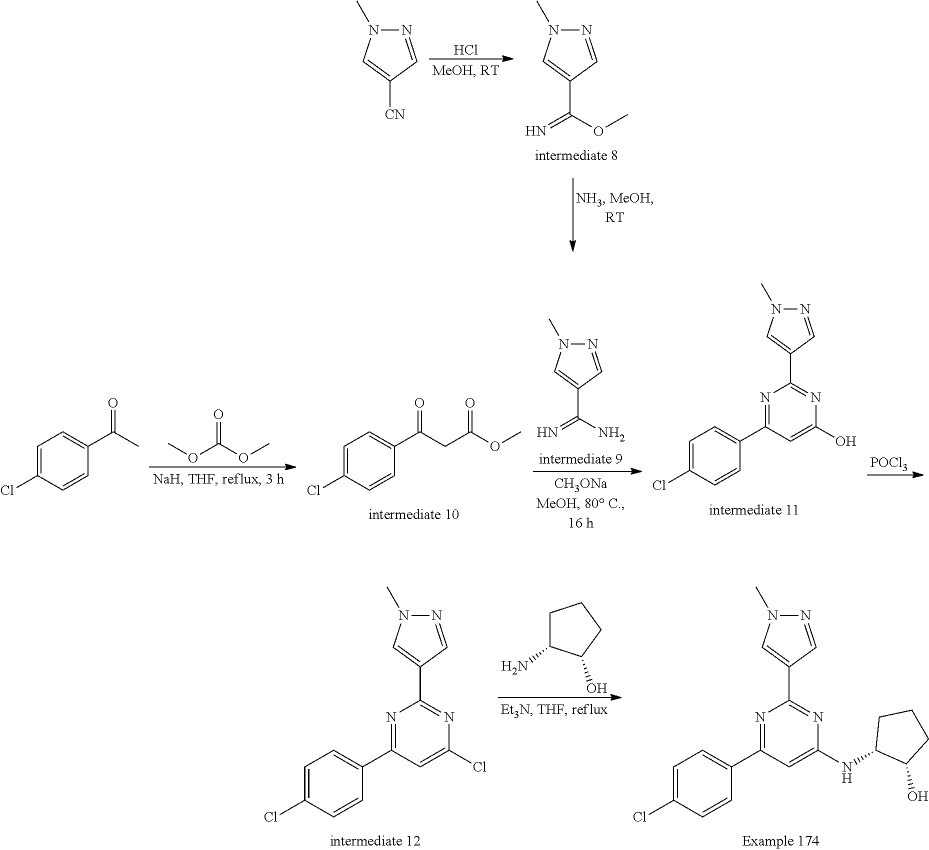

### Intermediate 8. methyl 1-methyl-1H-pyrazole-4-carbimidate

A suspension of 1-methyl-1H-pyrazole-4-carbonitrile (3.0 g, 28.04 mmol) in Hydrogen chloride-methanol solution (4 M HCl gas in MeOH, 30 mL) was stirred at room temperature for 16 hr. The solvent was removed in vacuo and the residue was purified via silica gel column chromatography (DCM/MeOH=20/1; V/V) to afford 1.7 g of the title compound.

MS (ESI, m/z): 140.1 [M+H]⁺

### Intermediate 9. 1-methyl-1H-pyrazole-4-carboximidamide

A solution of methyl 1-methyl-1H-pyrazole-4-carbimidate (2.7 g, 19.4 mmol) in 30 mL of ammonia-MeOH solution (7.0 M NH₃ in MeOH) was stirred at room temperature for 16 hr. The solvent was removed in vacuo and the residue was purified via reverse phase column chromatography (H₂O/MeOH=10/1; V/V) to afford 1.8 g of the title compound as a white solid.

MS (ESI, m/z): 125.1 [M+H]⁺

### Intermediate 10. methyl 3-(4-chlorophenyl)-3-oxopropanoate

To a solution of 1-(4-chlorophenyl)ethan-1-one (2.4 g, 15.5 mmol) in tetrahydrofuran (25 mL) was added sodium hydride (60%, 0.62 g, 15.5 mmol) at room temperature. A solution of dimethyl carbonate (0.7 g, 7.76 mmol) in tetrahydrofuran (5 mL) was added to the above reaction mixture during 5 minutes. The reaction mixture was heated at 70° C. for 2 hr. The reaction mixture was cooled to room temperature and quenched by saturated ammonium chloride aqueous solution. The mixture was acidified to pH=6.0 and the residue was extracted with dichloromethane (20 mL×3), dried over anhydrous sodium sulfate. The solids was filtered off and the filtrate was concentrated in vacuo. The residue was purified via silica gel column chromatography (Petroleum ether/EtOAc=20/1; V/V) to afford 2.3 g of the title compound as a yellow solid.

¹H NMR (400 MHz, DMSO-d₆) δ [ppm]=3.77 (s, 1H), 3.83 (s, 3H), 4.03 (s, 1H), 5.73 (s, 1H), 7.68 (d, J=8.3 Hz, 2H), 7.76 (d, J=8.2 Hz, 1H), 7.89 (d, J=8.2 Hz, 2H), 8.06 (d, J=8.1 Hz, 1H), 12.48 (s, 1H); MS (ESI, m/z): 213.1 [M+H]⁺

### Intermediate 11. 6-(4-chlorophenyl)-2-(1-methyl-1H-pyrazol-4-yl)pyrimidin-4-ol

To a solution of methyl 3-(4-chlorophenyl)-3-oxopropanoate (1.7 g, 8.13 mmol) in MeOH (20 mL) was added 1-methyl-1H-pyrazole-4-carboximidamide (1.0 g, 8.13 mmol) and sodium methoxide (527 mg, 9.576 mmol) at room temperature. The reaction mixture was heated at 80° C. under nitrogen for 16 hr. The residue was cooled to room temperature and acidified to pH=6.0. A white solid was formed. The solid was collected by filtration and dried in vacuo to give 1.1 g of the title compound as a white solid.

¹H NMR (400 MHz, DMSO-d₆) δ [ppm]=3.93 (s, 3H), 6.85 (s, 1H), 7.85 (d, J=8.3 Hz, 2H), 8.29 (s, 1H), 8.59 (s, 1H), 8.36 (d, J=8.1 Hz, 2H), 12.70 (s, 1H); MS (ESI, m/z): 287.1 [M+H]⁺

### Intermediate 12. 4-chloro-6-(4-chlorophenyl)-2-(1-methyl-1H-pyrazol-4-yl)pyrimidine

A solution of 6-(4-chlorophenyl)-2-(1-methyl-1H-pyrazol-4-yl)pyrimidin-4-ol (1.0 g, 3.43 mmol) in 10 mL of phosphorus oxychloride was heated at reflux for 13 hr. The mixture was concentrated in vacuo. The residue was poured into water and extracted with EtOAc (20 mL×2), washed with brine, dried and concentrated in vacuo. The residue was purified via column chromatography (DCM/MeOH=10/1; V/V) to afford 950 mg of the title compound.

¹H NMR (400 MHz, DMSO-d₆) δ [ppm]=3.94 (s, 3H), 7.93 (d, J=8.3 Hz, 2H), 8.14 (d, J=5.5 Hz, 2H), 8.53 (d, J=8.2 Hz, 2H), 8.56 (s, 1H); MS (ESI, m/z): 305.1 [M+H]⁺

### Example 174. (1S,2R)-2-((6-(4-chlorophenyl)-2-(1-methyl-1H-pyrazol-4-yl)pyrimidin-4-yl)amino)cyclopentan-1-ol

To a solution of 4-chloro-6-(4-chlorophenyl)-2-(1-methyl-1H-pyrazol-4-yl)pyrimidine (28 mg, 0.09 mmol) in tetrahydrofuran (4 mL) was added triethylamine (0.2 mL, 1.43 mmol) followed by (1S,2R)-2-aminocyclopentan-1-ol (20 mg, 0.20 mmol) at room temperature. The reaction mixture in sealed tube was heated at 120° C. for 4 h. and cooled to room temperature. The residue was filtered, evaporated in vacuo and isolated by Preparative HPLC to give 15 mg of the title compound.

¹H NMR (400 MHz, CD₃OD) δ [ppm]=1.95 (br, 1H), 2.05 (br, 1H), 3.41 (br, 3H), 3.49 (br, 3H), 4.03 (s, 3H), 6.98 (s, 1H), 7.67 (d, 2H), 7.90 (d, 2H), 8.33 (s, 1H), 8.59 (s, 1H); MS (ESI, m/z): 370.1 [M+H]⁺

### Example 175. (R)-1-(6-(4-chlorophenyl)-2-(1-methyl-1H-pyrazol-4-yl)pyrimidin-4-yl)piperidin-3-ol

Using (R)-piperidin-3-ol, the title compound was obtained as described for the example 174 (Scheme 2. General procedure B.).

¹H NMR (400 MHz, CD₃OD) δ [ppm]=1.69 (br, 2H), 1.77 (br, 2H), 1.99 (br, 1H), 2.03 (d, 2H), 3.89 (br, 1H), 3.93 (br, 2H), 4.01 (s, 3H), 7.08 (s, 1H), 7.64 (d, 2H), 7.88 (d, 2H), 8.32 (s, 1H), 8.58 (s, 1H); MS (ESI, m/z): 370.1 [M+H]⁺

### Example 176. 1-(6-(4-chlorophenyl)-2-(1-methyl-1H-pyrazol-4-yl)pyrimidin-4-yl)piperidin-4-ol

Using piperidin-4-ol, the title compound was obtained as described for the example 174 (Scheme 2. General procedure B.).

¹H NMR (400 MHz, CD₃OD) δ [ppm]=1.63-1.72 (m, 2H), 2.01-2.06 (m, 2H), 3.78 (br, 2H), 4.01 (s, 3H), 4.01-4.07 (m, 1H), 4.36 (br, 2H), 7.10 (s, 1H), 7.63 (d, 2H), 7.89 (d, 2H), 8.31 (s, 1H), 8.57 (s, 1H); MS (ESI, m/z): 370.1 [M+H]⁺

### Example 177. (1-(6-(4-chlorophenyl)-2-(1-methyl-1H-pyrazol-4-yl)pyrimidin-4-yl)piperidin-4-yl)methanol

Using piperidin-4-ylmethanol, the title compound was obtained as described for the example 174 (Scheme 2. General procedure B.).

¹H NMR (400 MHz, CD₃OD) δ [ppm]=1.30-1.40 (m, 4H), 1.89-2.04 (m, 4H), 3.21-3.28 (m, 1H), 3.48 (d, 2H), 4.01 (s, 3H), 7.08 (s, 1H), 7.64 (d, 2H), 7.88 (d, 2H), 8.30 (s, 1H), 8.56 (s, 1H); MS (ESI, m/z): 384.2 [M+H]⁺

### Example 178. 2-(1-(6-(4-chlorophenyl)-2-(1-methyl-1H-pyrazol-4-yl)pyrimidin-4-yl)piperidin-4-yl)ethan-1-ol

Using 2-(piperidin-4-yl)ethan-1-ol, the title compound was obtained as described for the example 174 (Scheme 2. General procedure B.).

¹H NMR (400 MHz, CD₃OD) δ [ppm]=1.28-1.37 (m, 4H), 1.55 (q, 2H), 1.91-2.00 (m, 4H), 3.25 (br, 1H), 3.67 (t, 2H), 4.01 (s, 3H), 7.08 (s, 1H), 7.64 (d, 2H), 7.87 (d, 2H), 8.31 (s, 1H), 8.58 (s, 1H); MS (ESI, m/z): 398.2 [M+H]⁺

### Example 179. 3-(1-(6-(4-chlorophenyl)-2-(1-methyl-1H-pyrazol-4-yl)pyrimidin-4-yl)piperidin-4-yl)propan-1-ol

Using 3-(piperidin-4-yl)propan-1-ol, the title compound was obtained as described for the example 174 (Scheme 2. General procedure B.).

¹H NMR (400 MHz, CD₃OD) δ [ppm]=1.23-1.41 (m, 4H), 1.57-1.65 (m, 2H), 1.69-1.80 (m, 1H), 1.98 (d, 2H), 3.21 (t, 2H), 3.57 (t, 2H), 4.00 (s, 3H), 4.83 (br, 2H), 7.05 (s, 1H), 7.63 (d, 2H), 7.88 (d, 2H), 8.29 (s, 1H), 8.55 (s, 1H); MS (ESI, m/z): 412.2 [M+H]⁺

### Example 180. 4-(4-chlorophenyl)-2-(1-methyl-1H-pyrazol-4-yl)-6-(4-methylpiperidin-1-yl)pyrimidine

Using 4-methylpiperidine, the title compound was obtained as described for the example 174 (Scheme 2. General procedure B.).

¹H NMR (400 MHz, CD₃OD) δ [ppm]=1.03 (d, 3H), 1.23-1.34 (m, 2H), 1.82-1.89 (m, 1H), 1.92 (d, 2H), 3.24 (br, 2H), 4.01 (s, 3H), 4.83 (br, 2H), 7.07 (s, 1H), 7.64 (d, 2H), 7.87 (d, 2H), 8.31 (s, 1H), 8.58 (s, 1H); MS (ESI, m/z): 368.2 [M+H]⁺

### Example 181. 4-(4-chlorophenyl)-2-(1-methyl-1H-pyrazol-4-yl)-6-(4-methylpiperazin-1-yl)pyrimidine

Using 1-methylpiperazine, the title compound was obtained as described for the example 174 (Scheme 2. General procedure B.).

¹H NMR (400 MHz, CD₃OD) δ [ppm]=3.00 (t, 3H), 3.59 (br, 8H), 4.01 (s, 3H), 7.22 (s, 1H), 7.63 (d, 2H), 7.95 (d, 2H), 8.33 (s, 1H), 8.59 (s, 1H); MS (ESI, m/z): 369.2 [M+H]⁺

### Example 182. 2-(4-(6-(4-chlorophenyl)-2-(1-methyl-1H-pyrazol-4-yl)pyrimidin-4-yl)piperazin-1-yl)ethan-1-ol

Using 2-(piperazin-1-yl)ethan-1-ol, the title compound was obtained as described for the example 174 (Scheme 2. General procedure B.).

¹H NMR (400 MHz, CD₃OD) δ [ppm]=3.39 (t, 2H), 3.60 (br, 4H), 3.96 (t, 2H), 4.01 (s, 3H), 4.34 (br, 4H), 7.22 (s, 1H), 7.63 (d, 2H), 7.96 (d, 2H), 8.33 (s, 1H), 8.59 (s, 1H); MS (ESI, m/z): 399.2 [M+H]⁺

### Example 183. (S)-1-(6-(4-chlorophenyl)-2-(1-methyl-1H-pyrazol-4-yl)pyrimidin-4-yl)pyrrolidin-3-ol

Using (S)-pyrrolidin-3-ol, the title compound was obtained as described for the example 174 (Scheme 2. General procedure B.).

¹H NMR (400 MHz, CD₃OD) δ [ppm]=2.10-2.30 (m, 3H), 3.82 (br, 2H), 4.01 (s, 3H), 4.03 (br, 1H), 4.63 (d, 1H), 6.83 (s, 1H), 7.65 (d, 2H), 7.90 (d, 2H), 8.30 (s, 1H), 8.55 (s, 1H); MS (ESI, m/z): 356.1 [M+H]⁺

### Example 184. 1-(6-(4-chlorophenyl)-2-(1-methyl-1H-pyrazol-4-yl)pyrimidin-4-yl)piperidine-4-carbonitrile

Using piperidine-4-carbonitrile, the title compound was obtained as described for the example 174 (Scheme 2. General procedure B.).

¹H NMR (400 MHz, CD₃OD) δ [ppm]=1.93-2.01 (m, 2H), 2.11-2.18 (m, 2H), 3.19-3.25 (m, 1H), 3.84-3.90 (m, 2H), 4.01 (s, 3H), 4.31 (br, 2H), 7.12 (s, 1H), 7.63 (d, 2H), 7.91 (d, 2H), 8.31 (s, 1H), 8.56 (s, 1H); MS (ESI, m/z): 379.1 [M+H]⁺

### Example 185. (R)-(1-(6-(4-chlorophenyl)-2-(1-methyl-1H-pyrazol-4-yl)pyrimidin-4-yl)piperidin-3-yl)methanol

Using (R)-piperidin-3-ylmethanol, the title compound was obtained as described for the example 174 (Scheme 2. General procedure B.).

¹H NMR (400 MHz, CD₃OD) δ [ppm]=1.44-1.52 (m, 1H), 1.62-1.71 (m, 1H), 1.87 (br, 1H), 1.93 (d, 2H), 3.18 (br, 1H), 3.39 (t, 1H), 3.48-3.52 (m, 1H), 3.58-3.62 (m, 1H), 4.01 (s, 3H), 4.59 (br, 2H), 7.08 (s, 1H), 7.63 (d, 2H), 7.88 (d, 2H), 8.31 (s, 1H), 8.57 (s, 1H); MS (ESI, m/z): 384.2 [M+H]J

### Example 186. (R)-1-(6-(4-chlorophenyl)-2-(1-methyl-1H-pyrazol-4-yl)pyrimidin-4-yl)pyrrolidin-3-ol

Using (R)-pyrrolidin-3-ol, the title compound was obtained as described for the example 174 (Scheme 2. General procedure B.).

¹H NMR (400 MHz, CD₃OD) δ [ppm]=2.10-2.30 (m, 3H), 3.82 (br, 2H), 4.01 (s, 3H), 4.03 (br, 1H), 4.63 (d, 1H), 6.83 (s, 1H), 7.65 (d, 2H), 7.90 (d, 2H), 8.30 (s, 1H), 8.55 (s, 1H); MS (ESI, m/z): 356.1 [M+H]⁺

### Example 187. (1S,3R)-3-((6-(4-chlorophenyl)-2-(1-methyl-1H-pyrazol-4-yl)pyrimidin-4-yl)amino)cyclopentan-1-ol

Using (1S,3R)-3-aminocyclopentan-1-ol, the title compound was obtained as described for the example 174 (Scheme 2. General procedure B.).

¹H NMR (400 MHz, CD₃OD) δ [ppm]=1.83-1.94 (m, 1H), 3.43 (br, 6H), 4.02 (s, 3H), 4.37 (br, 1H), 6.97 (s, 1H), 7.65 (d, 2H), 7.88 (d, 2H), 8.32 (s, 1H), 8.58 (s, 1H); MS (ESI, m/z): 370.1 [M+H]⁺

### Example 188. (R)-2-((6-(4-chlorophenyl)-2-(1-methyl-1H-pyrazol-4-yl)pyrimidin-4-yl)amino)butan-1-ol

Using (R)-2-aminobutan-1-ol, the title compound was obtained as described for the example 174 (Scheme 2. General procedure B.).

¹H NMR (400 MHz, CD₃OD) δ [ppm]=1.03 (t, 3H), 1.59-1.68 (m, 2H), 1.78-1.86 (m, 1H), 3.69-3.73 (m, 2H), 4.02 (s, 3H), 6.97 (s, 1H), 7.65 (d, 2H), 7.89 (d, 2H), 8.32 (s, 1H), 8.56 (s, 1H); MS (ESI, m/z): 358.1 [M+H]⁺

### Example 189. Trans-4-((6-(4-chlorophenyl)-2-(1-methyl-1H-pyrazol-4-yl)pyrimidin-4-yl)amino)cyclohexan-1-ol

Using Trans-4-aminocyclohexan-1-ol, the title compound was obtained as described for the example 174 (Scheme 2. General procedure B.).

¹H NMR (400 MHz, CD₃OD) δ [ppm]=1.46-1.52 (m, 2H), 2.01-2.05 (m, 2H), 2.13 (br, 1H), 3.43 (br, 4H), 3.63 (br, 1H), 4.02 (s, 3H), 6.97 (s, 1H), 7.65 (d, 2H), 7.89 (d, 2H), 8.31 (s, 1H), 8.56 (s, 1H); MS (ESI, m/z): 384.2 [M+H]⁺

### Example 190. 7-(6-(4-chlorophenyl)-2-(pyridin-3-yl)pyrimidin-4-yl)octahydro-2H-pyrano[2,3-c]pyridine

Using octahydro-2H-pyrano[2,3-c]pyridine and separation method of PREP. HPLC, the title compound was obtained as described for the example 1 (Scheme 1. General procedure A.).

¹H NMR (400 MHz, CD₃OD) δ [ppm]=1.29-1.35 (m, 2H), 1.54 (d, 1H), 1.72-1.80 (m, 2H), 1.85 (d, 1H), 1.95 (d, 1H), 2.10-2.20 (m, 1H), 2.98-3.08 (m, 2H), 3.20 (d, 1H), 3.53 (t, 2H), 3.92 (d, 1H), 7.17 (s, 1H), 7.46 (d, 2H), 8.06-8.09 (m, 1H), 8.11 (d, 2H), 8.87 (d, 1H), 9.37 (d, 1H), 9.63 (s, 1H); MS (ESI, m/z): 407.2 [M+H]⁺

### Example 191. 7-(6-(4-chlorophenyl)-2-(pyridin-3-yl)pyrimidin-4-yl)octahydro-2H-pyrano[2,3-c]pyridin-4-ol

Using octahydro-2H-pyrano[2,3-c]pyridin-4-ol and separation method of PREP. HPLC, the title compound was obtained as described for the example 1 (Scheme 1. General procedure A.).

¹H NMR (400 MHz, CD₃OD) δ [ppm]=1.56-1.60 (m, 1H), 1.66-1.74 (m, 1H), 1.77-1.83 (m, 1H), 1.88-1.92 (m, 1H), 1.94-2.04 (m, 1H), 2.14-2.21 (m, 1H), 2.99-3.11 (m, 1H), 3.18-3.27 (m, 1H), 3.51 (t, 1H), 3.61-3.73 (m, 1H), 3.78-3.86 (m, 1H), 3.88-4.00 (m, 2H), 7.23 (s, 1H), 7.50 (d, 2H), 8.12-8.19 (m, 3H), 8.91 (d, 1H), 9.47 (d, 1H), 9.69 (s, 1H); MS (ESI, m/z): 423.2 [M+H]⁺

### Example 192. (2R,3R)-3-((6-(4-chlorophenyl)-2-(pyridin-3-yl)pyrimidin-4-yl)amino)pentan-2-ol

Using (2R,3R)-3-aminopentan-2-ol and separation method of PREP. HPLC, the title compound was obtained as described for the example 1 (Scheme 1. General procedure A.).

¹H NMR (400 MHz, CD₃OD) δ [ppm]=1.02 (t, 3H), 1.21 (d, 3H), 1.58-1.68 (m, 2H), 1.79-1.85 (m, 1H), 2.01-2.05 (m, 1H), 4.00 (br, 1H), 4.36 (br, 1H), 7.02 (s, 1H), 7.54 (d, 2H), 7.98-8.02 (m, 1H), 8.14 (d, 2H), 8.84 (d, 1H), 9.34 (d, 1H), 9.67 (s, 1H); MS (ESI, m/z): 369.1 [M+H]⁺

### Example 193. 6-(4-chlorophenyl)-N-((1-methylpiperidin-4-yl)methyl)-2-(pyridin-3-yl)pyrimidin-4-amine

Using (1-methylpiperidin-4-yl)methanamine and separation method of PREP. HPLC, the title compound was obtained as described for the example 1 (Scheme 1. General procedure A.).

¹H NMR (400 MHz, CD₃OD) δ [ppm]=1.53-1.66 (m, 2H), 1.94-2.08 (m, 2H), 2.13 (d, 2H), 2.86 (s, 3H), 3.01 (t, 1H), 3.13 (t, 1H), 3.57 (br, 2H), 3.81-3.93 (m, 1H), 7.00 (s, 1H), 7.54 (d, 2H), 8.12-8.16 (m, 1H), 8.24 (d, 2H), 8.92 (d, 1H), 9.49 (d, 1H), 9.76 (s, 1H); MS (ESI, m/z): 394.2 [M+H]⁺

### Example 194. (R)-1-(6-(4-chlorophenyl)-2-(pyridin-3-yl)pyrimidin-4-yl)pyrrolidin-3-ol

Using (R)-pyrrolidin-3-ol) and separation method of PREP. HPLC, the title compound was obtained as described for the example 1 (Scheme 1. General procedure A.).

¹H NMR: (400 MHz, CD₃OD) δ [ppm]=2.17 (br, 2H), 3.70 (br, 2H), 3.86 (br, 2H), 4.59 (br, 1H), 6.95 (s, 1H), 7.52 (d, 2H), 8.07-8.11 (m, 1H), 8.19 (d, 2H), 8.89 (d, 1H), 9.43 (d, 1H), 9.69 (s, 1H); MS (ESI, m/z): 353.1 [M+H]⁺

### Example 195. (S)-6-(4-chlorophenyl)-N-(2-(methoxymethyl)pyrrolidin-1-yl)-2-(pyridin-3-yl)pyrimidin-4-amine

Using (S)-2-(methoxymethyl)pyrrolidin-1-amine and separation method of PREP. HPLC, the title compound was obtained as described for the example 1 (Scheme 1. General procedure A.).

¹H NMR: (400 MHz, CD₃OD) δ [ppm]=1.60 (br, 1H), 1.80-1.87 (m, 1H), 1.89-1.97 (m, 1H), 2.02-2.10 (m, 1H), 2.17-2.21 (m, 1H), 2.82-2.89 (m, 1H), 3.07 (br, 1H), 3.24 (s, 3H), 3.44 (t, 2H), 7.42 (s, 1H), 7.56 (d, 2H), 8.08-8.12 (m, 1H), 8.21 (d, 2H), 8.89 (d, 1H), 9.45 (d, 1H), 9.71 (s, 1H); MS (ESI, m/z): 396.2 [M+H]⁺

### Example 196. (S)-4-(4-chlorophenyl)-6-(3-fluoropyrrolidin-1-yl)-2-(pyridin-3-yl)pyrimidine

Using (S)-3-fluoropyrrolidine and separation method of PREP. HPLC, the title compound was obtained as described for the example 1 (Scheme 1. General procedure A.).

¹H NMR: (400 MHz, CD₃OD) δ [ppm]=2.24 (br, 2H), 3.79 (br, 2H), 3.87 (br, 2H), 4.17 (br, 1H), 6.98 (s, 1H), 7.53 (d, 2H), 8.03-8.06 (m, 1H), 8.23 (d, 2H), 8.86 (d, 1H), 9.40 (d, 1H), 9.71 (s, 1H); MS (ESI, m/z): 355.1 [M+H]⁺

### Example 197. 4-(4-chlorophenyl)-2-(pyridin-3-yl)-6-(2-(trifluoromethyl)pyrrolidin-1-yl)pyrimidine

Using 2-(trifluoromethyl)pyrrolidine and separation method of PREP. HPLC, the title compound was obtained as described for the example 1 (Scheme 1. General procedure A.).

¹H NMR: (400 MHz, CD₃OD) δ [ppm]=2.26 (br, 2H), 2.32 (br, 2H), 3.74 (br, 2H), 3.89 (br, 1H), 7.20 (s, 1H), 7.56 (d, 2H), 8.02-8.06 (m, 1H), 8.28 (d, 2H), 8.87 (d, 1H), 9.40 (d, 1H), 9.74 (s, 1H); MS (ESI, m/z): 405.1 [M+H]⁺

### Example 198. 4-(4-chlorophenyl)-6-(3,3-difluoropyrrolidin-1-yl)-2-(pyridin-3-yl)pyrimidine

Using 3,3-difluoropyrrolidine and separation method of PREP. HPLC, the title compound was obtained as described for the example 1 (Scheme 1. General procedure A.).

¹H NMR: (400 MHz, CD₃OD) δ [ppm]=2.58-2.68 (m, 2H), 3.95 (br, 2H), 4.11 (br, 2H), 7.06 (s, 1H), 7.55 (d, 2H), 8.04-8.07 (m, 1H), 8.28 (d, 2H), 8.87 (d, 1H), 9.44 (d, 1H), 9.75 (s, 1H); MS (ESI, m/z): 373.1 [M+H]⁺

### Example 199. 4-(1-(6-(4-chlorophenyl)-2-(pyridin-3-yl)pyrimidin-4-yl)pyrrolidin-3-yl)morpholine

Using 4-(pyrrolidin-3-yl)morpholine and separation method of PREP. HPLC, the title compound was obtained as described for the example 1 (Scheme 1. General procedure A.).

¹H NMR: (400 MHz, CD₃OD) δ [ppm]=2.44 (br, 1H), 2.68 (br, 1H), 3.52 (br, 4H), 3.70 (br, 2H), 3.99 (br, 5H), 4.14 (br, 2H), 7.06 (s, 1H), 7.54 (d, 2H), 8.16-8.19 (m, 1H), 8.25 (d, 2H), 8.94 (d, 1H), 9.57 (d, 1H), 9.80 (s, 1H); MS (ESI, m/z): 422.2 [M+H]⁺

### Example 200. 5-(((6-(4-chlorophenyl)-2-(pyridin-3-yl)pyrimidin-4-yl)amino)methyl)pyrrolidin-2-one

Using 5-(aminomethyl)pyrrolidin-2-one and separation method of PREP. HPLC, the title compound was obtained as described for the example 1 (Scheme 1. General procedure A.).

¹H NMR: (400 MHz, CD₃OD) δ [ppm]=2.00 (br, 2H), 2.32-2.38 (m, 2H), 3.73 (br, 2H), 4.04 (br, 1H), 7.01 (s, 1H), 7.54 (d, 2H), 8.07-8.10 (m, 1H), 8.17 (d, 2H), 8.88 (d, 1H), 9.46 (d, 1H), 9.73 (s, 1H); MS (ESI, m/z): 380.1 [M+H]⁺

### Example 201. Trans-6-(4-chlorophenyl)-2-(pyridin-3-yl)-N-(4-(pyrrolidin-1-yl)tetrahydrofuran-3-yl)pyrimidin-4-amine

Using Trans-4-(pyrrolidin-1-yl)tetrahydrofuran-3-amine and separation method of PREP. HPLC, the title compound was obtained as described for the example 1 (Scheme 1. General procedure A.).

¹H NMR: (400 MHz, CD₃OD) δ [ppm]=2.09 (br, 8H), 3.74-3.78 (m, 1H), 3.96-3.99 (m, 1H), 4.19-4.21 (m, 2H), 4.43-4.47 (m, 1H), 5.31 (br, 1H), 7.06 (s, 1H), 7.55 (d, 2H), 8.00-8.03 (m, 1H), 8.18 (d, 2H), 8.88 (d, 1H), 9.36 (d, 1H), 9.73 (s, 1H); MS (ESI, m/z): 422.2 [M+H]⁺

### Example 202. 6-(4-chlorophenyl)-N-((3S,4S)-4-methoxy-1-methylpyrrolidin-3-yl)-2-(pyridin-3-yl)pyrimidin-4-amine

Using (3S,4S)-4-methoxy-1-methylpyrrolidin-3-amine and separation method of PREP. HPLC, the title compound was obtained as described for the example 1 (Scheme 1. General procedure A.).

¹H NMR: (400 MHz, CD₃OD) δ [ppm]=1.59 (br, 1H), 1.90 (br, 1H), 2.02 (br, 1H), 3.06 (s, 3H), 3.55 (s, 3H), 3.72 (br, 1H), 3.84 (br, 2H), 7.10 (s, 1H), 7.53 (d, 2H), 8.18 (d, 2H), 8.19-8.27 (m, 1H), 8.95 (d, 1H), 9.55 (d, 1H), 9.78 (s, 1H); MS (ESI, m/z): 396.2 [M+H]⁺

### Example 203. (R)-6-(4-chlorophenyl)-N-(piperidin-3-yl)-2-(pyridin-3-yl)pyrimidin-4-amine

Using tert-butyl (R)-3-aminopiperidine-1-carboxylate and separation method of PREP. HPLC, the title compound was obtained as described for the example 54 (Scheme 1. General procedure A.).

¹H NMR: (400 MHz, CD₃OD) δ [ppm]=1.77-1.83 (m, 1H), 1.95-2.04 (m, 1H), 2.10-2.15 (m, 1H), 2.17-2.23 (m, 1H), 2.96-3.01 (m, 1H), 3.04-3.11 (m, 1H), 3.37-3.43 (m, 1H), 3.69-3.73 (m, 1H), 4.56 (br, 1H), 7.03 (s, 1H), 7.54 (d, 2H), 8.02-8.06 (m, 1H), 8.17 (d, 2H), 8.88 (d, 1H), 9.44 (d, 1H), 9.82 (s, 1H); MS (ESI, m/z): 366.1 [M+H]⁺

### Example 204. 6-(4-chlorophenyl)-N-(piperidin-3-yl)-2-(pyridin-3-yl)pyrimidin-4-amine

Using tert-butyl 3-aminopiperidine-1-carboxylate and separation method of PREP. HPLC, the title compound was obtained as described for the example 54 (Scheme 1. General procedure A.).

¹H NMR: (400 MHz, CD₃OD) δ [ppm]=1.77-1.83 (m, 1H), 1.95-2.04 (m, 1H), 2.10-2.15 (m, 1H), 2.17-2.23 (m, 1H), 2.96-3.01 (m, 1H), 3.04-3.11 (m, 1H), 3.37-3.43 (m, 1H), 3.69-3.73 (m, 1H), 4.56 (br, 1H), 7.03 (s, 1H), 7.54 (d, 2H), 8.02-8.06 (m, 1H), 8.17 (d, 2H), 8.88 (d, 1H), 9.44 (d, 1H), 9.82 (s, 1H); MS (ESI, m/z): 366.1 [M+H]⁺

### Example 205. 6-(4-chlorophenyl)-N-((3R,4R)-3-fluoropiperidin-4-yl)-2-(pyridin-3-yl)pyrimidin-4-amine

Using tert-butyl (3R,4R)-4-amino-3-fluoropiperidine-1-carboxylate and separation method of PREP. HPLC, the title compound was obtained as described for the example 54 (Scheme 1. General procedure A.).

¹H NMR: (400 MHz, CD₃OD) δ [ppm]=1.60 (br, 1H), 1.92 (br, 1H), 2.03 (br, 1H), 3.79 (br, 5H), 7.08 (s, 1H), 7.55 (d, 2H), 8.08-8.11 (m, 1H), 8.20 (d, 2H), 8.90 (d, 1H), 9.41 (d, 1H), 9.70 (s, 1H); MS (ESI, m/z): 384.1 [M+H]⁺

### Example 206. (S)-6-(4-chlorophenyl)-2-(pyridin-3-yl)-N-(pyrrolidin-3-ylmethyl)pyrimidin-4-amine

Using tert-butyl (R)-3-(aminomethyl)pyrrolidine-1-carboxylate and separation method of PREP. HPLC, the title compound was obtained as described for the example 54 (Scheme 1. General procedure A.).

¹H NMR: (400 MHz, CD₃OD) δ [ppm]=1.85-1.95 (m, 2H), 2.24-2.32 (m, 1H), 2.80-2.87 (m, 1H), 3.10-3.15 (m, 1H), 3.41-3.48 (m, 1H), 3.50-3.55 (m, 1H), 3.75 (br, 2H), 7.01 (s, 1H), 7.54 (d, 2H), 8.09-8.13 (m, 1H), 8.17 (d, 2H), 8.91 (d, 1H), 9.48 (d, 1H), 9.77 (s, 1H); MS (ESI, m/z): 366.1 [M+H]⁺

### Example 207. methyl (2R,4R)-4-((6-(4-chlorophenyl)-2-(pyridin-3-yl)pyrimidin-4-yl)amino)pyrrolidine-2-carboxylate

Using 1-(tert-butyl) 2-methyl (2R,4R)-4-aminopyrrolidine-1,2-dicarboxylate and separation method of PREP. HPLC, the title compound was obtained as described for the example 54 (Scheme 1. General procedure A.).

MS (ESI, m/z): 410.1 [M+H]⁺

### Example 208. (2R,4S)-4-((6-(4-chlorophenyl)-2-(pyridin-3-yl)pyrimidin-4-yl)amino)pyrrolidine-2-carboxylic acid

Using (2R,4S)-4-amino-1-(tert-butoxycarbonyl)pyrrolidine-2-carboxylic acid and separation method of PREP. HPLC, the title compound was obtained as described for the example 54 (Scheme 1. General procedure A.).

¹H NMR: (400 MHz, CD₃OD) δ [ppm]=1.61 (br, 2H), 2.04 (br, 2H), 2.20 (t, 1H), 3.35 (br, 1H), 7.01 (s, 1H), 7.55 (d, 2H), 7.78-7.81 (m, 1H), 8.18 (d, 2H), 8.76 (d, 1H), 9.12 (d, 1H), 9.66 (s, 1H); MS (ESI, m/z): 396.1 [M+H]⁺

### Example 209. Trans-4-((6-(4-chlorophenyl)-2-(pyridin-3-yl)pyrimidin-4-yl)amino)-1-isopropylpyrrolidin-3-ol

Using Trans-4-amino-1-isopropylpyrrolidin-3-ol and separation method of PREP. HPLC, the title compound was obtained as described for the example 1 (Scheme 1. General procedure A.).

¹H NMR: (400 MHz, CD₃OD) δ [ppm]=1.43 (t, 6H), 1.61 (br, 1H), 1.90 (br, 1H), 2.02 (br, 1H), 2.19 (t, 1H), 3.55 (br, 1H), 3.72 (br, 1H), 3.84 (br, 1H), 7.06 (s, 1H), 7.55 (d, 2H), 7.95-7.80 (m, 1H), 8.19 (d, 2H), 8.85 (d, 1H), 9.36 (d, 1H), 9.75 (s, 1H); MS (ESI, m/z): 410.2 [M+H]⁺

### Example 210. (R)-4-(3-(chloromethyl)pyrrolidin-1-yl)-6-(4-chlorophenyl)-2-(pyridin-3-yl)pyrimidine

Using (R)-3-(chloromethyl)pyrrolidine and separation method of PREP. HPLC, the title compound was obtained as described for the example 1 (Scheme 1. General procedure A.).

¹H NMR: (400 MHz, CD₃OD) δ [ppm]=1.98 (br, 1H), 2.28 (br, 1H), 2.79 (br, 1H), 3.54 (br, 1H), 3.68-3.78 (m, 4H), 4.02 (br, 1H), 6.88 (s, 1H), 7.50 (d, 2H), 8.08-8.11 (m, 1H), 8.18 (d, 2H), 8.89 (d, 1H), 9.43 (d, 1H), 9.67 (s, 1H); MS (ESI, m/z): 385.1 [M+H]⁺

### Example 211. (S)-4-(3-(chloromethyl)pyrrolidin-1-yl)-6-(4-chlorophenyl)-2-(pyridin-3-yl)pyrimidine

Using (S)-3-(chloromethyl)pyrrolidine and separation method of PREP. HPLC, the title compound was obtained as described for the example 1 (Scheme 1. General procedure A.).

¹H NMR: (400 MHz, CD₃OD) δ [ppm]=1.98 (br, 1H), 2.28 (br, 1H), 2.79 (br, 1H), 3.54 (br, 1H), 3.68-3.78 (m, 4H), 4.02 (br, 1H), 6.88 (s, 1H), 7.50 (d, 2H), 8.08-8.11 (m, 1H), 8.18 (d, 2H), 8.89 (d, 1H), 9.43 (d, 1H), 9.67 (s, 1H); MS (ESI, m/z): 385.1 [M+H]⁺

### Example 212. 1-(6-(4-chlorophenyl)-2-(pyridin-3-yl)pyrimidin-4-yl)pyrrolidine-3-carbonitrile

Using pyrrolidine-3-carbonitrile and separation method of PREP. HPLC, the title compound was obtained as described for the example 1 (Scheme 1. General procedure A.).

¹H NMR: (400 MHz, CD₃OD) δ [ppm]=2.44 (br, 1H), 2.53 (br, 1H), 3.57 (br, 1H), 3.84 (br, 2H), 4.06 (br, 2H), 7.06 (s, 1H), 7.55 (d, 2H), 8.08-8.12 (m, 1H), 8.27 (d, 2H), 8.89 (d, 1H), 9.49 (d, 1H), 9.76 (s, 1H); MS (ESI, m/z): 362.1 [M+H]⁺

### Example 213. (R)-1-((6-(4-chlorophenyl)-2-(pyridin-3-yl)pyrimidin-4-yl)amino)butan-2-ol

Using (R)-1-aminobutan-2-ol and separation method of PREP. HPLC, the title compound was obtained as described for the example 1 (Scheme 1. General procedure A.).

¹H NMR: (400 MHz, CD₃OD) δ [ppm]=1.05 (t, 3H), 1.48-1.59 (m, 1H), 1.61-1.70 (m, 1H), 3.55 (br, 1H), 3.72 (br, 1H), 3.76 (br, 1H), 6.99 (s, 1H), 7.51 (d, 2H), 8.09-8.13 (m, 3H), 8.90 (d, 1H), 9.44 (d, 1H), 9.69 (s, 1H); MS (ESI, m/z): 355.1 [M+H]⁺

### Example 214. (1R,3S)-3-((6-(4-chlorophenyl)-2-(pyridin-3-yl)pyrimidin-4-yl)amino)cyclopentan-1-ol

Using (1R,3S)-3-aminocyclopentan-1-ol and separation method of PREP. HPLC, the title compound was obtained as described for the example 1 (Scheme 1. General procedure A.).

¹H NMR: (400 MHz, CD₃OD) δ [ppm]=1.60-1.66 (m, 1H), 1.81-1.95 (m, 4H), 2.17-2.23 (m, 1H), 2.43-2.50 (m, 1H), 4.34-4.38 (m, 1H), 4.62 (br, 1H), 6.94 (s, 1H), 7.53 (d, 2H), 8.06-8.10 (m, 1H), 8.12 (d, 2H), 8.89 (d, 1H), 9.41 (d, 1H), 9.69 (s, 1H); MS (ESI, m/z): 367.1 [M+H]J

### Example 215. Cis-(4-((6-(4-chlorophenyl)-2-(pyridin-3-yl)pyrimidin-4-yl)amino)cyclohexyl)methanol

Using Cis-4-aminocyclohexan-1-ol and separation method of PREP. HPLC, the title compound was obtained as described for the example 1 (Scheme 1. General procedure A.).

¹H NMR: (400 MHz, CD₃OD) δ [ppm]=1.76-1.83 (m, 4H), 1.84-1.87 (m, 4H), 2.02 (br, 1H), 3.92 (br, 1H), 4.24 (br, 1H), 6.96 (s, 1H), 7.53 (d, 2H), 8.01-8.05 (m, 1H), 8.12 (d, 2H), 8.86 (d, 1H), 9.34 (d, 1H), 9.67 (s, 1H); MS (ESI, m/z): 381.1 [M+H]⁺

### Example 216. Cis-4-((6-(4-chlorophenyl)-2-(pyridin-3-yl)pyrimidin-4-yl)amino)cyclohexan-1-ol

Using Cis-(4-aminocyclohexyl)methanol and separation method of PREP. HPLC, the title compound was obtained as described for the example 1 (Scheme 1. General procedure A.).

¹H NMR: (400 MHz, CD₃OD) δ [ppm]=1.70-1.84 (m, 4H), 1.85-1.95 (m, 4H), 1.98-2.05 (m, 1H), 3.71-3.74 (m, 1H), 3.84-8.37 (m, 1H), 4.46 (br, 1H), 7.04 (s, 1H), 7.55 (d, 2H), 8.09-8.15 (m, 3H), 8.90 (d, 1H), 9.43 (d, 1H), 9.69 (s, 1H); MS (ESI, m/z): 395.2 [M+H]⁺

### Example 217. Trans-4-((6-(4-chlorophenyl)-2-(pyridin-3-yl)pyrimidin-4-yl)amino)cyclohexan-1-ol

Using Trans-(4-aminocyclohexyl)methanol and separation method of PREP. HPLC, the title compound was obtained as described for the example 1 (Scheme 1. General procedure A.).

¹H NMR: (400 MHz, CD₃OD) δ [ppm]=1.70-1.84 (m, 4H), 1.85-1.95 (m, 4H), 1.98-2.05 (m, 1H), 3.71-3.74 (m, 1H), 3.84-8.37 (m, 1H), 4.46 (br, 1H), 7.04 (s, 1H), 7.55 (d, 2H), 8.09-8.15 (m, 3H), 8.90 (d, 1H), 9.43 (d, 1H), 9.69 (s, 1H); MS (ESI, m/z): 395.2 [M+H]⁺

### Example 218. 4-(4-chlorophenyl)-6-(4-(2-methoxyethyl)piperazin-1-yl)-2-(pyridin-3-yl)pyrimidine

Using 1-(2-methoxyethyl)piperazine and separation method of PREP. HPLC, the title compound was obtained as described for the example 1 (Scheme 1. General procedure A.).

¹H NMR (400 MHz, CD₃OD) δ [ppm]=9.83 (d, J=2.0, 0.8 Hz, 1H), 9.59 (dd, J=8.2, 2.0, 1.5 Hz, 1H), 8.95 (d, J=5.7, 1.5, 0.7 Hz, 1H), 8.28 (d, 2H), 8.19 (dd, J=8.2, 5.7, 0.7 Hz, 1H), 7.54 (d, 2H), 7.44 (s, 1H), 3.82-3.77 (m, 4H), 3.77-3.50 (m, 4H), 3.49-3.43 (m, 7H); MS (ESI, m/z): 410.2 [M+H]⁺

### Example 219. (3S,4R)-1-(6-(4-chlorophenyl)-2-(pyridin-3-yl)pyrimidin-4-yl)-3-fluoropiperidin-4-ol

Using (3S,4R)-3-fluoropiperidin-4-ol and separation method of PREP. HPLC, the title compound was obtained as described for the example 1 (Scheme 1. General procedure A.).

¹H NMR (400 MHz, CD₃OD) δ [ppm]=9.74 (d, J=1.7, 0.8 Hz, 1H), 9.52 (dd, J=8.2, 1.9, 1.5 Hz, 1H), 8.92 (dd, J=5.7, 1.5, 0.8 Hz, 1H), 8.21 (dd, 2H), 8.19-8.14 (m, 1H), 7.51 (d, 2H), 7.30 (s, 1H), 4.56-4.47 (m, 1H), 4.47-4.33 (m, 1H), 4.17-4.06 (m, 1H), 4.04-3.94 (m, 1H), 3.94-3.82 (m, 1H), 3.82-3.68 (m, 1H), 2.18-2.05 (m, 1H), 1.74-1.60 (m, 1H); MS (ESI, m/z): 385.1 [M+H]⁺

### Example 220. (3R,4S)-1-(6-(4-chlorophenyl)-2-(pyridin-3-yl)pyrimidin-4-yl)-3-fluoropiperidin-4-ol

Using (3R,4S)-3-fluoropiperidin-4-ol and separation method of PREP. HPLC, the title compound was obtained as described for the example 1 (Scheme 1. General procedure A.).

¹H NMR (400 MHz, CD₃OD) δ [ppm]=9.74 (d, J=1.7, 0.8 Hz, 1H), 9.52 (dd, J=8.2, 1.9, 1.5 Hz, 1H), 8.92 (dd, J=5.7, 1.5, 0.8 Hz, 1H), 8.21 (dd, 2H), 8.19-8.14 (m, 1H), 7.51 (d, 2H), 7.30 (s, 1H), 4.56-4.47 (m, 1H), 4.47-4.33 (m, 1H), 4.17-4.06 (m, 1H), 4.04-3.94 (m, 1H), 3.94-3.82 (m, 1H), 3.82-3.68 (m, 1H), 2.18-2.05 (m, 1H), 1.74-1.60 (m, 1H); MS (ESI, m/z): 385.1 [M+H]⁺

### Example 221. (3R,4R)-1-(6-(4-chlorophenyl)-2-(pyridin-3-yl)pyrimidin-4-yl)-3-fluoropiperidin-4-ol

Using (3R,4R)-3-fluoropiperidin-4-ol and separation method of PREP. HPLC, the title compound was obtained as described for the example 1 (Scheme 1. General procedure A.).

¹H NMR (400 MHz, CD₃OD) δ [ppm]=9.74 (d, J=1.7, 0.8 Hz, 1H), 9.52 (dd, J=8.2, 1.9, 1.5 Hz, 1H), 8.92 (dd, J=5.7, 1.5, 0.8 Hz, 1H), 8.21 (dd, 2H), 8.19-8.14 (m, 1H), 7.51 (d, 2H), 7.30 (s, 1H), 4.56-4.47 (m, 1H), 4.47-4.33 (m, 1H), 4.17-4.06 (m, 1H), 4.04-3.94 (m, 1H), 3.94-3.82 (m, 1H), 3.82-3.68 (m, 1H), 2.18-2.05 (m, 1H), 1.74-1.60 (m, 1H); MS (ESI, m/z): 385.1 [M+H]⁺

### Example 222. (3S,4S)-1-(6-(4-chlorophenyl)-2-(pyridin-3-yl)pyrimidin-4-yl)-3-fluoropiperidin-4-ol

Using (3S,4S)-3-fluoropiperidin-4-ol and separation method of PREP. HPLC, the title compound was obtained as described for the example 1 (Scheme 1. General procedure A.).

¹H NMR (400 MHz, CD₃OD) δ [ppm]=9.74 (d, J=1.7, 0.8 Hz, 1H), 9.52 (dd, J=8.2, 1.9, 1.5 Hz, 1H), 8.92 (dd, J=5.7, 1.5, 0.8 Hz, 1H), 8.21 (dd, 2H), 8.19-8.14 (m, 1H), 7.51 (d, 2H), 7.30 (s, 1H), 4.56-4.47 (m, 1H), 4.47-4.33 (m, 1H), 4.17-4.06 (m, 1H), 4.04-3.94 (m, 1H), 3.94-3.82 (m, 1H), 3.82-3.68 (m, 1H), 2.18-2.05 (m, 1H), 1.74-1.60 (m, 1H); MS (ESI, m/z): 385.1 [M+H]⁺

### Example 223. 1-(4-(6-(4-chlorophenyl)-2-(pyridin-3-yl)pyrimidin-4-yl)piperazin-1-yl)-2-hydroxyethan-1-one

Using 2-hydroxy-1-(piperazin-1-yl)ethan-1-one and separation method of PREP. HPLC, the title compound was obtained as described for the example 1 (Scheme 1. General procedure A.).

¹H NMR (400 MHz, CD₃OD) δ [ppm]=9.73 (d, J=1.3 Hz, 1H), 9.39 (dd, J=8.1, 1.8 Hz, 1H), 8.85 (dd, J=5.5, 1.5 Hz, 1H), 8.26 (d, 2H), 8.02 (dd, 1H), 7.54 (d, 2H), 7.31 (s, 1H), 4.32 (s, 2H), 4.04-3.91 (m, 4H), 3.84-3.74 (m, 2H), 3.68-3.60 (m, 2H); MS (ESI, m/z): 410.1 [M+H]⁺

### Example 224. 2-(4-(6-(4-chlorophenyl)-2-(pyridin-3-yl)pyrimidin-4-yl)piperazin-1-yl)propan-1-ol

Using 2-(piperazin-1-yl)propan-1-ol and separation method of PREP. HPLC, the title compound was obtained as described for the example 1 (Scheme 1. General procedure A.).

¹H NMR (400 MHz, CD₃OD) δ [ppm]=9.83 (s, 1H), 9.59 (d, 1H), 8.95 (dd, J=5.6, 1.5, 0.7 Hz, 1H), 8.29 (d, 2H), 8.19 (dd, J=8.2, 5.7, 0.8 Hz, 1H), 7.54 (d, 2H), 7.45 (s, 1H), 3.96 (d, J=3.7 Hz, 1H), 3.92-3.79 (m, 2H), 3.80-3.70 (m, 2H), 3.60-3.50 (m, 2H), 3.45-3.35 (m, 1H), 2.04-1.74 (m, 3H), 1.42 (d, J=6.8 Hz, 3H); MS (ESI, m/z): 410.2 [M+H]⁺

### Example 225. N-(1-(6-(4-chlorophenyl)-2-(pyridin-3-yl)pyrimidin-4-yl)piperidin-4-yl)-2-methoxyacetamide

Using 2-methoxy-N-(piperidin-4-yl)acetamide and separation method of PREP. HPLC, the title compound was obtained as described for the example 1 (Scheme 1. General procedure A.).

¹H NMR (400 MHz, CD₃OD) δ [ppm]=9.75 (s, 1H), 9.52 (dd, J=8.2, 1.7 Hz, 1H), 8.92 (d, J=5.6, 1.5, 0.7 Hz, 1H), 8.23 (d, 2H), 8.15 (dd, J=8.2, 5.7, 0.7 Hz, 1H), 7.53 (d, 2H), 7.31 (s, 1H), 4.85-4.66 (m, 2H), 4.21-4.05 (m, 1H), 3.90 (s, 2H), 3.40 (s, 3H), 3.28-3.15 (m, 2H), 2.04 (d, J=12.8, 3.7 Hz, 2H), 1.70-1.53 (m, 2H); MS (ESI, m/z): 438.2 [M+H]⁺

### Example 226. (1-(2-(pyridin-3-yl)-6-(4-(trifluoromethyl)phenyl)pyrimidin-4-yl)piperidin-4-yl)methanol

Scheme for the preparation of the Compound of Example 226:

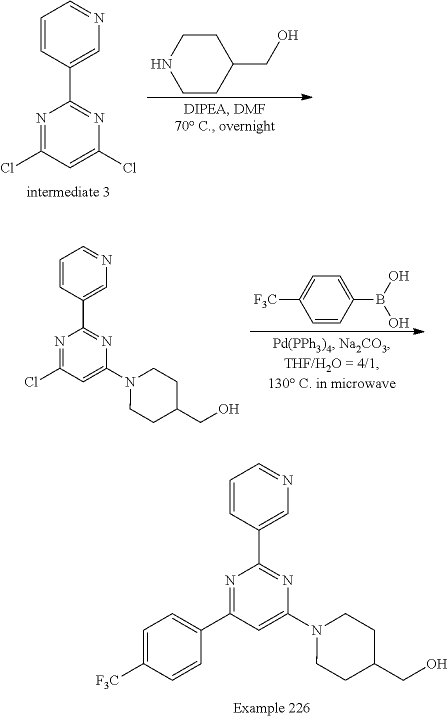 [SMILES: Clc1cc(Cl)nc(-c2cccnc2)n1 ; OCC1CCNCC1 ; OCC1CCN(c2cc(Cl)nc(-c3cccnc3)n2)CC1 ; OB(O)c1ccc(C(F)(F)F)cc1 ; OCC1CCN(c2cc(-c3ccc(C(F)(F)F)cc3)nc(-c3cccnc3)n2)CC1]

### Intermediate 13. (1-(6-chloro-2-(pyridin-3-yl)pyrimidin-4-yl)piperidin-4-yl)methanol

To a solution of 4,6-dichloro-2-(pyridin-3-yl)pyrimidine (3.0 g, 13.3 mmol) in DMF (50 mL) was added diisopropylethylamine (4.63 mL, 26.54 mmol) followed by piperidin-4-ylmethanol (2.01 g, 13.27 mmol) at room temperature. The reaction mixture was heated at 70° C. for overnight and cooled to room temperature. The reaction mixture was quenched with water and extracted three times with DCM. The organic layer was washed with brine, dried over anhydrous MgSO₄ and concentrated under reduced pressure. The crude product was purified by flash chromatography (silica gel, hexane/ethyl acetate, gradient) to give 1.0 g of the title compound.

### Example 226. (1-(2-(pyridin-3-yl)-6-(4-(trifluoromethyl)phenyl)pyrimidin-4-yl)piperidin-4-yl)methanol

To a solution (1-(6-chloro-2-(pyridin-3-yl)pyrimidin-4-yl)piperidin-4-yl)methanol (300 mg, 0.984 mmol), (4-(trifluoromethyl)phenyl)boronic acid (300 mg, 1.580 mmol), sodium carbonate (300 mg, 2.831 mmol) in 30 mL of tetrahydrofuran/H₂O (4/1) was added Pd(PPh₃)₄ (60 mg, 0.052 mmol). The mixture is heated under microwave at 80° C. for 120 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, hexane/ethyl acetate, gradient) to give 219 mg of the title compound. (Scheme 4. General procedure D.).

¹H NMR (600 MHz, DMSO-d₆) [ppm]=9.57 (s, 1H), 8.72 (d, J=8.0, 2.0 Hz, 1H), 8.69 (dd, J=4.8, 1.7 Hz, 1H), 8.52 (d, J=8.1 Hz, 2H), 7.88 (d, J=8.2 Hz, 2H), 7.54 (dd, J=7.9, 4.8 Hz, 1H), 7.45 (s, 1H), 4.88-4.58 (m, 2H), 4.53 (t, J=5.2 Hz, 1H), 3.29 (t, J=5.7 Hz, 2H), 3.01 (t, J=12.9 Hz, 2H), 1.80 (d, 2H), 1.77-1.67 (m, 1H), 1.16 (dd, J=12.5, 4.2 Hz, 2H); MS (ESI, m/z): 415.2 [M+H]⁺

### Example 227. (1-(2-(pyridin-3-yl)-6-(4-(trifluoromethoxy)phenyl)pyrimidin-4-yl)piperidin-4-yl)methanol

Using (4-(trifluoromethoxy)phenyl)boronic acid and separation method of PREP. HPLC, the title compound was obtained as described for the example 226 (Scheme 4. General procedure D.).

¹H NMR (400 MHz, CD₃OD) δ [ppm]=9.60 (s, 1H), 8.96 (d, J=8.2 Hz, 1H), 8.67 (d, J=4.9 Hz, 1H), 8.33 (dd, J=8.9, 3.5 Hz, 2H), 7.68-7.62 (m, 1H), 7.42 (d, J=8.4 Hz, 2H), 7.21 (s, 1H), 3.51-3.44 (m, 2H), 3.27-3.03 (m, 4H), 2.19 (t, J=7.6 Hz, OH), 1.99-1.79 (m, 3H), 1.41-1.22 (m, 2H); MS (ESI, m/z): 431.2 [M+H]⁺

### Example 228. (1-(6-(4-methoxyphenyl)-2-(pyridin-3-yl)pyrimidin-4-yl)piperidin-4-yl)methanol

Using (4-methoxyphenyl)boronic acid and separation method of PREP. HPLC, the title compound was obtained as described for the example 226 (Scheme 4. General procedure D.).

¹H NMR (400 MHz, CD₃OD) δ [ppm]=9.64 (s, 1H), 9.25 (d, J=8.1 Hz, 1H), 8.85 (dd, 1H), 8.15 (d, 2H), 8.01-7.96 (m, 1H), 7.21 (s, 1H), 7.10 (dd, J=8.9, 3.1 Hz, 2H), 4.33 (d, J=6.5 Hz, 2H), 3.89 (s, 3H), 3.20-3.07 (m, 4H), 2.33-2.15 (m, 1H), 2.01-1.90 (m, 2H), 1.50-1.35 (m, 2H); MS (ESI, m/z): 377.2 [M+H]⁺

### Example 229. (1-(2-(pyridin-3-yl)-6-(p-tolyl)pyrimidin-4-yl)piperidin-4-yl)methanol

Using p-tolylboronic acid and separation method of PREP. HPLC, the title compound was obtained as described for the example 226 (Scheme 4. General procedure D.).

¹H NMR (400 MHz, CD₃OD) δ [ppm]=9.66 (s, 1H), 9.31 (d, J=8.2, 1.8 Hz, 1H), 8.87 (dd, J=5.5, 1.5, 0.5 Hz, 1H), 8.07 (d, 2H), 8.05-8.00 (m, 1H), 7.37 (d, 2H), 7.25 (s, 1H), 4.89-4.72 (m, 2H), 4.33 (d, J=6.6 Hz, 2H), 3.23-3.06 (m, 2H), 2.44 (s, 3H), 2.31-2.14 (m, 1H), 1.96 (d, J=13.4 Hz, 2H), 1.51-1.34 (m, 2H); MS (ESI, m/z): 361.2 [M+H]⁺

### Example 230. (3R,4R)-1-(6-(4-chlorophenyl)-2-(pyridin-3-yl)pyrimidin-4-yl)pyrrolidine-3,4-diol

Using (3R,4R)-pyrrolidine-3,4-diol and separation method of PREP. HPLC, the title compound was obtained as described for the example 1 (Scheme 1. General procedure A.).

¹H NMR (400 MHz, CD₃OD) δ [ppm]=9.68 (d, J=2.1 Hz, 1H), 9.40 (dt, J=8.2, 1.8 Hz, 1H), 8.87 (d, J=5.5 Hz, 1H), 8.24-8.16 (m, 2H), 8.06 (dd, J=8.2, 5.6 Hz, 1H), 7.51 (dd, J=8.9, 2.2 Hz, 2H), 6.94 (d, J=8.4 Hz, 1H), 4.36-4.22 (m, 1H), 3.95-3.88 (m, 1H), 3.88-3.79 (m, 1H), 2.10-1.97 (m, 1H), 1.90 (dq, J=18.0, 10.8, 8.5 Hz, 1H), 1.70-1.41 (m, 1H); MS (ESI, m/z): 367.1 [M+H]⁺

### Example 231. (3R,4S)-1-(6-(4-chlorophenyl)-2-(pyridin-3-yl)pyrimidin-4-yl)pyrrolidine-3,4-diol

Using (3R,4S)-pyrrolidine-3,4-diol and separation method of PREP. HPLC, the title compound was obtained as described for the example 1 (Scheme 1. General procedure A.).

¹H NMR (400 MHz, CD₃OD) δ [ppm]=9.68 (d, J=2.1 Hz, 1H), 9.40 (dt, J=8.2, 1.8 Hz, 1H), 8.87 (d, J=5.5 Hz, 1H), 8.24-8.16 (m, 2H), 8.06 (dd, J=8.2, 5.6 Hz, 1H), 7.51 (dd, J=8.9, 2.2 Hz, 2H), 6.94 (d, J=8.4 Hz, 1H), 4.36-4.22 (m, 1H), 3.95-3.88 (m, 1H), 3.88-3.79 (m, 1H), 2.10-1.97 (m, 1H), 1.90 (dq, J=18.0, 10.8, 8.5 Hz, 1H), 1.70-1.41 (m, 1H); MS (ESI, m/z): 367.1 [M+H]J

### Example 232. 1-(3-(6-(4-chlorophenyl)-2-(pyridin-3-yl)pyrimidin-4-yl)tetrahydropyrimidin-1 (2H)-yl)ethan-1-one

Using 1-(tetrahydropyrimidin-1 (2H)-yl)ethan-1-one and separation method of PREP. HPLC, the title compound was obtained as described for the example 1 (Scheme 1. General procedure A.).

¹H NMR (400 MHz, CD₃OD) δ [ppm]=9.71 (d, J=6.8 Hz, 1H), 9.38 (d, J=7.8 Hz, 1H), 8.80 (d, J=5.0 Hz, 1H), 8.19 (d, J=8.5 Hz, 2H), 8.06-8.01 (m, 1H), 7.54 (d, J=8.6 Hz, 2H), 7.03 (s, 1H), 4.04-3.52 (m, 4H), 2.09-1.76 (m, 2H), 1.68-1.48 (m, 2H), 1.29 (s, 3H); MS (ESI, m/z): 394.1 [M+H]⁺

### Example 233. 1-(6-(4-chlorophenyl)-2-(pyridin-3-yl)pyrimidin-4-yl)-4-(hydroxymethyl)piperidin-3-ol

Using 4-(hydroxymethyl)piperidin-3-ol and separation method of PREP. HPLC, the title compound was obtained as described for the example 1 (Scheme 1. General procedure A.).

¹H NMR (400 MHz, CD₃OD) δ [ppm]=9.67 (s, 1H), 9.40 (dd, J=8.2, 4.3, 2.5 Hz, 1H), 8.88 (d, 1H), 8.15 (d, 2H), 8.12-8.04 (m, 1H), 7.50 (d, 2H), 7.23 (s, 1H), 3.93-3.78 (m, 1H), 3.78-3.61 (m, 1H), 3.61-3.46 (m, 1H), 3.35 (d, 2H), 3.13-2.98 (m, 1H), 1.97-1.82 (m, 1H), 1.81-1.37 (m, 3H); MS (ESI, m/z): 397.1 [M+H]⁺

### Example 233. 1-(6-(4-chlorophenyl)-2-(pyridin-3-yl)pyrimidin-4-yl)-4-(hydroxymethyl)piperidin-3-ol

Using 4-(hydroxymethyl)piperidin-3-ol and separation method of PREP. HPLC, the title compound was obtained as described for the example 1 (Scheme 1. General procedure A.).

¹H NMR (400 MHz, CD₃OD) δ [ppm]=9.67 (s, 1H), 9.40 (dd, J=8.2, 4.3, 2.5 Hz, 1H), 8.88 (d, 1H), 8.15 (d, 2H), 8.12-8.04 (m, 1H), 7.50 (d, 2H), 7.23 (s, 1H), 3.93-3.78 (m, 1H), 3.78-3.61 (m, 1H), 3.61-3.46 (m, 1H), 3.35 (d, 2H), 3.13-2.98 (m, 1H), 1.97-1.82 (m, 1H), 1.81-1.37 (m, 3H); MS (ESI, m/z): 397.1 [M+H]⁺

### Example 234. (3R,4R)-1-(6-(4-chlorophenyl)-2-(pyridin-3-yl)pyrimidin-4-yl)-4-fluoropyrrolidin-3-ol

Using (3R,4R)-4-fluoropyrrolidin-3-ol and separation method of PREP. HPLC, the title compound was obtained as described for the example 1 (Scheme 1. General procedure A.).

¹H NMR (400 MHz, CD₃OD) δ [ppm]=9.66 (s, 1H), 9.40 (d, J=8.2, 1.7 Hz, 1H), 8.87 (dd, J=5.7, 1.5 Hz, 1H), 8.17 (d, 2H), 8.07 (dd, 1H), 7.48 (d, 2H), 6.91 (s, 1H), 5.14 (dd, J=50.2, 15.3 Hz, 1H), 4.57-4.46 (m, 1H), 4.24-3.88 (m, 1H), 3.86-3.70 (m, 1H), 2.13-1.69 (m, 2H); MS (ESI, m/z): 371.1 [M+H]⁺

### Example 235. (3R,4S)-1-(6-(4-chlorophenyl)-2-(pyridin-3-yl)pyrimidin-4-yl)-4-fluoropyrrolidin-3-ol

Using (3R,4S)-4-fluoropyrrolidin-3-ol and separation method of PREP. HPLC, the title compound was obtained as described for the example 1 (Scheme 1. General procedure A.).

¹H NMR (400 MHz, CD₃OD) δ [ppm]=9.66 (s, 1H), 9.40 (d, J=8.2, 1.7 Hz, 1H), 8.87 (dd, J=5.7, 1.5 Hz, 1H), 8.17 (d, 2H), 8.07 (dd, 1H), 7.48 (d, 2H), 6.91 (s, 1H), 5.14 (dd, J=50.2, 15.3 Hz, 1H), 4.57-4.46 (m, 1H), 4.24-3.88 (m, 1H), 3.86-3.70 (m, 1H), 2.13-1.69 (m, 2H); MS (ESI, m/z): 371.1 [M+H]⁺

### Example 236. 1-(6-(4-chlorophenyl)-2-(pyridin-3-yl)pyrimidin-4-yl)-4-(hydroxymethyl)pyrrolidin-3-ol

Using 4-(hydroxymethyl)pyrrolidin-3-ol and separation method of PREP. HPLC, the title compound was obtained as described for the example 1 (Scheme 1. General procedure A.).

¹H NMR (400 MHz, CD₃OD) δ [ppm]=9.65 (s, 1H), 9.39 (d, J=8.3, 1.7 Hz, 1H), 8.87 (dd, J=5.6, 1.6 Hz, 1H), 8.15 (d, J=8.6, 2.4 Hz, 2H), 8.07 (dd, J=9.1, 5.7, 3.6 Hz, 1H), 7.48 (dd, J=8.7, 2.3 Hz, 2H), 6.87 (s, 1H), 4.06-3.77 (m, 1H), 3.77-3.62 (m, 1H), 3.62-3.39 (m, 1H), 2.68-2.38 (m, 1H), 2.31-1.99 (m, 2H), 1.99-1.73 (m, 1H), 1.73-1.37 (m, 1H); MS (ESI, m/z): 383.1 [M+H]⁺

### Example 237. N-(1-(6-(4-chlorophenyl)-2-(pyridin-3-yl)pyrimidin-4-yl)piperidin-4-yl)-2-hydroxypropanamide

Using 2-hydroxy-N-(piperidin-4-yl)propanamide and separation method of PREP. HPLC, the title compound was obtained as described for the example 1 (Scheme 1. General procedure A.).

¹H NMR (400 MHz, CD₃OD) δ [ppm]=9.76 (d, J=449.8 Hz, 1H), 9.53 (d, J=1746.2 Hz, 1H), 8.92 (d, J=5.5 Hz, 1H), 8.24 (d, 2H), 8.15 (dd, J=8.2, 5.7 Hz, 1H), 7.55 (dd, J=8.5, 1.6 Hz, 2H), 7.33 (s, 1H), 4.17-3.99 (m, 2H), 3.30-3.20 (m, 4H), 2.11-1.97 (m, 2H), 1.71-1.54 (m, 2H), 1.35 (d, J=6.8 Hz, 3H); MS (ESI, m/z): 438.2 [M+H]⁺

### Example 238. N-(1-(6-(4-chlorophenyl)-2-(pyridin-3-yl)pyrimidin-4-yl)piperidin-4-yl)-2-hydroxyacetamide

Using 2-hydroxy-N-(piperidin-4-yl)acetamide and separation method of PREP. HPLC, the title compound was obtained as described for the example 1 (Scheme 1. General procedure A.).

¹H NMR (400 MHz, CD₃OD) δ [ppm]=9.76 (s, 1H), 9.53 (d, 1H), 8.92 (d, J=5.6 Hz, 1H), 8.25 (d, 2H), 8.15 (dd, J=8.2, 5.6 Hz, 1H), 7.55 (d, J=8.6 Hz, 2H), 7.34 (s, 1H), 4.86-4.65 (m, 2H), 4.20-4.07 (m, 1H), 3.99 (s, 2H), 3.29-3.18 (m, 2H), 2.05 (d, J=12.8 Hz, 2H), 1.71-1.53 (m, 2H); MS (ESI, m/z): 424.2 [M+H]⁺

### Example 239. 2-((4-(6-(4-chlorophenyl)-2-(pyridin-3-yl)pyrimidin-4-yl)piperazin-1-yl)sulfonyl)ethan-1-ol

Using 2-(piperazin-1-ylsulfonyl)ethan-1-ol, the title compound was obtained as described for the example 1 (Scheme 1. General procedure A.).

¹H NMR (600 MHz, DMSO-d₆) δ [ppm]=9.59 (s, 1H), 8.75 (dd, J=8.0, 2.0 Hz, 1H), 8.70 (d, J=4.7 Hz, 1H), 8.37 (d, J=8.3 Hz, 2H), 7.61 (d, J=8.3 Hz, 2H), 7.55 (dd, J=8.0, 4.8 Hz, 1H), 7.45 (s, 1H), 5.04 (s, 1H), 3.97 (s, 4H), 3.75 (t, J=6.1 Hz, 2H), 3.31 (d, J=5.1 Hz, 4H), 3.23 (t, J=6.1 Hz, 2H); MS (ESI, m/z): 460.1 [M+H]⁺

### Example 240. (S)-(1-(6-(4-chlorophenyl)-2-(pyridin-3-yl)pyrimidin-4-yl)pyrrolidin-3-yl)methanol

Using (S)-pyrrolidin-3-ylmethanol, the title compound was obtained as described for the example 1 (Scheme 1. General procedure A.).

¹H NMR (400 MHz, CDCl₃) δ [ppm]=9.68 (s, 1H), 8.75 (dd, J=8.0, 1.9 Hz, 1H), 8.68-8.62 (m, 1H), 8.05 (d, 2H), 7.43 (d, 2H), 7.37 (dd, J=7.9, 4.8, 0.8 Hz, 1H), 6.55 (s, 1H), 4.10-3.85 (m, 1H), 3.85-3.64 (m, 3H), 3.62-3.40 (m, 2H), 2.72-2.51 (m, 1H), 2.26-2.07 (m, 1H), 1.99-1.76 (m, 1H); MS (ESI, m/z): 367.1 [M+H]⁺

### Example 241. N-((3R,4R)-1-(6-(4-chlorophenyl)-2-(pyridin-3-yl)pyrimidin-4-yl)-4-hydroxypyrrolidin-3-yl)acetamide

Using N-((3R,4R)-4-hydroxypyrrolidin-3-yl)acetamide, the title compound was obtained as described for the example 1 (Scheme 1. General procedure A.).

¹H NMR (600 MHz, CD₃OD) δ [ppm]=9.60 (s, 1H), 8.86 (d, J=7.9 Hz, 1H), 8.66-8.63 (m, 1H), 8.20 (d, 2H), 7.56 (s, 1H), 7.51 (d, 2H), 6.88 (s, 1H), 4.35 (d, J=28.7 Hz, 2H), 4.14-3.74 (m, 3H), 3.50 (s, 1H), 1.97 (s, 3H); MS (ESI, m/z): 410.1 [M+H]⁺

### Example 242. (3R,4R)-4-acetamido-1-(6-(4-chlorophenyl)-2-(pyridin-3-yl)pyrimidin-4-yl)pyrrolidin-3-yl acetate

Using (3R,4R)-4-acetamidopyrrolidin-3-yl acetate, the title compound was obtained as described for the example 1 (Scheme 1. General procedure A.).

¹H NMR (600 MHz, CD₃OD) δ [ppm]=9.52 (s, 1H), 8.79 (d, J=7.9 Hz, 1H), 8.60 (s, 1H), 8.15 (d, J=8.5 Hz, 2H), 7.51 (dd, J=7.4, 5.0 Hz, 1H), 7.48 (d, J=8.5 Hz, 2H), 6.81 (s, 1H), 5.29 (s, 1H), 4.51 (s, 1H), 4.19-3.38 (m, 4H), 2.11 (s, 3H), 1.98 (s, 3H); MS (ESI, m/z): 452.1 [M+H]⁺

### Example 243. N-((3R,4S)-1-(6-(4-chlorophenyl)-2-(pyridin-3-yl)pyrimidin-4-yl)-4-hydroxypyrrolidin-3-yl)acetamide

Using N-((3R,4S)-4-hydroxypyrrolidin-3-yl)acetamide, the title compound was obtained as described for the example 1 (Scheme 1. General procedure A.).

¹H NMR (600 MHz, DMSO-d₆) δ [ppm]=9.60 (s, 1H), 8.74 (d, J=7.8 Hz, 1H), 8.69 (d, J=4.7 Hz, 1H), 8.36 (d, 2H), 8.13 (dd, J=26.2, 7.0 Hz, 1H), 7.59 (dd, J=8.7, 2.8 Hz, 2H), 7.54 (t, J=6.7 Hz, 1H), 7.07 (d, J=6.2 Hz, 1H), 5.48 (dd, J=55.9, 3.7 Hz, 1H), 4.16 (d, J=37.3 Hz, 2H), 3.93-3.66 (m, 3H), 3.47 (d, J=11.1 Hz, 1H), 1.81 (d, J=2.7 Hz, 3H); MS (ESI, m/z): 410.1 [M+H]⁺

### Example 244. N-((3S,4R)-1-(6-(4-chlorophenyl)-2-(pyridin-3-yl)pyrimidin-4-yl)-4-hydroxypyrrolidin-3-yl)acetamide

Using N-((3S,4R)-4-hydroxypyrrolidin-3-yl)acetamide, the title compound was obtained as described for the example 1 (Scheme 1. General procedure A.).

¹H NMR (600 MHz, DMSO-d₆) δ [ppm]=9.60 (s, 1H), 8.74 (d, J=7.8 Hz, 1H), 8.69 (d, J=4.7 Hz, 1H), 8.36 (d, 2H), 8.13 (dd, J=26.2, 7.0 Hz, 1H), 7.59 (dd, J=8.7, 2.8 Hz, 2H), 7.54 (t, J=6.7 Hz, 1H), 7.07 (d, J=6.2 Hz, 1H), 5.48 (dd, J=55.9, 3.7 Hz, 1H), 4.16 (d, J=37.3 Hz, 2H), 3.93-3.66 (m, 3H), 3.47 (d, J=11.1 Hz, 1H), 1.81 (d, J=2.7 Hz, 3H); MS (ESI, m/z): 410.1 [M+H]⁺

### Example 245. N-((3S,4S)-1-(6-(4-chlorophenyl)-2-(pyridin-3-yl)pyrimidin-4-yl)-4-hydroxypyrrolidin-3-yl)acetamide

Using N-((3S,4S)-4-hydroxypyrrolidin-3-yl)acetamide, the title compound was obtained as described for the example 1 (Scheme 1. General procedure A.).

¹H NMR (600 MHz, DMSO-d₆) δ [ppm]=9.60 (s, 1H), 8.74 (d, J=7.8 Hz, 1H), 8.69 (d, J=4.7 Hz, 1H), 8.36 (d, 2H), 8.13 (dd, J=26.2, 7.0 Hz, 1H), 7.59 (dd, J=8.7, 2.8 Hz, 2H), 7.54 (t, J=6.7 Hz, 1H), 7.07 (d, J=6.2 Hz, 1H), 5.48 (dd, J=55.9, 3.7 Hz, 1H), 4.16 (d, J=37.3 Hz, 2H), 3.93-3.66 (m, 3H), 3.47 (d, J=11.1 Hz, 1H), 1.81 (d, J=2.7 Hz, 3H); MS (ESI, m/z): 410.1 [M+H]⁺

### Example 246. (1-(6-(4-chloro-3-fluorophenyl)-2-(pyridin-3-yl)pyrimidin-4-yl)piperidin-4-yl)methanol

Using (4-chloro-3-fluorophenyl)boronic acid, the title compound was obtained as described for the example 226 (Scheme 4. General procedure D.).

¹H NMR (600 MHz, DMSO-d₆) δ [ppm]=9.58 (s, 1H), 8.77-8.68 (m, 2H), 8.39 (dd, J=11.0, 2.0 Hz, 1H), 8.25 (dd, J=8.4, 1.8 Hz, 1H), 7.75 (t, J=14.9 Hz, 1H), 7.54 (dd, J=7.8, 4.7 Hz, 1H), 7.43 (s, 1H), 4.75 (s, 1H), 4.56-4.45 (m, 1H), 3.30 (t, J=5.7 Hz, 2H), 3.01 (t, J=12.5 Hz, 2H), 1.84-1.68 (m, 3H), 1.33-1.26 (m, 1H), 1.22-1.12 (m, 2H); MS (ESI, m/z): 399.1[M+H]⁺

### Example 247. (3S,4R)-1-(6-(4-chlorophenyl)-2-(pyridin-3-yl)pyrimidin-4-yl)-4-(hydroxymethyl)piperidin-3-ol

Using (3S,4R)-4-(hydroxymethyl)piperidin-3-ol, the title compound was obtained as described for the example 1 (Scheme 1. General procedure A.).

¹H NMR (400 MHz, DMSO-d₆) δ [ppm]=9.57 (dd, J=2.1, 0.9 Hz, 1H), 8.71 (dd, J=8.0, 2.0 Hz, 1H), 8.68 (dd, J=4.8, 1.7 Hz, 1H), 8.33 (d, J=8.7 Hz, 2H), 7.61-7.56 (m, 2H), 7.53 (dd, J=7.9, 4.8, 0.9 Hz, 1H), 7.32 (s, 1H), 4.55 (d, J=4.3 Hz, 1H), 4.43 (t, J=5.2 Hz, 1H), 3.95 (s, 1H), 3.53-3.42 (m, 1H), 3.16-3.07 (m, 1H), 3.03-2.90 (m, 1H), 2.48-2.40 (m, 1H), 1.82-1.69 (m, 1H), 1.59-1.48 (m, 2H); MS (ESI, m/z): 397.1 [M+H]⁺

### Example 248. (3S,4R)-4-(hydroxymethyl)-1-(2-(pyridin-3-yl)-6-(4-(trifluoromethyl)phenyl)pyrimidin-4-yl)piperidin-3-ol

Scheme for the preparation of the Compound of Example 248:

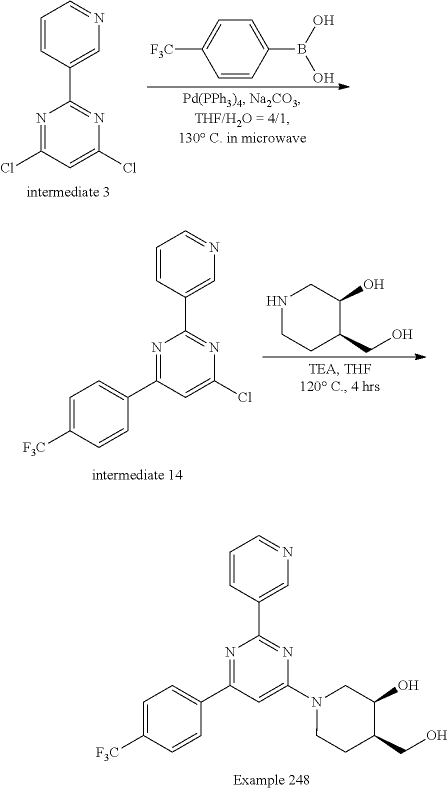 [SMILES: Clc1cc(Cl)nc(-c2cccnc2)n1 ; OB(O)c1ccc(C(F)(F)F)cc1 ; FC(F)(F)c1ccc(-c2cc(Cl)nc(-c3cccnc3)n2)cc1 ; OC[C@H]1CCNC[C@H]1O ; OC[C@H]1CCN(c2cc(-c3ccc(C(F)(F)F)cc3)nc(-c3cccnc3)n2)C[C@H]1O]

### Intermediate 14. 4-chloro-2-(pyridin-3-yl)-6-(4-(trifluoromethyl)phenyl)pyrimidine

To a mixture of 4,6-Dichloro-2-pyridin-3-yl-pyrimidine (1.03 g, 4.6 mmol), (4-(trifluoromethyl)phenyl)boronic acid (0.80 g, 4.2 mmol) and sodium carbonate (1.01 g, 9.5 mmol) in 50 mL of tetrahydrofuran/H₂O (4/1) was added Pd(PPh₃)₄ (203 mg, 0.18 mmol). The mixture is heated under microwave at 65° C. for 20 minutes, cooled to room temperature and extracted three times with EtOAc (50 mL). The organic layer was dried over anhydrous MgSO₄ and concentrated under reduced pressure. The crude product was purified by silicagel column chromatography to give 1.02 g of the title compound.

¹H NMR (600 MHz, CDCl₃) δ [ppm]=7.52 (d, 2H), 7.74 (s, 1H), 7.79 (dd, 1H), 8.06 (d, 2H), 9.02 (d, 1H), 9.12 (d, 1H), 10.16 (s, 1H); MS (ESI, m/z): 336.1 [M+H]⁺

### Example 248. (3S,4R)-4-(hydroxymethyl)-1-(2-(pyridin-3-yl)-6-(4-(trifluoromethyl)phenyl)pyrimidin-4-yl)piperidin-3-ol

To a solution of 4-chloro-2-(pyridin-3-yl)-6-(4-(trifluoromethyl)phenyl)pyrimidine (50 mg, 0.15 mmol) in tetrahydrofuran (6 mL) was added triethylamine (0.3 mL, 2.15 mmol) followed by (3S,4R)-4-(hydroxymethyl)piperidin-3-ol (39 mg, 0.30 mmol) at room temperature. The reaction mixture in sealed tube was heated at 120° C. for 4 h. and cooled to room temperature. The residue was filtered, evaporated in vacuo and isolated by Preparative HPLC to give 50 mg of the title compound (Scheme 1. General procedure A.).

¹H NMR (400 MHz, DMSO-d₆) δ [ppm]=9.58 (s, 1H), 8.72 (dd, J=8.0, 2.0 Hz, 1H), 8.69 (dd, J=4.8, 1.7 Hz, 1H), 8.50 (d, J=8.1 Hz, 2H), 7.88 (d, J=8.2 Hz, 2H), 7.54 (dd, J=8.0, 4.8, 0.9 Hz, 1H), 7.40 (s, 1H), 4.57 (d, J=4.2 Hz, 1H), 4.44 (t, J=5.2 Hz, 1H), 3.96 (s, 1H), 3.52-3.41 (m, 1H), 3.31-3.24 (m, 1H), 3.20-3.08 (m, 1H), 3.08-2.90 (m, 1H), 1.81-1.70 (m, 1H), 1.57-1.52 (m, 2H); MS (ESI, m/z): 431.2 [M+H]⁺

### Example 249. ((3S,4R)-1-(6-(4-chlorophenyl)-2-(pyridin-3-yl)pyrimidin-4-yl)-3-fluoropiperidin-4-yl)methanol

Using ((3S,4R)-3-fluoropiperidin-4-yl)methanol, the title compound was obtained as described for the example 1 (Scheme 1. General procedure A.).

¹H NMR (400 MHz, DMSO-d₆) δ [ppm]=9.57 (s, 1H), 8.75-8.66 (m, 2H), 8.36 (d, 2H), 7.59 (d, 2H), 7.54 (dd, J=7.9, 4.9, 0.9 Hz, 1H), 7.41 (s, 1H), 5.00 (d, J=47.8 Hz, 1H), 4.73 (t, J=5.1 Hz, 1H), 3.51-3.32 (m, 1H), 3.33-3.21 (m, 1H), 3.21-3.09 (m, 1H), 3.09-2.96 (m, 1H), 2.53-2.38 (m, 1H), 2.01-1.82 (m, 1H), 1.66 (d, J=13.3 Hz, 1H), 1.48-1.33 (m, 1H); MS (ESI, m/z): 399.1 [M+H]⁺

### Example 250. ((3S,4R)-3-fluoro-1-(2-(pyridin-3-yl)-6-(4-(trifluoromethyl)phenyl)pyrimidin-4-yl)piperidin-4-yl)methanol

Using ((3S,4R)-3-fluoropiperidin-4-yl)methanol, the title compound was obtained as described for the example 248 (Scheme 1. General procedure A.).

¹H NMR (400 MHz, DMSO-d₆) δ [ppm]=9.58 (s, 1H), 8.76-8.66 (m, 2H), 8.53 (d, 2H), 7.89 (d, 2H), 7.55 (dd, J=7.9, 4.8, 0.9 Hz, 1H), 7.50 (s, 1H), 5.01 (d, J=47.8 Hz, 1H), 4.73 (t, J=5.1 Hz, 1H), 3.48-3.38 (m, 1H), 3.37-3.25 (m, 1H), 3.24-3.12 (m, 1H), 3.05 (t, J=12.9 Hz, 1H), 2.48-2.42 (m, 1H), 2.04-1.79 (m, 1H), 1.67 (d, J=13.3 Hz, 1H), 1.49-1.32 (m, 1H); MS (ESI, m/z): 433.2 [M+H]⁺

### Example 251. ((3R,4S)-1-(6-(4-chlorophenyl)-2-(pyridin-3-yl)pyrimidin-4-yl)-3-fluoropiperidin-4-yl)methanol

Using ((3R,4S)-3-fluoropiperidin-4-yl)methanol, the title compound was obtained as described for the example 1 (Scheme 1. General procedure A.).

¹H NMR (600 MHz, DMSO-d₆) δ [ppm]=9.58 (s, 1H), 8.73 (dd, J=8.0, 1.9 Hz, 1H), 8.70 (s, 1H), 8.38 (d, 2H), 7.59 (d, 2H), 7.55 (dd, J=7.9, 4.7 Hz, 1H), 7.46 (s, 1H), 4.86-4.72 (m, 1H), 4.68 (s, 1H), 4.60-4.35 (m, 1H), 3.60-3.55 (m, 1H), 3.53-3.46 (m, 1H), 3.25-3.18 (m, 1H), 2.00-1.84 (m, 3H), 1.45 (dd, J=14.0, 10.3 Hz, 1H); MS (ESI, m/z): 399.1 [M+H]⁺

### Example 252. ((3R,4S)-3-fluoro-1-(2-(pyridin-3-yl)-6-(4-(trifluoromethyl)phenyl)pyrimidin-4-yl)piperidin-4-yl)methanol

Using ((3R,4S)-3-fluoropiperidin-4-yl)methanol, the title compound was obtained as described for the example 248 (Scheme 1. General procedure A.).

¹H NMR (600 MHz, DMSO-d₆) δ [ppm]=9.60-9.57 (m, 1H), 8.73 (dd, J=7.9, 2.0 Hz, 1H), 8.70 (dd, J=4.7, 1.7 Hz, 1H), 8.55 (d, J=8.2 Hz, 2H), 7.89 (d, J=8.2 Hz, 2H), 7.58-7.52 (m, 2H), 4.89-4.72 (m, 1H), 4.68 (t, J=5.3 Hz, 1H), 4.62-4.44 (m, 1H), 3.61-3.55 (m, 1H), 3.54-3.48 (m, 1H), 3.41-3.34 (m, 1H), 3.28-3.17 (m, 1H), 1.99-1.85 (m, 2H), 1.52-1.42 (m, 1H); MS (ESI, m/z): 433.2 [M+H]⁺

### Example 253. ((3R,4R)-1-(6-(4-chlorophenyl)-2-(pyridin-3-yl)pyrimidin-4-yl)-3-fluoropiperidin-4-yl)methanol

Using ((3R,4R)-3-fluoropiperidin-4-yl)methanol, the title compound was obtained as described for the example 1 (Scheme 1. General procedure A.).

¹H NMR (600 MHz, DMSO-d₆) δ [ppm]=9.58 (s, 1H), 8.73 (dd, J=8.0, 1.9 Hz, 1H), 8.70 (s, 1H), 8.38 (d, 2H), 7.59 (d, 2H), 7.55 (dd, J=7.9, 4.7 Hz, 1H), 7.46 (s, 1H), 4.86-4.72 (m, 1H), 4.68 (s, 1H), 4.60-4.35 (m, 1H), 3.60-3.55 (m, 1H), 3.53-3.46 (m, 1H), 3.25-3.18 (m, 1H), 2.00-1.84 (m, 3H), 1.45 (dd, J=14.0, 10.3 Hz, 1H); MS (ESI, m/z): 399.1 [M+H]⁺

### Example 254. ((3R,4R)-3-fluoro-1-(2-(pyridin-3-yl)-6-(4-(trifluoromethyl)phenyl)pyrimidin-4-yl)piperidin-4-yl)methanol

Using ((3R,4R)-3-fluoropiperidin-4-yl)methanol, the title compound was obtained as described for the example 248 (Scheme 1. General procedure A.).

¹H NMR (600 MHz, DMSO-d₆) δ [ppm]=9.60-9.57 (m, 1H), 8.73 (dd, J=7.9, 2.0 Hz, 1H), 8.70 (dd, J=4.7, 1.7 Hz, 1H), 8.55 (d, J=8.2 Hz, 2H), 7.89 (d, J=8.2 Hz, 2H), 7.58-7.52 (m, 2H), 4.89-4.72 (m, 1H), 4.68 (t, J=5.3 Hz, 1H), 4.62-4.44 (m, 1H), 3.61-3.55 (m, 1H), 3.54-3.48 (m, 1H), 3.41-3.34 (m, 1H), 3.28-3.17 (m, 1H), 1.99-1.85 (m, 2H), 1.52-1.42 (m, 1H); MS (ESI, m/z): 433.2 [M+H]⁺

### Example 255. (3S,4R)-1-(6-(4-chlorophenyl)-2-(pyridin-3-yl)pyrimidin-4-yl)-4-(hydroxymethyl)pyrrolidin-3-ol

Using (3S,4R)-4-(hydroxymethyl)pyrrolidin-3-ol, the title compound was obtained as described for the example 1 (Scheme 1. General procedure A.).

¹H NMR (400 MHz, DMSO-d₆) δ [ppm]=9.58 (s, 1H), 8.72 (d, J=8.0, 2.0 Hz, 1H), 8.68 (d, J=4.7 Hz, 1H), 8.34 (d, J=8.6, 1.6 Hz, 2H), 7.58 (d, J=8.5, 1.7 Hz, 2H), 7.53 (dd, J=7.9, 4.8, 0.8 Hz, 1H), 7.00 (d, J=12.6 Hz, 1H), 5.02 (dd, J=34.8, 4.2 Hz, 1H), 4.61-4.57 (m, 1H), 4.37 (d, J=24.3 Hz, 1H), 3.99-3.83 (m, 1H), 3.78-3.61 (m, 2H), 3.57-3.50 (m, 1H), 3.48-3.24 (m, 1H); MS (ESI, m/z): 383.1 [M+H]⁺

### Example 256. (3S,4R)-4-(hydroxymethyl)-1-(2-(pyridin-3-yl)-6-(4-(trifluoromethyl)phenyl)pyrimidin-4-yl)pyrrolidin-3-ol

Using (3S,4R)-4-(hydroxymethyl)pyrrolidin-3-ol, the title compound was obtained as described for the example 248 (Scheme 1. General procedure A.).

¹H NMR (400 MHz, DMSO-d₆) δ [ppm]=9.59 (s, 1H), 8.74 (dd, J=8.0, 1.9 Hz, 1H), 8.69 (dd, J=4.8, 1.7 Hz, 1H), 8.51 (d, J=8.1 Hz, 2H), 7.88 (d, 2H), 7.54 (dd, J=7.9, 4.8, 0.9 Hz, 1H), 7.10 (d, J=13.8 Hz, 1H), 5.19 (dd, J=36.7, 4.3 Hz, 1H), 4.85-4.73 (m, 1H), 4.23 (d, J=22.9 Hz, 1H), 3.92-3.83 (m, 1H), 3.81-3.69 (m, 1H), 3.63 (d, J=12.1 Hz, 1H), 3.55-3.45 (m, 1H), 3.46-3.35 (m, 1H), 2.40-2.22 (m, 1H); MS (ESI, m/z): 417.2 [M+H]⁺

### Example 257. (3R,4R)-1-(6-(4-chlorophenyl)-2-(pyridin-3-yl)pyrimidin-4-yl)-4-(hydroxymethyl)pyrrolidin-3-ol

Using (3R,4R)-4-(hydroxymethyl)pyrrolidin-3-ol, the title compound was obtained as described for the example 1 (Scheme 1. General procedure A.).

¹H NMR (400 MHz, DMSO-d₆) δ [ppm]=9.58 (s, 1H), 8.72 (d, J=8.0, 2.0 Hz, 1H), 8.68 (d, J=4.7 Hz, 1H), 8.34 (d, J=8.6, 1.6 Hz, 2H), 7.58 (d, J=8.5, 1.7 Hz, 2H), 7.53 (dd, J=7.9, 4.8, 0.8 Hz, 1H), 7.00 (d, J=12.6 Hz, 1H), 5.02 (dd, J=34.8, 4.2 Hz, 1H), 4.61-4.57 (m, 1H), 4.37 (d, J=24.3 Hz, 1H), 3.99-3.83 (m, 1H), 3.78-3.61 (m, 2H), 3.57-3.50 (m, 1H), 3.48-3.24 (m, 1H); MS (ESI, m/z): 383.1 [M+H]⁺

### Example 258. (3R,4R)-4-(hydroxymethyl)-1-(2-(pyridin-3-yl)-6-(4-(trifluoromethyl)phenyl)pyrimidin-4-yl)pyrrolidin-3-ol

Using (3R,4R)-4-(hydroxymethyl)pyrrolidin-3-ol, the title compound was obtained as described for the example 248 (Scheme 1. General procedure A.).

¹H NMR (400 MHz, DMSO-d₆) δ [ppm]=9.59 (s, 1H), 8.74 (dd, J=8.0, 1.9 Hz, 1H), 8.69 (dd, J=4.8, 1.7 Hz, 1H), 8.51 (d, J=8.1 Hz, 2H), 7.88 (d, 2H), 7.54 (dd, J=7.9, 4.8, 0.9 Hz, 1H), 7.10 (d, J=13.8 Hz, 1H), 5.19 (dd, J=36.7, 4.3 Hz, 1H), 4.85-4.73 (m, 1H), 4.23 (d, J=22.9 Hz, 1H), 3.92-3.83 (m, 1H), 3.81-3.69 (m, 1H), 3.63 (d, J=12.1 Hz, 1H), 3.55-3.45 (m, 1H), 3.46-3.35 (m, 1H), 2.40-2.22 (m, 1H); MS (ESI, m/z): 417.2 [M+H]⁺

### Example 259. (3R,4R)-1-(6-(4-chlorophenyl)-2-(pyridin-3-yl)pyrimidin-4-yl)-4-(hydroxymethyl)piperidin-3-ol

Using (3R,4R)-4-(hydroxymethyl)piperidin-3-ol, the title compound was obtained as described for the example 1 (Scheme 1. General procedure A.).

¹H NMR (400 MHz, DMSO-d₆) δ [ppm]=9.57 (dd, J=2.3, 0.8 Hz, 1H), 8.72 (dd, J=7.9, 2.2, 1.7 Hz, 1H), 8.69 (dd, J=4.8, 1.7 Hz, 1H), 8.35 (d, 2H), 7.58 (d, 2H), 7.54 (dd, J=7.9, 4.8, 0.9 Hz, 1H), 7.35 (s, 1H), 5.01 (d, J=5.0 Hz, 1H), 4.43 (t, J=5.1 Hz, 1H), 3.67-3.58 (m, 1H), 3.44-3.37 (m, 1H), 3.35-3.22 (m, 1H), 2.98 (t, J=12.8 Hz, 1H), 2.81-2.67 (m, 1H), 1.88-1.79 (m, 1H), 1.68-1.50 (m, 1H), 1.42-1.22 (m, 1H); MS (ESI, m/z): 397.1 [M+H]⁺

### Example 260. (3R,4R)-4-(hydroxymethyl)-1-(2-(pyridin-3-yl)-6-(4-(trifluoromethyl)phenyl)pyrimidin-4-yl)piperidin-3-ol

Using (3R,4R)-4-(hydroxymethyl)piperidin-3-ol, the title compound was obtained as described for the example 248 (Scheme 1. General procedure A.).

¹H NMR (400 MHz, DMSO-d₆) δ [ppm]=9.59 (s, 1H), 8.76-8.67 (m, 2H), 8.52 (d, 2H), 7.88 (d, 2H), 7.55 (dd, J=7.9, 4.8, 0.9 Hz, 1H), 7.44 (s, 1H), 5.02 (d, J=4.9 Hz, 1H), 4.44 (s, 1H), 3.69-3.58 (m, 1H), 3.48-3.23 (m, 1H), 3.01 (t, J=12.8 Hz, 1H), 2.87-2.70 (m, 1H), 2.56-2.50 (m, 1H), 1.86 (dd, J=13.6, 3.7 Hz, 1H), 1.66-1.51 (m, 1H), 1.30 (dd, J=12.6, 4.3 Hz, 1H); MS (ESI, m/z): 431.2 [M+H]⁺

### Example 261. (3S,4S)-1-(6-(4-chlorophenyl)-2-(pyridin-3-yl)pyrimidin-4-yl)-4-(hydroxymethyl)piperidin-3-ol

Using (3S,4S)-4-(hydroxymethyl)piperidin-3-ol, the title compound was obtained as described for the example 1 (Scheme 1. General procedure A.).

¹H NMR (400 MHz, DMSO-d₆) δ [ppm]=9.57 (dd, J=2.3, 0.8 Hz, 1H), 8.72 (dd, J=7.9, 2.2, 1.7 Hz, 1H), 8.69 (dd, J=4.8, 1.7 Hz, 1H), 8.35 (d, 2H), 7.58 (d, 2H), 7.54 (dd, J=7.9, 4.8, 0.9 Hz, 1H), 7.35 (s, 1H), 5.01 (d, J=5.0 Hz, 1H), 4.43 (t, J=5.1 Hz, 1H), 3.67-3.58 (m, 1H), 3.44-3.37 (m, 1H), 3.35-3.22 (m, 1H), 2.98 (t, J=12.8 Hz, 1H), 2.81-2.67 (m, 1H), 1.88-1.79 (m, 1H), 1.68-1.50 (m, 1H), 1.42-1.22 (m, 1H); MS (ESI, m/z): 397.1 [M+H]⁺

### Example 262. (3S,4S)-4-(hydroxymethyl)-1-(2-(pyridin-3-yl)-6-(4-(trifluoromethyl)phenyl)pyrimidin-4-yl)piperidin-3-ol

Using (3S,4S)-4-(hydroxymethyl)piperidin-3-ol, the title compound was obtained as described for the example 248 (Scheme 1. General procedure A.).

¹H NMR (400 MHz, DMSO-d₆) δ [ppm]=9.59 (s, 1H), 8.76-8.67 (m, 2H), 8.52 (d, 2H), 7.88 (d, 2H), 7.55 (dd, J=7.9, 4.8, 0.9 Hz, 1H), 7.44 (s, 1H), 5.02 (d, J=4.9 Hz, 1H), 4.44 (s, 1H), 3.69-3.58 (m, 1H), 3.48-3.23 (m, 1H), 3.01 (t, J=12.8 Hz, 1H), 2.87-2.70 (m, 1H), 2.56-2.50 (m, 1H), 1.86 (dd, J=13.6, 3.7 Hz, 1H), 1.66-1.51 (m, 1H), 1.30 (dd, J=12.6, 4.3 Hz, 1H); MS (ESI, m/z): 431.2 [M+H]⁺

### Example 263. (S)-1-(2-(4-methylpyridin-3-yl)-6-(4-(trifluoromethyl)phenyl)pyrimidin-4-yl)pyrrolidin-3-ol

Scheme for the preparation of the Compound of Example 263:

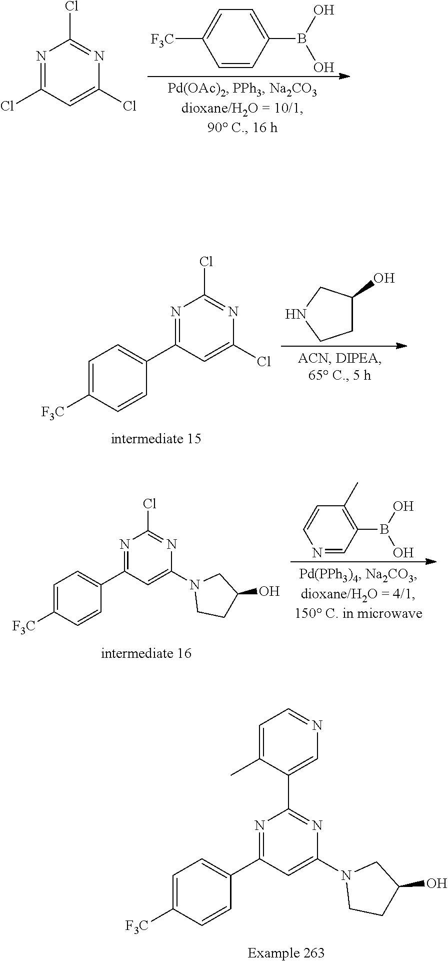

### Intermediate 15. 2,4-dichloro-6-(4-(trifluoromethyl)phenyl)pyrimidine

To a solution of 2,4,6-trichloropyrimidine (5 g, 27.5 mmol) in 1,4-dioxane (50 mL) and H₂O (5 mL) was added (4-(trifluoromethyl)phenyl)boronic acid (3.61 g, 19.25 mmol), Na₂CO₃ (3.79 g, 35.75 mmol), Palladium acetate (617.5 mg, 2.75 mmol) and PPh₃ (721.3 mg, 2.75 mmol) at room temperature under Nitrogen. The reaction mixture was heated and stirred at 90° C. under N₂ for 16 hr. The mixture was cooled to room temperature and the residue was extracted with EtOAc (20 mL×3), washed with brine. The combined organic layer was dried over anhydrous sodium sulfate. The mixture was filtered and concentrated in vacuo. The residue was purified via silica gel column chromatography (Petroleum ether/EtOAc=20/1; V/V) to afford 3.2 g of the title compound.

### Intermediate 16. (S)-1-(2-chloro-6-(4-(trifluoromethyl)phenyl)pyrimidin-4-yl)pyrrolidin-3-ol

To a solution of 2,4-dichloro-6-(4-(trifluoromethyl)phenyl)pyrimidine (2 g, 68.5 mmol) in ACN (20 mL) was added (S)-pyrrolidin-3-ol (716 mg, 82.2 mmol) and DIPEA (2.66 g, 205.5 mmol) at room temperature. The reaction mixture was heated at 65° C. for 5 h. The mixture was concentrated in vacuo. The residue was purified via silica gel column chromatography (Petroleum ether/EtOAc=2/1; V/V) to afford 573 mg of the title compound.

¹H NMR (400 MHz, DMSO-d₆) δ [ppm]=1.83-2.14 (m, 2H), 3.39-3.72 (m, 4H), 4.43 (d, J=27.3 Hz, 1H), 7.11 (d, J=7.3 Hz, 1H), 7.87 (d, J=8.0 Hz, 2H), 8.32 (d, J=7.0 Hz, 2H); MS (ESI, m/z): 344.2 [M+H]⁺

### Example 263. (S)-1-(2-(4-methylpyridin-3-yl)-6-(4-(trifluoromethyl)phenyl)pyrimidin-4-yl)pyrrolidin-3-ol

To a (S)-1-(2-chloro-6-(4-(trifluoromethyl)phenyl)pyrimidin-4-yl)pyrrolidin-3-ol (34 mg, 0.098 mmol), (4-methylpyridin-3-yl)boronic acid (27 mg, 0.20 mmol), sodium carbonate (20.8 mg, 0.20 mmol) in 10 mL of 1,4-dioxane/H₂O (4/1) was added Pd(PPh₃)₄ (11.4 mg, 0.01 mmol). The mixture is heated under microwave at 150° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 30 mg of the title compound. (Scheme 3. General procedure C.).

¹H NMR (400 MHz, DMSO-d₆) δ [ppm]=9.06 (s, 1H), 8.48 (d, J=5.0 Hz, 1H), 8.43 (d, J=8.2 Hz, 2H), 7.88 (d, J=8.3 Hz, 2H), 7.34 (d, J=5.1, 0.7 Hz, 1H), 7.11-7.05 (m, 1H), 5.08 (d, J=39.2 Hz, 1H), 4.44 (d, J=28.6 Hz, 1H), 3.83-3.41 (m, 3H), 2.64 (s, 3H), 2.13-1.85 (m, 2H); MS (ESI, m/z): 401.2 [M+H]⁺

### Example 264. (3R,4S)-1-(6-(4-chlorophenyl)-2-(pyridin-3-yl)pyrimidin-4-yl)-4-(hydroxymethyl)piperidin-3-ol

Using (3R,4S)-4-(hydroxymethyl)piperidin-3-ol, the title compound was obtained as described for the example 1 (Scheme 1. General procedure A.).

¹H NMR (400 MHz, DMSO-d₆) δ [ppm]=9.57 (dd, J=2.3, 0.8 Hz, 1H), 8.72 (dd, J=7.9, 2.2, 1.7 Hz, 1H), 8.69 (dd, J=4.8, 1.7 Hz, 1H), 8.35 (d, 2H), 7.58 (d, 2H), 7.54 (dd, J=7.9, 4.8, 0.9 Hz, 1H), 7.35 (s, 1H), 5.01 (d, J=5.0 Hz, 1H), 4.43 (t, J=5.1 Hz, 1H), 3.67-3.58 (m, 1H), 3.44-3.37 (m, 1H), 3.35-3.22 (m, 1H), 2.98 (t, J=12.8 Hz, 1H), 2.81-2.67 (m, 1H), 1.88-1.79 (m, 1H), 1.68-1.50 (m, 1H), 1.42-1.22 (m, 1H); MS (ESI, m/z): 397.1 [M+H]⁺

### Example 265. (3R,4S)-4-(hydroxymethyl)-1-(2-(pyridin-3-yl)-6-(4-(trifluoromethyl)phenyl)pyrimidin-4-yl)piperidin-3-ol

Using (3R,4S)-4-(hydroxymethyl)piperidin-3-ol, the title compound was obtained as described for the example 248 (Scheme 1. General procedure A.).

¹H NMR (400 MHz, DMSO-d₆) δ [ppm]=9.59 (s, 1H), 8.76-8.67 (m, 2H), 8.52 (d, 2H), 7.88 (d, 2H), 7.55 (dd, J=7.9, 4.8, 0.9 Hz, 1H), 7.44 (s, 1H), 5.02 (d, J=4.9 Hz, 1H), 4.44 (s, 1H), 3.69-3.58 (m, 1H), 3.48-3.23 (m, 1H), 3.01 (t, J=12.8 Hz, 1H), 2.87-2.70 (m, 1H), 2.56-2.50 (m, 1H), 1.86 (dd, J=13.6, 3.7 Hz, 1H), 1.66-1.51 (m, 1H), 1.30 (dd, J=12.6, 4.3 Hz, 1H); MS (ESI, m/z): 431.2 [M+H]⁺

### Example 266. (3S,4S)-1-(6-(4-chlorophenyl)-2-(pyridin-3-yl)pyrimidin-4-yl)-4-(hydroxymethyl)pyrrolidin-3-ol

Using (3S,4S)-4-(hydroxymethyl)pyrrolidin-3-ol, the title compound was obtained as described for the example 1 (Scheme 1. General procedure A.).

¹H NMR (400 MHz, DMSO-d₆) δ [ppm]=9.58 (s, 1H), 8.72 (d, J=8.0, 2.0 Hz, 1H), 8.68 (d, J=4.7 Hz, 1H), 8.34 (d, J=8.6, 1.6 Hz, 2H), 7.58 (d, J=8.5, 1.7 Hz, 2H), 7.53 (dd, J=7.9, 4.8, 0.8 Hz, 1H), 7.00 (d, J=12.6 Hz, 1H), 5.02 (dd, J=34.8, 4.2 Hz, 1H), 4.61-4.57 (m, 1H), 4.37 (d, J=24.3 Hz, 1H), 3.99-3.83 (m, 1H), 3.78-3.61 (m, 2H), 3.57-3.50 (m, 1H), 3.48-3.24 (m, 1H); MS (ESI, m/z): 383.1 [M+H]⁺

### Example 267. (3S,4S)-4-(hydroxymethyl)-1-(2-(pyridin-3-yl)-6-(4-(trifluoromethyl)phenyl)pyrimidin-4-yl)pyrrolidin-3-ol

Using (3S,4S)-4-(hydroxymethyl)pyrrolidin-3-ol, the title compound was obtained as described for the example 248 (Scheme 1. General procedure A.).

¹H NMR (400 MHz, DMSO-d₆) δ [ppm]=9.59 (s, 1H), 8.74 (dd, J=8.0, 1.9 Hz, 1H), 8.69 (dd, J=4.8, 1.7 Hz, 1H), 8.51 (d, J=8.1 Hz, 2H), 7.88 (d, 2H), 7.54 (dd, J=7.9, 4.8, 0.9 Hz, 1H), 7.10 (d, J=13.8 Hz, 1H), 5.19 (dd, J=36.7, 4.3 Hz, 1H), 4.85-4.73 (m, 1H), 4.23 (d, J=22.9 Hz, 1H), 3.92-3.83 (m, 1H), 3.81-3.69 (m, 1H), 3.63 (d, J=12.1 Hz, 1H), 3.55-3.45 (m, 1H), 3.46-3.35 (m, 1H), 2.40-2.22 (m, 1H); MS (ESI, m/z): 417.2 [M+H]⁺

### Example 268. (1-(6-(4-morpholinophenyl)-2-(pyridin-3-yl)pyrimidin-4-yl)piperidin-4-yl)methanol

Using (4-morpholinophenyl)boronic acid, the title compound was obtained as described for the example 226 (Scheme 4. General procedure D.).

¹H NMR (600 MHz, CDCl₃) δ [ppm]=9.69 (s, 1H), 8.76 (dd, J=7.9, 1.9 Hz, 1H), 8.65 (dd, J=4.7, 1.7 Hz, 1H), 8.07 (d, J=8.9 Hz, 2H), 7.37 (dd, J=7.9, 4.8, 0.9 Hz, 1H), 6.98 (d, J=8.9 Hz, 2H), 6.81 (s, 1H), 4.74-4.58 (m, 2H), 3.92-3.83 (m, 4H), 3.55 (d, J=6.2 Hz, 2H), 3.30-3.22 (m, 4H), 3.00 (t, J=12.8, 2.6 Hz, 2H), 1.90 (d, J=13.9 Hz, 2H), 1.88-1.81 (m, 1H), 1.39-1.26 (m, 2H); MS (ESI, m/z): 432.2 [M+H]⁺

### Example 269. (S)-1-(2-(2-methylpyridin-3-yl)-6-(4-(trifluoromethyl)phenyl)pyrimidin-4-yl)pyrrolidin-3-ol

Using (2-methylpyridin-3-yl)boronic acid, the title compound was obtained as described for the example 263 (Scheme 3. General procedure C.).

¹H NMR (600 MHz, CDCl₃) δ [ppm]=8.54 (dd, J=4.8, 1.8 Hz, 1H), 8.31 (dd, J=7.7, 1.8 Hz, 1H), 8.18 (d, J=8.0 Hz, 2H), 7.73 (d, J=8.0 Hz, 2H), 7.25-7.22 (m, 1H), 6.66 (s, 1H), 4.69 (s, 1H), 3.83-3.68 (m, 4H), 2.92 (s, 3H), 2.28-2.10 (m, 2H); MS (ESI, m/z): 401.2 [M+H]⁺

### Example 270. (S)-3-(4-(3-hydroxypyrrolidin-1-yl)-6-(4-(trifluoromethyl)phenyl)pyrimidin-2-yl)pyridin-2-ol

Using (2-hydroxypyridin-3-yl)boronic acid, the title compound was obtained as described for the example 263 (Scheme 3. General procedure C.).

¹H NMR (600 MHz, DMSO-d₆) δ [ppm]=8.37 (d, J=7.7 Hz, 2H), 7.93 (d, J=7.8 Hz, 2H), 7.40 (dd, J=9.1, 6.6, 2.2 Hz, 1H), 7.33 (dd, J=6.4, 2.2, 0.8 Hz, 1H), 6.29 (d, J=9.2, 1.0 Hz, 1H), 6.13 (t, J=6.5, 1.1 Hz, 1H), 5.15 (d, J=42.0 Hz, 1H), 4.47 (d, J=26.1 Hz, 1H), 3.76-3.57 (m, 3H), 2.18-1.90 (m, 2H); MS (ESI, m/z): 403.1 [M+H]⁺

### Example 271. 5-chloro-2-(6-(4-(hydroxymethyl)piperidin-1-yl)-2-(pyridin-3-yl)pyrimidin-4-yl)phenol

Using (4-chloro-2-hydroxyphenyl)boronic acid, the title compound was obtained as described for the example 226 (Scheme 4. General procedure D.).

¹H NMR (600 MHz, CD₃OD) δ [ppm]=9.41 (s, 1H), 8.91 (d, 1H), 8.85 (s, 1H), 7.94-7.86 (m, 2H), 7.19 (s, 1H), 6.94-6.89 (m, 2H), 3.47 (d, J=6.2 Hz, 2H), 3.09 (t, J=13.0 Hz, 2H), 1.98-1.90 (m, 2H), 1.90-1.82 (m, 1H), 1.38-1.22 (m, 4H); MS (ESI, m/z): 397.1 [M+H]⁺

### Example 272. tert-butyl (S)-4-(6-(4-chlorophenyl)-2-(pyridin-3-yl)pyrimidin-4-yl)-2-(hydroxymethyl)piperazine-1-carboxylate

Using tert-butyl (S)-2-(hydroxymethyl)piperazine-1-carboxylate, the title compound was obtained as described for the example 1 (Scheme 1. General procedure A.).

¹H NMR (400 MHz, DMSO-d₆) δ [ppm]=9.58 (dd, J=2.2, 0.9 Hz, 1H), 8.73 (dd, J=8.0, 2.0 Hz, 1H), 8.69 (dd, J=4.8, 1.7 Hz, 1H), 8.33 (d, J=8.7 Hz, 2H), 7.60 (d, J=8.8 Hz, 2H), 7.54 (dd, J=7.9, 4.8, 0.9 Hz, 1H), 7.32 (s, 1H), 4.90 (s, 1H), 4.08 (s, 1H), 3.86 (d, J=11.1 Hz, 1H), 3.49-3.26 (m, 5H), 3.25-3.05 (m, 1H), 1.42 (s, 9H); MS (ESI, m/z): 482.2 [M+H]⁺

### Example 273. 2-chloro-5-(6-(4-(hydroxymethyl)piperidin-1-yl)-2-(pyridin-3-yl)pyrimidin-4-yl)phenol

Using (4-chloro-3-hydroxy-phenyl)boronic acid, the title compound was obtained as described for the example 226 (Scheme 4. General procedure D.).

¹H NMR (400 MHz, DMSO-d₆) δ [ppm]=10.35 (s, 1H), 9.58 (d, J=1.9 Hz, 1H), 8.72-8.64 (m, 2H), 8.03 (d, J=2.1 Hz, 1H), 7.76-7.69 (m, 1H), 7.58-7.50 (m, 1H), 7.46 (d, J=8.4 Hz, 1H), 7.27 (s, 1H), 4.70 (s, 1H), 4.52 (t, J=5.3 Hz, 1H), 3.29 (dd, J=11.1, 5.5 Hz, 2H), 3.00 (t, J=11.9 Hz, 2H), 1.85-1.68 (m, 3H), 1.33-1.26 (m, 1H), 1.22-1.10 (m, 2H); MS (ESI, m/z): 397.1 [M+H]⁺

### Example 274. N-(4-(6-(4-(hydroxymethyl)piperidin-1-yl)-2-(pyridin-3-yl)pyrimidin-4-yl)phenyl)methanesulfonamide

Using [4-(methanesulfonamido)phenyl]boronic acid, the title compound was obtained as described for the example 226 (Scheme 4. General procedure D.).

¹H NMR (400 MHz, DMSO-d₆) δ [ppm]=9.89 (s, 1H), 9.65-9.58 (m, 2H), 8.78-8.64 (m, 2H), 8.24-8.22 (m, 1H), 8.03-7.90 (m, 1H), 7.48 (t, J=7.9 Hz, 1H), 7.32-7.16 (m, 2H), 4.72 (s, 1H), 4.52 (t, J=5.3 Hz, 1H), 3.30 (t, J=5.7 Hz, 2H), 3.07 (s, 3H), 2.99-2.93 (m, 2H), 1.86-1.68 (m, 3H), 1.33-1.26 (m, 1H), 1.22-1.15 (m, 2H); MS (ESI, m/z): 440.2 [M+H]⁺

### Example 275. (1-(6-(4-(4-methylpiperazin-1-yl)phenyl)-2-(pyridin-3-yl)pyrimidin-4-yl)piperidin-4-yl)methanol

Using [4-(4-methylpiperazin-1-yl)phenyl]boronic acid, the title compound was obtained as described for the example 226 (Scheme 4. General procedure D.).

¹H NMR (600 MHz, DMSO-d₆) δ [ppm]=9.55 (d, J=1.6 Hz, 1H), 8.72-8.63 (m, 2H), 8.19 (d, J=8.9 Hz, 2H), 7.53 (dd, J=7.7, 4.9 Hz, 1H), 7.19 (s, 1H), 7.03 (d, J=9.0 Hz, 2H), 4.70 (s, 1H), 4.52 (t, J=5.3 Hz, 1H), 3.31-3.24 (m, 6H), 2.96 (t, J=11.9 Hz, 2H), 2.47 (m, 4H), 2.24 (s, 3H), 1.84-1.68 (m, 3H), 1.30-1.26 (m, 1H), 1.18-1.11 (m, 2H); MS (ESI, m/z): 445.3 [M+H]⁺

### Example 276. (1-(6-(3-fluoro-4-morpholinophenyl)-2-(pyridin-3-yl)pyrimidin-4-yl)piperidin-4-yl)methanol

Using (3-fluoro-4-morpholino-phenyl)boronic acid, the title compound was obtained as described for the example 226 (Scheme 4. General procedure D.).

¹H NMR (400 MHz, DMSO-d₆) δ [ppm]=9.60-9.51 (m, 1H), 8.76-8.63 (m, 2H), 8.18-8.10 (m, 2H), 7.54-7.50 (m, 1H), 7.30 (s, 1H), 7.16-7.08 (m, 1H), 4.74 (s, 1H), 4.52 (t, J=5.3 Hz, 1H), 3.87-3.65 (m, 4H), 3.32-3.26 (m, 2H), 3.16-3.03 (m, 4H), 2.98 (t, J=11.7 Hz, 2H), 1.84-1.69 (m, 3H), 1.29 (m, 1H), 1.20-1.15 (m, 2H); MS (ESI, m/z): 450.2 [M+H]⁺

### Example 277. (1-(2-(pyridin-3-yl)-6-(2,4,6-trifluorophenyl)pyrimidin-4-yl)piperidin-4-yl)methanol

Using (2,4,6-trifluorophenyl)boronic acid, the title compound was obtained as described for the example 226 (Scheme 4. General procedure D.).

¹H NMR (400 MHz, DMSO-d₆) δ [ppm]=9.53 (s, 1H), 8.79-8.61 (m, 2H), 8.24-8.15 (m, 1H), 7.83-7.66 (m, 1H), 7.54-7.48 (m, 1H), 7.14 (s, 1H), 4.63 (s, 1H), 4.52 (t, J=5.3 Hz, 1H), 3.29 (t, J=5.7 Hz, 2H), 3.02 (t, J=12.1 Hz, 2H), 1.88-1.66 (m, 3H), 1.27 (m, 1H), 1.15 (m, 2H); MS (ESI, m/z): 401.2 [M+H]⁺

### Example 278. (1-(2-(pyridin-3-yl)-6-(4-((tetrahydro-2H-pyran-2-yl)oxy)phenyl)pyrimidin-4-yl)piperidin-4-yl)methanol

Using (4-tetrahydropyran-2-yloxyphenyl)boronic acid, the title compound was obtained as described for the example 226 (Scheme 4. General procedure D.).

¹H NMR (600 MHz, DMSO-d₆) δ [ppm]=9.56 (d, J=1.5 Hz, 1H), 8.75-8.66 (m, 2H), 8.26 (d, J=5.9 Hz, 2H), 7.55-7.48 (m, 1H), 7.25 (s, 1H), 7.14 (d, J=5.9 Hz, 2H), 5.60 (t, J=3.2 Hz, 1H), 4.72 (s, 1H), 4.52 (t, J=5.3 Hz, 1H), 3.84-3.71 (m, 1H), 3.63-3.53 (m, 1H), 3.30 (t, J=13.4 Hz, 2H), 2.98 (t, J=11.9 Hz, 2H), 1.91-1.53 (m, 9H), 1.30-1.26 (m, 1H), 1.19-1.11 (m, 2H); MS (ESI, m/z): 447.2 [M+H]⁺

### Example 279. (S)-(4-(2-(pyridin-3-yl)-6-(4-(trifluoromethyl)phenyl)pyrimidin-4-yl)morpholin-2-yl)methanol

Using (S)-morpholin-2-ylmethanol, the title compound was obtained as described for the example 248 (Scheme 1. General procedure A.).

¹H NMR (400 MHz, DMSO-d₆) δ [ppm]=9.60 (dd, J=2.2, 0.9 Hz, 1H), 8.74 (dd, J=7.9, 2.2, 1.7 Hz, 1H), 8.70 (dd, J=4.8, 1.7 Hz, 1H), 8.53 (d, J=8.0, 0.8 Hz, 2H), 7.89 (d, J=8.2, 0.7 Hz, 2H), 7.55 (dd, J=7.9, 4.8, 0.9 Hz, 1H), 7.46 (s, 1H), 4.88 (t, J=5.6 Hz, 1H), 4.56-4.52 (m, 1H), 4.04-3.96 (m, 1H), 3.64-3.45 (m, 5H), 3.11 (t, J=12.4 Hz, 1H), 2.88 (t, J=11.2 Hz, 1H); MS (ESI, m/z): 417.2 [M+H]⁺

### Example 280. (R)-(4-(2-(pyridin-3-yl)-6-(4-(trifluoromethyl)phenyl)pyrimidin-4-yl)morpholin-2-yl)methanol

Using (R)-morpholin-2-ylmethanol, the title compound was obtained as described for the example 248 (Scheme 1. General procedure A.).

¹H NMR (400 MHz, DMSO-d₆) δ [ppm]=9.60 (dd, J=2.2, 0.9 Hz, 1H), 8.74 (dd, J=7.9, 2.2, 1.7 Hz, 1H), 8.70 (dd, J=4.8, 1.7 Hz, 1H), 8.53 (d, J=8.0, 0.8 Hz, 2H), 7.89 (d, J=8.2, 0.7 Hz, 2H), 7.55 (dd, J=7.9, 4.8, 0.9 Hz, 1H), 7.46 (s, 1H), 4.88 (t, J=5.6 Hz, 1H), 4.56-4.52 (m, 1H), 4.04-3.96 (m, 1H), 3.64-3.45 (m, 5H), 3.11 (t, J=12.4 Hz, 1H), 2.88 (t, J=11.2 Hz, 1H); MS (ESI, m/z): 417.2 [M+H]⁺

### Example 281. ((3S,4S)-3-fluoro-1-(2-(pyridin-3-yl)-6-(4-(trifluoromethyl)phenyl)pyrimidin-4-yl)piperidin-4-yl)methanol

Using ((3S,4S)-3-fluoropiperidin-4-yl)methanol, the title compound was obtained as described for the example 248 (Scheme 1. General procedure A.).

¹H NMR (400 MHz, DMSO-d₆) δ [ppm]=9.59 (dd, J=2.2, 0.9 Hz, 1H), 8.73 (dd, J=8.0, 2.0 Hz, 1H), 8.70 (dd, J=4.8, 1.7 Hz, 1H), 8.55 (d, J=7.9, 0.8 Hz, 2H), 7.89 (d, J=8.8, 0.8 Hz, 2H), 7.59-7.52 (m, 2H), 4.87-4.72 (m, 1H), 4.68 (t, J=5.3 Hz, 1H), 4.64-4.56 (m, 1H), 4.53-4.43 (m, 1H), 3.63-3.46 (m, 2H), 3.42-3.33 (m, 1H), 3.29-3.17 (m, 1H), 2.01-1.83 (m, 2H), 1.54-1.40 (m, 1H); MS (ESI, m/z): 433.2 [M+H]⁺

### Example 282. ((3S,4S)-1-(6-(4-chlorophenyl)-2-(pyridin-3-yl)pyrimidin-4-yl)-3-fluoropiperidin-4-yl)methanol

Using ((3S,4S)-3-fluoropiperidin-4-yl)methanol, the title compound was obtained as described for the example 1 (Scheme 1. General procedure A.).

¹H NMR (400 MHz, DMSO-d₆) δ [ppm]=9.57 (d, J=1.5 Hz, 1H), 8.72 (dd, J=7.9, 1.9 Hz, 1H), 8.69 (dd, J=4.8, 1.7 Hz, 1H), 8.38 (d, J=8.8 Hz, 2H), 7.59 (d, J=8.7 Hz, 2H), 7.54 (dd, J=8.0, 4.8, 0.9 Hz, 1H), 7.46 (s, 1H), 4.85-4.71 (m, 1H), 4.67 (t, J=5.3 Hz, 1H), 4.63-4.54 (m, 1H), 4.53-4.40 (m, 1H), 3.64-3.54 (m, 1H), 3.54-3.45 (m, 1H), 3.40-3.28 (m, 1H), 3.28-3.15 (m, 1H), 1.99-1.83 (m, 2H), 1.53-1.37 (m, 1H); MS (ESI, m/z): 399.1 [M+H]⁺

### Example 283. (3S,4S)-1-(2-(pyridin-3-yl)-6-(4-(trifluoromethyl)phenyl)pyrimidin-4-yl)pyrrolidine-3,4-diol

Using (3S,4S)-pyrrolidine-3,4-diol, the title compound was obtained as described for the example 248 (Scheme 1. General procedure A.).

¹H NMR (400 MHz, DMSO-d₆) δ [ppm]=9.60 (d, J=2.1 Hz, 1H), 8.74 (dd, J=8.0, 2.0 Hz, 1H), 8.69 (dd, J=4.8, 1.7 Hz, 1H), 8.51 (d, J=8.1 Hz, 2H), 7.89 (d, J=8.2 Hz, 2H), 7.55 (dd, J=7.9, 4.8 Hz, 1H), 7.11 (s, 1H), 5.34-5.29 (m, 1H), 5.26-5.20 (m, 1H), 4.14 (s, 1H), 4.09 (s, 1H), 3.77 (d, J=2.7 Hz, 2H), 3.71 (dd, J=11.5, 4.2 Hz, 1H), 3.48 (d, J=11.3 Hz, 1H); MS (ESI, m/z): 403.1 [M+H]⁺

### Example 284. (3S,4S)-1-(6-(4-chlorophenyl)-2-(pyridin-3-yl)pyrimidin-4-yl)pyrrolidine-3,4-diol

Using (3S,4S)-pyrrolidine-3,4-diol, the title compound was obtained as described for the example 1 (Scheme 1. General procedure A.).

¹H NMR (400 MHz, DMSO-d₆) δ [ppm]=9.59 (dd, J=2.2, 0.9 Hz, 1H), 8.73 (dd, J=7.9, 1.9 Hz, 1H), 8.68 (dd, J=4.8, 1.7 Hz, 1H), 8.34 (d, J=8.7 Hz, 2H), 7.59 (d, J=8.6 Hz, 2H), 7.54 (dd, J=8.0, 4.8, 0.9 Hz, 1H), 7.02 (s, 1H), 5.31 (d, J=3.6 Hz, 1H), 5.22 (d, J=3.3 Hz, 1H), 4.13 (s, 1H), 4.08 (s, 1H), 3.76 (d, J=2.8 Hz, 2H), 3.69 (dd, J=11.3, 4.2 Hz, 1H), 3.46 (d, J=11.2 Hz, 1H); MS (ESI, m/z): 369.1 [M+H]⁺

### Example 285. (1-(2-(1-methyl-1H-pyrazol-4-yl)-6-(4-morpholinophenyl)pyrimidin-4-yl)piperidin-4-yl)methanol

Scheme for the preparation of the Compound of Example 285:

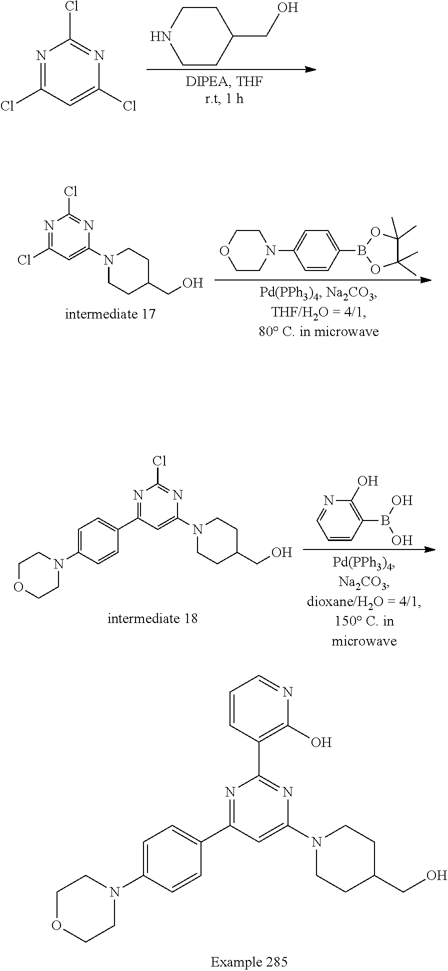

### Intermediate 17. (1-(2,6-dichloropyrimidin-4-yl)piperidin-4-yl)methanol

To a solution 2,4,6-trichloropyrimidine (600 mg, 2.56 mmol), piperidin-4-ylmethanol (310 mg, 3.60 mmol) in 15 mL of tetrahydrofuran was added DIPEA (2.46 ml, 14.16 mmol). The reaction mixture was stirred for 60 minutes at room temperature and extracted three times with DCM (30 mL). The organic layer was washed with brine, dried over anhydrous MgSO₄ and concentrated under reduced pressure. The crude product was purified by flash chromatography (silica gel, hexane/ethyl acetate, gradient) to give 500 mg of the title compound.

### Intermediate 18. (1-(2-chloro-6-(4-morpholinophenyl)pyrimidin-4-yl)piperidin-4-yl)methanol

To a solution (1-(2,6-dichloropyrimidin-4-yl)piperidin-4-yl)methanol (600 mg, 2.56 mmol), 4-(4-(4,4,5,5-tetramethyl-1,3,2-dioxaborolan-2-yl)phenyl)morpholine (570 mg, 2.56 mmol), sodium carbonate (543 mg, 5.13 mmol) in 15 mL of tetrahydrofuran/H₂O (4/1) was added Pd(PPh₃)₄ (296 mg, 0.26 mmol). The mixture is heated under microwave at 80° C. for 120 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, hexane/ethyl acetate, gradient) to give 200 mg of the title compound.

### Example 285. 3-(4-(4-(hydroxymethyl)piperidin-1-yl)-6-(4-morpholinophenyl)pyrimidin-2-yl)pyridin-2-ol

To a solution (1-(2-chloro-6-(4-morpholinophenyl)pyrimidin-4-yl)piperidin-4-yl)methanol (200 mg, 0.68 mmol), 3-pyridylboronic acid (85 mg, 0.70 mmol), sodium carbonate (144 mg, 1.36 mmol) in 15 mL of 1,4-dioxane/H₂O (4/1) was added Pd(PPh₃)₄ (78 mg, 0.08 mmol). The mixture is heated under microwave at 150° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 120 mg of the title compound. (Scheme 5. General procedure E.).

¹H NMR (600 MHz, DMSO-d₆) δ [ppm]=9.57 (s, 1H), 9.13 (s, 1H), 8.87-8.82 (m, 1H), 8.72 (d, J=7.3 Hz, 1H), 8.67 (dd, J=4.7, 1.7 Hz, 1H), 7.54-7.50 (m, 1H), 7.36-7.30 (m, 1H), 7.09 (d, J=15.3 Hz, 1H), 5.08 (dd, J=60.5, 3.3 Hz, 1H), 4.43 (d, J=35.5 Hz, 1H), 3.76-3.52 (m, 4H), 2.13-1.89 (m, 2H); MS (ESI, m/z): 448.2 [M+H]⁺

### Example 286. (1-(2-(1-methyl-1H-pyrazol-4-yl)-6-(4-morpholinophenyl)pyrimidin-4-yl)piperidin-4-yl)methanol

Using (1-methyl-1H-pyrazol-4-yl)boronic acid, the title compound was obtained as described for the example 285 (Scheme 5. General procedure E.).

¹H NMR (600 MHz, DMSO-d₆) δ[ppm]=9.94 (s, 1H), 9.62 (s, 1H), 9.02-8.91 (m, 2H), 8.85-8.84 (m, 1H), 8.45 (s, 1H), 7.77-7.71 (m, 1H), 6.98-6.88 (m, 1H), 6.79 (s, 1H), 4.46 (d, J=36.0 Hz, 1H), 3.85-3.56 (m, 6H), 3.32-3.20 (m, 2H), 2.84 (s, 6H), 2.12-1.92 (m, 2H); MS (ESI, m/z): 435.2 [M+H]⁺

### Example 287. (1-(6-(3-fluoro-4-morpholinophenyl)-2-(1-methyl-1H-pyrazol-4-yl)pyrimidin-4-yl)piperidin-4-yl)methanol

Scheme for the preparation of the Compound of Example 287:

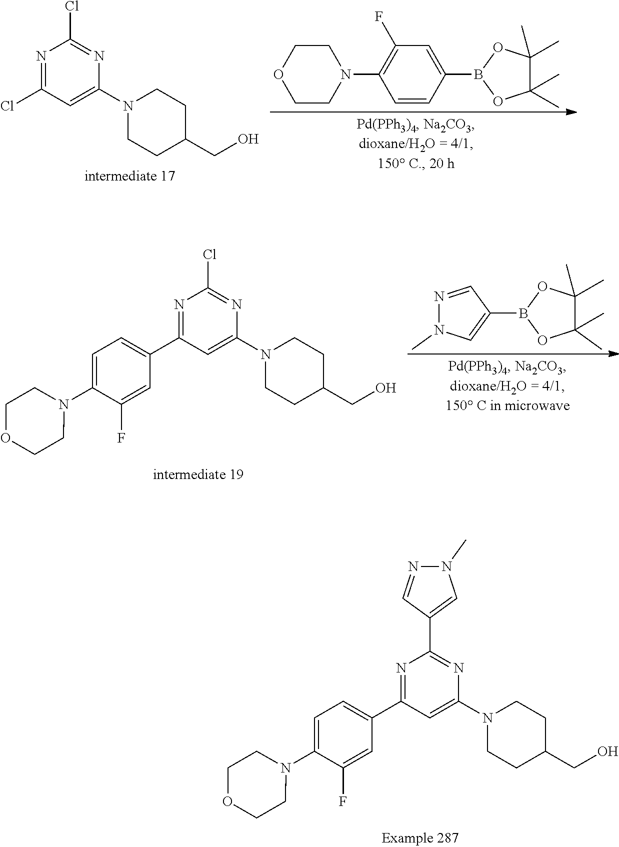

### Intermediate 19. (1-(2-chloro-6-(3-fluoro-4-morpholinophenyl)pyrimidin-4-yl)piperidin-4-yl)methanol

To a solution (1-(2,6-dichloropyrimidin-4-yl)piperidin-4-yl)methanol (300 mg, 1.14 mmol), 4-(2-fluoro-4-(4,4,5,5-tetramethyl-1,3,2-dioxaborolan-2-yl)phenyl)morpholine (527 mg, 1.72 mmol), sodium carbonate (243 mg, 2.29 mmol) in 12 mL of 1,4-dioxane/H₂O (4/1) was added Pd(PPh₃)₄ (132 mg, 0.11 mmol). The mixture is refluxed at 150° C. for 20 hours, cooled to room temperature and extracted three times with DCM (30 mL). The organic layer was washed with brine, dried over anhydrous MgSO₄ and concentrated under reduced pressure. The crude product was purified by flash chromatography (silica gel, DCM/MeOH, gradient) to give 40 mg of the title compound.

### Example 287. (1-(6-(3-fluoro-4-morpholinophenyl)-2-(1-methyl-1H-pyrazol-4-yl)pyrimidin-4-yl)piperidin-4-yl)methanol

To a (1-(2-chloro-6-(3-fluoro-4-morpholinophenyl)pyrimidin-4-yl)piperidin-4-yl)methanol (40 mg, 0.098 mmol), 1-methyl-4-(4,4,5,5-tetramethyl-1,3,2-dioxaborolan-2-yl)-1H-pyrazole (41 mg, 0.20 mmol), sodium carbonate (20.8 mg, 0.20 mmol) in 10 mL of 1,4-dioxane/H₂O (4/1) was added Pd(PPh₃)₄ (11.4 mg, 0.01 mmol). The mixture is heated under microwave at 150° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 30 mg of the title compound (Scheme 5. General procedure E.).

¹H NMR (600 MHz, DMSO-d₆) δ [ppm]=8.31 (s, 1H), 8.05-8.00 (m, 2H), 7.98 (d, J=0.5 Hz, 1H), 7.10-7.02 (m, 2H), 4.63 (s, 1H), 4.49 (t, J=5.3 Hz, 1H), 3.86 (s, 3H), 3.76-3.65 (m, 4H), 3.25 (t, J=5.7 Hz, 2H), 3.12-3.01 (m, 4H), 2.86 (t, J=11.9 Hz, 2H), 1.77-1.62 (m, 3H), 1.28-1.22 (m, 1H), 1.12-1.04 (m, 2H); MS (ESI, m/z): 453.2 [M+H]⁺

### Example 288. (1-(6-(1H-indazol-5-yl)-2-(pyridin-3-yl)pyrimidin-4-yl)piperidin-4-yl)methanol

Using 5-(4,4,5,5-tetramethyl-1,3,2-dioxaborolan-2-yl)-1H-indazole, the title compound was obtained as described for the example 226 (Scheme 4. General procedure D.).

¹H NMR (600 MHz, DMSO-d₆) δ [ppm]=13.24 (s, 1H), 9.61 (d, J=1.7 Hz, 1H), 8.80 (s, 1H), 8.78-8.60 (m, 2H), 8.35 (dd, J=8.8, 1.5 Hz, 1H), 8.22 (s, 1H), 7.64 (d, J=8.8 Hz, 1H), 7.55 (dd, J=7.9, 4.8 Hz, 1H), 7.38 (s, 1H), 4.75 (s, 1H), 4.54 (t, J=5.3 Hz, 1H), 3.30 (t, J=5.7 Hz, 2H), 3.00 (t, J=12.0 Hz, 2H), 1.86-1.67 (m, 3H), 1.30-1.26 (m, 5.7 Hz, 1H), 1.21-1.12 (m, 2H); MS (ESI, m/z): 387.2 [M+H]⁺

### Example 289. (1-(6-(6-morpholinopyridin-3-yl)-2-(pyridin-3-yl)pyrimidin-4-yl)piperidin-4-yl)methanol

Using 4-[5-(4,4,5,5-tetramethyl-1,3,2-dioxaborolan-2-yl)-2-pyridyl]morpholine, the title compound was obtained as described for the example 226 (Scheme 4. General procedure D.).

¹H NMR (600 MHz, DMSO-d₆) δ [ppm]=9.55 (s, 1H), 9.10 (d, J=2.3 Hz, 1H), 8.74-8.64 (m, 2H), 8.45 (dd, J=9.0, 2.5 Hz, 1H), 7.55-7.47 (m, 1H), 7.25 (s, 1H), 6.95 (d, J=9.0 Hz, 1H), 4.71 (s, 1H), 4.53 (t, J=5.3 Hz, 1H), 3.78-3.65 (m, 4H), 3.65-3.52 (m, 4H), 3.29 (t, J=5.7 Hz, 2H), 2.97 (t, J=12.2 Hz, 2H), 1.86-1.67 (m, 3H), 1.30-1.26 (m, 5.7 Hz, 1H), 1.20-1.10 (m, 2H); MS (ESI, m/z): 433.2 [M+H]⁺

### Example 290. 5-(6-(4-(hydroxymethyl)piperidin-1-yl)-2-(pyridin-3-yl)pyrimidin-4-yl)indolin-2-one

Using 5-(4,4,5,5-tetramethyl-1,3,2-dioxaborolan-2-yl)indolin-2-one, the title compound was obtained as described for the example 226 (Scheme 4. General procedure D.).

¹H NMR (600 MHz, DMSO-d₆) δ [ppm]=10.63 (s, 1H), 9.57 (s, 1H), 8.78-8.61 (m, 2H), 8.24-8.20 (m, 2H), 7.26-7.24 (m, 1H), 7.25 (s, 1H), 6.94 (d, J=10.4 Hz, 1H), 4.72 (s, 1H), 4.53 (t, J=5.3 Hz, 1H), 3.59 (s, 2H), 3.30 (t, J=12.4 Hz, 2H), 2.98 (t, J=12.4 Hz, 2H), 1.84-1.65 (m, 3H), 1.30-1.26 (m, 5.7 Hz, 1H), 1.20-1.10 (m, 2H); MS (ESI, m/z): 402.2 [M+H]⁺

### Example 291. 4-(4-(6-(4-(hydroxymethyl)piperidin-1-yl)-2-(pyridin-3-yl)pyrimidin-4-yl)phenyl)morpholin-3-one

Using 4-[4-(4,4,5,5-tetramethyl-1,3,2-dioxaborolan-2-yl)phenyl]morpholin-3-one, the title compound was obtained as described for the example 226 (Scheme 4. General procedure D.).

¹H NMR (600 MHz, DMSO-d₆) δ [ppm]=9.58-9.56 (m, 1H), 8.74-8.68 (m, 2H), 8.35 (d, J=9.0 Hz, 2H), 7.57 (d, J=9.0 Hz, 2H), 7.55-7.53 (m, 1H), 7.35 (s, 1H), 7.16-7.08 (m, 1H), 4.67 (s, 1H), 4.25 (s, 2H), 4.02-4.00 (m, 2H), 3.83-3.81 (m, 2H), 3.30 (t, J=6.0 Hz, 2H), 3.00 (t, J=12.6 Hz, 2H), 1.82-1.70 (m, 3H), 1.30-1.26 (m, 5.7 Hz, 1H), 1.20-1.12 (m, 2H); MS (ESI, m/z): 446.2 [M+H]⁺

### Example 292. 4-(6-(4-(hydroxymethyl)piperidin-1-yl)-2-(pyridin-3-yl)pyrimidin-4-yl)benzoic acid

Using 4-(4,4,5,5-tetramethyl-1,3,2-dioxaborolan-2-yl)benzoic acid, the title compound was obtained as described for the example 226 (Scheme 4. General procedure D.).

¹H NMR (600 MHz, DMSO-d₆) δ [ppm]=13.16 (s, 1H), 9.59 (s, 1H), 8.78-8.68 (m, 2H), 8.44 (d, J=8.4 Hz, 2H), 8.07 (d, J=8.4 Hz, 2H), 7.55 (dd, J=7.7, 4.8 Hz, 1H), 7.43 (s, 1H), 4.75 (s, 1H), 4.54 (t, J=5.3 Hz, 1H), 3.30 (t, J=5.7 Hz, 2H), 3.01 (t, J=12.4 Hz, 2H), 1.84-1.70 (m, 3H), 1.30-1.26 (m, 5.7 Hz, 1H), 1.22-1.12 (m, 2H); MS (ESI, m/z): 391.2 [M+H]⁺

### Example 293. 4 (1-(6-(1-methyl-1H-pyrazol-3-yl)-2-(pyridin-3-yl)pyrimidin-4-yl)piperidin-4-yl)methanol

Using 1-methyl-3-(4,4,5,5-tetramethyl-1,3,2-dioxaborolan-2-yl)pyrazole, the title compound was obtained as described for the example 226 (Scheme 4. General procedure D.).

¹H NMR (600 MHz, DMSO-d₆) δ [ppm]=9.55-9.53 (m, 1H), 8.68-8.84 (m, 2H), 8.49 (s, 1H), 8.21 (s, 1H), 7.55-7.47 (m, 1H), 7.05 (s, 1H), 4.64 (s, 1H), 4.53 (t, J=5.3 Hz, 1H), 3.90 (s, 3H), 3.30 (t, J=8.5 Hz, 2H), 2.95 (t, J=12.0 Hz, 2H), 1.85-1.66 (m, 3H), 1.32-1.26 (m, 1H), 1.18-1.10 (m, 2H); MS (ESI, m/z): 427.2 [M+H]⁺

### Example 294. (1-(6-(5-fluoropyridin-3-yl)-2-(pyridin-3-yl)pyrimidin-4-yl)piperidin-4-yl)methanol

Using 3-fluoro-5-(4,4,5,5-tetramethyl-1,3,2-dioxaborolan-2-yl)pyridine, the title compound was obtained as described for the example 226 (Scheme 4. General procedure D.).

¹H NMR (600 MHz, DMSO-d₆) δ [ppm]=9.60 (s, 1H), 9.39 (s, 1H), 8.77-8.71 (m, 3H), 8.62-8.59 (m, 1H), 7.56-7.52 (m, 1H), 7.51 (s, 1H), 4.72 (s, 1H), 4.53 (t, J=5.3 Hz, 1H), 3.30 (t, J=8.5 Hz, 2H), 3.02 (t, J=12.0 Hz, 2H), 1.75-1.40 (m, 3H), 1.32-1.26 (m, 1H), 1.20-1.10 (m, 2H); MS (ESI, m/z): 366.2 [M+H]⁺

### Example 295. (S)-3-(4-(3-hydroxypyrrolidin-1-yl)-6-(6-morpholinopyridin-3-yl)pyrimidin-2-yl)pyridin-2-ol

Scheme for the preparation of the Compound of Example 295:

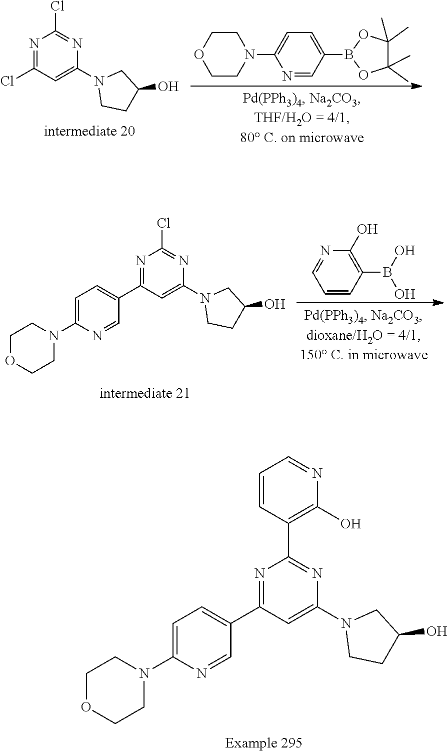 [SMILES: O[C@H]1CCN(c2cc(Cl)nc(Cl)n2)C1 ; CC1(C)OB(c2ccc(N3CCOCC3)nc2)OC1(C)C ; O[C@H]1CCN(c2cc(-c3ccc(N4CCOCC4)nc3)nc(Cl)n2)C1 ; OB(O)c1cccnc1O ; Oc1ncccc1-c1nc(-c2ccc(N3CCOCC3)nc2)cc(N2CC[C@H](O)C2)n1]

### Intermediate 20. (S)-1-(2,6-dichloropyrimidin-4-yl)pyrrolidin-3-ol

To a solution of 2, 4, 6-trichloropyrimidine (1 g, 5.45 mmol) in CH₃CN (20 mL) were added DIPEA (3.25 g, 27.26 mmol) and (S)-3-Hydroxypyrrolidine hydrochloride (712 mg, 8.18 mmol) at room temperature. The reaction mixture was stirred at 65° C. for 16 h. TLC showed the reaction was complete. The reaction mixture cooled to room temperature and quenched by water (30 mL). The mixture was extracted with dichloromethane (40 mL×3). The combined organic layers were washed with brine (30 mL). The organic layers were dried over anhydrous sodium sulfate and filtered. The filtrate was concentrated in vacuo. The residue was purified via silica gel column chromatography (Petroleum ether/EtOAc=5/1; V/V) to give 800 mg of the title compound.

¹H NMR (400 MHz, DMSO-d₆) δ [ppm]=2.07-2.23 (m, 2H), 3.31-3.69 (m, 4H), 3.78-3.81 (m, 1H), 4.66 (d, J=25.1 Hz, 1H), 6.25 (s, 1H); MS (ESI, m/z): 233.0 [M+H]⁺

### Intermediate 21. (S)-1-(2-chloro-6-(6-morpholinopyridin-3-yl)pyrimidin-4-yl)pyrrolidin-3-ol

To a (S)-1-(2,6-dichloropyrimidin-4-yl)pyrrolidin-3-ol (300 mg, 1.28 mmol), 4-(5-(4,4,5,5-tetramethyl-1,3,2-dioxaborolan-2-yl)pyridin-2-yl)morpholine (533 mg, 2.56 mmol), sodium carbonate (272 mg, 2.56 mmol) in 20 mL of THF/H₂O (4/1) was added Pd(PPh₃)₄ (148 mg, 0.13 mmol). The mixture is heated under microwave at 80° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 140 mg of the title compound.

### Example 295. (S)-3-(4-(3-hydroxypyrrolidin-1-yl)-6-(6-morpholinopyridin-3-yl)pyrimidin-2-yl)pyridin-2-ol

To a (S)-1-(2-chloro-6-(6-morpholinopyridin-3-yl)pyrimidin-4-yl)pyrrolidin-3-ol (130 mg, 0.36 mmol), (2-hydroxypyridin-3-yl)boronic acid (100 mg, 0.72 mmol), sodium carbonate (76 mg, 0.72 mmol) in 10 mL of 1,4-dioxane/H₂O (4/1) was added Pd(PPh₃)₄ (20.8 mg, 0.036 mmol). The mixture is heated under microwave at 150° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 13 mg of the title compound (Scheme 5. General procedure E.).

¹H NMR (400 MHz, DMSO-d₆) [ppm]=12.00 (s, 1H), 9.15 (d, J=2.2 Hz, 1H), 8.81 (dd, J=7.2, 2.2 Hz, 1H), 8.47 (dd, J=8.9, 2.3 Hz, 1H), 7.76 (s, 1H), 7.62-7.51 (m, 1H), 6.89 (d, J=8.9 Hz, 1H), 6.43 (t, J=7.2 Hz, 1H), 5.01 (s, 1H), 4.50-4.35 (m, 1H), 3.74-3.68 (m, 4H), 3.66-3.58 (m, 2H), 3.58-3.52 (m, 4H), 3.50-3.40 (m, 2H), 2.10-1.85 (m, 2H); MS (ESI, m/z): 421.2 [M+H]⁺

### Example 296. (S)-1-(6-(4-fluoro-3-nitrophenyl)-2-(pyridin-3-yl)pyrimidin-4-yl)pyrrolidin-3-ol

### Example 297. (S)-1-(6-(4-morpholino-3-nitrophenyl)-2-(pyridin-3-yl)pyrimidin-4-yl)pyrrolidin-3-ol

### Example 298. (S)-1-(6-(3-amino-4-morpholinophenyl)-2-(pyridin-3-yl)pyrimidin-4-yl)pyrrolidin-3-ol

### Example 299. (S)—N-(5-(6-(3-hydroxypyrrolidin-1-yl)-2-(pyridin-3-yl)pyrimidin-4-yl)-2-morpholinophenyl)acetamide

Scheme for the preparation of the Compound of Example 299:

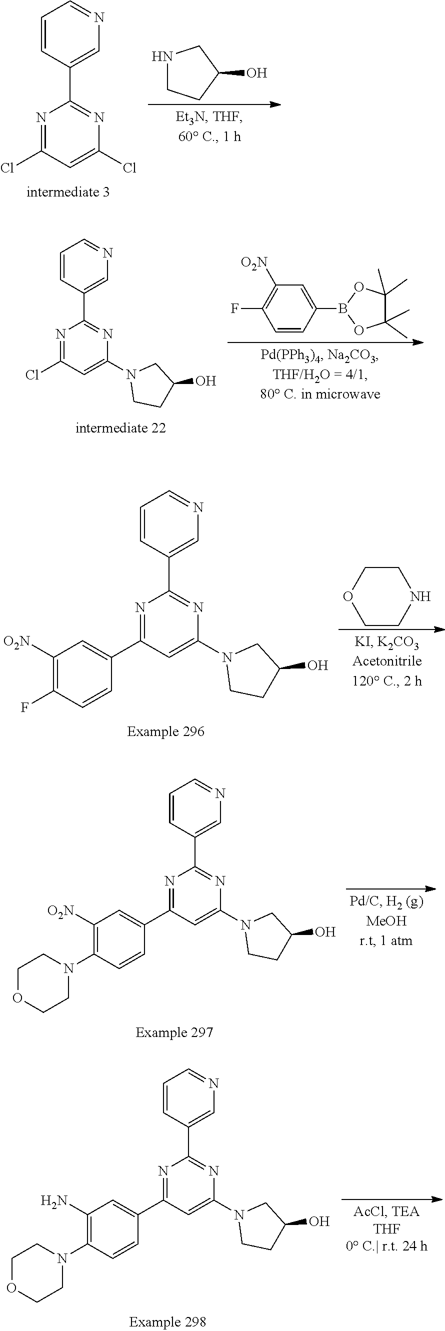

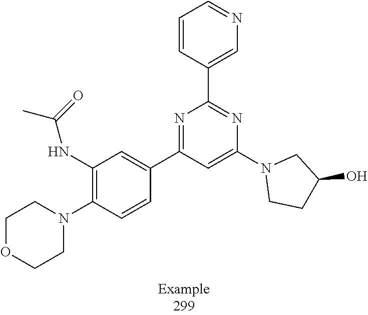 [SMILES: CC(=O)Nc1cc(-c2cc(N3CC[C@H](O)C3)nc(-c3cccnc3)n2)ccc1N1CCOCC1]

### Intermediate 22. (S)-1-(6-chloro-2-(pyridin-3-yl)pyrimidin-4-yl)pyrrolidin-3-ol

To a solution 4,6-dichloro-2-(3-pyridyl)pyrimidine (800 mg, 3.54 mmol), (3S)-pyrrolidin-3-ol (310 mg, 3.60 mmol) in 15 mL of tetrahydrofuran was added DIPEA (2.46 ml, 14.16 mmol). The reaction mixture was heated at 60° C. for 60 minutes, cooled to room temperature and extracted three times with DCM (30 mL). The organic layer was washed with brine, dried over anhydrous MgSO₄ and concentrated under reduced pressure. The crude product was purified by flash chromatography (silica gel, hexane/ethyl acetate, gradient) to give 500 mg of the title compound.

¹H NMR (600 MHz, DMSO-d₆) δ [ppm]=9.38 (dd, J=6.1, 1.7 Hz, 1H), 8.67 (dd, J=4.7, 1.7 Hz, 1H), 8.55-8.48 (m, 1H), 7.52-7.46 (m, 1H), 6.58 (d, J=8.9 Hz, 1H), 5.07 (dd, J=52.4, 3.6 Hz, 1H), 4.39 (d, J=29.2 Hz, 1H), 3.70-3.57 (m, 2H), 3.50-3.41 (m, 2H), 2.04-1.87 (m, 2H); MS (ESI, m/z): 277.1 [M+H]⁺

### Example 296. (S)-1-(6-(4-fluoro-3-nitrophenyl)-2-(pyridin-3-yl)pyrimidin-4-yl)pyrrolidin-3-ol

To a solution (S)-1-(6-chloro-2-(pyridin-3-yl)pyrimidin-4-yl)pyrrolidin-3-ol (250 mg, 0.90 mmol), 2-(4-fluoro-3-nitrophenyl)-4,4,5,5-tetramethyl-1,3,2-dioxaborolane (290 mg, 1.08 mmol), sodium carbonate (287 mg, 2.71 mmol) in 15 mL of THF/H₂O (4/1) was added Pd(PPh₃)₄ (104 mg, 0.09 mmol). The mixture is heated under microwave at 80° C. for 120 minutes, cooled to room temperature and extracted three times with DCM (30 mL). The organic layer was washed with brine, dried over anhydrous MgSO₄ and concentrated under reduced pressure. The crude product was purified by flash chromatography (silica gel, DCM/MeOH, gradient) to give 120 mg of the title compound (Scheme 4. General procedure D.).

¹H NMR (600 MHz, DMSO-d₆) δ [ppm]=9.56 (s, 1H), 9.00 (s, 1H), 8.78-8.60 (m, 3H), 7.73 (t, J=9.8 Hz, 1H), 7.54-7.51 (m, 1H), 7.14 (d, J=11.1 Hz, 1H), 5.09 (dd, J=57.8, 3.5 Hz, 1H), 4.48-4.32 (m, 1H), 3.83-3.54 (m, 4H), 2.11-1.90 (m, 2H); MS (ESI, m/z): 382.1 [M+H]⁺

### Example 297. (S)-1-(6-(4-morpholino-3-nitrophenyl)-2-(pyridin-3-yl)pyrimidin-4-yl)pyrrolidin-3-ol

To a solution (S)-1-(6-(4-fluoro-3-nitrophenyl)-2-(pyridin-3-yl)pyrimidin-4-yl)pyrrolidin-3-ol (100 mg, 0.26 mmol), morpholine (115 mg, 1.31 mmol), K₂CO₃ (43 mg, 0.31 mmol) in 8 mL of acetonitrile was added KI (4.4 mg, 0.026 mmol). The mixture is heated under microwave at 120° C. for 60 minutes, cooled to room temperature and extracted three times with DCM (30 mL). The organic layer was washed with brine, dried over anhydrous MgSO₄ and concentrated under reduced pressure. The crude product was purified by flash chromatography (silica gel, DCM/MeOH, gradient) to give 104 mg of the title compound.

¹H NMR (600 MHz, DMSO-d₆) δ [ppm]=9.56 (s, 1H), 8.77-8.59 (m, 3H), 8.50-8.48 (m, 1H), 7.54-7.50 (m, 1H), 7.39 (d, J=8.7 Hz, 1H), 7.02 (d, J=11.7 Hz, 1H), 5.07 (d, J=57.0 Hz, 1H), 4.43 (d, J=36.5 Hz, 1H), 3.89-3.51 (m, 8H), 3.13-3.02 (m, 4H), 1.96 (dd, J=27.3, 19.6 Hz, 2H); MS (ESI, m/z): 449.2 [M+H]⁺

### Example 298. (S)-1-(6-(3-amino-4-morpholinophenyl)-2-(pyridin-3-yl)pyrimidin-4-yl)pyrrolidin-3-ol

To a solution (S)-1-(6-(4-morpholino-3-nitrophenyl)-2-(pyridin-3-yl)pyrimidin-4-yl)pyrrolidin-3-ol (100 mg, 0.22 mmol) in MeOH 8 mL was added Pd/C (10 mg, 5% wet) and bubbled with H₂ gas. The mixture is stirred for overnight at room temperature under H₂ gas using balloon. The mixture was filtered through Celite 545 pad and filtrate was concentrated under reduced pressure. The crude product was purified by flash chromatography (silica gel, DCM/MeOH, gradient) to give 54 mg of the title compound.

¹H NMR (600 MHz, DMSO-d₆) δ [ppm]=9.57 (s, 1H), 8.74-8.64 (m, 2H), 7.65 (d, J=2.1 Hz, 1H), 7.53-7.49 (m, 1H), 7.42 (dd, J=8.3, 1.7 Hz, 1H), 6.99 (dd, J=31.4, 8.2 Hz, 1H), 6.78-6.66 (m, 1H), 5.05 (dd, J=52.4, 3.5 Hz, 1H), 4.93 (s, 2H), 4.42 (d, J=34.5 Hz, 1H), 3.88-3.48 (m, 8H), 2.86-2.81 (m, 4H), 2.11-1.89 (m, 2H); MS (ESI, m/z): 419.2 [M+H]⁺

### Example 299. (S)—N-(5-(6-(3-hydroxypyrrolidin-1-yl)-2-(pyridin-3-yl)pyrimidin-4-yl)-2-morpholinophenyl)acetamide

To a solution (S)-1-(6-(3-amino-4-morpholinophenyl)-2-(pyridin-3-yl)pyrimidin-4-yl)pyrrolidin-3-ol (50 mg, 0.12 mmol) in 8 mL of THF was added TEA (24 mg, 0.24 mmol). And then acetyl chloride (10 mg, 0.13 mmol) was added to the mixture at 0° C. The reaction mixture was stirred for overnight at room temperature, quenched with water and extracted with DCM (10 mL). The organic layer was washed with brine, dried over anhydrous MgSO₄ and concentrated under reduced pressure. The crude product was purified by flash chromatography (silica gel, DCM/MeOH, gradient) to give 11 mg of the title compound.

¹H NMR (600 MHz, DMSO-d₆) δ [ppm]=9.55 (s, 1H), 9.04 (s, 1H), 8.70-8.62 (m, 2H), 7.95 (s, 1H), 7.56-7.48 (m, 1H), 7.20 (d, J=8.3 Hz, 1H), 6.84-6.76 (m, 1H), 5.06 (dd, J=52.4, 3.5 Hz, 1H), 4.43 (d, J=33.7 Hz, 1H), 3.89-3.42 (m, 8H), 2.90-2.82 (m, 4H), 2.13 (s, 3H), 2.00-1.88 (m, 2H); MS (ESI, m/z): 461.2 [M+H]⁺

### Example 300. (S)-1-(6-(6-fluoropyridin-3-yl)-2-(pyridin-3-yl)pyrimidin-4-yl)pyrrolidin-3-ol

### Example 301. (S)-1-(6-(6-((2-(dimethylamino)ethyl)amino)pyridin-3-yl)-2-(pyridin-3-yl)pyrimidin-4-yl)pyrrolidin-3-ol

Scheme for the preparation of the Compound of Example 301:

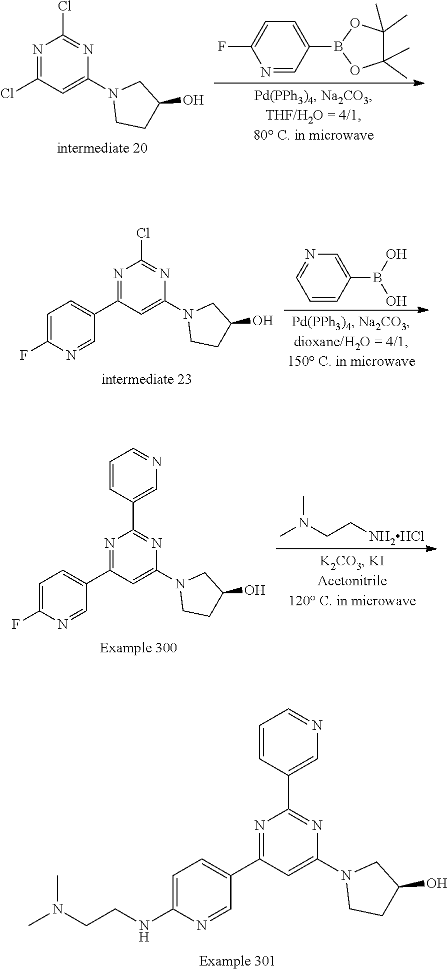

### Intermediate 23. (S)-1-(2-chloro-6-(6-fluoropyridin-3-yl)pyrimidin-4-yl)pyrrolidin-3-ol

To a solution (S)-1-(2,6-dichloropyrimidin-4-yl)pyrrolidin-3-ol (600 mg, 2.56 mmol), 2-fluoro-5-(4,4,5,5-tetramethyl-1,3,2-dioxaborolan-2-yl)pyridine (570 mg, 2.56 mmol), sodium carbonate (543 mg, 5.13 mmol) in 15 mL of tetrahydrofuran/H₂O (4/1) was added Pd(PPh₃)₄ (296 mg, 0.26 mmol). The mixture is heated under microwave at 80° C. for 120 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, hexane/ethyl acetate, gradient) to give 200 mg of the title compound.

### Example 300. (S)-1-(6-(6-fluoropyridin-3-yl)-2-(pyridin-3-yl)pyrimidin-4-yl)pyrrolidin-3-ol

To a solution (S)-1-(2-chloro-6-(6-fluoropyridin-3-yl)pyrimidin-4-yl)pyrrolidin-3-ol (200 mg, 0.68 mmol), 3-pyridylboronic acid (85 mg, 0.70 mmol), sodium carbonate (144 mg, 1.36 mmol) in 15 mL of 1,4-dioxane/H₂O (4/1) was added Pd(PPh₃)₄ (78 mg, 0.08 mmol). The mixture is heated under microwave at 150° C. for 120 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, DCM/MeOH, gradient) to give 120 mg of the title compound (Scheme 4. General procedure D.).

¹H NMR (600 MHz, DMSO-d₆) δ [ppm]=9.57 (s, 1H), 9.13 (s, 1H), 8.87-8.82 (m, 1H), 8.72 (d, J=7.3 Hz, 1H), 8.67 (dd, J=4.7, 1.7 Hz, 1H), 7.54-7.50 (m, 1H), 7.36-7.30 (m, 1H), 7.09 (d, J=15.3 Hz, 1H), 5.08 (dd, J=60.5, 3.3 Hz, 1H), 4.43 (d, J=35.5 Hz, 1H), 3.76-3.52 (m, 4H), 2.13-1.89 (m, 2H); MS (ESI, m/z): 338.1 [M+H]⁺

### Example 301. (S)-1-(6-(6-((2-(dimethylamino)ethyl)amino)pyridin-3-yl)-2-(pyridin-3-yl)pyrimidin-4-yl)pyrrolidin-3-ol

To a solution (S)-1-(6-(6-fluoropyridin-3-yl)-2-(pyridin-3-yl)pyrimidin-4-yl)pyrrolidin-3-ol (50 mg, 0.15 mmol), N¹,N¹-dimethylethane-1,2-diamine hydrochloride (17 mg, 0.18 mmol), K₂CO₃ (25 mg, 0.18 mmol) in 8 mL of acetonitrile was added KI (2.5 mg, 0.015 mmol). The mixture is heated under microwave at 120° C. for 60 minutes, cooled to room temperature and extracted three times with EtOAc (30 mL). The organic layer was washed with brine, dried over anhydrous MgSO₄ and concentrated under reduced pressure. The crude product was purified by flash chromatography (silica gel, DCM/MeOH, gradient) to give 25 mg of the title compound.

¹H NMR (600 MHz, DMSO-d₆) δ [ppm]=9.94 (s, 1H), 9.62 (s, 1H), 9.02-8.91 (m, 2H), 8.85-8.84 (m, 1H), 8.45 (s, 1H), 7.77-7.71 (m, 1H), 6.98-6.88 (m, 1H), 6.79 (s, 1H), 4.46 (d, J=36.0 Hz, 1H), 3.85-3.56 (m, 6H), 3.32-3.20 (m, 2H), 2.84 (s, 6H), 2.12-1.92 (m, 2H); MS (ESI, m/z): 406.2 [M+H]⁺

### Example 302. (3S)-1-(6-(6-(2,6-dimethylmorpholino)pyridin-3-yl)-2-(pyridin-3-yl)pyrimidin-4-yl)pyrrolidin-3-ol

Using 2,6-dimethylmorpholine, the title compound was obtained as described for the example 301.

¹H NMR (600 MHz, DMSO-d₆) δ [ppm]=9.54 (s, 1H), 9.03 (d, J=2.4 Hz, 1H), 8.70 (d, J=8.0 Hz, 1H), 8.65 (dd, J=4.7, 1.7 Hz, 1H), 8.38 (d, J=7.5 Hz, 1H), 7.54-7.46 (m, 1H), 6.94 (d, J=9.0 Hz, 1H), 6.90-6.82 (m, 1H), 5.05 (d, J=58.9 Hz, 1H), 4.42 (d, J=34.0 Hz, 1H), 4.27 (d, J=11.3 Hz, 2H), 3.83-3.34 (m, 8H), 2.04-1.88 (m, 2H), 1.15 (d, J=6.2 Hz, 6H); MS (ESI, m/z): 433.2 [M+H]⁺

### Example 303. 5-chloro-2-(6-(4-((2-hydroxyethyl)sulfonyl)piperazin-1-yl)-2-(pyridin-3-yl)pyrimidin-4-yl)phenol

Scheme for the preparation of the Compound of Example 303:

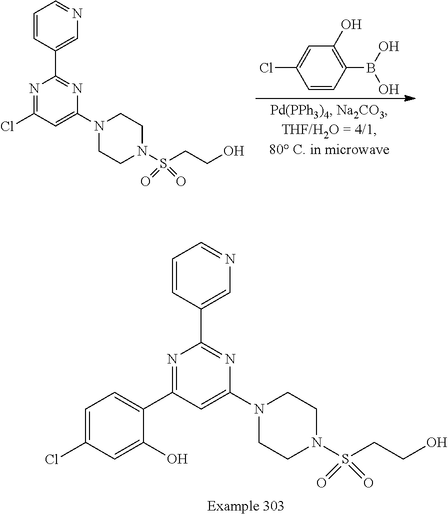 [SMILES: O=S(=O)(CCO)N1CCN(c2cc(Cl)nc(-c3cccnc3)n2)CC1 ; OB(O)c1ccc(Cl)cc1O ; O=S(=O)(CCO)N1CCN(c2cc(-c3ccc(Cl)cc3O)nc(-c3cccnc3)n2)CC1]

### Example 303. 5-chloro-2-(6-(4-((2-hydroxyethyl)sulfonyl)piperazin-1-yl)-2-(pyridin-3-yl)pyrimidin-4-yl)phenol

To a 2-((4-(6-chloro-2-(pyridin-3-yl)pyrimidin-4-yl)piperazin-1-yl)sulfonyl)ethan-1-ol (30 mg, 0.076 mmol), (4-chloro-2-hydroxyphenyl)boronic acid (17 mg, 0.1 mmol), sodium carbonate (25 mg, 0.24 mmol) in 10 mL of THF/H₂O (4/1) was added Pd(PPh₃)₄ (9 mg, 0.008 mmol). The mixture is heated under microwave at 80° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 17 mg of the title compound (Scheme 4. General procedure D.).

¹H NMR (600 MHz, DMSO-d) δ [ppm]=9.35 (d, J=1.9 Hz, 1H), 8.74 (dd, J=4.7, 1.5 Hz, 1H), 8.53-8.46 (m, 1H), 8.27-8.20 (m, 1H), 7.62-7.55 (m, 1H), 7.47 (s, 1H), 7.03-6.96 (m, 2H), 5.04 (t, J=5.4 Hz, 1H), 4.05-3.90 (m, 4H), 3.74-3.70 (m, 2H), 3.31-3.28 (m, 4H), 3.22 (t, J=6.1 Hz, 2H); MS (ESI, m/z): 476.1 [M+H]⁺

### Example 304. (S)-3-(4-(4-chloro-2-hydroxyphenyl)-6-(3-hydroxypyrrolidin-1-yl)pyrimidin-2-yl)pyridin-2-ol

Scheme for the preparation of the Compound of Example 304:

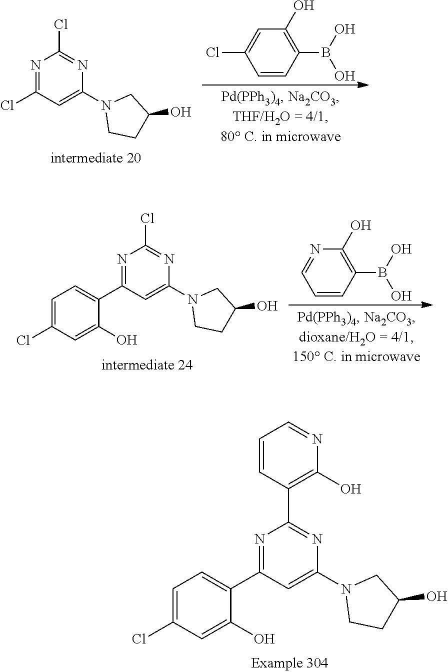 [SMILES: O[C@H]1CCN(c2cc(Cl)nc(Cl)n2)C1 ; OB(O)c1ccc(Cl)cc1O ; Oc1cc(Cl)ccc1-c1cc(N2CC[C@H](O)C2)nc(Cl)n1 ; OB(O)c1cccnc1O ; Oc1cc(Cl)ccc1-c1cc(N2CC[C@H](O)C2)nc(-c2cccnc2O)n1]

### Intermediate 24. (S)-1-(2-chloro-6-(4-chloro-2-hydroxyphenyl)pyrimidin-4-yl)pyrrolidin-3-ol

To a (S)-1-(2,6-dichloropyrimidin-4-yl)pyrrolidin-3-ol (600 mg, 2.56 mmol), (4-chloro-2-hydroxyphenyl)boronic acid (442 mg, 2.56 mmol), sodium carbonate (815 mg, 7.69 mmol) in 20 mL of THF/H₂O (4/1) was added Pd(PPh₃)₄ (296 mg, 0.26 mmol). The mixture is heated under microwave at 80° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 50 mg of the title compound.

### Example 304. (S)-3-(4-(4-chloro-2-hydroxyphenyl)-6-(3-hydroxypyrrolidin-1-yl)pyrimidin-2-yl)pyridin-2-ol

To a (S)-1-(2-chloro-6-(4-chloro-2-hydroxyphenyl)pyrimidin-4-yl)pyrrolidin-3-ol (50 mg, 0.15 mmol), (2-hydroxypyridin-3-yl)boronic acid (42.6 mg, 0.31 mmol), sodium carbonate (48.7 mg, 0.46 mmol) in 10 mL of dioxane/H₂O (4/1) was added Pd(PPh₃)₄ (17.7 mg, 0.015 mmol). The mixture is heated under microwave at 150° C. for 120 minutes, cooled to room temperature and extracted with DCM. The organic layer was washed with brine, dried over anhydrous MgSO₄ and concentrated under reduced pressure. The crude product was purified by flash chromatography (silica gel, DCM/MeOH, gradient) to give 1.8 mg of the title compound (Scheme 5. General procedure E.).

¹H NMR (600 MHz, DMSO-d₆) δ [ppm]=13.01 (s, 1H), 8.89 (s, 1H), 8.01 (d, J=6.8 Hz, 2H), 7.21-6.98 (m, 3H), 6.70 (s, 1H), 5.32-5.14 (m, 1H), 4.49 (d, J=34.7 Hz, 1H), 3.85-3.57 (m, 4H), 2.13-1.95 (m, 2H); MS (ESI, m/z): 385.1 [M+H]⁺

### Example 305. (S)-1-(6-(4-aminophenyl)-2-(pyridin-3-yl)pyrimidin-4-yl)pyrrolidin-3-ol

Using 4-(4,4,5,5-tetramethyl-1,3,2-dioxaborolan-2-yl)aniline, the title compound was obtained as described for the example 296 (Scheme 4. General procedure D.).

¹H NMR (600 MHz, DMSO-d₆) δ[ppm]=9.57 (s, 1H), 8.75-8.70 (m, 1H), 8.67 (dd, J=4.7, 1.7 Hz, 1H), 8.01 (d, J=8.6 Hz, 2H), 7.54-7.50 (m, 1H), 6.73 (s, 1H), 6.66 (d, J=8.6 Hz, 2H), 5.62 (s, 2H), 5.12-4.98 (m, 1H), 4.42 (s, 1H), 3.384-3.50 (m, 4H), 2.11-1.87 (m, 2H); MS (ESI, m/z): 385.1 [M+H]⁺

### Example 306. 4-(4-chlorophenyl)-2-(pyridin-3-yl)-6-(4-(vinylsulfonyl)piperazin-1-yl)pyrimidine

Using 1-(vinylsulfonyl)piperazine, the title compound was obtained as described for the example 1 (Scheme 1. General procedure A.).

¹H NMR (600 MHz, CDCl₃) δ [ppm]=9.66 (s, 1H), 8.73 (dd, J=8.0, 2.0 Hz, 1H), 8.69 (dd, J=4.8, 1.8 Hz, 1H), 8.06 (d, 2H), 7.47 (d, 2H), 7.40 (dd, J=7.9, 4.8 Hz, 1H), 6.84 (s, 1H), 6.44 (dd, J=16.6, 9.9 Hz, 1H), 6.31 (d, J=16.6 Hz, 1H), 6.09 (d, J=9.9 Hz, 1H), 3.96 (t, J=5.0 Hz, 4H), 3.31 (t, J=5.1 Hz, 4H); MS (ESI, m/z): 442.1 [M+H]⁺

### Example 307. (1-(6-(2,4-dichlorophenyl)-2-(pyridin-3-yl)pyrimidin-4-yl)piperidin-4-yl)methanol

Using (2,4-dichlorophenyl)boronic acid, the title compound was obtained as described for the example 226 (Scheme 4. General procedure D.).

¹H NMR (400 MHz, DMSO-d₆) δ [ppm]=9.46 (dd, J=2.2, 0.9 Hz, 1H), 8.69-8.65 (m, 1H), 8.61 (dt, J=8.0, 1.9 Hz, 1H), 7.77 (d, J=2.1 Hz, 1H), 7.71 (d, J=8.3 Hz, 1H), 7.57 (dd, J=8.3, 2.1 Hz, 1H), 7.51 (dd, J=8.0, 4.8, 0.9 Hz, 1H), 7.03 (s, 1H), 4.65-4.31 (m, 2H), 3.28 (d, J=5.8 Hz, 2H), 3.04-2.94 (m, 2H), 1.85-1.65 (m, 3H), 1.20-1.04 (m, 2H); MS (ESI, m/z): 415.1 [M+H]⁺

### Example 308. (S)-(4-(2-(pyridin-3-yl)-6-(4-(trifluoromethyl)phenyl)pyrimidin-4-yl)piperazin-2-yl)methanol

Using (S)-piperazin-2-ylmethanol, the title compound was obtained as described for the example 248 (Scheme 1. General procedure A.).

¹H NMR (400 MHz, DMSO-d₆) δ [ppm]=9.59 (dd, J=2.2, 0.9 Hz, 1H), 8.73 (dt, J=7.9, 1.9 Hz, 1H), 8.70 (dd, J=4.8, 1.7 Hz, 1H), 8.55-8.48 (m, 2H), 7.93-7.85 (m, 2H), 7.55 (dd, J=7.9, 4.8, 0.9 Hz, 1H), 7.43 (s, 1H), 4.90-4.76 (m, 1H), 4.59-4.54 (m, 2H), 3.56-3.38 (m, 1H), 3.12-2.96 (m, 2H), 2.83-2.68 (m, 3H); MS (ESI, m/z): 416.2 [M+H]⁺

### Example 309. (R)-(4-(2-(pyridin-3-yl)-6-(4-(trifluoromethyl)phenyl)pyrimidin-4-yl)piperazin-2-yl)methanol

Using (R)-piperazin-2-ylmethanol, the title compound was obtained as described for the example 248 (Scheme 1. General procedure A.).

¹H NMR (400 MHz, DMSO-d₆) δ [ppm]=9.59 (dd, J=2.2, 0.9 Hz, 1H), 8.73 (dt, J=7.9, 1.9 Hz, 1H), 8.70 (dd, J=4.8, 1.7 Hz, 1H), 8.55-8.48 (m, 2H), 7.93-7.85 (m, 2H), 7.55 (dd, J=7.9, 4.8, 0.9 Hz, 1H), 7.43 (s, 1H), 4.90-4.76 (m, 1H), 4.59-4.54 (m, 2H), 3.56-3.38 (m, 1H), 3.12-2.96 (m, 2H), 2.83-2.68 (m, 3H); MS (ESI, m/z): 416.2 [M+H]⁺

### Example 310. (R)-1-(2-(pyridin-3-yl)-6-(4-(trifluoromethyl)phenyl)pyrimidin-4-yl)pyrrolidine-3-carboxylic acid

Using (R)-pyrrolidine-3-carboxylic acid, the title compound was obtained as described for the example 248 (Scheme 1. General procedure A.).

¹H NMR (600 MHz, DMSO-d₆) δ[ppm]=2.10-2.39 (m, 2H), 3.61-4.08 (m, 4H), 7.10-7.20 (m, 1H), 7.55-7.57 (m, 1H), 7.89-7.91 (m, 2H), 8.54 (br, 2H), 8.70-8.71 (m, 1H), 8.74-8.76 (m, 1H), 9.61 (s, 1H); MS (ESI, m/z): 415.2 [M+H]⁺

### Example 311. (R)-1-(6-(4-chlorophenyl)-2-(pyridin-3-yl)pyrimidin-4-yl)pyrrolidine-3-carboxylic acid

Using (R)-pyrrolidine-3-carboxylic acid, the title compound was obtained as described for the example 1 (Scheme 1. General procedure A.).

¹H NMR (600 MHz, DMSO-d₆) δ [ppm]=2.10-2.30 (m, 2H), 3.34 (br, 2H), 3.50-3.95 (m, 2H), 7.02-7.10 (m, 1H), 7.53-7.55 (m, 1H), 7.59 (d, 2H), 8.35 (br, 2H), 8.69-8.70 (m, 1H), 8.73 (d, 1H), 9.58 (s, 1H); MS (ESI, m/z): 381.1 [M+H]⁺

### Example 312. (R)-1-(2-(1-methyl-1H-pyrazol-4-yl)-6-(4-(trifluoromethyl)phenyl)pyrimidin-4-yl)pyrrolidine-3-carboxylic acid

Using (R)-pyrrolidine-3-carboxylic acid, the title compound was obtained as described for the example 357 (Scheme 2. General procedure B.).

¹H NMR (400 MHz, DMSO-d₆) δ [ppm]=1.23 (br, 4H), 3.35 (s, 3H), 3.91 (s, 2H), 6.87 (br, 1H), 7.86 (d, 2H), 8.03 (s, 1H), 8.34 (s, 1H), 8.44 (br, 2H); MS (ESI, m/z): 418.2 [M+H]⁺

### Example 313. (R)-1-(6-(4-chlorophenyl)-2-(1-methyl-1H-pyrazol-4-yl)pyrimidin-4-yl)pyrrolidine-3-carboxylic acid

Using (R)-pyrrolidine-3-carboxylic acid, the title compound was obtained as described for the example 174 (Scheme 2. General procedure B.).

¹H NMR (400 MHz, DMSO-d₆) δ [ppm]=1.20-1.23 (m, 4H), 3.35 (s, 3H), 3.90 (s, 2H), 6.87 (br, 1H), 7.56 (d, 2H), 8.01 (s, 1H), 8.27 (br, 2H), 8.33 (s, 1H); MS (ESI, m/z): 384.1 [M+H]⁺

### Example 314. (R)-2-(2-(pyridin-3-yl)-6-(4-(trifluoromethyl)phenyl)pyrimidin-4-yl)isoxazolidin-4-ol

Using (R)-isoxazolidin-4-ol, the title compound was obtained as described for the example 248 (Scheme 1. General procedure A.).

¹H NMR (600 MHz, DMSO-d₆) δ [ppm]=3.94-4.01 (m, 1H), 4.02-4.11 (m, 1H), 4.76 (br, 1H), 5.45 (m, 1H), 7.58-7.59 (m, 2H), 7.91 (d, 2H), 8.51 (d, 2H), 8.73-8.77 (m, 2H), 9.61 (br, 1H); MS (ESI, m/z): 389.2 [M+H]⁺

### Example 315. (S)-1-(6-(6-morpholinopyridin-3-yl)-2-(pyridin-3-yl)pyrimidin-4-yl)pyrrolidin-3-ol

Using (6-morpholinopyridin-3-yl)boronic acid, the title compound was obtained as described for the example 296 (Scheme 4. General procedure D.).

¹H NMR (600 MHz, DMSO-d₆) [ppm]=1.94-2.09 (m, 2H), 3.57-3.59 (m, 4H), 3.69-0.3.73 (m, 4H), 4.42-4.74 (m, 2H), 5.02-5.12 (m, 2H), 6.90-6.96 (d, 2H), 7.51-7.54 (m, 1H), 8.42 (d, 1H), 8.67 (d, 1H), 8.73 (d, 1H), 9.07 (br, 1H), 9.56 (s, 1H); MS (ESI, m/z): 405.2 [M+H]⁺

### Example 316. (S)-1-(6-(4-chloro-2-hydroxyphenyl)-2-(pyridin-3-yl)pyrimidin-4-yl)pyrrolidin-3-ol

Using (4-chloro-2-hydroxyphenyl)boronic acid, the title compound was obtained as described for the example 296 (Scheme 4. General procedure D.).

¹H NMR (600 MHz, DMSO-d₆) [ppm]=1.94-2.03 (m, 2H), 3.60-3.77 (m, 2H), 4.43-4.49 (m, 2H), 6.98-7.03 (m, 2H), 7.08 (d, 1H), 7.59-7.61 (m, 1H), 8.20-8.23 (m, 1H), 8.49-8.50 (m, 1H), 8.74-8.75 (m, 1H), 9.36 (br, 1H); MS (ESI, m/z): 369.1 [M+H]⁺

### Example 317. (S)-3-(6-(3-hydroxypyrrolidin-1-yl)-2-(pyridin-3-yl)pyrimidin-4-yl)pyridin-2-ol

Using (2-hydroxypyridin-3-yl)boronic acid, the title compound was obtained as described for the example 296 (Scheme 4. General procedure D.).

¹H NMR (600 MHz, DMSO-d₆) δ [ppm]=9.63 (d, J=5.6 Hz, 1H), 9.17 (s, 1H), 8.88 (d, J=8.5 Hz, 2H), 8.74 (dd, J=4.9, 1.7 Hz, 1H), 7.64 (dd, J=7.9, 5.0 Hz, 1H), 7.37 (dd, J=8.9, 2.5 Hz, 1H), 7.13 (d, J=14.7 Hz, 1H), 4.46 (d, J=35.8 Hz, 1H), 3.77-3.55 (m, 3H), 2.02-1.93 (m, 2H), 1.48-1.41 (m, 1H); MS (ESI, m/z): 336.1 [M+H]⁺

### Example 318. (1-(6-(6-fluoropyridin-3-yl)-2-(pyridin-3-yl)pyrimidin-4-yl)piperidin-4-yl)methanol

Using (6-fluoropyridin-3-yl)boronic acid, the title compound was obtained as described for the example 226 (Scheme 4. General procedure D.).

¹H NMR (600 MHz, DMSO-d₆) δ [ppm]=9.68 (d, J=2.2 Hz, 1H), 9.20 (d, J=2.5 Hz, 1H), 9.07 (d, J=8.0 Hz, 1H), 8.89 (td, J=8.2, 2.6 Hz, 1H), 8.84 (dd, J=5.1, 1.7 Hz, 1H), 7.82 (dd, J=8.2, 5.2 Hz, 1H), 7.51 (s, 1H), 7.38 (dd, J=8.6, 2.8 Hz, 1H), 4.83-4.66 (m, 2H), 4.31 (d, J=6.6 Hz, 2H), 3.13-2.96 (m, 2H), 2.22-2.12 (m, 1H), 1.85-1.79 (m, 2H), 1.33-1.24 (m, 2H); MS (ESI, m/z): 366.2 [M+H]⁺

### Example 319. 1-(6-(4-chlorophenyl)-2-(pyridin-3-yl)pyrimidin-4-yl)azetidin-3-ol

Using azetidin-3-ol hydrochloride, the title compound was obtained as described for the example 1 (Scheme 1. General procedure A.).

¹H NMR (400 MHz, DMSO-d₆) δ [ppm]=3.94 (dd, J=9.8, 4.3 Hz, 2H), 4.40-4.42 (m, 2H), 4.64-4.69 (m, 1H), 5.84 (d, J=6.4 Hz, 1H), 6.97 (s, 1H), 7.58-7.50 (m, 1H), 7.66-7.58 (m, 2H), 8.38-8.27 (m, 2H), 8.76-8.65 (m, 2H), 9.57 (d, J=1.3 Hz, 1H); MS (ESI, m/z): 339.3 [M+H]⁺

### Example 320. (1-(6-(4-chlorophenyl)-2-(pyridin-3-yl)pyrimidin-4-yl)azetidin-3-yl)methanol

Using azetidin-3-ylmethanol hydrochloride, the title compound was obtained as described for the example 1 (Scheme 1. General procedure A.).

¹H NMR (400 MHz, DMSO-d₆) δ [ppm]=2.83-2.96 (m, 1H), 3.63 (d, J=6.0 Hz, 2H), 3.94 (dd, J=8.6, 5.5 Hz, 2H), 4.21 (t, J=8.6 Hz, 2H), 4.87 (s, 1H), 6.94 (s, 1H), 7.50-7.57 (m, 1H), 7.57-7.65 (m, 2H), 8.28-8.36 (m, 2H), 8.70 (m, 2H), 9.563 (s, 1H); MS (ESI, m/z): 353.2 [M+H]⁺

### Example 321. 4-(4-chlorophenyl)-2-(1-methyl-1H-pyrazol-4-yl)-6-(4-(methylsulfonyl)piperazin-1-yl)pyrimidine

Scheme for the preparation of the Compound of Example 321:

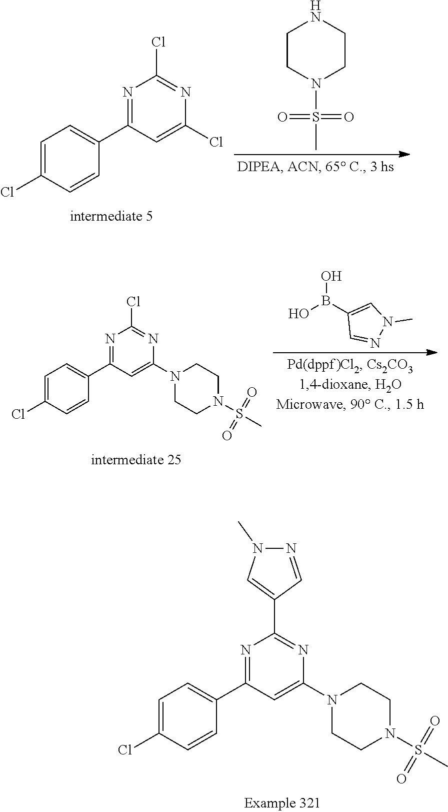

### Intermediate 25. (S)-1-(2-chloro-6-(4-chlorophenyl)pyrimidin-4-yl)pyrrolidin-3-ol

To a solution of 2,4-dichloro-6-(4-chlorophenyl)pyrimidine (300 mg, 1.16 mmol) in acetonitrile (5 mL) was added 1-(methylsulfonyl)piperazine (286 mg, 1.74 mmol) and DIPEA (448 mg, 3.48 mmol) at room temperature. The reaction mixture was heated at 65° C. for 3 hr. TLC showed the starting material consumed completely. After the reaction mixture was cooled to room temperature, the mixture was concentrated in vacuo. The residue was extracted with DCM (30 mL×2), washed with water (30 mL), dried and concentrated in vacuo. The residue was purified via column chromatography (Petroleum ether/EtOAc=1/1; V/V) to afford 160 mg of the title compound.

MS (ESI, m/z): 387.2 [M+H]⁺

### Example 321. 4-(4-chlorophenyl)-2-(1-methyl-1H-pyrazol-4-yl)-6-(4-(methylsulfonyl)piperazin-1-yl)pyrimidine

To a solution of 2-chloro-4-(4-chlorophenyl)-6-(4-(methylsulfonyl)piperazin-1-yl)pyrimidine (100 mg, 0.26 mmol) in 1.4-dioxane (2.5 mL) and H₂O (0.5 mL) was added (1-methyl-1H-pyrazol-4-yl)boronic acid (49 mg, 0.39 mmol), Pd(dppf)Cl₂ (15 mg, 0.021 mmol) and Cs₂CO₃ (250 mg, 0.77 mmol) at room temperature under Nitrogen. The reaction mixture was heated at 90° C. via microwave irradiation for 1.5 hr under nitrogen. LCMS showed the starting material consumed completely. After the reaction mixture was cooled to room temperature, the mixture was concentrated in vacuo. The residue was extracted with EtOAc (30 mL×2), washed with water (30 mL), dried and concentrated in vacuo. The residue was purified via column chromatography (Petroleum ether/EtOAc=2/3; V/V) to afford 60 mg of the title compound (Scheme 3. General procedure C.).

¹H NMR (400 MHz, CDCl₃) δ [ppm]=2.82 (s, 3H), 3.32-3.41 (m, 4H), 3.87-3.95 (m, 4H), 3.97 (s, 3H), 6.69 (s, 1H), 7.45 (d, J=8.5 Hz, 2H), 8.01 (d, J=8.5 Hz, 2H), 8.12 (s, 1H), 8.17 (s, 1H); MS (ESI, m/z): 433.4 [M+H]⁺

### Example 322. (S)-1-(6-(4-chlorophenyl)-2-(3-hydroxyphenyl)pyrimidin-4-yl)pyrrolidin-3-ol formate

Scheme for the preparation of the Compound of Example 322:

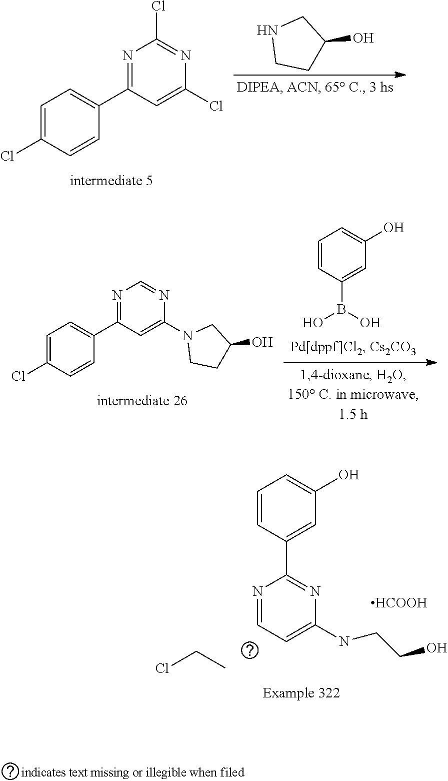

### Intermediate 26. (S)-1-(2-chloro-6-(4-chlorophenyl)pyrimidin-4-yl)pyrrolidin-3-ol

To a solution of 2,4-dichloro-6-(4-chlorophenyl)pyrimidine (600 mg, 2.33 mmol) in CH₃CN (30 mL) was added (S)-pyrrolidin-3-ol (243.0 mg, 2.79 mmol), DIPEA (451.0 mg, 3.495 mmol) at room temperature. The reaction mixture was heated and stirred at 65° C. for 3 hr. TLC showed the starting material was consumed completely. The solvent was removed in vacuo and the residue was purified via column chromatography (Petroleum ether/EtOAc=1/1; V/V) to afford 490 mg of the title compound.

MS (ESI, m/z): 310.1 [M+H]⁺

### Example 322. (S)-1-(6-(4-chlorophenyl)-2-(3-hydroxyphenyl)pyrimidin-4-yl)pyrrolidin-3-ol

To a solution of (S)-1-(2-chloro-6-(4-chlorophenyl)pyrimidin-4-yl)pyrrolidin-3-ol (100 mg, 0.324 mmol) in 1,4-dioxane (2 mL) and water (0.2 mL) was added (3-hydroxyphenyl)boronic acid (67 mg, 0.485 mmol), Pd(dppf)Cl₂ (23.7 mg, 0.0324 mmol) and Cs₂CO₃ (316.7 mg, 0.972 mmol) at room temperature under Nitrogen. The reaction mixture was heated at 85° C. via microwave irradiation for 45 min under Nitrogen atmosphere. The reaction mixture was concentrated in vacuo. The residue was purified via reverse phase column chromatography eluted with CH₃CN:H₂O (0.1% Formic acid) to afford 60.8 mg of the title compound (Scheme 3. General procedure C.).

¹H NMR (400 MHz, DMSO-d₆) δ [ppm]=2.19-1.85 (m, 2H), 3.94-3.53 (m, 4H), 4.44 (s, 1H), 5.07 (m, 1H), 6.91-6.82 (m, 1H), 6.95 (s, 1H), 7.28 (t, J=8.0 Hz, 1H), 7.60 (d, J=8.6 Hz, 2H), 7.92 (dd, J=4.0, 2.2 Hz, 2H), 8.18 (s, 0.28H), 8.30 (d, J=8.6 Hz, 2H), 9.50 (s, 1H); MS (ESI, m/z): 368.3 [M+H]⁺

### Example 323. (S)-1-(6-(4-chlorophenyl)-2-(5-fluoropyridin-3-yl)pyrimidin-4-yl)pyrrolidin-3-ol

Using (5-fluoropyridin-3-yl)boronic acid, the title compound was obtained as described for the example 322 (Scheme 3. General procedure C.).

¹H NMR (400 MHz, DMSO-d₆) δ [ppm]=2.23-1.80 (m, 2H), 3.91-3.40 (m, 4H), 4.56-4.35 (m, 1H), 5.06 (s, 1H), 7.05 (s, 1H), 7.59 (d, J=8.6 Hz, 2H), 8.35 (d, J=8.4 Hz, 2H), 8.52 (d, J=9.8 Hz, 1H), 8.71 (d, J=2.9 Hz, 1H), 9.46 (s, 1H); MS (ESI, m/z): 371.3 [M+H]⁺

### Example 324. (S)-1-(6-(4-chlorophenyl)-2-(pyridin-4-yl)pyrimidin-4-yl)pyrrolidin-3-ol

Using pyridin-4-ylboronic acid, the title compound was obtained as described for the example 322 (Scheme 3. General procedure C.).

¹H NMR (400 MHz, DMSO-d₆) δ [ppm]=2.20-1.81 (m, 2H), 3.90-3.47 (m, 4H), 4.47 (d, J=19.1 Hz, 1H), 5.08 (s, 1H), 7.07 (s, 1H), 7.60 (d, J=8.6 Hz, 2H), 8.17 (s, 1H), 8.41-8.27 (m, 4H), 8.75 (d, J=5.7 Hz, 2H); MS (ESI, m/z): 353.2 [M+H]⁺

### Example 325. 4-(4-chlorophenyl)-2-(5-fluoropyridin-3-yl)-6-(4-(methylsulfonyl)piperazin-1-yl)pyrimidine

Using (5-fluoropyridin-3-yl)boronic acid, the title compound was obtained as described for the example 321 (Scheme 3. General procedure C.).

¹H NMR (400 MHz, DMSO-d₆) δ [ppm]=2.92 (s, 3H), 3.20-3.30 (m, 4H), 4.02 (s, 4H), 7.50 (s, 1H), 7.61 (d, J=8.6 Hz, 2H), 8.40 (d, J=8.7 Hz, 2H), 8.52-8.60 (m, 1H), 8.73 (d, J=2.8 Hz, 1H), 9.41-9.54 (m, 1H); MS (ESI, m/z): 448.4 [M+H]⁺

### Example 326. 4-(4-chlorophenyl)-6-(4-(methylsulfonyl)piperidin-1-yl)-2-(pyridin-3-yl)pyrimidine

Scheme for the preparation of the Compound of Example 326:

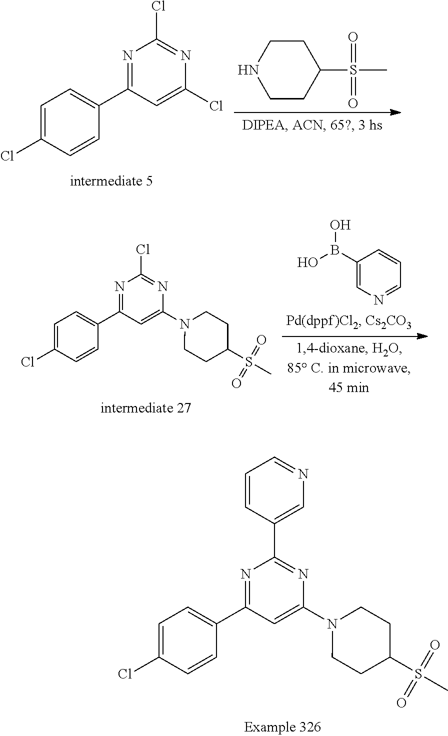

### Intermediate 27. 2-chloro-4-(4-chlorophenyl)-6-(4-(methylsulfonyl)piperazin-1-yl)pyrimidine

To a solution of 2,4-dichloro-6-(4-chlorophenyl)pyrimidine (300 mg, 1.16 mmol) in ACN (5 mL) was added 4-(methylsulfonyl)piperidine (284 mg, 1.74 mmol) and DIPEA (448.9 mg, 3.48 mmol) at room temperature. The reaction mixture was heated at 65° C. for 6 hr. The mixture was concentrated in vacuo. The residue was purified via silica gel column chromatography (Petroleum ether/EtOAc=2/1; V/V) to afford 250 mg of the title compound.

MS (ESI, m/z): 386.1 [M+H]⁺

### Example 326. 4-(4-chlorophenyl)-6-(4-(methylsulfonyl)piperidin-1-yl)-2-(pyridin-3-yl)pyrimidine

To a solution of 2-chloro-4-(4-chlorophenyl)-6-(4-(methylsulfonyl)piperazin-1-yl)pyrimidine (250 mg, 0.65 mmol) in 1,4-dioxane (4 mL) and H₂O (0.8 mL) was added pyridin-3-ylboronic acid (119.8 mg, 0.97 mmol), Cs₂CO₃ (635.3 mg, 1.95 mmol) and Pd(dppf)Cl₂ (53 mg, 0.065 mmol) at room temperature under Nitrogen. The reaction mixture was heated and stirred at 85° C. via microwave irradiation for 45 minutes under N₂ atmosphere. The mixture was cooled to room temperature and the residue was extracted with EtOAc (20 mL×3), washed with brine. The combined organic layer was dried over anhydrous sodium sulfate. The mixture was filtered and concentrated in vacuo. The residue was purified via silica gel column chromatography (DCM/MeOH=20/1; V/V) to afford 110 mg of the title compound (Scheme 3. General procedure C.).

¹H NMR (400 MHz, DMSO-d₆) δ [ppm]=1.55-1.72 (m, 2H), 2.17 (d, J=10.9 Hz, 2H), 2.97 (s, 3H), 3.10 (t, J=12.1 Hz, 2H), 3.39-3.59 (m, 1H), 4.74-5.02 (m, 2H), 7.55 (ddd, J=7.9, 4.8, 0.7 Hz, 1H), 7.58-7.67 (m, 2H), 8.32-8.44 (m, 2H), 8.67-8.79 (m, 2H), 9.59 (d, J=1.4 Hz, 1H); MS (ESI, m/z): 429.3 [M+H]⁺

### Example 327. (S)-1-(2-(5-fluoropyridin-3-yl)-6-(4-(trifluoromethyl)phenyl)pyrimidin-4-yl)pyrrolidin-3-ol

Using (5-fluoropyridin-3-yl)boronic acid, the title compound was obtained as described for the example 263 (Scheme 3. General procedure C.).

¹H NMR (400 MHz, DMSO-d₆) δ [ppm]=2.15-1.92 (m, 2H), 3.70 (m, 4H), 4.47 (d, J=23.6 Hz, 1H), 5.10 (d, J=36.4 Hz, 1H), 7.14 (d, J=7.5 Hz, 1H), 7.89 (d, J=8.3 Hz, 2H), 8.53 (d, J=8.1 Hz, 3H), 8.73 (d, J=2.9 Hz, 1H), 9.48 (s, 1H); MS (ESI, m/z): 405.4 [M+H]⁺

### Example 328. 4-(4-chlorophenyl)-6-(4-(cyclopropylsulfonyl)piperazin-1-yl)-2-(pyridin-3-yl)pyrimidine

Scheme for the preparation of the Compound of Example 328:

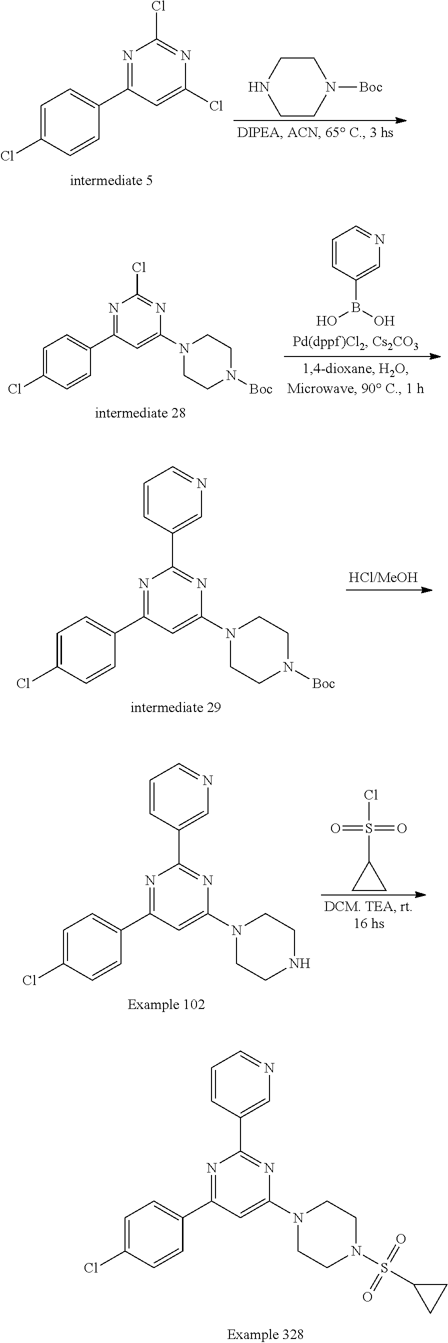

### Intermediate 28. tert-butyl 4-(2-chloro-6-(4-chlorophenyl)pyrimidin-4-yl)piperazine-1-carboxylate

To a solution of 2,4-dichloro-6-(4-chlorophenyl)pyrimidine (2 g, 7.75 mmol) in acetonitrile (20 mL) added tert-butyl piperazine-1-carboxylate (2.1 g, 11.63 mmol) and DIPEA (3 g, 23.25 mmol) at room temperature. The reaction mixture was heated at 65° C. for 4 hr under nitrogen. LCMS showed the starting material consumed completely. After the reaction mixture was cooled to room temperature, the mixture was extracted with DCM (60 mL×2), washed with water (65 mL), dried over anhydrous sodium sulfate. The solid was filtered off and the filtrate was concentrated in vacuo. The residue was purified via silica gel column chromatography (Petroleum ether/EtOAc=4/1; V/V) to afford 1.8 g of the title compound.

MS (ESI, m/z): 409.4 [M+H]⁺

### Intermediate 29. tert-butyl 4-(6-(4-chlorophenyl)-2-(pyridin-3-yl)pyrimidin-4-yl)piperazine-1-carboxylate

To a solution of tert-butyl 4-(2-chloro-6-(4-chlorophenyl)pyrimidin-4-yl)piperazine-1-carboxylate (1.2 g, 2.94 mmol) in 1,4-dioxane (20 mL) and H₂O (4 mL) was added pyridin-3-ylboronic acid (543 mg, 4.41 mmol), Pd(dppf)Cl₂ (172 mg, 0.235 mmol) and CsCO₃ (2.8 g, 8.82 mmol) at room temperature under Nitrogen. The reaction mixture was stirred at 100° C. via microwave irradiation for 3 hr under nitrogen atmosphere. LCMS showed the starting material consumed completely. After the reaction mixture was cooled to room temperature, the mixture was extracted with EtOAc (50 mL×2), washed with water (65 mL), dried over anhydrous sodium sulfate. The solid was filtered off and the filtrate was concentrated in vacuo. The residue was purified via silica gel column chromatography (Petroleum ether/EtOAc=65/35; V/V) to afford 720 mg of the title compound.

¹H NMR (400 MHz, CDCl₃) δ [ppm]=1.51 (s, 9H), 3.56-3.66 (m, 4H), 3.78-3.91 (m, 4H), 6.85 (s, 1H), 7.48 (d, J=8.4 Hz, 3H), 8.07 (d, J=8.5 Hz, 2H), 8.70 (s, 1H), 8.85 (d, J=7.5 Hz, 1H), 9.70 (s, 1H); MS (ESI, m/z): 452.2 [M+H]⁺

### Example 102. 4-(4-chlorophenyl)-6-(piperazin-1-yl)-2-(pyridin-3-yl)pyrimidine

To a solution of tert-butyl 4-(6-(4-chlorophenyl)-2-(pyridin-3-yl)pyrimidin-4-yl)piperazine-1-carboxylate (720 mg, 1.6 mmol) in MeOH—HCl (4 N HCl gas in MeOH, 12 mL) was stirred at room temperature for 2 hr. TLC showed the starting material consumed completely. The reaction mixture was concentrated in vacuo to afford 700 mg of the title compound.

MS (ESI, m/z): 352.1 [M+H]⁺

### Example 328. 4-(4-chlorophenyl)-6-(4-(cyclopropylsulfonyl)piperazin-1-yl)-2-(pyridin-3-yl)pyrimidine

To a solution of 4-(4-chlorophenyl)-6-(piperazin-1-yl)-2-(pyridin-3-yl)pyrimidine (100 mg, 0.285 mmol) in DCM (3 mL) added cyclopropanesulfonyl chloride (60 mg, 0.427 mmol) and Et₃N (144 mg, 1.425 mmol) at room temperature. The mixture was stirred at room temperature for 16 hr. LCMS showed the starting material consumed completely. The reaction mixture was extracted with DCM (30 mL×2), washed with water (35 mL), dried over anhydrous sodium sulfate. The solid was filtered off and the filtrate was concentrated in vacuo. The residue was purified by prep-HPLC to afford 16 mg of the title compound.

¹H NMR (400 MHz, CDCl₃) δ [ppm]=0.96-1.10 (m, 2H), 1.16-1.32 (m, 2H), 2.21-2.36 (m, 1H), 3.49 (s, 4H), 3.98 (s, 4H), 6.92 (s, 1H), 7.50 (d, J=8.4 Hz, 2H), 7.75 (s, 1H), 8.06 (d, J=8.5 Hz, 2H), 8.83 (s, 1H), 9.15 (d, J=7.1 Hz, 1H), 9.75 (s, 1H); MS (ESI, m/z): 456.4 [M+H]⁺

### Example 329. (S)-1-(6-(4-chlorophenyl)-2-(pyridazin-4-yl)pyrimidin-4-yl)pyrrolidin-3-ol

Scheme for the preparation of the Compound of Example 329:

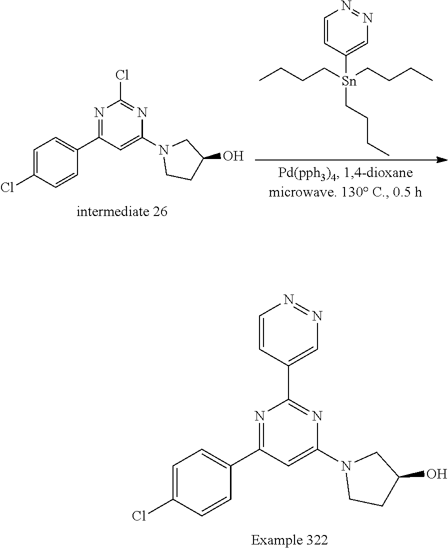 [SMILES: O[C@H]1CCN(c2cc(-c3ccc(Cl)cc3)nc(Cl)n2)C1 ; CCC[CH2][Sn]([CH2]CCC)([CH2]CCC)[c]1ccnnc1 ; O[C@H]1CCN(c2cc(-c3ccc(Cl)cc3)nc(-c3ccnnc3)n2)C1]

### Example 329. (S)-1-(6-(4-chlorophenyl)-2-(pyridazin-4-yl)pyrimidin-4-yl)pyrrolidin-3-ol

To a solution of (S)-1-(6-(4-chlorophenyl)-2-(pyridazin-4-yl)pyrimidin-4-yl)pyrrolidin-3-ol (100 mg, 0.3226 mmol) in 1,4-dioxane (3 mL) was added 4-(tributylstannyl)pyridazine (167 mg, 0.4516 mmol), Pd(PPh₃)₄ (37 mg, 0.0322 mmol) at room temperature under Nitrogen. The reaction mixture was stirred via microwave irradiation at 130° C. for 0.5 hr under Nitrogen atmosphere. TLC showed the starting material consumed completely. The mixture was cooled to room temperature and extracted with EtOAc (30 mL×2), washed with water (35 mL), dried over anhydrous sodium sulfate. The solid was filtered off and the filtrate was concentrated in vacuo. The residue was purified by prep-HPLC to afford 19.5 mg of the title compound.

¹H NMR (400 MHz, CDCl₃) δ [ppm]=2.23 (s, 2H), 3.54-4.18 (m, 4H), 4.73 (s, 1H), 6.64 (s, 1H), 7.48 (d, J=8.6 Hz, 2H), 8.05 (d, J=8.6 Hz, 2H), 8.42 (dd, J=5.3, 2.1 Hz, 1H), 9.28 (dd, J=5.3, 1.1 Hz, 1H), 10.15 (s, 1H); MS (ESI, m/z): 354 [M+H]⁺

### Example 330. 1-(6-(4-chlorophenyl)-2-(pyridin-3-yl)pyrimidin-4-yl)azepan-4-ol

Using azepan-4-ol hydrochloride, the title compound was obtained as described for the example 1 (Scheme 1. General procedure A.).

¹H NMR (400 MHz, DMSO-d₆) δ [ppm]=1.56 (s, 1H), 1.63-1.79 (m, 3H), 1.93-2.06 (m, 2H), 3.56-4.08 (m, 5H), 4.57 (s, 1H), 7.17 (s, 1H), 7.55 (dd, J=7.8, 4.8 Hz, 1H), 7.57-7.69 (m, 2H), 8.34 (d, J=8.6 Hz, 2H), 8.61-8.79 (m, 2H), 9.58 (s, 1H); MS (ESI, m/z): 381.4 [M+H]⁺

### Example 331. 2-(6-(4-chlorophenyl)-2-(pyridin-3-yl)pyrimidin-4-yl)-5-(methylsulfonyl)-2,5-diazabicyclo[2.2.1]heptane

Using 2-(methylsulfonyl)-2,5-diazabicyclo[2.2.1]heptane, the title compound was obtained as described for the example 1 (Scheme 1. General procedure A.).

¹H NMR (400 MHz, CDCl₃) δ [ppm]=2.00-2.17 (m, 2H), 2.93 (s, 3H), 3.40-3.96 (m, 4H), 4.71 (s, 1H), 5.42 (s, 1H), 6.59 (s, 1H), 7.43 (dd, J=7.8, 4.9 Hz, 1H), 7.48 (d, J=8.6 Hz, 2H), 8.08 (d, J=8.6 Hz, 2H), 8.71 (d, J=3.2 Hz, 1H), 8.79 (dt, J=8.0, 1.7 Hz, 1H), 9.69 (s, 1H); MS (ESI, m/z): 442.40 [M+H]⁺

### Example 332. (S)-1-(6-(4-chlorophenyl)-2-(2-methyl-2H-tetrazol-5-yl)pyrimidin-4-yl)pyrrolidin-3-ol

Scheme for the preparation of the Compound of Example 332:

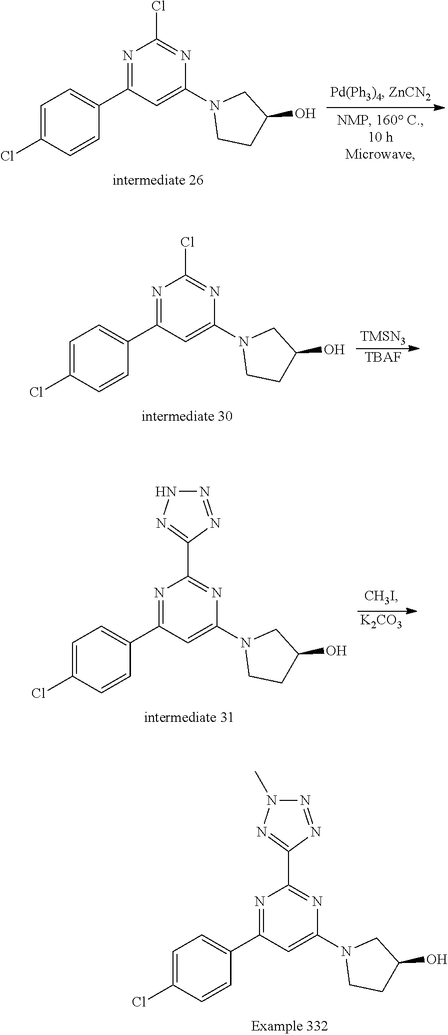

### Intermediate 30. (S)-4-(4-chlorophenyl)-6-(3-hydroxypyrrolidin-1-yl)pyrimidine-2-carbonitrile

To a solution of (S)-1-(2-chloro-6-(4-chlorophenyl)pyrimidin-4-yl)pyrrolidin-3-ol (500 mg, 1.62 mmol) in NMP (6 mL) was added Zn(CN)₂ (285 mg, 2.42 mmol), Pd(PPh₃)₄ (279.6 mg, 0.24 mmol) at room temperature under Nitrogen. The reaction mixture was heated and stirred at 160° C. via microwave irradiation for 1 h under Nitrogen atmosphere. The reaction mixture was cooled to room temperature and diluted with water. The aqueous layer was extracted with EtOAc (10 mL×2). The combined organic layer was washed with brine, dried over Na₂SO₄ filtered and concentrated in vacuo. The residue was purified via silica gel column chromatography (Petroleum ether/EtOAc=1/1; V/V) to afford 300 mg of the title compound.

MS (ESI, m/z): 301.2 [M+H]⁺

### Intermediate 31. (S)-1-(6-(4-chlorophenyl)-2-(2H-tetrazol-5-yl)pyrimidin-4-yl)pyrrolidin-3-ol

To a solution of (S)-4-(4-chlorophenyl)-6-(3-hydroxypyrrolidin-1-yl)pyrimidine-2-carbonitrile (300 mg, 1.0 mmol) in THF (15 mL) was added TMSN₃ (230 mg, 3.0 mmol) and TBAF (130.8 mg, 0.5 mmol) at room temperature. The reaction mixture was heated at 96° C. in a sealed tube for 16 hr. The reaction mixture was cooled to room temperature and diluted with water. The aqueous layer was extracted with EtOAc (15 mL×2). The combined organic layer was washed with brine, dried over Na₂SO₄. Filtered and concentrated in vacuo to afford 80 mg of the title compound.

MS (ESI, m/z): 344.3 [M+H]⁺

### Example 332. (S)-1-(6-(4-chlorophenyl)-2-(2-methyl-2H-tetrazol-5-yl)pyrimidin-4-yl)pyrrolidin-3-ol

To a solution of (S)-1-(6-(4-chlorophenyl)-2-(2H-tetrazol-5-yl)pyrimidin-4-yl)pyrrolidin-3-ol (80 mg, 0.23 mmol) in acetone (40 mL) was added CH₃I (49 mg, 0.35 mmol) and K₂CO₃ (35.4 mg, 0.26 mmol) at room temperature. The reaction mixture was stirred at room temperature for 13 hr. The reaction mixture was filtered and the filtrate was concentrated in vacuo. The residue was purified via column chromatography (DCM/MeOH=10/1; V/V) to afford 5.6 mg of the title compound.

¹H NMR (400 MHz, DMSO-d₆) δ [ppm]=2.00 (m, 7.3 Hz, 2H), 3.87-3.45 (m, 4H), 4.47 (m, 4H), 5.11 (m, 1H), 7.17 (d, J=7.6 Hz, 1H), 7.62 (d, J=8.4 Hz, 2H), 8.28 (d, J=6.5 Hz, 2H); MS (ESI, m/z): 358.0 [M+H]⁺

### Example 333. (S)-1-(6-(4-chlorophenyl)-2-(2-methyl-2H-tetrazol-5-yl)pyrimidin-4-yl)pyrrolidin-3-ol

Scheme for the preparation of the Compound of Example 333:

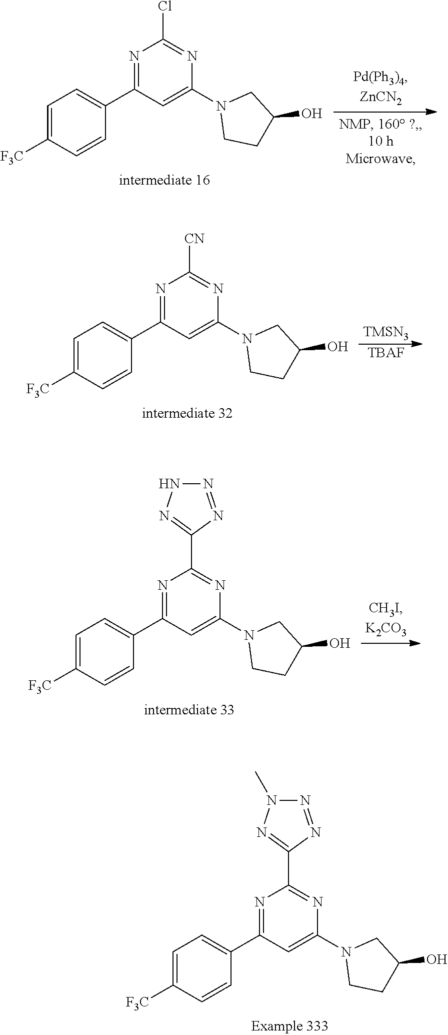

### Intermediate 32. (S)-4-(3-hydroxypyrrolidin-1-yl)-6-(4-(trifluoromethyl)phenyl)pyrimidine-2-carbonitrile

To a solution of (S)-1-(2-chloro-6-(4-(trifluoromethyl)phenyl)pyrimidin-4-yl)pyrrolidin-3-ol (300 mg, 0.836 mmol) in NMP (6 mL) was added Zn(CN)₂ (147.22 mg, 1.254 mmol), Pd(Ph₃)₄ (144.5 mg, 0.125 mmol) at room temperature under Nitrogen. The reaction mixture was heated and stirred at 160° C. via microwave irradiation for 1 h under Nitrogen atmosphere. The reaction mixture was cooled to room temperature and diluted with water. The aqueous layer was extracted with EtOAc (10 mL×2). The combined organic layer was washed with brine, dried over Na₂SO₄. Filtered and concentrated in vacuo. The residue was purified via silica gel column chromatography (Petroleum ether/EtOAc=1/1; V/V) to afford 411.1 mg of the title compound.

MS (ESI, m/z): 335.1 [M+H]⁺

### Intermediate 33. (S)-1-(2-(2H-tetrazol-5-yl)-6-(4-(trifluoromethyl)phenyl)pyrimidin-4-yl)pyrrolidin-3-ol

To a solution of (S)-4-(3-hydroxypyrrolidin-1-yl)-6-(4-(trifluoromethyl)phenyl)pyrimidine-2-carbonitrile (910 mg, 2.72 mmol) in THF (15 mL) was added TMSN₃ (941.6 mg, 8.174 mmol) and TBAF (355.6 mg, 1.36 mmol) at room temperature. The reaction mixture was heated at 96° C. in a sealed tube for 16 hr. The reaction mixture was cooled to room temperature and diluted with water. The aqueous layer was extracted with EtOAc (15 mL×2). The combined organic layer was washed with brine, dried over Na₂SO₄. Filtered and concentrated in vacuo to afford 990 mg of the title compound.

MS (ESI, m/z): 378.2 [M+H]⁺

### Example 333. (S)-1-(2-(2-methyl-2H-tetrazol-5-yl)-6-(4-(trifluoromethyl)phenyl)pyrimidin-4-yl)pyrrolidin-3-ol

To a solution of (S)-1-(2-(2H-tetrazol-5-yl)-6-(4-(trifluoromethyl)phenyl)pyrimidin-4-yl)pyrrolidin-3-ol (150 mg, 0.3998 mmol) in acetone (20 mL) was added CH₃I (84.7 mg, 0.597 mmol) and K₂CO₃ (109.8 mg, 0.796 mmol) at room temperature. The reaction mixture was stirred at room temperature for 13 hr. The reaction mixture was filtered and the filtrate was concentrated in vacuo. The residue was purified via column chromatography (DCM/MeOH=10/1; V/V) to afford 28.8 mg of the title compound.

¹H NMR (400 MHz, DMSO-d₆) δ [ppm]=2.18-1.89 (m, 2H), 3.88-3.45 (m, 4H), 4.460 (s, 3H), 4.507 (s, 1H), 5.13 (d, J=36.2 Hz, 1H), 7.26 (d, J=7.3 Hz, 1H), 7.92 (d, J=8.2 Hz, 2H), 8.45 (d, J=6.8 Hz, 2H); MS (ESI, m/z): 392.2 [M+H]⁺

### Example 334. 4-(4-chlorophenyl)-2-(2-methyl-2H-tetrazol-5-yl)-6-(4-(methylsulfonyl)piperazin-1-yl)pyrimidine

Scheme for the preparation of the Compound of Example 334:

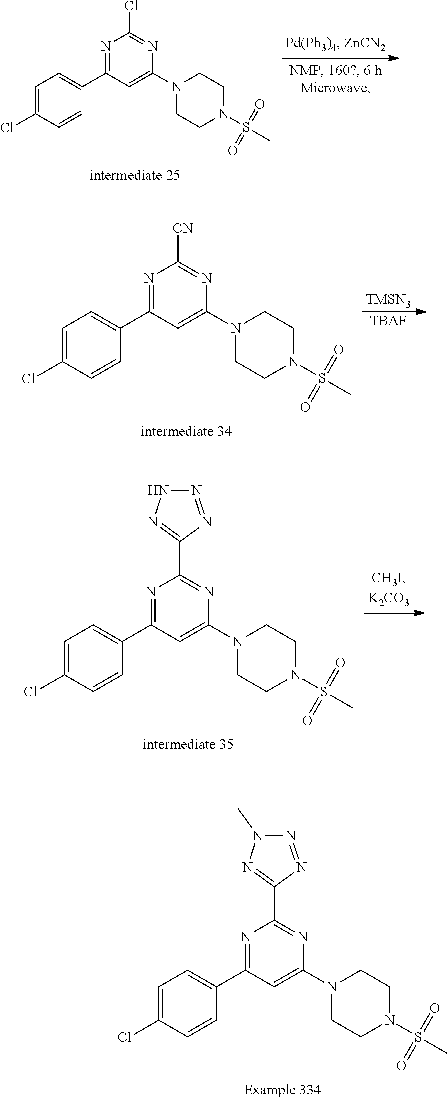 [SMILES: C=C/C(Cl)=C\C=C\c1cc(N2CCN(S(C)(=O)=O)CC2)nc(Cl)n1 ; CS(=O)(=O)N1CCN(c2cc(-c3ccc(Cl)cc3)nc(C#N)n2)CC1 ; "TMSN3" ; "TBAF" ; CS(=O)(=O)N1CCN(c2cc(-c3ccc(Cl)cc3)nc(-c3nn[nH]n3)n2)CC1 ; Cn1nnc(-c2nc(-c3ccc(Cl)cc3)cc(N3CCN(S(C)(=O)=O)CC3)n2)n1]

### Intermediate 34. 4-(4-chlorophenyl)-6-(4-(methylsulfonyl)piperazin-1-yl)pyrimidine-2-carbonitrile

To a solution of 2-chloro-4-(4-chlorophenyl)-6-(4-(methylsulfonyl)piperazin-1-yl)pyrimidine (300 mg, 0.813 mmol) in NMP (6 mL) was added Zn(CN)₂ (143.23 mg, 1.22 mmol), Pd(Ph₃)₄ (141.0 mg, 0.122 mmol) at room temperature under Nitrogen. The reaction mixture was heated and stirred at 160° C. via microwave irradiation for 1 h under Nitrogen atmosphere. The reaction mixture was cooled to room temperature and diluted with water. The aqueous layer was extracted with EtOAc (10 mL×2). The combined organic layer was washed with brine, dried over Na₂SO₄. Filtered and concentrated in vacuo. The residue was purified via silica gel column chromatography (Petroleum ether/EtOAc=1/1; V/V) to afford 431.1 mg of the title compound.

MS (ESI, m/z): 378.1 [M+H]⁺

### Intermediate 35. 4-(4-chlorophenyl)-6-(4-(methylsulfonyl)piperazin-1-yl)-2-(2H-tetrazol-5-yl)pyrimidine

To a solution of 4-(4-chlorophenyl)-6-(4-(methylsulfonyl)piperazin-1-yl)pyrimidine-2-carbonitrile (500 mg, 1.33 mmol) in THF (15 mL) was added TMSN₃ (450.8 mg, 3.98 mmol) and TBAF (174.4 mg, 0.67 mmol) at room temperature. The reaction mixture was heated at 96° C. in a sealed tube for 16 hr. The reaction mixture was cooled to room temperature and diluted with water. The aqueous layer was extracted with EtOAc (15 mL×2). The combined organic layer was washed with brine, dried over Na₂SO₄, filtered and concentrated in vacuo. The residue was purified via silica gel column chromatography (Petroleum ether/EtOAc=1/100; V/V) to afford 250 mg of the title compound.

MS (ESI, m/z): 421.0 [M+H]⁺

### Example 334. 4-(4-chlorophenyl)-2-(2-methyl-2H-tetrazol-5-yl)-6-(4-(methylsulfonyl)piperazin-1-yl)pyrimidine

To a solution of 4-(4-chlorophenyl)-6-(4-(methylsulfonyl)piperazin-1-yl)-2-(2H-tetrazol-5-yl)pyrimidine (150 mg, 0.357 mmol) in acetone (20 mL) was added CH₃I (76.1 mg, 0.536 mmol) and K₂CO₃ (98.5 mg, 0.714 mmol) at room temperature. The reaction mixture was stirred at room temperature for 13 hr. The reaction mixture was filtered and the filtrate was concentrated in vacuo. The residue was purified via reverse phase column chromatography eluted with CH₃CN:H₂O (0.1% Formic acid) to afford 21.0 mg of the title compound.

¹H NMR (400 MHz, DMSO-d₆) δ [ppm]=2.93 (s, 3H), 3.30-3.21 (m, 4H), 4.40-3.92 (m, 4H), 4.44 (s, 3H), 7.62 (t, J=1.9 Hz, 2H), 7.64 (d, J=1.9 Hz, 1H), 8.34-8.28 (m, 2H); MS (ESI, m/z): 435.0 [M+H]⁺

### Example 335. 4-(4-chlorophenyl)-6-(4-((2-fluoroethyl)sulfonyl)piperazin-1-yl)-2-(pyridin-3-yl)pyrimidine

Scheme for the preparation of the Compound of Example 335:

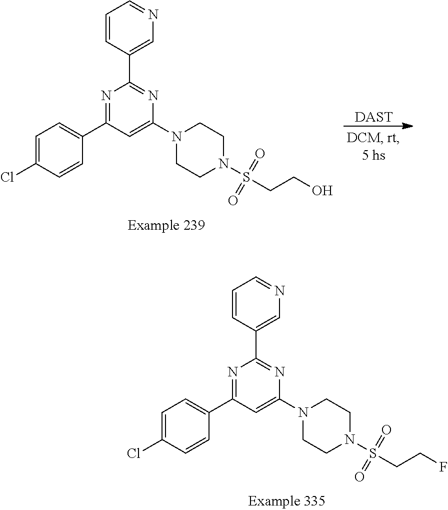 [SMILES: O=S(=O)(CCO)N1CCN(c2cc(-c3ccc(Cl)cc3)nc(-c3cccnc3)n2)CC1 ; "DAST" ; O=S(=O)(CCF)N1CCN(c2cc(-c3ccc(Cl)cc3)nc(-c3cccnc3)n2)CC1]

### Example 335. 4-(4-chlorophenyl)-6-(4-((2-fluoroethyl)sulfonyl)piperazin-1-yl)-2-(pyridin-3-yl)pyrimidine

To a solution of 2-((4-(6-(4-chlorophenyl)-2-(pyridin-3-yl)pyrimidin-4-yl)piperazin-1-yl)sulfonyl)ethanol (74 mg, 0.16 mmol) in DCM (3 mL) was added DAST (52 mg, 0.32 mmol) at 0° C. The reaction mixture was stirred at room temperature for 5 hr under nitrogen. LCMS showed the starting material consumed completely. The mixture was quenched by H₂O at 0° C., extracted with EtOAc (25 mL×2), washed with water (30 mL), dried over anhydrous sodium sulfate. The solid was filtered off and the filtrate was concentrated in vacuo. The residue was purified by prep-TLC (DCM/EtOAc/MeOH=20/1/1; V/V/V) to afford 12 mg of the title compound.

¹H NMR (400 MHz, CDCl₃) δ [ppm]=3.35 (t, J=5.2 Hz, 1H), 3.42 (t, J=5.2 Hz, 1H), 3.45-3.52 (m, 4H), 3.92-3.99 (m, 4H), 4.78 (t, J=5.2 Hz, 1H), 4.90 (t, J=5.2 Hz, 1H), 6.86 (s, 1H), 7.43-7.47 (m, 1H), 7.49 (d, J=8.6 Hz, 2H), 8.07 (d, J=8.6 Hz, 2H), 8.71 (d, J=3.6 Hz, 1H), 8.80 (d, J=8.0 Hz, 1H), 9.69 (s, 1H); MS (ESI, m/z): 462.0 [M+H]⁺

### Example 336. (S)-1-(6-(4-chlorophenyl)-2-(isoxazol-4-yl)pyrimidin-4-yl)pyrrolidin-3-ol

Scheme for the preparation of the Compound of Example 336:

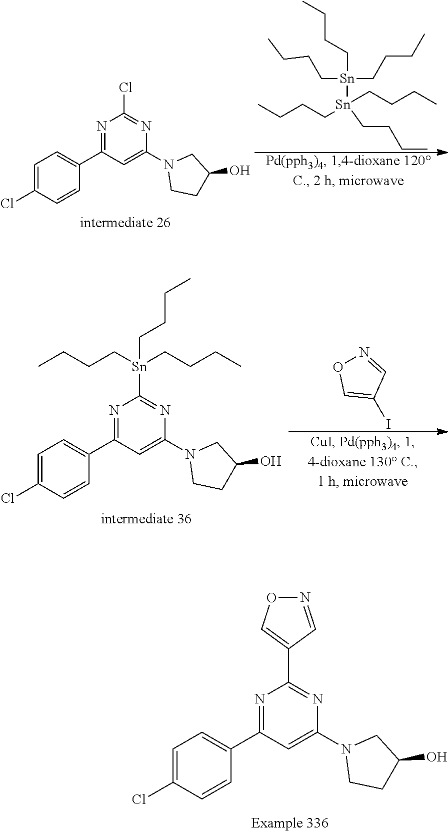 [SMILES: O[C@H]1CCN(c2cc(-c3ccc(Cl)cc3)nc(Cl)n2)C1 ; CCC[CH2][Sn]([CH2]CCC)([CH2]CCC)[Sn]([CH2]CCC)([CH2]CCC)[CH2]CCC ; CCC[CH2][Sn]([CH2]CCC)([CH2]CCC)[c]1nc(-c2ccc(Cl)cc2)cc(N2CC[C@H](O)C2)n1 ; Ic1cnoc1 ; O[C@H]1CCN(c2cc(-c3ccc(Cl)cc3)nc(-c3cnoc3)n2)C1]

### Intermediate 36. (S)-1-(6-(4-chlorophenyl)-2-(tributylstannyl)pyrimidin-4-yl)pyrrolidin-3-ol

To a solution of (S)-1-(2-chloro-6-(4-chlorophenyl)pyrimidin-4-yl)pyrrolidin-3-ol (200 mg, 0.647 mmol) in 1,4-dioxane (10 mL) was added 1,1,1,2,2,2-hexabutyldistannane (450.6 mg, 0.777 mmol) and Pd(PPh₃)₄ (75.1 mg, 0.065 mmol) at room temperature under Nitrogen. The reaction mixture was stirred at 120° C. via microwave irradiation for 2 hr under nitrogen atmosphere. LCMS showed the starting material consumed completely. The mixture was filtered and concentrated in vacuo. The residue was purified via silica gel column chromatography (DCM/MeOH=10/1; V/V) to afford 231 mg of the title compound.

MS (ESI, m/z): 566.2 [M+H]⁺

### Example 336. (S)-1-(6-(4-chlorophenyl)-2-(isoxazol-4-yl)pyrimidin-4-yl)pyrrolidin-3-ol

To a solution of (S)-1-(6-(4-chlorophenyl)-2-(tributylstannyl)pyrimidin-4-yl)pyrrolidin-3-ol (150 mg, 0.265 mmol) in 1,4-dioxane (2 mL) was added 4-iodoisoxazole (67.3 mg, 0.345 mmol), CuI (15 mg, 0.08 mmol) and Pd(PPh₃)₄ (46.2 mg, 0.04 mmol) at room temperature under Nitrogen. The reaction mixture was stirred at 130° C. via microwave irradiation for 2 hr under nitrogen atmosphere. LCMS showed the starting material consumed completely. The mixture was filtered and concentrated in vacuo. The residue was purified via Prep-HPLC to afford 25 mg of the title compound.

¹H NMR (400 MHz, DMSO-d₆) δ [ppm]=2.03 (m, J=34.2 Hz, 2H), 3.44-3.94 (m, 4H), 4.41-4.47 (m, 1H), 5.02-5.10 (m, 1H), 6.94 (s, 1H), 7.57 (d, J=8.6 Hz, 2H), 8.30 (d, J=8.5 Hz, 2H), 9.13 (s, 1H), 9.58 (s, 1H); MS (ESI, m/z): 343.0 [M+H]⁺

### Example 337. (1-(6-(4-chlorophenyl)-2-(pyridin-3-yl)pyrimidin-4-yl)-4-(methylsulfonyl)piperidin-4-yl)methanol

Using (4-(methylsulfonyl)piperidin-4-yl)methanol hydrochloride, the title compound was obtained as described for the example 1 (Scheme 1. General procedure A.).

¹H NMR (400 MHz, DMSO-d₆) δ [ppm]=1.89 (d, J=13.8 Hz, 2H), 1.95-2.09 (m, 2H), 2.98 (s, 3H), 3.40 (dd, J=17.8, 6.8 Hz, 2H), 3.96 (d, J=5.1 Hz, 2H), 4.55 (s, 2H), 5.56 (t, J=5.1 Hz, 1H), 7.41 (s, 1H), 7.52-7.58 (m, 1H), 7.58-7.64 (m, 2H), 8.34-8.41 (m, 2H) 8.70 (dd, J=4.8, 1.7 Hz, 1H), 8.72-8.77 (m, 1H), 9.59 (d, J=1.5 Hz, 1H); MS (ESI, m/z): 459.0 [M+H]⁺

### Example 338. 1-(6-(4-chlorophenyl)-2-(pyridin-3-yl)pyrimidin-4-yl)azepan-3-ol

Using azepan-3-ol, the title compound was obtained as described for the example 1 (Scheme 1. General procedure A.).

¹H NMR (400 MHz, CDCl₃) δ [ppm]=1.41-1.56 (m, 1H), 1.57-1.69 (m, 1H), 1.74-1.84 (m, 1H), 1.84-1.95 (m, 3H), 3.50 (s, 1H), 3.79 (dd, J=14.7, 3.8 Hz, 1H), 3.86-3.97 (m, 1H), 4.21 (s, 1H), 4.43 (s, 1H), 6.85 (s, 1H), 7.37-7.55 (m, 3H), 8.04-8.11 (m, 2H), 8.70 (d, J=3.8 Hz, 1H), 8.77 (s, 1H), 9.70 (s, 1H); MS (ESI, m/z): 381.2 [M+H]⁺

### Example 339. 4-(4-chlorophenyl)-6-(4-((difluoromethyl)sulfonyl)piperazin-1-yl)-2-(pyridin-3-yl)pyrimidine

Using 1-((difluoromethyl)sulfonyl)piperazine, the title compound was obtained as described for the example 1 (Scheme 1. General procedure A.).

¹H NMR (400 MHz, CDCl₃) δ [ppm]=3.70 (t, J=4.9 Hz, 4H), 3.97 (s, 4H), 6.28 (t, J=53.7 Hz, 1H), 6.88 (s, 1H), 7.49 (d, J=8.6 Hz, 3H), 8.07 (d, J=8.7 Hz, 2H), 8.72-8.73 (m, 1H), 8.83-8.85 (m, 1H), 9.69 (s, 1H); MS (ESI, m/z): 466.0 [M+H]⁺

### Example 340. (S)-1-(2-(pyridin-3-yl)-6-(4-(trifluoromethyl)phenyl)pyrimidin-4-yl)pyrrolidin-3-ol

Using (S)-pyrrolidin-3-ol, the title compound was obtained as described for the example 248 (Scheme 2. General procedure B.).

¹H NMR (400 MHz, DMSO-d₆) δ [ppm]=2.17-1.87 (m, 2H), 3.90-3.43 (m, 4H), 4.47 (d, J=21.3 Hz, 1H), 5.09 (d, J=36.9 Hz, 1H), 7. 11 (s, 1H), 7.54-7.57 (m, 1H), 7.89 (d, J=8.3 Hz, 2H), 8.51 (d, J=8.1 Hz, 2H), 8.69-8.71 (m, 2H), 9.61 (s, 1H); MS (ESI, m/z): 387.2 [M+H]⁺

### Example 341. 4-(4-(methylsulfonyl)piperazin-1-yl)-2-(pyridin-3-yl)-6-(4-(trifluoromethyl)phenyl)pyrimidine

Using 1-(methylsulfonyl)piperazine, the title compound was obtained as described for the example 248 (Scheme 2. General procedure B.).

¹H NMR (400 MHz, DMSO-d₆) δ [ppm]=2.93 (s, 3H), 3.23-3. 13 (m, 4H), 4.03 (s, 4H), 7.52-7.61 (m, 2H), 2H), 7.91 (d, J=8.3 Hz, 2H), 8.55 (d, J=8.2 Hz, 2H), 8.72 (dd, J=4.8, 1.7 Hz, 1H), 8.74-8.79 (m, 1H), 9.62 (d, J=1.6 Hz, 1H); MS (ESI, m/z): 464.0 [M+H]⁺

### Example 342. (S)-1-(6-(4-chlorophenyl)-2-(5,6-difluoropyridin-3-yl)pyrimidin-4-yl)pyrrolidin-3-ol

Using 5-bromo-2,3-difluoropyridine, the title compound was obtained as described for the example 336.

¹H NMR (400 MHz, CDCl₃) δ [ppm]=2.20 (s, 2H), 3.78 (s, 4H), 4.72 (s, 1H), 6.63 (s, 1H), 7.47 (d, J=7.8 Hz, 2H), 8.05 (d, J=8.1 Hz, 2H), 8.57-8.72 (m, 1H), 9.09 (s, 1H); MS (ESI, m/z): 389.2 [M+H]⁺

### Example 343. (3S,4R)-1-(2-(pyridin-3-yl)-6-(4-(trifluoromethyl)phenyl)pyrimidin-4-yl)pyrrolidine-3,4-diol

Using (3S,4R)-pyrrolidine-3,4-diol hydrochloride, the title compound was obtained as described for the example 248 (Scheme 2. General procedure B.).

¹H NMR (400 MHz, DMSO-d₆) δ [ppm]=3.43 (dd, J=10.2, 5.4 Hz, 1H), 3.63 (dd, J=11.9, 4.1 Hz, 1H), 3.74 (dd, J=10.3, 5.9 Hz, 1H), 3.84 (dd, J=11.7, 5.0 Hz, 1H), 4.14-4. 22 (m, 1H), 4.26 (m, 1H), 5.04 (d, J=4.6 Hz, 1H), 5.11 (d, J=5.3 Hz, 1H), 7.11 (s, 1H), 7.56 (dd, J=7.6, 4.7 Hz, 1H), 7.89 (d, J=8.3 Hz, 2H), 8.52 (d, J=8.2 Hz, 2H), 8.70 (dd, J=4.7, 1.5 Hz, 1H), 8.75 (dt, J=8.0, 1.9 Hz, 1H), 9.60 (d, J=1.5 Hz, 1H); MS (ESI, m/z): 403.4 [M+H]⁺

### Example 344. (S)-1-(6-(4-chlorophenyl)-[2,5′-bipyrimidin]-4-yl)pyrrolidin-3-ol

Using 5-bromopyrimidine, the title compound was obtained as described for the example 336.

¹H NMR (400 MHz, DMSO-d₆) δ [ppm]=1.84-2.20 (m, 2H), 3.41-3.91 (m, 4H), 4.47 (d, J=20.7 Hz, 1H), 5.09 (d, J=36.4 Hz, 1H), 7.06 (d, J=6.6 Hz, 1H), 7.59 (d, J=8.4 Hz, 2H), 8.35 (d, J=8.1 Hz, 2H), 9.32 (s, 1H), 9.68 (s, 2H); MS (ESI, m/z): 354.2 [M+H]⁺

### Example 345. (S)-1-(6-(4-chlorophenyl)-2-(6-fluoropyridin-3-yl)pyrimidin-4-yl)pyrrolidin-3-ol

Using 5-bromo-2-fluoropyridine, the title compound was obtained as described for the example 336.

*¹H NMR (400 MHz, CDCl₃) δ [ppm]=2.22 (s, 2H), 3.42-4.20 (m, 4H), 4.73 (s, 1H), 6.64 (s, 1H), 6.96-7.07 (m, 1H), 7.44 (s, 2H), 8.06 (d, J=6.4 Hz, 2H), 8.89 (t, J=7.2 Hz, 1H), 9.29 (s, 1H); MS (ESI, m/z): 371.4 [M+H]⁺

### Example 346. (S)-1-(6-(4-chlorophenyl)-2-(2-fluoropyridin-3-yl)pyrimidin-4-yl)pyrrolidin-3-ol

Using 3-bromo-2-fluoropyridine, the title compound was obtained as described for the example 336.

*¹H NMR (400 MHz, CDCl₃) δ [ppm]=2.17 (s, 2H), 3.72 (s, 4H), 4.65 (s, 1H), 6.57 (s, 1H), 7.23-7.34 (m, 1H), 7.43 (d, J=8.6 Hz, 2H), 8.02 (d, J=8.6 Hz, 2H), 8.24-8.30 (m, 1H), 8.57-8.67 (m, 1H); MS (ESI, m/z): 371.4 [M+H]⁺

### Example 347. (S)-1-(6-(4-chlorophenyl)-2-(pyridin-2-yl)pyrimidin-4-yl)pyrrolidin-3-ol

Using 2-(tributylstannyl)pyridine, the title compound was obtained as described for the example 329.

¹H NMR (400 MHz, DMSO-d₆) δ [ppm]=1.84-2.17 (m, 2H), 3.42-3.95 (m, 4H), 4.44 (s, 1H), 5.06 (d, J=35.4 Hz, 1H), 7.04 (s, 1H), 7.51 (s, 1H), 7.60 (d, J=8.5 Hz, 2H), 7.96 (s, 1H), 8.31 (d, J=7.8 Hz, 2H), 8.43 (d, J=7.1 Hz, 1H), 8.74 (d, J=3.3 Hz, 1H); MS (ESI, m/z): 353.2 [M+H]⁺

### Example 348. 2-((4-(6-(4-chlorophenyl)-2-(1-methyl-1H-pyrazol-4-yl)pyrimidin-4-yl)piperazin-1-yl)sulfonyl)ethanol

Using 2-(piperazin-1-ylsulfonyl)ethan-1-ol, the title compound was obtained as described for the example 174 (Scheme 2. General procedure B.).

¹H NMR (400 MHz, DMSO-d₆) δ [ppm]=3.24 (t, J=6.1 Hz, 2H), 3.30 (d, J=3.8 Hz, 4H), 3.76 (m, 2H), 3.90-3.904 (m, 7H), 5.03 (t, J=5.4 Hz, 1H), 7.23 (s, 1H), 7.58 (d, J=8.6 Hz, 2H), 8.05 (s, 1H), 8.30 (d, J=8.6 Hz, 2H), 8.37 (s, 1H); MS (ESI, m/z): 463.4 [M+H]⁺

### Example 349. 2-((4-(2-(pyridin-3-yl)-6-(4-(trifluoromethyl)phenyl)pyrimidin-4-yl)piperazin-1-yl)sulfonyl)ethan-1-ol

Using 2-(piperazin-1-ylsulfonyl)ethan-1-ol, the title compound was obtained as described for the example 248 (Scheme 1. General procedure A.).

¹H NMR (400 MHz, DMSO-d₆) δ [ppm]=3.25 (t, J=6.1 Hz, 2H), 3.33-3.36 (m, 4H), 3.73-3.79 (m, 2H), 3.96-4.04 (m, 4H), 5.04 (t, J=5.4 Hz, 1H), 7.52-7.58 (m, 2H), 7.91 (d, J=8.3 Hz, 2H), 8.55 (d, J=8.2 Hz, 2H), 8.70-8.78 (m, 2H), 9.61 (d, J=1.6 Hz, 1H); MS (ESI, m/z): 494.4 [M+H]⁺

### Example 350. (S)-1-(2-(isothiazol-4-yl)-6-(4-(trifluoromethyl)phenyl)pyrimidin-4-yl)pyrrolidin-3-ol

Scheme for the preparation of the Compound of Example 350:

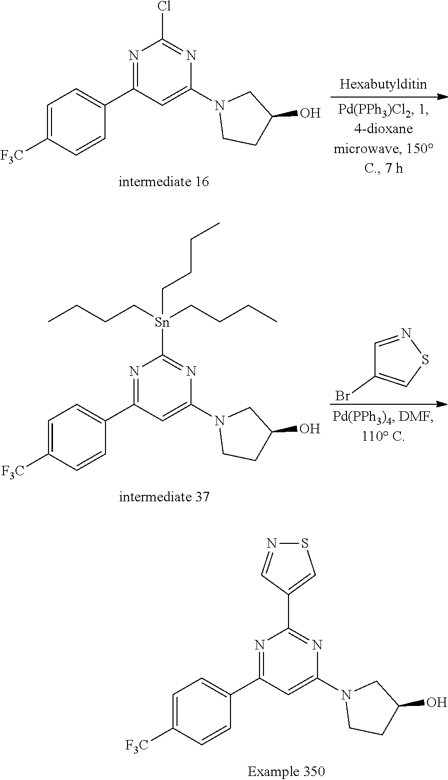 [SMILES: O[C@H]1CCN(c2cc(-c3ccc(C(F)(F)F)cc3)nc(Cl)n2)C1 ; CCC[CH2][Sn]([CH2]CCC)([CH2]CCC)[c]1nc(-c2ccc(C(F)(F)F)cc2)cc(N2CC[C@H](O)C2)n1 ; Brc1cnsc1 ; O[C@H]1CCN(c2cc(-c3ccc(C(F)(F)F)cc3)nc(-c3cnsc3)n2)C1]

### Intermediate 37. (S)-1-(6-(4-chlorophenyl)-2-(tributylstannyl)pyrimidin-4-yl)pyrrolidin-3-ol

To a solution of (S)-1-(2-chloro-6-(4-(trifluoromethyl)phenyl)pyrimidin-4-yl)pyrrolidin-3-ol (500 mg, 1.46 mmol) in 1,4-dioxane (5 mL) was added Hexabutylditin (1.01 g, 1.75 mmol) and Pd(PPh₃)Cl₂ (102 mg, 0.15 mmol) at room temperature under nitrogen. The reaction mixture was stirred at 150° C. via microwave irradiation for 7 h under nitrogen atmosphere. LCMS showed the starting material was consumed and produced the desired compound. The reaction mixture was cooled to room temperature and filtrated through a pad of celite. The filtrate was concentrated in vacuo. The residue was purified via silica gel chromatography eluted to afford 200 mg of the title compound.

MS (ESI, m/z): 600.3 [M+H]⁺

### Example 350. (S)-1-(2-(isothiazol-4-yl)-6-(4-(trifluoromethyl)phenyl)pyrimidin-4-yl)pyrrolidin-3-ol

To a solution of (S)-1-(2-(tributylstannyl)-6-(4-(trifluoromethyl)phenyl)pyrimidin-4-yl)pyrrolidin-3-ol (200 mg, 0.33 mmol) in 1,4-dioxane (6 mL) was added 4-bromoisothiazole (71 mg, 0.43 mmol), Pd(PPh₃)₄ (58 mg, 0.05 mmol), CuI (19 mg, 0.1 mmol) at room temperature under Nitrogen. The reaction mixture was stirred at 130° C. via microwave irradiation for 3 h under Nitrogen atmosphere. LCMS showed the starting material was consumed and produced the desired compound. The reaction mixture was concentrated in vacuo. The residue was diluted with water (20 ml), extracted with EtOAc (20 mL×3), the combined organic layers were washed with brine, dried and concentrated in vacuo. The residue was purified via prep-HPLC to afford 14.3 mg of the title compound.

¹H NMR (400 MHz, DMSO-d₆) δ [ppm]=1.90-2.16 (m, 2H), 3.44-3.84 (m, 4H), 4.46 (d, J=22.2 Hz, 1H), 5.07 (s, 1H), 7.05 (s, 1H), 7.88 (d, J=8.2 Hz, 2H), 8.48 (d, J=8.0 Hz, 2H), 9.26 (s, 1H), 9.68 (s, 1H); MS (ESI, m/z): 393.3[M+H]⁺.

### Example 351. (4-(methylsulfonyl)-1-(2-(pyridin-3-yl)-6-(4-(trifluoromethyl)phenyl)pyrimidin-4-yl)piperidin-4-yl)methanol

Using (4-(methylsulfonyl)piperidin-4-yl)methanol hydrochloride, the title compound was obtained as described for the example 248 (Scheme 1. General procedure A.).

¹H NMR (400 MHz, DMSO-d₆) δ [ppm]=1.90 (d, J=13.8 Hz, 2H), 1.98-2.11 (m, 2H), 2.98 (s, 3H), 3.43 (t, J=11.3 Hz, 2H), 3.97 (d, J=4.9 Hz, 2H), 4.56 (s, 2H), 5.57 (t, J=5.0 Hz, 1H), 7.49 (s, 1H), 7.56 (dd, J=7.7, 4.9 Hz, 1H), 7.90 (d, J=8.1 Hz, 2H), 8.54 (d, J=8.0 Hz, 2H), 8.72 (d, J=4.5 Hz, 1H), 8.75 (d, J=7.9 Hz, 1H), 9.61 (s, 1H); MS (ESI, m/z): 493.2 [M+H]⁺

### Example 352. 1-(4-(6-(4-chlorophenyl)-2-(pyridin-3-yl)pyrimidin-4-yl)piperazin-1-yl)-3-hydroxypropan-1-one

Using 3-hydroxy-1-(piperazin-1-yl)propan-1-one hydrochloride, the title compound was obtained as described for the example 1 (Scheme 1. General procedure A.).

¹H NMR (400 MHz, DMSO-d₆) δ [ppm]=2.57 (t, J=6.5 Hz, 2H), 3.61-3.71 (m, 6H), 3.81-3.98 (m, 4H), 4.57 (t, J=5.2 Hz, 1H), 7.40 (s, 1H), 7.52-7.58 (m, 1H), 7.61 (d, J=8.6 Hz, 2H), 8.14 (s, 1H), 8.37 (d, J=8.6 Hz, 2H), 8.67-8.77 (m, 2H), 9.60 (d, J=1.7 Hz, 1H). MS (ESI, m/z): 424.2 [M+H]⁺

### Example 353. 2-((4-(2-(1-methyl-1H-pyrazol-4-yl)-6-(4-(trifluoromethyl)phenyl)pyrimidin-4-yl)piperazin-1-yl)sulfonyl)ethanol

Scheme for the preparation of the Compound of Example 353:

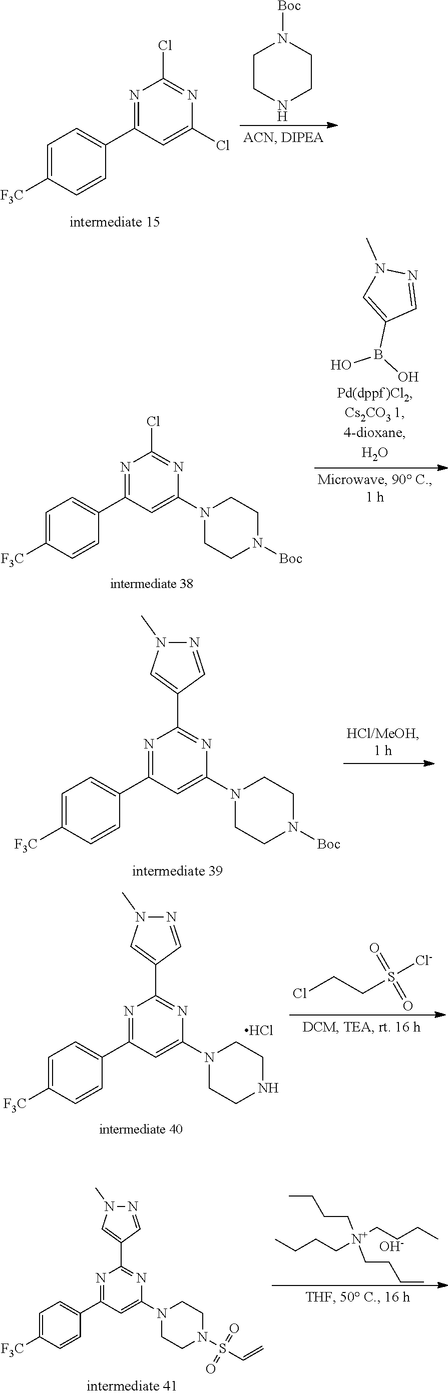

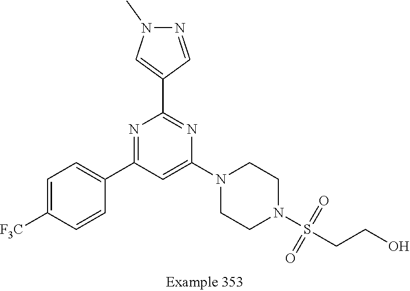 [SMILES: Cn1cc(-c2nc(-c3ccc(C(F)(F)F)cc3)cc(N3CCN(S(=O)(=O)CCO)CC3)n2)cn1]

### Intermediate 38. tert-butyl 4-(2-chloro-6-(4-(trifluoromethyl)phenyl)pyrimidin-4-yl)piperazine-1-carboxylate

To a solution of 2,4-dichloro-6-(4-(trifluoromethyl)phenyl)pyrimidine (2 g, 6.85 mmol) in ACN (20 mL) was added tert-butyl piperazine-1-carboxylate (1.9 g, 10.3 mmol) and DIPEA (2.65 g, 20.1 mmol) at room temperature. The reaction mixture was heated at 85° C. for 16 h. The mixture was concentrated in vacuo and the residue was extracted with EtOAc (20 mL×3), washed with brine, dried over anhydrous sodium sulfate, filtered and concentrated in vacuo. The residue was purified via silica gel column chromatography (Petroleum ether/EtOAc=5/1; V/V) to afford 780 mg of the title compound.

### Intermediate 39. tert-butyl 4-(2-(1-methyl-1H-pyrazol-4-yl)-6-(4-(trifluoromethyl)phenyl)pyrimidin-4-yl)piperazine-1-carboxylate

To a solution of tert-butyl 4-(2-chloro-6-(4-(trifluoromethyl)phenyl)pyrimidin-4-yl)piperazine-1-carboxylate (1 g, 2.45 mmol) in 1,4-dioxane (4 mL) and H₂O (0.8 mL) was added (1-methyl-1H-pyrazol-4-yl)boronic acid (479 mg, 3.8 mmol), Cs₂CO₃ (2.39 g, 7.35 mmol) and Pd(dppf)Cl₂ (163 mg, 0.2 mmol) at room temperature under Nitrogen. The reaction mixture was heated and stirred at 85° C. for 5 hr. The mixture was concentrated in vacuo and the residue was extracted with EtOAc (20 mL×3), washed with brine, dried over anhydrous sodium sulfate, filtered and concentrated in vacuo. The residue was purified via silica gel column chromatography (Petroleum ether/EtOAc=5/1; V/V) to afford 400 mg of the title compound.

MS (ESI, m/z): 489.2 [M+H]⁺

### Intermediate 40. 2-(1-methyl-1H-pyrazol-4-yl)-4-(piperazin-1-yl)-6-(4-(trifluoromethyl)phenyl)pyrimidine hydrochloride

To a solution of tert-butyl 4-(2-(1-methyl-1H-pyrazol-4-yl)-6-(4-(trifluoromethyl)phenyl)pyrimidin-4-yl)piperazine-1-carboxylate (400.0 mg, 0.88 mmol) in 10 mL of MeOH—HCl (4 N HCl gas in MeOH) was stirred at room temperature for 2 h. The mixture was concentrated in vacuo to give 400 mg of the title compound.

### Intermediate 41. 2-(1-methyl-1H-pyrazol-4-yl)-4-(4-(trifluoromethyl)phenyl)-6-(4-(vinylsulfonyl)piperazin-1-yl)pyrimidine

To a solution of 2-(1-methyl-1H-pyrazol-4-yl)-4-(piperazin-1-yl)-6-(4-(trifluoromethyl)phenyl)pyrimidine hydrochloride (400 mg, 1.13 mmol) in DCM (10 Ml) was added TEA (570.7 mg, 5.65 mmol) at 0° C. 2-chloroethanesulfonyl chloride (221.7 mg, 1.36 mmol) was dropwised to the above reaction mixture at 0° C. The reaction mixture was stirred at room temperature for 16 h. The mixture was extracted with dichloromethane (20 mL×3), washed with brine, dried and concentrated in vacuo. The residue was purified via silica gel column chromatography (Petroleum ether/EtOAc=1/1; V/V) to afford 120 mg of the title compound.

MS (ESI, m/z): 479.2 [M+H]⁺

### Example 353. 2-((4-(2-(1-methyl-1H-pyrazol-4-yl)-6-(4-(trifluoromethyl)phenyl)pyrimidin-4-yl)piperazin-1-yl)sulfonyl)ethanol

To a solution of 2-(1-methyl-1H-pyrazol-4-yl)-4-(4-(trifluoromethyl)phenyl)-6-(4-(vinylsulfonyl)piperazin-1-yl)pyrimidine (400 mg, 0.84 mmol) in 10 mL of THF was added tetrabutylammonium hydroxide (1.08 mg, 1.67 mmol) at room temperature. The reaction mixture was stirred at 50° C. for 16 h. The reaction mixture was cooled to room temperature and extracted with EtOAc (20 mL×3), dried and concentrated in vacuo. The obtained solid was slurried in EtOAc (10 mL). A white solid was formed. The solid was collected by filtration and dried in vacuo to give 33.7 mg of the title compound.

¹H NMR (400 MHz, DMSO-d₆) δ [ppm]=2.00 (m, 2H), 3.24 (t, J=6.1 Hz, 4H), 3.76 (m, 2H), 3.91 (s, 7H), 5.04 (t, J=5.4 Hz, 1H), 7.31 (s, 1H), 7.88 (d, J=8.3 Hz, 2H), 8.06 (s, 1H), 8.38 (s, 1H), 8.46 (d, J=8.2 Hz, 2H); MS (ESI, m/z): 497.2 [M+H]⁺

### Example 354. 1-(6-(4-chlorophenyl)-2-(pyridin-3-yl)pyrimidin-4-yl)-3-(dimethylamino)piperidin-4-ol

Using 3-(dimethylamino)piperidin-4-ol, the title compound was obtained as described for the example 1 (Scheme 1. General procedure A.).

¹H NMR (400 MHz, DMSO-d₆) δ [ppm]=1.37-1.46 (m, 1H), 1.96-2.00 (m, 1H), 2.25-2.26 (m, 1H), 2.41 (s, 6H), 3.23-3.26 (m, 4H), 3.82-3.84 (m, 1H), 4.39-4.50 (m, 1H), 7.37 (s, 1H), 7.55 (dd, J=7.9, 4.8 Hz, 1H), 7.59 (d, J=8.6 Hz, 2H), 8.32-8.43 (m, 2H), 8.49-8.87 (m, 2H), 9.57 (d, J=1.8 Hz, 1H); MS (ESI, m/z): 410.2 [M+H]⁺

### Example 355. 1-(6-(4-chlorophenyl)-2-(pyridin-3-yl)pyrimidin-4-yl)-5-(dimethylamino)piperidin-3-ol

Using 5-(dimethylamino)piperidin-3-ol, the title compound was obtained as described for the example 1 (Scheme 1. General procedure A.).

¹H NMR (400 MHz, CDCl₃) δ [ppm]=1.79-1.86 (m, 1H), 2.15-2.22 (m, 1H), 2.48 (s, 6H), 2.96 (t, J=11.1 Hz, 1H), 3.12-3.35 (m, 2H), 4.33 (s, 1H), 4.45-4.62 (m, 1H), 4.62-4.77 (m, 1H), 6.96 (s, 1H), 7.33-7.39 (m, 1H), 7.46 (d, J=8.4 Hz, 2H), 8.07 (d, J=8.4 Hz, 2H), 8.66-8.74 (m, 2H), 9.64 (s, 1H); MS (ESI, m/z): 410.2 [M+H]⁺

### Example 356. (1-(6-(4-chlorophenyl)-2-(1-methyl-1H-pyrazol-4-yl)pyrimidin-4-yl)-4-(methylsulfonyl)piperidin-4-yl)methanol

Using (4-(methylsulfonyl)piperidin-4-yl)methanol hydrochloride, the title compound was obtained as described for the example 174 (Scheme 2. General procedure B.).

¹H NMR (400 MHz, DMSO-d₆) δ [ppm]=1.89-1.93 (m, 2H), 1.98-2.09 (m, 2H), 2.99 (s, 3H), 3.44-3.54 (m, 2H), 3.94 (s, 5H), 4.53 (s, 2H), 7.24 (s, 1H), 7.67 (d, J=8.4 Hz, 2H), 8.11 (d, J=7.8 Hz, 2H), 8.27 (s, 1H), 8.63 (s, 1H); MS (ESI, m/z): 462.0 [M+H]⁺

### Example 357. (1-(2-(1-methyl-1H-pyrazol-4-yl)-6-(4-(trifluoromethyl)phenyl)pyrimidin-4-yl)-4-(methylsulfonyl)piperidin-4-yl)methanol

Scheme for the preparation of the Compound of Example 357:

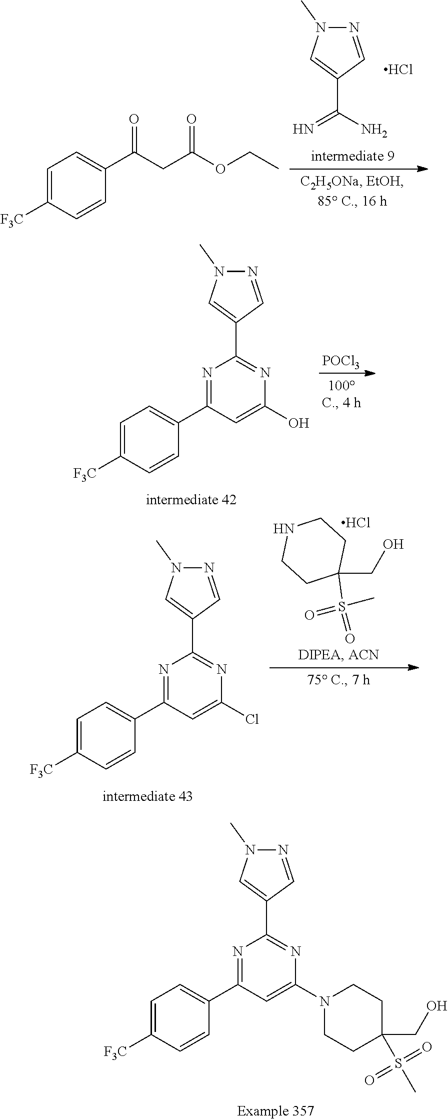

### Intermediate 42. 2-(1-methyl-1H-pyrazol-4-yl)-6-(4-(trifluoromethyl)phenyl)pyrimidin-4-ol

To a solution of methyl 3-oxo-3-(4-(trifluoromethyl)phenyl)propanoate (2.0 g, 8.13 mmol) in MeOH (20 mL) was added 1-methyl-1H-pyrazole-4-carboximidamide (1.0 g, 8.13 mmol) and sodium methoxide (527 mg, 9.576 mmol) at room temperature. The reaction mixture was heated at 80° C. under nitrogen for 16 hr. The mixture was cooled to room temperature and acidified to pH=6.0. A white solid was formed. The solid was collected by filtration and dried in vacuo to give 1.1 g of the title compound.

MS (ESI, m/z): 321.1 [M+H]⁺

### Intermediate 43. 4-chloro-2-(1-methyl-1H-pyrazol-4-yl)-6-(4-(trifluoromethyl)phenyl)pyrimidine

To a solution of 2-(1-methyl-1H-pyrazol-4-yl)-6-(4-(trifluoromethyl)phenyl)pyrimidin-4-ol (1.1 g, 3.43 mmol) in 10 mL of phosphorus oxychloride was heated at reflux for 13 hr. The mixture was concentrated in vacuo. The residue was poured into water and extracted with EtOAc (20 mL×2), washed with brine, dried and concentrated in vacuo. The residue was purified via column chromatography (DCM/MeOH=10/1; V/V) to afford 950 mg of the title compound.

MS (ESI, m/z): 339.2 [M+H]⁺

### Example 357. (1-(2-(1-methyl-1H-pyrazol-4-yl)-6-(4-(trifluoromethyl)phenyl)pyrimidin-4-yl)-4-(methylsulfonyl)piperidin-4-yl)methanol

To a solution of 4-chloro-2-(1-methyl-1H-pyrazol-4-yl)-6-(4-(trifluoromethyl)phenyl)pyrimidine (40 mg, 0.12 mmol) in ACN (5 mL) was added (4-(methylsulfonyl)piperidin-4-yl)methanol hydrochloride (32 mg, 0.14 mmol) and DIPEA (46.4 mg, 0.36 mmol) at room temperature. The reaction mixture was heated and stirred at 75° C. for 6 h. The residue was cooled to room temperature and extracted with EtOAc (20 mL×3). The combined organic layer was washed with brine, dried over anhydrous sodium sulfate, filtered and concentrated in vacuo. The residue was purified via Prep-HPLC to afford 8.2 mg of the title compound (Scheme 2. General procedure B.).

¹H NMR (400 MHz, DMSO-d₆) δ [ppm]=1.85 (d, J=13.7 Hz, 2H), 1.94-2.03 (m, 2H), 2.97 (s, 3H), 3.36 (s, 2H), 3.91 (s, 3H), 3.95 (s, 2H), 4.50 (s, 2H), 5.55 (s, 1H), 7.27 (s, 1H), 7.87 (d, J=8.4 Hz, 2H), 8.05 (s, 1H), 8.37 (s, 1H), 8.46 (d, J=8.2 Hz, 2H); MS (ESI, m/z): 496.2 [M+H]⁺

### Example 358. (1-(6-(4-chlorophenyl)-2-(pyridin-3-yl)pyrimidin-4-yl)-3-(dimethylamino)piperidin-4-yl)methanol

Using (3-(dimethylamino)piperidin-4-yl)methanol, the title compound was obtained as described for the example 1 (Scheme 1. General procedure A.).

¹H NMR (400 MHz, DMSO-d₆) δ [ppm]=1.48-1.59 (m, 1H), 1.92-2.04 (m, 1H), 2.13-2.30 (m, 2H), 2.34 (s, 6H), 3.05-3.24 (m, 1H), 3.37-3.49 (m, 2H), 3.61-3.78 (m, 2H), 4.15 (s, 1H), 4.46-5.00 (m, 1H), 7.38 (s, 1H), 7.55 (dd, J=7.9, 4.9 Hz, 1H), 7.60 (d, J=8.6 Hz, 2H), 8.36 (d, J=8.6 Hz, 2H), 8.67-8.74 (m, 2H), 9.57 (d, J=1.5 Hz, 1H); MS (ESI, m/z): 424.2[M+H]⁺

### Example 359. 2-((4-(6-(4-chlorophenyl)-2-(pyridin-3-yl)pyrimidin-4-yl)-3-methylpiperazin-1-yl)sulfonyl)ethan-1-ol

Using 2-((3-methylpiperazin-1-yl)sulfonyl)ethan-1-ol, the title compound was obtained as described for the example 1 (Scheme 2. General procedure B.).

¹H NMR (400 MHz, DMSO-d₆) δ [ppm]=1.27 (d, J=6.6 Hz, 3H), 2.95-3.06 (m, 1H), 3.13-3.21 (m, 1H), 3.22-3.29 (m, 3H), 3.50 (d, J=12.0 Hz, 1H), 3.68 (d, J=11.6 Hz, 1H), 3.78 (t, J=5.4 Hz, 2H), 4.66 (s, 1H), 5.06 (s, 2H), 7.40 (s, 1H), 7.56 (dd, J=7.9, 4.8 Hz, 1H), 7.61 (d, J=8.6 Hz, 2H), 8.37 (d, J=8.6 Hz, 2H), 8.68-8.78 (m, 2H), 9.60 (d, J=1.9 Hz, 1H); MS (ESI, m/z): 474.2[M+H]⁺

### Example 360. 2-((1-(6-(4-chlorophenyl)-2-(pyridin-3-yl)pyrimidin-4-yl)piperidin-4-yl)amino)ethanol

Scheme for the preparation of the Compound of Example 360:

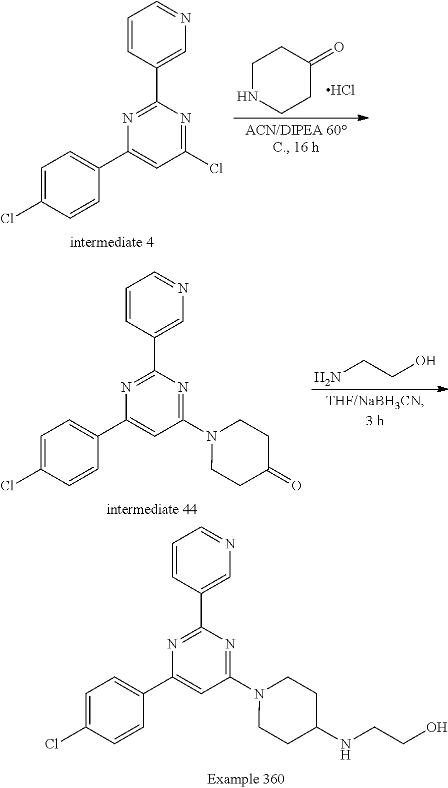

### Intermediate 44. 1-(6-(4-chlorophenyl)-2-(pyridin-3-yl)pyrimidin-4-yl)piperidin-4-one

To a solution of 4-chloro-6-(4-chlorophenyl)-2-(pyridin-3-yl)pyrimidine (400.0 mg, 1.33 mmol) in ACN (5 mL) was added DIPEA (515 mg, 4.0 mmol) and piperidin-4-one hydrochloride (270 mg, 1.99 mmol) at room temperature. The reaction mixture was heated and stirred at 75° C. for 16 hr. The mixture was concentrated in vacuo. The residue was poured into water and extracted with EtOAc (20 mL×2), washed with brine. The combined organic layer was dried and concentrated in vacuo. The obtained crude product was purified via column chromatography (Petroleum ether/EtOAc=2/1; V/V) to afford 330 mg of the title compound (Scheme 1. General procedure A.).

MS (ESI, m/z): 365.2 [M+H]⁺

### Example 360. 2-((1-(6-(4-chlorophenyl)-2-(pyridin-3-yl)pyrimidin-4-yl)piperidin-4-yl)amino)ethanol

To a solution of 1-(6-(4-chlorophenyl)-2-(pyridin-3-yl)pyrimidin-4-yl)piperidin-4-one (330.0 mg, 0.906 mmol) in THF (15 mL) was added 2-aminoethanol (83 mg, 1.36 mmol) and 2 drops of CH₃COOH. The reaction mixture was stirred at r.t. for 16 hr. And then NaBH(OAc)₃ (576.0 mg, 2.72 mmol) was added to the above reaction mixture at room temperature. The reaction mixture was stirred at r.t. for 3 hr. The residue was poured into water and extracted with EtOAc (20 mL×2), washed with brine. The combined organic layer was dried and concentrated in vacuo. The obtained crude product was purified via Prep-HPLC to afford 204.3 mg of the title compound.

¹H NMR (400 MHz, DMSO-d₆) δ [ppm]=1.64-1.72 (m, 2H), 2.25 (d, J=10.8 Hz, 2H), 3.05-3.11 (m, 4H), 3.45 (s, 1H), 3.72-3.75 (t, J=4.9 Hz, 2H), 4.88 (s, 2H), 7.54 (s, 1H), 7.62 (d, J=8.5 Hz, 2H), 8.18 (dd, J=7.8, 5.8 Hz, 1H), 0.8.41 (d, J=8.5 Hz, 2H), 9.04 (d, J=5.2 Hz, 1H), 9.30 (s, 2H), 9.44 (d, J=8.1 Hz, 1H), 9.71 (s, 1H); MS (ESI, m/z): 410.2 [M+H]⁺

### Example 361. 1-(4-(6-(4-chlorophenyl)-2-(pyridin-3-yl)pyrimidin-4-yl)piperazin-1-yl)-4-hydroxybutan-1-one

Scheme for the preparation of the Compound of Example 361:

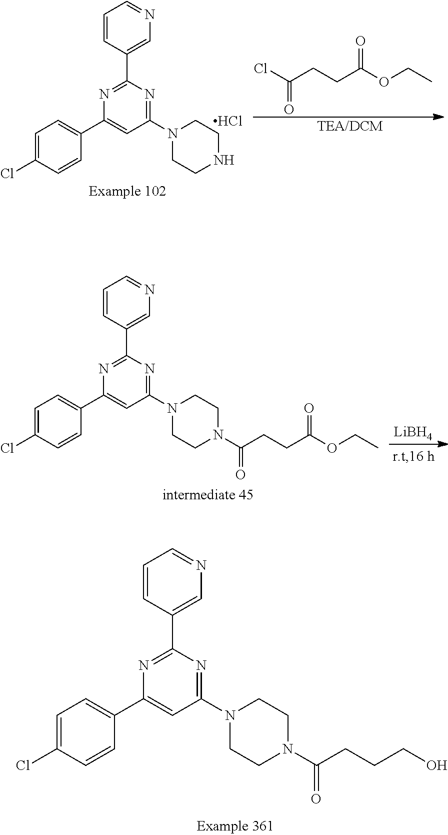

### Intermediate 45. ethyl 4-(4-(6-(4-chlorophenyl)-2-(pyridin-3-yl)pyrimidin-4-yl)piperazin-1-yl)-4-oxobutanoate

To a solution of 4-(4-chlorophenyl)-6-(piperazin-1-yl)-2-(pyridin-3-yl)pyrimidine hydrochloride (450 mg, 0.57 mmol) in 10 mL of DCM was added TEA (585.8 mg, 1.39 mmol) at 0° C. Ethyl 4-chloro-4-oxobutanoate (585.8 mg, 5.8 mmol) was added dropwised to the above reaction mixture at 0° C. The reaction mixture was stirred at room temperature for 3 hr. The residue was extracted with dichloromethane (20 mL×3), washed with brine, dried and concentrated in vacuo. The residue was purified via silica gel column chromatography (Petroleum ether/EtOAc=2/1; V/V) to afford 300 mg of the title compound.

MS (ESI, m/z): 480.2 [M+H]⁺

### Example 361. 1-(4-(6-(4-chlorophenyl)-2-(pyridin-3-yl)pyrimidin-4-yl)piperazin-1-yl)-4-hydroxybutan-1-one

To a suspension of ethyl 4-(4-(6-(4-chlorophenyl)-2-(pyridin-3-yl)pyrimidin-4-yl)piperazin-1-yl)-4-oxobutanoate (300 mg, 0.63 mmol) in diethel ether (10 mL) was added LiBH₄ (17.6 mg, 0.81 mmol) and MeOH (26 mg, 0.81 mmol) at 0° C. The reaction mixture was stirred at room temperature for 2 hr. Methanol was added dropwise to the above reaction mixture until no bubbles are generated. The solvent was removed in vacuo and the residue was purified via Prep-HPLC to afford 15.9 mg of the title compound.

¹H NMR (400 MHz, DMSO-d₆) δ [ppm]=1.61-1.78 (m, 2H), 2.43 (t, J=7.4 Hz, 2H), 3.44 (t, J=6.1 Hz, 2H), 3.58-3.68 (m, 4H), 3.89 (d, J=27.3 Hz, 4H), 4.49 (s, 1H), 7.40 (s, 1H), 7.55 (dd, J=7.9, 4.8 Hz, 1H), 7.61 (d, J=8.6 Hz, 2H), 8.37 (d, J=8.6 Hz, 2H), 8.65-8.82 (m, 2H), 9.60 (d, J=1.4 Hz, 1H); MS (ESI, m/z): 438.2 [M+H]⁺

### Example 362. 3-((4-(6-(4-chlorophenyl)-2-(pyridin-3-yl)pyrimidin-4-yl)piperazin-1-yl)sulfonyl)propan-1-ol

Scheme for the preparation of the Compound of Example 362:

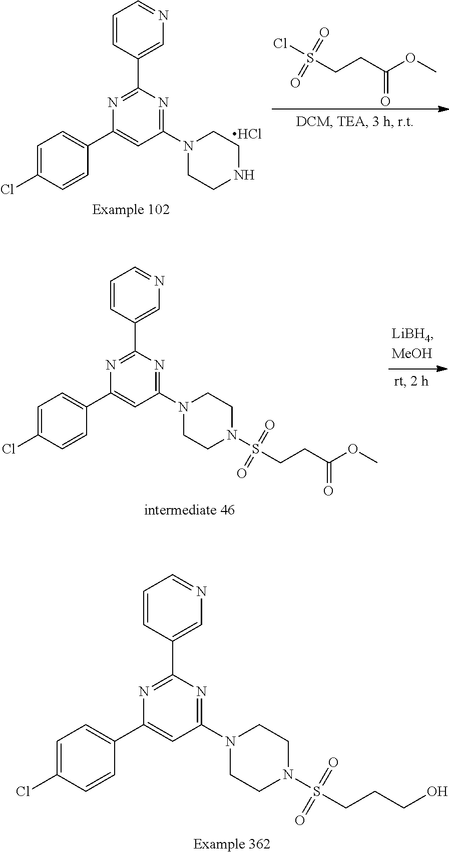

### Intermediate 46. Methyl 3-((4-(6-(4-chlorophenyl)-2-(pyridin-3-yl)pyrimidin-4-yl)piperazin-1-yl)sulfonyl)propanoate

To a solution of 4-(4-chlorophenyl)-6-(piperazin-1-yl)-2-(pyridin-3-yl)pyrimidine hydrochloride (200 mg, 0.57 mmol) in 10 mL of DCM was added TEA (172.71 mg, 1.71 mmol) at 0° C. Methyl 3-(chlorosulfonyl)propanoate (127 mg, 0.68 mmol) was dropwised to the above reaction mixture at 0° C. The reaction mixture was stirred at room temperature for 16 h. The reaction mixture was extracted with dichloromethane (20 mL×3), washed with brine, dried over anhydrous sodium sulfate and concentrated in vacuo. The residue was purified via silica gel column chromatography (Petroleum ether/EtOAc=2/1; V/V) to afford 110 mg of the title compound.

MS (ESI, m/z): 501.2 [M+H]⁺

### Example 362. 3-((4-(6-(4-chlorophenyl)-2-(pyridin-3-yl)pyrimidin-4-yl)piperazin-1-yl)sulfonyl)propan-1-ol

To a suspension of 3-((4-(6-(4-chlorophenyl)-2-(pyridin-3-yl)pyrimidin-4-yl)piperazin-1-yl)sulfonyl)propanoate (110 mg, 0.22 mmol) in diethyl ether (10 mL) was added LiBH₄ (6.2 mg, 0.29 mmol) and MeOH (9.28 mg, 0.29 mmol) at 0° C. The reaction mixture was stirred at room temperature for 2 h. Methanol was added dropwise to the above reaction mixture at 0° C. until no bubbles are generated. The solvent was removed in vacuo and the residue was purified via Prep-HPLC to afford 30.6 mg of the title compound.

¹H NMR (400 MHz, DMSO-d₆) δ [ppm]=1.80-1.86 (m, 2H), 3.08-3.15 (m, 2H), 3.30-3.36 (m, 4H), 3.48 (t, J=6.2 Hz, 2H), 3.99 (s, 4H), 7.47 (s, 1H), 7.59-7.67 (m, 3H), 8.38 (d, J=8.6 Hz, 2H), 8.76 (dd, J=4.9, 1.5 Hz, 1H), 8.85 (d, J=8.0 Hz, 1H), 9.62 (d, J=1.6 Hz, 1H); MS (ESI, m/z): 474.0 [M+H]⁺

### Example 363. 1-(4-(6-(4-chlorophenyl)-2-(pyridin-3-yl)pyrimidin-4-yl)piperazin-1-yl)-3,4-dihydroxybutan-1-one

### Example 364. 1-(4-(6-(4-chlorophenyl)-2-(pyridin-3-yl)pyrimidin-4-yl)piperazin-1-yl)-2,3-dihydroxybutan-1-one

Scheme for the preparation of the Compound of Example 363 and 364:

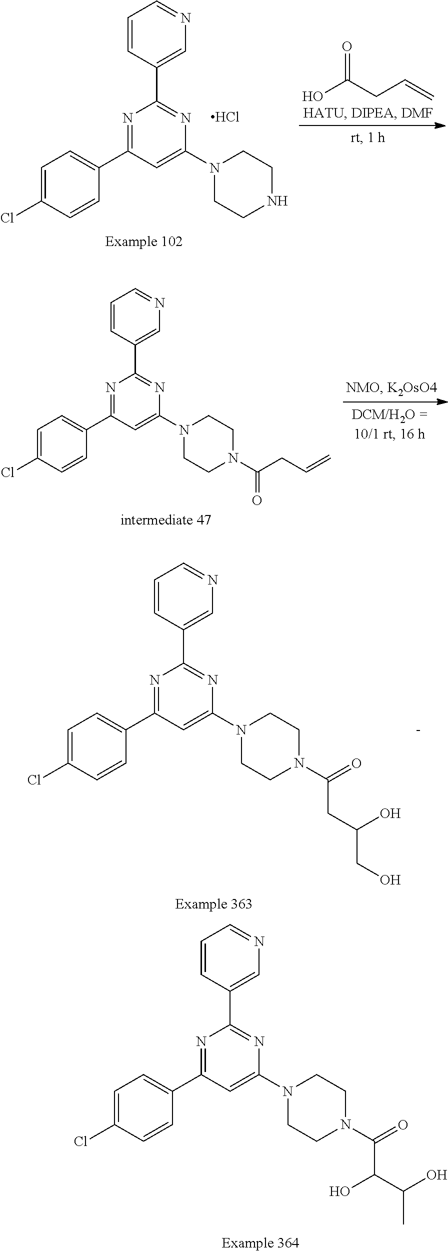

### Intermediate 47. 1-(4-(6-(4-chlorophenyl)-2-(pyridin-3-yl)pyrimidin-4-yl)piperazin-1-yl)but-3-en-1-one

To a solution of 4-(4-chlorophenyl)-6-(piperazin-1-yl)-2-(pyridin-3-yl)pyrimidine (400 mg, 0.114 mmol) in DMF (10 mL) was added but-3-enoic acid (147 mg, 0.17 mmol), HATU (864 mg, 2.28 mmol), DIPEA (440 mg, 3.41 mmol) at room temperature. The reaction mixture was stirred at room temperature for 2 hr. LCMS showed the starting material was consumed and produced the desired compound. The reaction mixture was diluted with water (20 mL), extracted with EtOAc (10 mL×2). The combined organic layers were washed with brine, dried and concentrated in vacuo. The residue was purified via silica gel column chromatography (Petroleum ether/EtOAc=1/1; V/V) to afford 420 mg of the title compound.

MS (ESI, m/z): 420.2 [M+H]⁺

### Example 363. 1-(4-(6-(4-chlorophenyl)-2-(pyridin-3-yl)pyrimidin-4-yl)piperazin-1-yl)-3,4-dihydroxybutan-1-one

### Example 364. 1-(4-(6-(4-chlorophenyl)-2-(pyridin-3-yl)pyrimidin-4-yl)piperazin-1-yl)-2,3-dihydroxybutan-1-one

To a solution of 1-(4-(6-(4-chlorophenyl)-2-(pyridin-3-yl)pyrimidin-4-yl)piperazin-1-yl)but-3-en-1-one (200 mg, 0.48 mmol) in DCM (15 mL) and H₂O (2 mL) was added NMO (83.8 mg, 0.72 mmol) and K₂OSO₄ (17.5 mg, 0.048 mmol) at room temperature. The reaction mixture was heated and stirred at 35° C. for 16 hr. LCMS showed the starting material was consumed and produced the desired compound. The reaction mixture was filtrated, the filtrate was concentrated in vacuo. The residue was diluted with water and extracted with EtOAc (20 mL×2). The combined organic layer was dried and concentrated in vacuo. The obtained crude product was purified via prep-HPLC to afford 8.2 mg of the example 363 as a white solid and 8.3 mg of the example 364 as a white solid.

Example 363: ¹H NMR (400 MHz, CDCl₃) δ [ppm]=2.55-2.70 (m, 2H), 3.58-3.72 (m, 3H), 3.74-3.91 (m, 5H), 3.91-4.04 (m, 2H), 4.18-4.25 (m, 1H), 6.86 (s, 1H), 7.43-7.53 (m, 3H), 8.08 (d, J=8.6 Hz, 2H), 8.72 (d, J=3.9 Hz, 1H), 8.84 (d, J=7.9 Hz, 1H), 9.70 (s, 1H); MS (ESI, m/z): 454.2 [M+H]⁺

Example 364: ¹H NMR (400 MHz, DMSO-d₆) δ [ppm]=1.08 (d, J=6.3 Hz, 3H), 3.58-3.78 (m, 4H), 3.81-4.00 (m, 5H), 4.23 (s, 1H), 4.68 (s, 1H), 4.86 (s, 1H), 7.41 (s, 1H), 7.55 (dd, J=7.9, 4.8 Hz, 1H), 7.61 (d, J=8.6 Hz, 2H), 8.37 (d, J=8.6 Hz, 2H), 8.68-8.77 (m, 2H), 9.60 (d, J=1.6 Hz, 1H); MS (ESI, m/z): 454.2 [M+H]⁺

### Example 365. 4-(6-(4-chlorophenyl)-2-(pyridin-3-yl)pyrimidin-4-yl)-6-methylpiperazin-2-one

Using 6-methylpiperazin-2-one, the title compound was obtained as described for the example 1 (Scheme 1. General procedure A.).

¹H NMR (400 MHz, DMSO-d₆) δ [ppm]=1.20 (d, J=6.4 Hz, 3H), 3.42-3.53 (m, 1H), 3.61-3.70 (m, 1H), 4.17 (d, J=17.8 Hz, 1H), 4.31-4.53 (m, 2H), 7.40 (s, 1H), 7.55 (dd, J=7.7, 4.9 Hz, 1H), 7.61 (d, J=8.6 Hz, 2H), 8.26 (s, 1H), 8.40 (d, J=8.6 Hz, 2H), 8.71 (s, 1H), 8.73-8.81 (m, 1H), 9.60 (s, 1H); MS (ESI, m/z): 380.2 [M+H]⁺

### Example 366. (S)-3-(4-(3-hydroxypyrrolidin-1-yl)-6-(6-morpholinopyridin-3-yl)pyrimidin-2-yl)pyridin-2-ol

Scheme for the preparation of the Compound of Example 366:

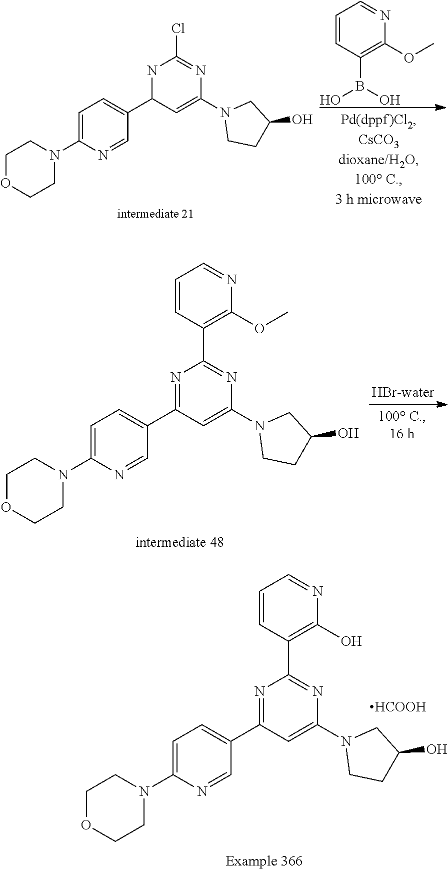

### Intermediate 48. (S)-1-(2-(2-methoxypyridin-3-yl)-6-(6-morpholinopyridin-3-yl)pyrimidin-4-yl)pyrrolidin-3-ol

To a solution of (S)-1-(2-chloro-6-(6-morpholinopyridin-3-yl)pyrimidin-4-yl)pyrrolidin-3-ol (130 mg, 0.36 mmol) in 1,4-dioxane (5 mL) and H₂O (1 mL) was added (2-methoxypyridin-3-yl)boronic acid (110 mg, 0.72 mmol), Cs₂CO₃ (350 mg, 1.08 mmol) and Pd(dppf)Cl₂ (58.5 mg, 0.07 mmol) at room temperature under nitrogen. The reaction mixture was stirred at 110° C. via microwave irradiation for 3 h under nitrogen atmosphere. LCMS showed the starting material was consumed and produced the desired compound. The reaction mixture was concentrated in vacuo. The residue was purified via silica gel column chromatography (DCM/MeOH=10/1; V/V) to afford 80 mg of the title compound.

MS (ESI, m/z): 435.2 [M+H]⁺

### Example 366. (S)-3-(4-(3-hydroxypyrrolidin-1-yl)-6-(6-morpholinopyridin-3-yl)pyrimidin-2-yl)pyridin-2-ol

To a suspension of (S)-1-(2-(2-methoxypyridin-3-yl)-6-(6-morpholinopyridin-3-yl)pyrimidin-4-yl)pyrrolidin-3-ol (120 mg, 0.27 mmol) in HBr solution (40% HBr in H₂O, 4 mL) was stirred 100° C. for 10 h in a sealed tube. LCMS showed the starting material was consumed and produced the desired compound. The reaction mixture was cooled to room temperature and concentrated in vacuo. The residue was purified via prep-HPLC to afford 13.2 mg of the example 363 as a white solid and 8.3 mg of the title compound.

¹H NMR (400 MHz, DMSO-d₆) δ [ppm]=1.89-2.16 (m, 2H), 3.44-3.88 (m, 12H), 4.47 (d, J=14.8 Hz, 1H), 5.14 (d, J=28.6 Hz, 1H), 6.88-6.99 (m, 2H), 7.01 (d, J=9.1 Hz, 1H), 8.18 (s, 1H), 8.14 (s, 1H), 8.26 (dd, J=8.9, 2.1 Hz, 1H), 8.74 (s, 1H), 8.93 (d, J=2.4 Hz, 1H); MS (ESI, m/z): 421.2 [M+H]⁺

### Example 367. (S)-4-(4-(6-(3-hydroxypyrrolidin-1-yl)-2-(pyridin-3-yl)pyrimidin-4-yl)phenyl)morpholin-3-one

Scheme for the preparation of the Compound of Example 367:

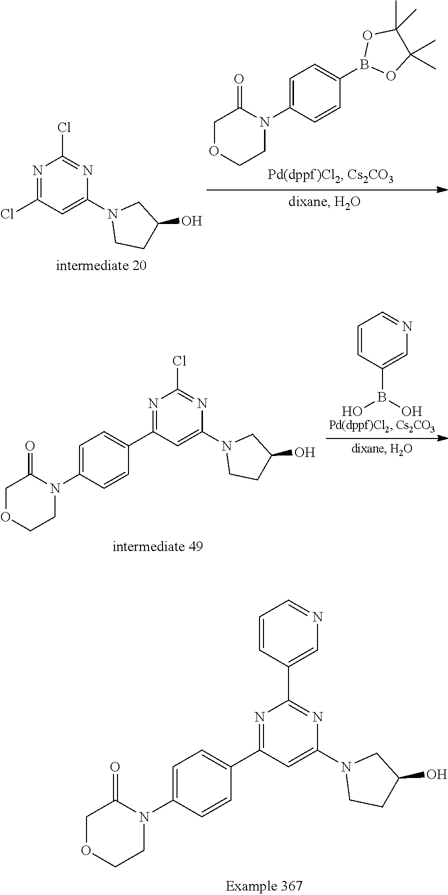 [SMILES: O[C@H]1CCN(c2cc(Cl)nc(Cl)n2)C1 ; CC1(C)OB(c2ccc(N3CCOCC3=O)cc2)OC1(C)C ; O=C1COCCN1c1ccc(-c2cc(N3CC[C@H](O)C3)nc(Cl)n2)cc1 ; OB(O)c1cccnc1 ; O=C1COCCN1c1ccc(-c2cc(N3CC[C@H](O)C3)nc(-c3cccnc3)n2)cc1]

### Intermediate 49. (S)-4-(4-(2-chloro-6-(3-hydroxypyrrolidin-1-yl)pyrimidin-4-yl)phenyl)morpholin-3-one

To a mixture of (S)-1-(2,6-dichloropyrimidin-4-yl)pyrrolidin-3-ol (60 mg, 0.26 mmol) in dioxane (3 mL) and water (0.5 mL) were added 4-(4-(4,4,5,5-tetramethyl-1,3,2-dioxaborolan-2-yl)phenyl)morpholin-3-one (93 mg, 0.31 mmol), Cs₂CO₃ (250 mg, 0.77 mmol) and 1′-Bis(diphenylphosphino)ferrocene-palladium(II)dichloride dichloromethane complex (41 mg, 0.05 mmol) under Nitrogen at room temperature. The mixture was degassed and purged with N₂ three times. The reaction mixture was heated at 90° C. under Nitrogen for 16 h. LCMS showed the reaction was complete. The mixture was filtered. The filtrate was concentrated in vacuo. The residue was purified by prep-TLC (EtOAc/MeOH=20/1, V/V) to give 10 mg of the title compound.

MS (ESI, m/z): 475.0 [M+H]⁺

### Example 367. (S)-4-(4-(6-(3-hydroxypyrrolidin-1-yl)-2-(pyridin-3-yl)pyrimidin-4-yl)phenyl)morpholin-3-one

To a mixture of (S)-4-(4-(2-chloro-6-(3-hydroxypyrrolidin-1-yl)pyrimidin-4-yl)phenyl)morpholin-3-one (100 mg, 0.27 mmol) in dioxane (3 mL) and H₂O (0.5 mL) were added pyridin-3-ylboronic acid (65 mg, 0.55 mmol), Cs₂CO₃ (173 mg, 0.54 mmol) and Pd(dppf)Cl₂ (22 mg, 0.03 mmol) at 10° C. The reaction mixture was degassed and purged with N₂ three times. The reaction was stirred at 100° C. for 16 h. LCMS showed the reaction was complete. The reaction was cooled to room temperature and quenched by water (10 mL). The residue was extracted with EtOAc (15 mL×3). The organic layers were combined and washed with brine (10 mL). The organic layer was dried over anhydrous sodium sulfate and filtered. The filtrate was concentrated in vacuo. The residue was purified by prep-HPLC to give 8.7 mg of the title compound.

¹H NMR (400 MHz, DMSO-d₆) δ [ppm]=1.94-2.13 (m, 2H), 3.41-3.77 (m, 4H), 3.81-3.83 (m, 2H), 4.00-4.02 (m, 2H), 4.25 (s, 2H), 4.44-4.48 (m, 1H), 5.08 (d, J=35.2 Hz, 1H), 7.00 (s, 1H), 7.57-7.59 (m, 1H), 7.58 (d, J=8.5 Hz, 2H), 8.32-8.34 (m, 2H), 8.67-8.70 (m, 1H), 8.75 (d, J=7.9 Hz, 1H), 9.60 (s, 1H); MS (ESI, m/z): 418.2 [M+H]⁺

### Example 368. 2-((4-(6-(4-chlorophenyl)-2-(isothiazol-4-yl)pyrimidin-4-yl)piperazin-1-yl)sulfonyl)ethanol

Scheme for the preparation of the Compound of Example 368:

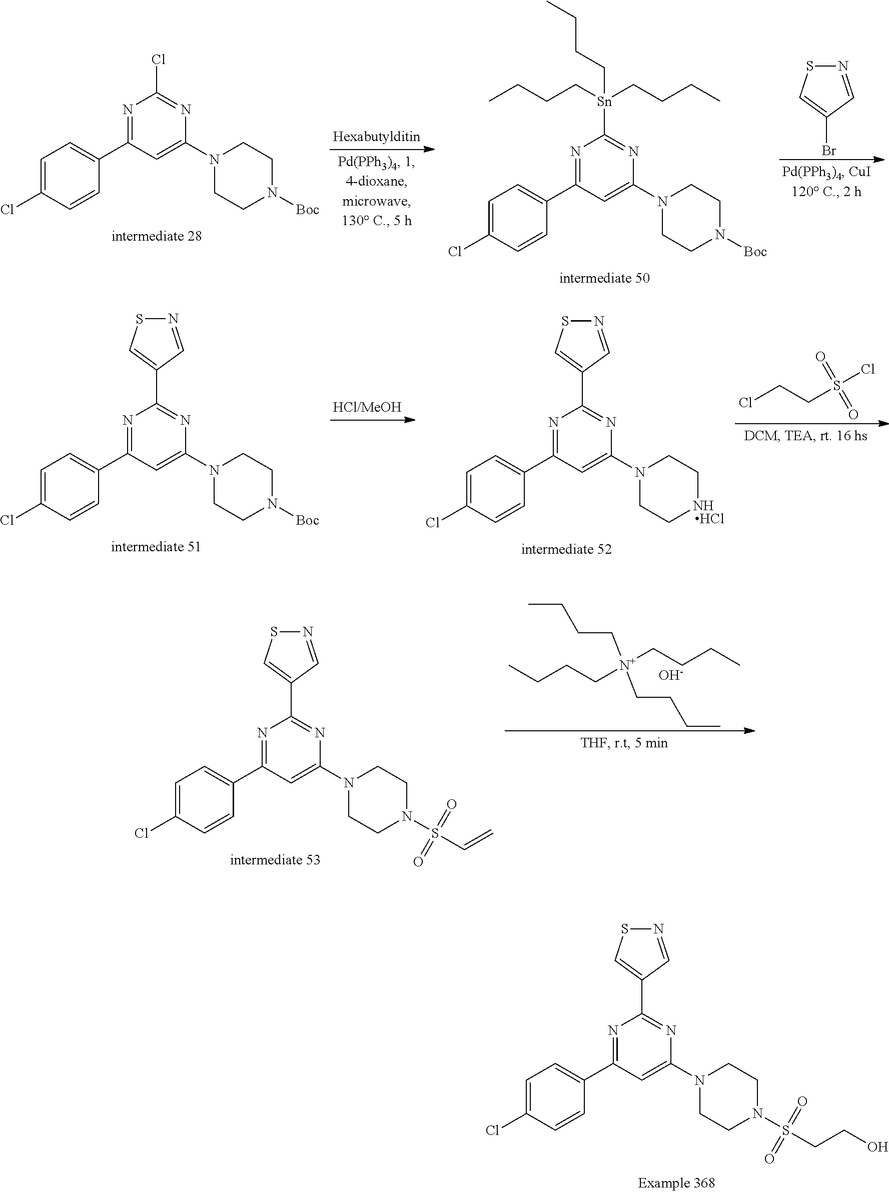

### Intermediate 50. tert-butyl 4-(6-(4-chlorophenyl)-2-(tributylstannyl)pyrimidin-4-yl)piperazine-1-carboxylate

To a solution of tert-butyl 4-(2-chloro-6-(4-chlorophenyl)pyrimidin-4-yl)piperazine-1-carboxylate (204 mg, 0.5 mmol) in 1,4-dioxane (8 mL) was added Hexabutylditin (319 mg, 0.55 mmol), Pd(PPh₃)₄ (86.7 mg, 0.075 mmol) at room temperature under Nitrogen. The reaction mixture was heated and stirred at 130° C. for 3.5 hr under Nitrogen. TLC showed the starting material consumed completely. The mixture was concentrated in vacuo and the residue was purified via silica gel column chromatography (DCM/MeOH=10/1; V/V) to afford 1.1 g of the title compound.

¹H NMR (400 MHz, CDCl₃) δ [ppm]=0.87-0.94 (m, 9H), 1.11-1.15 (m, 4H), 1.29-1.39 (m, 8H), 1.49 (s, 9H), 1.60-1.68 (m, 6H), 3.53-3.56 (m, 4H), 3.70-3.72 (m, 4H), 6.69 (s, 1H), 7.41 (d, J=8.6 Hz, 2H), 7.97 (d, J=8.6 Hz, 2H); MS (ESI, m/z): 665.3 [M+H]⁺

### Intermediate 51. tert-butyl 4-(6-(4-chlorophenyl)-2-(isothiazol-4-yl)pyrimidin-4-yl)piperazine-1-carboxylate

To a solution of tert-butyl 4-(6-(4-chlorophenyl)-2-(tributylstannyl)pyrimidin-4-yl)piperazine-1-carboxylate (550 mg, 0.832 mmol) in 1,4-dioxane (12 mL) was added 4-bromoisothiazole (91.1 mg, 0.555 mmol), Pd(PPh₃)₄ (144.5 mg, 0.125 mmol) and CuI (23.8 mg, 0.1255 mmol) at room temperature under Nitrogen atmosphere. The reaction mixture was heated and stirred at 120° C. under N₂ for 2 hr. LCMS showed the starting material was consumed completely. After the reaction mixture was cooled to room temperature. The mixture was concentrated in vacuo and the residue was purified via silica gel column chromatography (Petroleum ether/EtOAc=5/1; V/V) to afford 270 mg of the title compound.

MS (ESI, m/z): 458.1 [M+H]⁺

### Intermediate 52. 4-(4-(4-chlorophenyl)-6-(piperazin-1-yl)pyrimidin-2-yl)isothiazole

To a solution of tert-butyl 4-(6-(4-chlorophenyl)-2-(isothiazol-4-yl)pyrimidin-4-yl)piperazine-1-carboxylate (270 mg, 0.6 mmol) in Hydrogen chloride-Methanol solution (4 M HCl gas in MeOH, 5 mL) was stirred at room temperature for 2 h. The mixture was concentrated in vacuo to give 250 mg of the title compound.

MS (ESI, m/z): 358.1 [M+H]⁺

### Intermediate 53. 4-(4-(4-chlorophenyl)-6-(4-(vinylsulfonyl)piperazin-1-yl)pyrimidin-2-yl)isothiazole

To a solution of 4-(4-(4-chlorophenyl)-6-(piperazin-1-yl)pyrimidin-2-yl)isothiazole (250 mg, 0.63 mmol) in 5 mL dichloromethan was added TEA (318 mg, 3.15 mmol) at 0° C. 2-chloroethanesulfonyl chloride (123 mg, 0.76 mmol) was added dropwise to the above reaction mixture at 0° C. The reaction mixture was stirred at room temperature for 16 h. TLC showed the starting material consumed completely. The mixture was concentrated in vacuo and the residue was purified via silica gel column chromatography (DCM/MeOH=10/1; V/V) to afford 120 mg of the title compound.

MS (ESI, m/z): 448.1 [M+H]⁺

### Example 368. 2-((4-(6-(4-chlorophenyl)-2-(isothiazol-4-yl)pyrimidin-4-yl)piperazin-1-yl)sulfonyl)ethanol

To a solution of 4-(4-(4-chlorophenyl)-6-(4-(vinylsulfonyl)piperazin-1-yl)pyrimidin-2-yl)isothiazole (120 mg, 0.27 mmol) in 10 mL THF was added tetrabutylammonium hydroxide (310 mg, 0.30 mmol) at room temperature. The reaction mixture was stirred at 50° C. for 4 h. TLC showed the starting material was consumed completely. The mixture was cooled to room temperature. The mixture was concentrated in vacuo and the residue was purified via Prep-HPLC to afford 19.3 mg of the title compound.

¹H NMR (400 MHz, DMSO-d₆) δ [ppm]=3.25 (t, J=6.1 Hz, 2H), 3.32-3.28 (m, 4H), 3.76 (dd, J=11.2, 5.6 Hz, 2H), 3.96 (s, 4H), 5.03 (t, J=5.3 Hz, 1H), 7.39 (s, 1H), 7.60 (d, J=8.5 Hz, 2H), 8.35 (d, J=8.6 Hz, 2H), 9.28 (s, 1H), 9.73 (s, 1H); MS (ESI, m/z): 466.0 [M+H]⁺

### Example 369. 3-((4-(6-(4-chlorophenyl)-2-(pyridin-3-yl)pyrimidin-4-yl)piperazin-1-yl)sulfonyl)propane-1,2-diol

Scheme for the preparation of the Compound of Example 369:

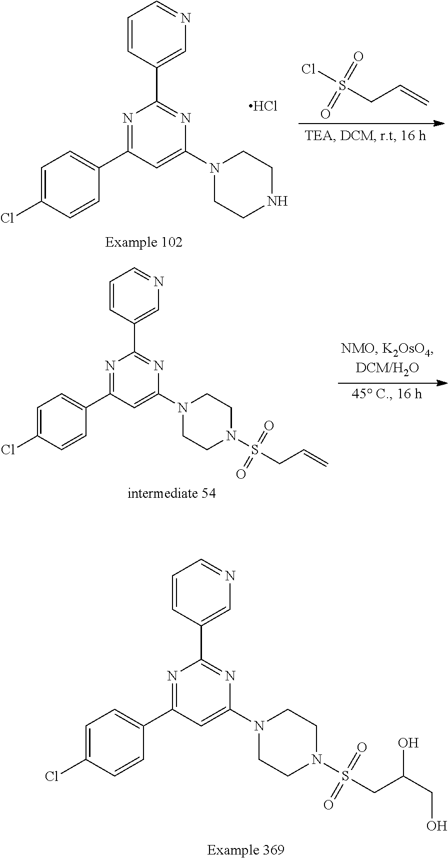

### Intermediate 54. 4-(4-(allylsulfonyl)piperazin-1-yl)-6-(4-chlorophenyl)-2-(pyridin-3-yl)pyrimidine

To a solution of 4-(4-chlorophenyl)-6-(piperazin-1-yl)-2-(pyridin-3-yl)pyrimidine hydrochloride (500 mg, 1.42 mmol) in DCM (10 mL) was added TEA (719 mg, 7.12 mmol) at room temperature. Prop-2-ene-1-sulfonyl chloride (400 mg, 2.85 mmol) was dropwised to the above reaction mixture at 0° C. The reaction mixture was stirred at room temperature for 16 h. LCMS showed the starting material was consumed and produced the desired compound. The reaction mixture concentrated in vacuo. The residue was purified via silica gel column chromatography (Petroleum ether/EtOAc=1/1; V/V) to afford 900 mg of the title compound.

MS (ESI, m/z): 456.2 [M+H]⁺

### Example 369. 3-((4-(6-(4-chlorophenyl)-2-(pyridin-3-yl)pyrimidin-4-yl)piperazin-1-yl)sulfonyl)propane-1,2-diol

To a solution of 4-(4-(allylsulfonyl)piperazin-1-yl)-6-(4-chlorophenyl)-2-(pyridin-3-yl)pyrimidine (870 mg, 1.91 mmol) in DCM (30 mL) and H₂O (3 mL) was added NMO (672 mg, 5.74 mmol) and K₂OSO₄ (140 mg, 0.38 mmol) at room temperature. The reaction mixture was heated and stirred at 45° C. for 16 hr. The mixture was concentrated in vacuo The residue was diluted with water and extracted with EtOAc (20 mL×2), washed with brine. The combined organic layer was dried and concentrated in vacuo. The obtained crude product was purified via prep-HPLC to afford 30.5 mg of the title compound.

¹H NMR (400 MHz, DMSO-d₆) δ [ppm]=3.07 (dd, J=14.7, 8.3 Hz, 1H), 3.19-3.46 (m, 7H), 3.85-3.93 (m, 1H), 3.94-4.13 (m, 4H), 7.51 (s, 1H), 7.63 (d, J=8.6 Hz, 2H), 7.90 (dd, J=7.8, 5.3 Hz, 1H), 8.40 (d, J=8.6 Hz, 2H), 8.89 (d, J=5.1 Hz, 1H), 9.13 (d, J=8.0 Hz, 1H), 9.68 (d, J=1.8 Hz, 1H); MS (ESI, m/z): 490.2 [M+H]⁺

### Example 370. 2-((4-(2-(isothiazol-4-yl)-6-(4-(trifluoromethyl)phenyl)pyrimidin-4-yl)piperazin-1-yl)sulfonyl)ethanol

Scheme for the preparation of the Compound of Example 370:

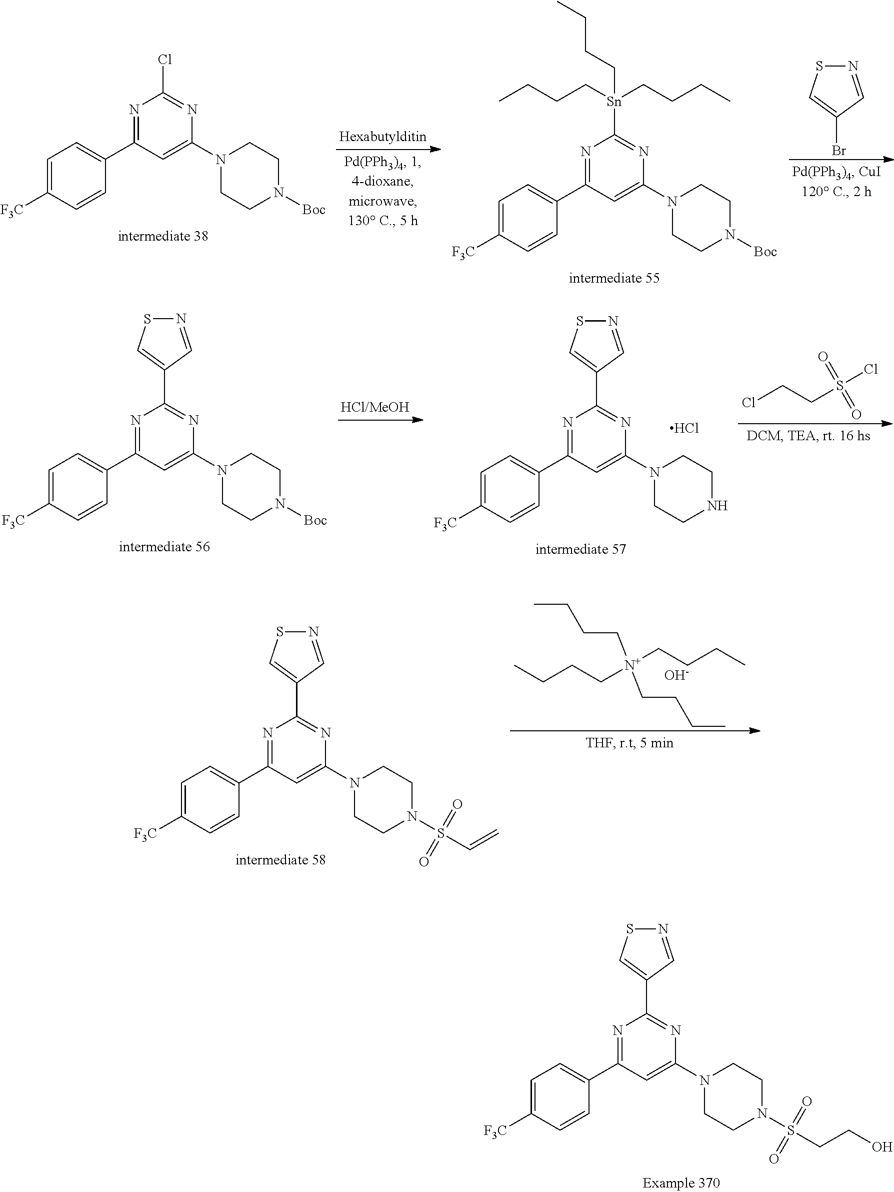

### Intermediate 55. tert-butyl 4-(6-(4-chlorophenyl)-2-(tributylstannyl)pyrimidin-4-yl)piperazine-1-carboxylate

To a solution of tert-butyl 4-(2-chloro-6-(4-(trifluoromethyl)phenyl)pyrimidin-4-yl)piperazine-1-carboxylate (652 mg, 1.47 mmol) in 1,4-dioxane (10 mL) was added Hexabutylditin (1.54 g, 2.65 mmol), Pd(PPh₃)₄ (255 mg, 0.221 mmol) at room temperature under Nitrogen. The reaction mixture was heated and stirred at 130° C. for 5 hr under Nitrogen. The residue was cooled to room temperature and KF aq was added to the reaction mixture stirring for another 30 min. The mixture was concentrated in vacuo and the residue was purified via silica gel column chromatography (Petroleum ether/EtOAc=5/1; V/V) to afford 1.4 g of the title compound.

¹H NMR (400 MHz, CDCl₃) δ [ppm]=0.94-0.84 (m, 9H), 1.15 (dd, J=9.2, 6.9 Hz, 5H), 1.43-1.31 (m, 7H), 1.50 (s, 9H), 1.74-1.58 (m, 6H), 3.61-3.49 (m, 4H), 3.78-3.67 (m, 4H), 6.75 (s, 1H), 7.26 (s, 1H), 7.70 (d, J=8.2 Hz, 2H), 8.13 (d, J=8.1 Hz, 2H); MS (ESI, m/z): 699.3 [M+H]⁺

### Intermediate 56. tert-butyl 4-(2-(isothiazol-4-yl)-6-(4-(trifluoromethyl)phenyl)pyrimidin-4-yl)piperazine-1-carboxylate

To a solution of tert-butyl 4-(2-(tributylstannyl)-6-(4-(trifluoromethyl)phenyl)pyrimidin-4-yl)piperazine-1-carboxylate (467 mg, 0.67 mmol) in 1,4-dioxane (10 mL) was added 4-bromoisothiazole (91.56 mg, 0.56 mmol), Pd(PPh₃)₄ (116.2 mg, 0.1005 mmol) and CuI (19.2 mg, 0.1005 mmol) at room temperature under Nitrogen atmosphere. The reaction mixture was stirred at 120° C. via microwave irradiation for 3 hr under nitrogen atmosphere. LCMS showed the starting material consumed completely. After the reaction mixture was cooled to room temperature and 5 mL of Sat. KF aq. was added to the reaction mixture stirring for another 30 min. The mixture was concentrated in vacuo and the residue was purified via silica gel column chromatography (DCM/MeOH=10/1; V/V) to afford 141 mg of the title compound.

MS (ESI, m/z): 492.2 [M+H]⁺

### Intermediate 57. 4-(4-(piperazin-1-yl)-6-(4-(trifluoromethyl)phenyl)pyrimidin-2-yl)isothiazole hydrochloride

To a solution of tert-butyl 4-(2-(isothiazol-4-yl)-6-(4-(trifluoromethyl)phenyl)pyrimidin-4-yl)piperazine-1-carboxylate (141 mg, 0.28 mmol) in Hydrogen chloride-Methanol solution (4 M HCl gas in MeOH, 5 mL) was stirred at room temperature for 2 h. The mixture was concentrated in vacuo to give 160 mg of the title compound.

MS (ESI, m/z): 392.2 [M+H]⁺

### Intermediate 58. 4-(4-(4-(trifluoromethyl)phenyl)-6-(4-(vinylsulfonyl)piperazin-1-yl)pyrimidin-2-yl)isothiazole

To a solution of 4-(4-(piperazin-1-yl)-6-(4-(trifluoromethyl)phenyl)pyrimidin-2-yl)isothiazole (110 mg, 0.22 mmol) in 10 mL of dichloromethan was added TEA (111 mg, 1.1 mmol) at 0° C. 2-chloroethanesulfonyl chloride (43.8 mg, 0.27 mmol) was added dropwise to the above reaction mixture at 0° C. The reaction mixture was stirred at room temperature for 16 h. TLC showed the starting material consumed completely. The mixture was concentrated in vacuo and the residue was purified via silica gel column chromatography (DCM/MeOH=10/1; V/V) to afford 80 mg of the title compound.

MS (ESI, m/z): 482.1 [M+H]⁺

### Example 370. 2-((4-(2-(isothiazol-4-yl)-6-(4-(trifluoromethyl)phenyl)pyrimidin-4-yl)piperazin-1-yl)sulfonyl)ethanol

To a solution of 4-(4-(4-(trifluoromethyl)phenyl)-6-(4-(vinylsulfonyl)piperazin-1-yl)pyrimidin-2-yl)isothiazole (100 mg, 0.2 mmol) in 5 mL THF was added tetrabutylammonium hydroxide (208 mg, 0.2 mmol) at room temperature. The reaction mixture was stirred at 50° C. for 16 h. TLC showed the starting material consumed completely. The mixture was cooled to room temperature. The mixture was concentrated in vacuo and the residue was purified via silica gel column chromatography (DCM/MeOH=10/1; V/V) to afford 80 mg of the title compound.

¹H NMR (400 MHz, DMSO-d₆) δ [ppm]=3.25 (t, J=6.1 Hz, 2H), 3.34 (s, 4H), 3.76 (q, J=6.0 Hz, 2H), 3.97 (s, 4H), 5.04 (t, J=5.4 Hz, 1H), 7.47 (s, 1H), 7.90 (d, J=8.3 Hz, 2H), 8.52 (d, J=8.1 Hz, 2H), 9.30 (s, 1H), 9.75 (s, 1H); MS (ESI, m/z): 500.2 [M+H]⁺

### Example 371. (S)-2-(4-(6-(4-chlorophenyl)-2-(pyridin-3-yl)pyrimidin-4-yl)piperazin-2-yl)ethanol

Using (S)-2-(piperazin-2-yl)ethan-1-ol, the title compound was obtained as described for the example 1 (Scheme 1. General procedure A.).

¹H NMR (400 MHz, CDCl₃) δ [ppm]=1.82-1.73 (m, 2H), 2.97-2.85 (m, 2H), 3.15-3.02 (m, 2H), 3.22-3.16 (m, 1H), 3.96-3.84 (m, 2H), 4.45 (dd, J=31.6, 11.1 Hz, 2H), 6.82 (s, 1H), 7.39 (dd, J=7.9, 4.8 Hz, 1H), 7.49-7.45 (m, 2H), 8.09-8.04 (m, 2H), 8.69 (dd, J=4.8, 1.7 Hz, 1H), 8.74 (dt, J=8.0, 1.9 Hz, 1H), 9.68 (d, J=1.5 Hz, 1H); MS (ESI, m/z): 396.2 [M+H]⁺

### Example 372. (S)-4-(5-(6-(3-hydroxypyrrolidin-1-yl)-2-(pyridin-3-yl)pyrimidin-4-yl)pyridin-2-yl)morpholin-3-one

Using (6-(3-oxomorpholino)pyridin-3-yl)boronic acid, the title compound was obtained as described for the example 296 (Scheme 4. General procedure D.).

¹H NMR (400 MHz, DMSO-d₆) δ [ppm]=1.84-2.21 (m, 2H), 3.62-3.73 (m, 4H), 4.04 (s, 4H), 4.32 (s, 2H), 4.47 (d, J=21.9 Hz, 1H), 5.09 (d, J=38.1 Hz, 1H), 7.07 (s, 1H), 7.54 (dd, J=7.9, 4.8 Hz, 1H), 8.22 (d, J=8.8 Hz, 1H), 8.72-8.63 (m, 2H), 8.75 (d, J=7.9 Hz, 1H), 9.33 (d, J=2.0 Hz, 1H), 9.60 (s, 1H); MS (ESI, m/z): 419.0 [M+H]⁺

### Example 373. (S)-3-(4-(3-fluoro-4-morpholinophenyl)-6-(3-hydroxypyrrolidin-1-yl)pyrimidin-2-yl)pyridin-2-ol

Scheme for the preparation of the Compound of Example 373:

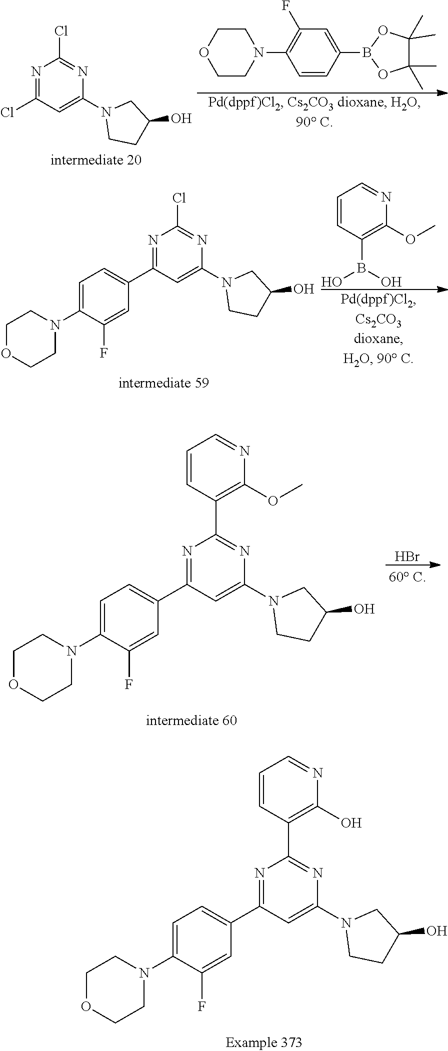

### Intermediate 59. (S)-1-(2-chloro-6-(3-fluoro-4-morpholinophenyl)pyrimidin-4-yl)pyrrolidin-3-ol

To a solution of (S)-1-(2,6-dichloropyrimidin-4-yl)pyrrolidin-3-ol (500 mg, 2.1 mmol) in dioxane (10 mL) and H₂O (2 mL) were added 4-(2-fluoro-4-(4,4,5,5-tetramethyl-1,3,2-dioxaborolan-2-yl)phenyl)morpholine (789 mg, 2.56 mmol), Cs₂CO₃ (1.39 g, 4.2 mmol) and Pd(dppf)Cl₂ (174 mg, 0.21 mmol) at room temperature. The mixture was degassed and purged with under Nitrogen three times. The reaction mixture was heated and stirred at 90° C. for 16 h under Nitrogen atmosphere. LCMS showed the reaction was complete. The reaction mixture was cooled to room temperature. The mixture was filtered. The filtrate was concentrated in vacuo.

The residue was purified via silica gel column chromatography (Petroleum ether/EtOAc=1/2; V/V) to give 220 mg of the title compound.

MS (ESI, m/z): 379.0 [M+H]⁺

### Intermediate 60. (S)-1-(6-(3-fluoro-4-morpholinophenyl)-2-(2-methoxypyridin-3-yl)pyrimidin-4-yl)pyrrolidin-3-ol

To a solution of (S)-1-(2-chloro-6-(3-fluoro-4-morpholinophenyl)pyrimidin-4-yl)pyrrolidin-3-ol (120 mg, 0.32 mmol) in dioxane (3 mL) and H₂O (0.5 mL) were added (2-methoxypyridin-3-yl)boronic acid (72 mg, 0.48 mmol), Cs₂CO₃ (206 mg, 0.63 mmol) and Pd(dppf)Cl₂ (25 mg, 0.031 mmol) at room temperature. The mixture was degassed and purged with under Nitrogen three times. The reaction mixture was heated and stirred at 90° C. for 16 h under Nitrogen atmosphere. LCMS showed the reaction was complete. The reaction mixture was cooled to room temperature and filtered. The filtrate was concentrated in vacuo. The residue was purified via silica gel column chromatography (Petroleum ether/EtOAc=1/2; V/V) to give 150 mg of the title compound.

MS (ESI, m/z): 452.1 [M+H]⁺

### Example 373. (S)-3-(4-(3-fluoro-4-morpholinophenyl)-6-(3-hydroxypyrrolidin-1-yl)pyrimidin-2-yl)pyridin-2-ol

To a solution of (S)-1-(6-(3-fluoro-4-morpholinophenyl)-2-(2-methoxypyridin-3-yl)pyrimidin-4-yl)pyrrolidin-3-ol was dissolved in a solution of HBr in H₂O (5 mL, 48%). The reaction mixture was heated and stirred at 100° C. for 16 hr. LCMS showed the reaction was complete. The reaction mixture was concentrated in vacuo. The residue was purified via column chromatography (DCM/MeOH=10/1; V/V) to afford 41.8 mg of the title compound (Scheme 5. General procedure E.).

¹H NMR (400 MHz, DMSO-d₆) δ [ppm]=1.99-2.17 (m, 2H), 3.19-3.21 (m, 4H), 3.58-3.99 (m, 8H), 4.51 (d, J=24.8 Hz, 1H), 5.26 (d, J=34.2 Hz, 1H), 6.76-6.91 (m, 1H), 7.17 (d, J=6.8 Hz, 1H), 7.28 (t, J=8.7 Hz, 1H), 7.68-7.84 (m, 1H), 7.96 (d, J=14.5 Hz, 1H), 8.07-8.25 (m, 1H), 8.78-8.99 (m, 1H); MS (ESI, m/z): 438.2 [M+H]⁺

### Example 374. (S)-1-(6-(4-((2-(dimethylamino)ethyl)amino)phenyl)-2-(pyridin-3-yl)pyrimidin-4-yl)pyrrolidin-3-ol

Using N¹,N¹-dimethyl-N²-(4-(4,4,5,5-tetramethyl-1,3,2-dioxaborolan-2-yl)phenyl)ethane-1,2-diamine and separation method of PREP. HPLC, the title compound was obtained as described for the example 296 (Scheme 4. General procedure D.).

¹H NMR (400 MHz, DMSO-d₆) δ [ppm]=1.89-2.13 (m, 2H), 2.20 (s, 6H), 2.47 (t, J=6.6 Hz, 2H), 3.18 (dd, J=12.2, 6.3 Hz, 2H), 3.46-3.90 (m, 4H), 4.44 (s, 1H), 5.04 (s, 1H), 5.95 (t, J=5.3 Hz, 1H), 6.70 (d, J=8.7 Hz, 2H), 6.75 (s, 1H), 7.52 (dd, J=7.9, 4.8 Hz, 1H), 8.07 (d, J=8.6 Hz, 2H), 8.67 (d, J=3.6 Hz, 1H), 8.72 (d, J=7.9 Hz, 1H), 9.57 (s, 1H); MS (ESI, m/z): 405.0 [M+H]⁺

### Example 375. (S)-1-(6-(4-(2-(dimethylamino)ethoxy)phenyl)-2-(pyridin-3-yl)pyrimidin-4-yl)pyrrolidin-3-ol

Using N,N-dimethyl-2-(4-(4,4,5,5-tetramethyl-1,3,2-dioxaborolan-2-yl)phenoxy)ethan-1-amine and separation method of PREP. HPLC, the title compound was obtained as described for the example 296 (Scheme 4. General procedure D.).

¹H NMR (400 MHz, DMSO-d₆) δ [ppm]=1.91-2.06 (m, 2H), 2.35 (s, 6H), 2.80-2.83 (m, 2H), 3.26-3.70 (m, 4H), 4.20 (t, J=5.6 Hz, 2H), 4.44-4.47 (m, 1H), 5.08 (d, J=32.8 Hz, 1H), 6.90 (s, 1H), 7.09 (d, J=8.8 Hz, 2H), 7.54 (dd, J=7.9, 4.8 Hz, 1H), 8.26 (d, J=8.8 Hz, 2H), 8.68 (dd, J=4.7, 1.5 Hz, 1H), 8.73 (d, J=7.9 Hz, 1H), 9.59 (d, J=1.2 Hz, 1H); MS (ESI, m/z): 406.2 [M+H]⁺

### Example 376. (S)-1-(6-(4-((2-hydroxyethyl)amino)phenyl)-2-(pyridin-3-yl)pyrimidin-4-yl)pyrrolidin-3-ol

Scheme for the preparation of the Compound of Example 376:

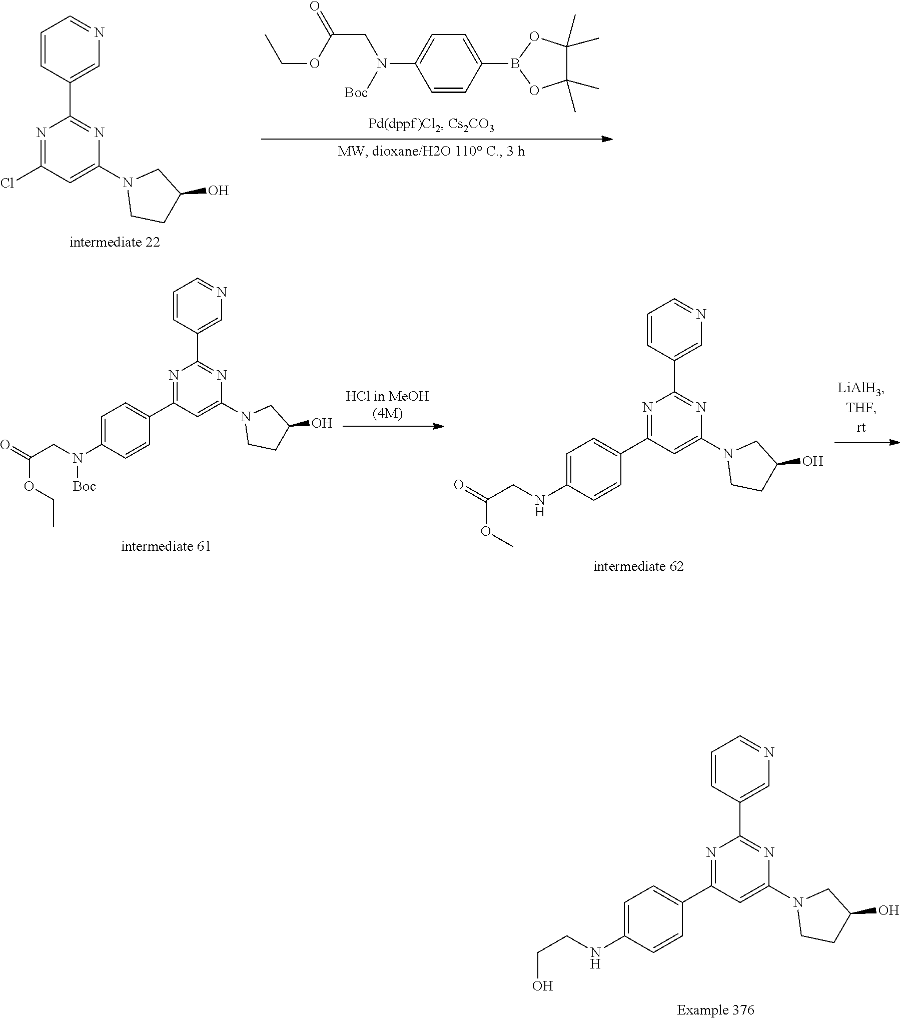 [SMILES: O[C@H]1CCN(c2cc(Cl)nc(-c3cccnc3)n2)C1 ; CCOC(=O)CN(C(=O)OC(C)(C)C)c1ccc(B2OC(C)(C)C(C)(C)O2)cc1 ; CCOC(=O)CN(C(=O)OC(C)(C)C)c1ccc(-c2cc(N3CC[C@H](O)C3)nc(-c3cccnc3)n2)cc1 ; COC(=O)CNc1ccc(-c2cc(N3CC[C@H](O)C3)nc(-c3cccnc3)n2)cc1 ; OCCNc1ccc(-c2cc(N3CC[C@H](O)C3)nc(-c3cccnc3)n2)cc1]

### Intermediate 61. ethyl (S)—N-(tert-butoxycarbonyl)-N-(4-(6-(3-hydroxypyrrolidin-1-yl)-2-(pyridin-3-yl)pyrimidin-4-yl)phenyl)glycinate

To a solution of (S)-1-(6-chloro-2-(pyridin-3-yl)pyrimidin-4-yl)pyrrolidin-3-ol (250 mg, 0.91 mmol) in 1,4-dioxane (5 mL) and H₂O (1 mL) was added ethyl N-(tert-butoxycarbonyl)-N-(4-(4,4,5,5-tetramethyl-1,3,2-dioxaborolan-2-yl)phenyl)glycinate (513 mg, 1.27 mmol), Cs₂CO₃ (883 mg, 2.72 mmol) and Pd(dppf)Cl₂ (147 mg, 0.18 mmol) at room temperature under nitrogen.

The reaction mixture was stirred at 110° C. via microwave irradiation for 2.5 h under Nitrogen atmosphere. LCMS showed the starting material was consumed and produced the desired compound. The reaction mixture was concentrated in vacuo. The residue was purified via reverse phase column chromatography (MeOH/H₂O=1/1; V/V) to afford 440 mg of the title compound.

MS (ESI, m/z): 520.0 [M+H]⁺

### Intermediate 62. methyl (S)-(4-(6-(3-hydroxypyrrolidin-1-yl)-2-(pyridin-3-yl)pyrimidin-4-yl)phenyl)glycinate

To a solution of (S)-ethyl 2-((tert-butoxycarbonyl)(4-(6-(3-hydroxypyrrolidin-1-yl)-2-(pyridin-3-yl)pyrimidin-4-yl)phenyl)amino)acetate (240 mg, 0.46 mmol) in 4N hydrochloric acid methanol solution (10 mL) was stirred at room temperature for 2 hr. LC-MS showed the starting material was consumed and produced the desired compound. The reaction mixture was concentrated in vacuo and used in the next step without further purification.

MS (ESI, m/z): 405.8 [M+H]⁺

### Example 376. (S)-1-(6-(4-((2-hydroxyethyl)amino)phenyl)-2-(pyridin-3-yl)pyrimidin-4-yl)pyrrolidin-3-ol

To a solution of methyl (S)-(4-(6-(3-hydroxypyrrolidin-1-yl)-2-(pyridin-3-yl)pyrimidin-4-yl)phenyl)glycinate (200 mg, 0.49 mmol) in THF (10 mL) was added LiAlH₄ (56 mg, 1.48 mmol) at 0° C. The reaction mixture was stirred at room temperature for 1 hr. LCMS showed the starting material was consumed and produced the desired compound. The mixture was quenched with H₂O. The aqueous layer was extracted with EtOAc (10 mL×2). The combined organic layer was washed with brine, dried over Na₂SO₄. The mixture was filtered and concentrated in vacuo. The residue was purified via prep-HPLC to afford 13.8 mg of the title compound as a white solid.

¹H NMR (400 MHz, DMSO-d₆) δ [ppm]=1.88-2.14 (m, 2H), 3.18 (q, J=5.8 Hz, 2H), 3.48-3.95 (m, 6H), 4.44 (s, 1H), 4.74 (s, 1H), 5.04 (s, 1H), 6.09 (t, J=5.5 Hz, 1H), 6.70 (d, J=8.8 Hz, 2H), 6.74 (s, 1H), 7.52 (dd, J=7.8, 4.8 Hz, 1H), 8.06 (d, J=8.7 Hz, 2H), 8.18 (s, 1H), 8.67 (d, J=3.6 Hz, 1H), 8.72 (dt, J=7.9, 1.8 Hz, 1H), 9.58 (s, 1H); MS (ESI, m/z): 378.0 [M+H]⁺

In Vitro XRE-Luciferase Reporter Assay (In Vitro Assay 1, 2, 3) AhR activation leads the induction of target gene expression such as CYP1A1 and CYPIB1 by AhR binding to AhR-responsive DNA elements also known as xenobiotics responsive elements (XREs). The assay for measuring AhR activity herein is the luciferase assay using cell lines transfected with luciferase reporter plasmid containing XREs at the upstream of the reporter gene. Cells transfected with XRE-luciferase reporter (XRE-Luc), plasmid drive luciferase activity reflecting activation and inhibition of AhR in the cells. In addition to transfection with XRE-reporter vector, cells were co-transfected with Nano-luciferase reporter gene construct (Nano-Luc), containing constitutively active promoter as internal control. Kynurenine (an endogenous AhR agonist), was used to stimulate cells to test antagonistic properties of the compounds. The half-maximal inhibitory concentration (IC₅₀), or half-maximal effective concentration (EC₅₀), value was calculated using nonlinear regression (four parameters), with Prism8.0 software (GraphPad).

In Vitro Assay 1: Antagonism in Human Cell Line

HepG2 (human hepatoma cell line) cell line with a XRE-luciferase reporter either transiently or stably (Invivogen) were plated in complete medium and incubated at 37° C. in a CO₂ incubator. After 24 hours, cells were treated with kynurenine (50* or 200 μM) alone (negative control) or with test compounds for 6 hours. Luciferase activity was measured with a commercial kit such as the Promega Luciferase kit or Invivogen Luciferase kit. Relative luciferase activity (Firefly/Nano-Luc) was used to calculate IC₅₀ values. The relative luciferase activity was further normalized with kynurenine alone group as the maximum control and the vehicle group as the minimum control. The AhR antagonistic potency of the example compounds is listed in Table 1 below. (IC₅₀ values are grouped as A, B, C and D, whereby A: IC₅₀<0.01 μM; B: 0.01<IC₅₀<0.1 μM; C: 0.1<IC₅₀<1.0 μM; D: IC₅₀>1.0 μM)

In Vitro Assay 2: Antagonism in Mouse Cell Line

Hepa1c1c7 (murine liver cancer cell line) cells co-transfected with XRE-Luc and Nano-Luc plasmids were plated in complete medium and incubated overnight at 37° C. in a CO₂ incubator. Following incubation, cells were treated with AhR activating ligands such as kynurenic acid, kynurenine (#) with or without test compounds for 6 hours. Firefly luciferase and Nano-luciferase activity was measured using Nano-glo Luciferase kit (Promega) and relative luciferase activity (Firefly/Nano-Luc) was used to calculate IC₅₀ values. The relative luciferase activity was further normalized with agonists alone group as the maximum control and the vehicle group as the minimum control. The AhR antagonistic potency of the example compounds is listed in Table 1 below. (IC₅₀ Values are grouped as A, B, C and D, whereby A: IC₅₀<0.01 μM; B: 0.01<IC₅₀<0.1 μM; C: 0.1<IC₅₀<1.0 μM; D: IC₅₀>1.0 μM)

In Vitro Assay 3: Agonism in Human Cell Line

HepG2 (human hepatoma cell line) cells co-transfected with XRE-Luc and Nano-Luc plasmids were plated in tryptophan free medium containing 1% of dialyzed fetal bovine serum and incubated overnight at 37° C. in a CO₂ incubator. After 24 hours, cells were treated for 6 hours with test compounds or not. Firefly luciferase and Nano-luciferase activity was measured using Nano-glo Luciferase kit (Promega) and relative luciferase activity (Firefly/Nano-Luc) was used to calculate EC₅₀ values. As a positive control, cells were incubated with TCDD.

(EC₅₀ Values are grouped as A, B, C and D, whereby A: EC₅₀<0.1 μM; B: 0.1<EC₅₀<1.0 μM; C: 1.0<EC₅₀<10 μM; D: EC₅₀>10 μM)

TABLE 1
Results of in vitro XRE-luciferase activity assay.

| | Assay 1: AhR- | Assay 2: AhR- | Assay 3: AhR- |
|---|---|---|---|
| | Luc Human | Luc Mouse | Luc Human |
| | Antagonism | Antagonism | Agonism |
| Example | (IC₅₀, nM) | (IC₅₀, nM) | (EC₅₀, nM) |
| Example 1 | B* | C# | 12.63 (A) |
| Example 2 | C* | — | — |
| Example 3 | C* | — | — |
| Example 4 | C* | — | — |
| Example 5 | D* | — | — |
| Example 6 | C* | — | — |
| Example 7 | D* | — | — |
| Example 8 | C* | — | — |
| Example 9 | C* | — | — |
| Example 10 | C* | — | — |
| Example 11 | B* | — | — |
| Example 12 | C* | — | — |
| Example 13 | D* | — | — |
| Example 14 | D* | — | — |
| Example 15 | D* | — | — |
| Example 16 | D* | — | — |
| Example 17 | D* | — | — |
| Example 18 | D* | — | — |
| Example 19 | C* | — | — |
| Example 20 | D* | — | — |
| Example 21 | C* | — | — |
| Example 22 | C* | — | — |
| Example 23 | D* | — | — |
| Example 24 | C* | — | — |
| Example 25 | D* | — | — |
| Example 26 | D* | — | — |
| Example 27 | A* | — | — |
| Example 28 | D* | — | — |
| Example 29 | B* | — | — |
| Example 30 | B* | — | — |
| Example 31 | C* | — | — |
| Example 32 | C* | — | — |
| Example 33 | C* | — | — |
| Example 34 | B* | — | — |
| Example 35 | D* | — | — |
| Example 36 | B* | — | — |
| Example 37 | D* | — | — |
| Example 38 | A* | — | — |
| Example 39 | B* | — | — |
| Example 40 | B* | — | — |
| Example 41 | A* | — | — |
| Example 42 | A* | — | — |
| Example 43 | A* | — | — |
| Example 44 | A* | — | — |
| Example 45 | B* | — | — |
| Example 46 | C* | — | — |
| Example 47 | D* | — | — |
| Example 48 | D* | — | — |
| Example 49 | D* | — | — |
| Example 50 | D* | — | — |
| Example 51 | C* | — | — |
| Example 52 | C* | — | — |
| Example 53 | D* | — | — |
| Example 54 | D* | — | — |
| Example 55 | D* | — | — |
| Example 56 | C* | — | — |
| Example 57 | D* | — | — |
| Example 58 | D* | — | — |
| Example 59 | D* | — | — |
| Example 60 | D* | — | — |
| Example 61 | D* | — | — |
| Example 62 | D* | — | — |
| Example 63 | D* | — | — |
| Example 64 | C* | — | — |
| Example 65 | C* | — | — |
| Example 66 | D* | — | — |
| Example 67 | C* | — | — |
| Example 68 | B* | — | — |
| Example 69 | C* | — | — |
| Example 70 | D* | — | 757.7 (B) |
| Example 71 | B* | — | — |
| Example 72 | D* | — | — |
| Example 73 | D* | — | — |
| Example 74 | C* | — | — |
| Example 75 | D* | — | — |
| Example 76 | D* | — | — |
| Example 77 | D* | — | — |
| Example 78 | D* | — | — |
| Example 79 | D* | — | — |
| Example 80 | D* | — | — |
| Example 81 | A* | — | — |
| Example 82 | A* | — | — |
| Example 83 | B* | — | — |
| Example 84 | D* | — | — |
| Example 85 | C* | — | — |
| Example 86 | B* | — | — |
| Example 87 | B* | — | — |
| Example 88 | D* | — | — |
| Example 89 | B* | — | — |
| Example 90 | B* | — | — |
| Example 91 | B* | — | — |
| Example 92 | B* | C# | >30,000 (D) |
| Example 93 | B* | — | — |
| Example 94 | B* | C# | >30,000 (D) |
| Example 95 | B* | — | — |
| Example 96 | A* | — | — |
| Example 97 | A* | — | — |
| Example 98 | D* | — | — |
| Example 99 | B* | — | — |
| Example 100 | D* | — | — |
| Example 101 | C* | — | — |
| Example 102 | C* | — | — |
| Example 103 | D* | — | — |
| Example 104 | D* | — | — |
| Example 105 | A* | C# | >30,000 (D) |
| Example 106 | B* | — | — |
| Example 107 | A | B# | >30,000 (D) |
| Example 108 | A* | — | — |
| Example 109 | D* | — | — |
| Example 110 | A* | — | — |
| Example 111 | D* | — | — |
| Example 112 | D* | — | — |
| Example 113 | D* | — | — |
| Example 114 | D* | — | — |
| Example 115 | D* | — | — |
| Example 116 | D* | — | — |
| Example 117 | C* | — | — |
| Example 118 | B* | — | 1580 (C) |
| Example 119 | D* | — | — |
| Example 120 | A* | — | — |
| Example 121 | D* | — | — |
| Example 122 | B* | — | — |
| Example 123 | D* | — | — |
| Example 124 | D* | — | — |
| Example 125 | C* | — | — |
| Example 126 | D* | — | — |
| Example 127 | D* | — | — |
| Example 128 | D* | — | — |
| Example 129 | D* | — | — |
| Example 130 | D* | — | — |
| Example 131 | D* | — | — |
| Example 132 | C* | — | — |
| Example 133 | D* | — | — |
| Example 134 | D* | — | — |
| Example 135 | B* | — | — |
| Example 136 | C* | — | — |
| Example 137 | A | A | >30,000 (D) |
| Example 138 | A* | — | — |
| Example 139 | B* | — | — |
| Example 140 | D* | — | — |
| Example 141 | D* | — | — |
| Example 142 | C* | — | — |
| Example 143 | A* | C# | >30,000 (D) |
| Example 144 | B* | — | 55.94 (A) |
| Example 145 | C* | — | — |
| Example 146 | B* | — | — |
| Example 147 | A* | — | — |
| Example 148 | B* | — | — |
| Example 149 | D* | — | — |
| Example 150 | A* | — | — |
| Example 151 | D* | — | — |
| Example 152 | C* | — | — |
| Example 153 | D* | — | — |
| Example 154 | A* | B# | >30,000 (D) |
| Example 155 | D* | — | — |
| Example 156 | A* | — | — |
| Example 157 | D* | — | — |
| Example 158 | C* | — | — |
| Example 159 | C* | — | — |
| Example 160 | D* | — | — |
| Example 161 | D* | — | — |
| Example 162 | A* | C# | >30,000 (D) |
| Example 163 | B* | — | — |
| Example 164 | C* | — | — |
| Example 165 | D* | — | — |
| Example 166 | B* | — | — |
| Example 167 | D* | — | — |
| Example 168 | D* | — | — |
| Example 169 | D* | — | — |
| Example 170 | B* | — | >30,000 (D) |
| Example 171 | B* | — | — |
| Example 172 | A* | — | — |
| Example 173 | C* | — | — |
| Example 174 | C* | — | — |
| Example 175 | B* | — | — |
| Example 176 | D* | — | — |
| Example 177 | B* | — | — |
| Example 178 | C* | — | — |
| Example 179 | C* | — | — |
| Example 180 | D* | — | — |
| Example 181 | C* | — | — |
| Example 182 | C* | — | — |
| Example 183 | C* | — | — |
| Example 184 | C* | — | — |
| Example 185 | C* | — | — |
| Example 186 | D* | — | — |
| Example 187 | C* | — | — |
| Example 188 | C* | — | — |
| Example 189 | C* | — | — |
| Example 190 | B* | — | — |
| Example 191 | B* | — | — |
| Example 192 | C* | — | — |
| Example 193 | C* | — | — |
| Example 194 | A* | — | — |
| Example 195 | D* | — | — |
| Example 196 | B* | — | — |
| Example 197 | D* | — | — |
| Example 198 | B* | — | — |
| Example 199 | C* | — | — |
| Example 200 | D* | — | — |
| Example 201 | D* | — | — |
| Example 202 | D* | — | — |
| Example 203 | C* | — | — |
| Example 204 | D* | — | — |
| Example 205 | D* | — | — |
| Example 206 | D* | — | — |
| Example 207 | D* | — | — |
| Example 208 | D* | — | — |
| Example 209 | D* | — | — |
| Example 210 | B* | — | — |
| Example 211 | B* | — | — |
| Example 212 | B* | — | — |
| Example 213 | B* | — | — |
| Example 214 | B* | — | — |
| Example 215 | B* | — | — |
| Example 216 | B* | — | — |
| Example 217 | B* | — | — |
| Example 218 | A* | — | — |
| Example 219 | A* | — | — |
| Example 220 | B* | — | — |
| Example 221 | B* | — | — |
| Example 222 | A* | — | — |
| Example 223 | A* | — | — |
| Example 224 | A* | — | >30,000 (D) |
| Example 225 | A* | — | >30,000 (D) |
| Example 226 | A | B# | >30,000 (D) |
| Example 227 | B | — | — |
| Example 228 | B | — | — |
| Example 229 | B | — | >30,000 (D) |
| Example 230 | B* | B# | — |
| Example 231 | A | B# | >30,000 (D) |
| Example 232 | A* | D# | — |
| Example 233 | A* | — | — |
| Example 234 | A* | — | — |
| Example 235 | A* | B# | 3563 (C) |
| Example 236 | A* | C# | 40.06 (A) |
| Example 237 | B | — | — |
| Example 238 | B | — | — |
| Example 239 | A | A | >30,000 (D) |
| Example 240 | A | — | — |
| Example 241 | B | — | — |
| Example 242 | B | — | — |
| Example 243 | A | B# | — |
| Example 244 | D | — | — |
| Example 245 | B | — | — |
| Example 246 | A | — | >30,000 (D) |
| Example 247 | B | — | — |
| Example 248 | B | — | — |
| Example 249 | B | — | — |
| Example 250 | A | — | — |
| Example 251 | A | — | — |
| Example 252 | A | — | — |
| Example 253 | A | — | — |
| Example 254 | A | — | — |
| Example 255 | B | — | — |
| Example 256 | A | — | — |
| Example 257 | A | — | — |
| Example 258 | A | A | — |
| Example 259 | A | — | — |
| Example 260 | A | — | >30,000 (D) |
| Example 261 | A | — | — |
| Example 262 | A | — | >30,000 (D) |
| Example 263 | A | — | — |
| Example 264 | B | — | — |
| Example 265 | A | — | — |
| Example 266 | A | — | — |
| Example 267 | A | — | — |
| Example 268 | A | A# | >30,000 (D) |
| Example 269 | A | — | — |
| Example 270 | A | — | — |
| Example 271 | A | — | — |
| Example 272 | B | — | — |
| Example 273 | B | — | >30,000 (D) |
| Example 274 | D | — | — |
| Example 275 | B | — | — |
| Example 276 | A | — | >30,000 (D) |
| Example 277 | C | — | — |
| Example 278 | B | — | >30,000 (D) |
| Example 279 | A | — | — |
| Example 280 | A | B | — |
| Example 281 | A | — | — |
| Example 282 | A | C | >30,000 (D) |
| Example 283 | A | B | >30,000 (D) |
| Example 284 | A | — | — |
| Example 285 | B | — | — |
| Example 286 | C | — | — |
| Example 287 | C | — | — |
| Example 288 | C | — | — |
| Example 289 | A | — | >30,000 (D) |
| Example 290 | D | — | — |
| Example 291 | D | — | — |
| Example 292 | D | — | — |
| Example 293 | D | — | — |
| Example 294 | C | — | — |
| Example 295 | D | — | — |
| Example 296 | D | — | — |
| Example 297 | D | — | — |
| Example 298 | B | — | — |
| Example 299 | D | — | — |
| Example 300 | C | — | — |
| Example 301 | C | — | — |
| Example 302 | D | — | — |
| Example 303 | A | — | — |
| Example 304 | A | — | — |
| Example 305 | D | — | — |
| Example 306 | B | — | — |
| Example 307 | B | — | — |
| Example 308 | C | — | — |
| Example 309 | B | — | — |
| Example 310 | C | — | — |
| Example 311 | C | — | — |
| Example 312 | — | D | — |
| Example 313 | D | — | — |
| Example 314 | A | — | >30,000 (D) |
| Example 315 | A | — | — |
| Example 316 | A | — | — |
| Example 317 | D | — | — |
| Example 318 | B | — | — |
| Example 319 | B | — | — |
| Example 320 | B | — | — |
| Example 321 | C | — | — |
| Example 322 | D | — | — |
| Example 323 | A | B# | >30,000 (D) |
| Example 324 | D | — | — |
| Example 325 | B | — | — |
| Example 326 | B | — | — |
| Example 327 | A | B# | 1724 (C) |
| Example 328 | B | — | — |
| Example 329 | C | — | — |
| Example 330 | B | — | — |
| Example 331 | A | — | — |
| Example 332 | D | — | — |
| Example 333 | D | — | — |
| Example 334 | D | — | — |
| Example 335 | A | — | — |
| Example 336 | C | — | — |
| Example 337 | A | — | — |
| Example 338 | C | — | — |
| Example 339 | B | — | — |
| Example 340 | A | — | — |
| Example 341 | D | — | — |
| Example 342 | D | — | — |
| Example 343 | D | — | — |
| Example 344 | A | — | — |
| Example 345 | C | — | — |
| Example 346 | B | — | — |
| Example 347 | D | — | — |
| Example 348 | B | — | — |
| Example 349 | A | — | — |
| Example 350 | A | — | — |
| Example 351 | D | — | — |
| Example 352 | A | B# | >30,000 (D) |
| Example 353 | B | — | — |
| Example 354 | D | — | — |
| Example 355 | C | — | — |
| Example 356 | B | — | — |
| Example 357 | A | — | — |
| Example 358 | C | — | — |
| Example 359 | B | — | — |
| Example 360 | D | — | — |
| Example 361 | A | — | >30,000 (D) |
| Example 362 | A | — | — |
| Example 363 | A | — | — |
| Example 364 | A | — | — |
| Example 365 | C | — | — |
| Example 366 | D | — | — |
| Example 367 | D | — | — |
| Example 368 | B | — | — |
| Example 369 | B | — | — |
| Example 370 | B | — | — |
| Example 371 | C | — | — |
| Example 372 | C | — | — |
| Example 373 | B | — | — |
| Example 374 | D | — | — |
| Example 375 | D | — | — |
| Example 376 | C | — | — |

In Vitro Assay 4: Endogenous AhR Activity Assay

HepG2 cells were seeded in 12-well plate (3×10⁵ cells/well). A day after seeding, the cells were treated with TCDD (10 nM) alone or with compounds (123 nM) for 4 hours. Total RNA was extracted using Trizol (Thermo Fisher Scientific). cDNA synthesis and quantitative RT-PCR (qRT-PCR) assays were performed using PrimeScript™ RT Master Mix (TAKARA) and TB Green™ Premix Ex Taq™ II (TAKARA) in accordance with manufacturer's instruction. For the measurement of endogenous AhR activity, relative mRNA levels of CYP1A1 and CYP1B1 were quantitated relative to β-actin mRNA by the comparative Ct (ΔΔCt) method. The percent inhibitions were calculated according to:

$$( 1 - \frac{\begin{matrix}Relative mRNA level of compound treated group - \\ Relative mRNA level of vehicle group\end{matrix}}{\begin{matrix}Relative mRNA level of TCDD treated group - \\ Relative mRNA level of vehicle group\end{matrix}} ) \times 100 \% = \% inhibition$$

The endogenous AhR antagonistic potency of the example compounds is listed in Table 2 below.

TABLE 2
Results of in vitro endogenous AhR activity assay.

| | CYP1A1 (% | CYP1B1 (% |
|---|---|---|
| Compound_ID | Inhibition) | Inhibition) |
| Example 1 | 60.03 | 91.96 |
| Example 27 | 68.37 | 90.33 |
| Example 42 | 98.78 | 99.27 |
| Example 43 | 97.13 | 99.97 |
| Example 94 | 63.08 | 92.13 |
| Example 105 | 59.20 | 88.37 |
| Example 137 | 91.56 | 97.70 |
| Example 146 | 41.75 | 77.22 |
| Example 226 | 107.66 | 101.03 |
| Example 228 | 107.33 | 101.04 |
| Example 229 | 107.68 | 100.99 |
| Example 231 | 105.66 | 100.71 |
| Example 232 | 100.01 | 99.82 |
| Example 235 | 105.90 | 100.84 |
| Example 239 | 107.55 | 101.15 |
| Example 243 | 89.74 | |
| Example 258 | 100.76 | |
| Example 268 | 101.81 | |
| Example 323 | 107.59 | 99.82 |
| Example 327 | 105.83 | 100.68 |
| Example 339 | 84.65 | |
| Example 349 | 97.59 | |
| Example 352 | 87.22 | |

1. A compound of Formula (I), or an enantiomer, diastereomer, racemate, solvate, hydrate, or pharmaceutically acceptable salt thereof: 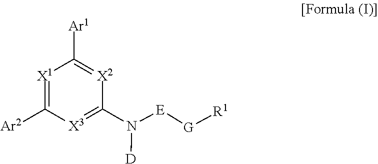
wherein:
X¹, X² and X³ are each independently CR², N or NR³;
Ar¹ and Ar² are each independently selected from substituted or unsubstituted mono- or bicyclic C₆₋₁₀ aryl, substituted or unsubstituted mono- or bicyclic C₅₋₁₀ heteroaryl and substituted or unsubstituted mono- or bicyclic C₃₋₁₀ heterocycloalkyl;
D is H, halo, cyano, hydroxy, amino, substituted or unsubstituted C₁₋₅ alkyl, mono- or bicyclic C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, mono- or bicyclic C₃₋₁₀ heterocycloalkyl, mono- or bicyclic C₃₋₁₀ heteroaryl,
E is absent (direct bond), amino, substituted or unsubstituted C₁₋₅ alkyl, mono- or bicyclic C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, mono- or bicyclic C₃₋₁₀ heterocycloalkyl, mono- or bicyclic C₃₋₁₀ heteroaryl,
or D and E, together with the atoms to which they are attached, are combined to form substituted or unsubstituted mono- or bicyclic C₃₋₁₀ heterocycloalkyl ring;
G is absent (direct bond), H, halo, cyano, hydroxy, amino, nitro, ether (—O—), thioether (—S—), sulfinyl (—SO—), sulfonyl(—SO₂—), sulfonylamido(—SO₂NR⁴—), aminosulfonyl(—NR⁴SO₂—), carbonyl(—(CO)—), amido(—(CO)NR⁴—), reverse amido(—NR⁴(CO)—), ester (—(CO)O—), substituted or unsubstituted mono- or bicyclic C₃₋₁₀ cycloalkyl, substituted or unsubstituted mono- or bicyclic C₃₋₁₀ heterocycloalkyl, substituted or unsubstituted mono- or bicyclic C₆₋₁₀ aryl and substituted or unsubstituted mono- or bicyclic C₅₋₁₀ heteroaryl;
R¹ is absent, H, halo, cyano, hydroxy, amino, N(R⁵)₂, OR⁵, substituted or unsubstituted C₁₋₅ alkyl, C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, substituted or unsubstituted mono- or bicyclic C₃₋₁₀ heterocycloalkyl and substituted or unsubstituted mono- or bicyclic C₅₋₁₀ heteroaryl;
R² is H, halo, cyano, hydroxy and C₁₋₃ alkyl;
R³ is H, halo, cyano, hydroxyl and amino; and
R⁴ is H, substituted or unsubstituted C₁₋₅ alkyl, substituted or unsubstituted C₁₋₅ alkoxy and substituted or unsubstituted C₁₋₅ alkyl carboxylic acid; and
R⁵ is H, substituted or unsubstituted C₁₋₅ alkyl, substituted or unsubstituted C₁₋₅ alkoxy and substituted or unsubstituted C₁₋₅ alkyl carboxylic acid.

2. The compound, or an enantiomer, diastereomer, racemate, solvate, hydrate, or pharmaceutically acceptable salt thereof according to claim 1,
wherein the Ar¹ is substituted or unsubstituted monocyclic C₅₋₇ heteroaryl comprising one or more hetero atoms selected from the group consisting of N, O and S.

3. The compound, or an enantiomer, diastereomer, racemate, solvate, hydrate, or pharmaceutically acceptable salt thereof according to claim 1, the Ar¹ is monocyclic C₅₋₆ heteroaryl comprising one or two hetero atoms selected from the group consisting of N, O and S, which is unsubstituted or substituted with C₁₋₃ alkyl.

4. The compound, or an enantiomer, diastereomer, racemate, solvate, hydrate, or pharmaceutically acceptable salt thereof according to claim 1, the Ar¹ is pyrazole or pyridine which is unsubstituted or substituted with methyl.

5. The compound, or an enantiomer, diastereomer, racemate, solvate, hydrate, or pharmaceutically acceptable salt thereof according to claim 1, the Ar² is mono- or bicyclic C₆₋₁₀ aryl comprising one or more hetero atoms selected from the group consisting of N, O and S, which is unsubstituted or substituted with halo.

6. The compound, or an enantiomer, diastereomer, racemate, solvate, hydrate, or pharmaceutically acceptable salt thereof according to claim 1, the Ar² is phenyl which is unsubstituted or substituted with chloro.

7. The compound, or an enantiomer, diastereomer, racemate, solvate, hydrate, or pharmaceutically acceptable salt thereof according to claim 1, the D is H or C₁₋₃ alkyl.

8. The compound, or an enantiomer, diastereomer, racemate, solvate, hydrate, or pharmaceutically acceptable salt thereof according to claim 1, the E is absent (direct bond), amino, substituted or unsubstituted C₁₋₄ alkyl, mono- or bicyclic C₃₋₈ cycloalkyl, C₁₋₄ alkylhydroxy, C₁₋₄ alkenylhydroxy, C₁₋₄ alkynylhydroxy, C₁₋₄ alkylamine, C₁₋₄ alkenylamine, C₁₋₄ alkynylamine, mono- or bicyclic C₃₋₈ heterocycloalkyl, mono- or bicyclic C₃₋₈ heteroaryl, wherein the mono- or bicyclic C₃₋₈ heterocycloalkyl and mono- or bicyclic C₃₋₈ heteroaryl comprises one or more heteroatoms selected from the group consisting of N, O and S.

9. The compound, or an enantiomer, diastereomer, racemate, solvate, hydrate, or pharmaceutically acceptable salt thereof according to claim 1, the D and E, together with the atoms to which they are attached, is combined to form substituted or unsubstituted mono- or bicyclic C₃₋₁₀ heterocycloalkyl ring one or more hetero atoms selected from the group consisting of N, O and S.

10. The compound, or an enantiomer, diastereomer, racemate, solvate, hydrate, or pharmaceutically acceptable salt thereof according to claim 9, the mono- or bicyclic C₃₋₁₀ heterocycloalkyl ring is unsubstituted or substituted pyrrolidine, piperidine, morpholine, thiomorpholine, piperazine, or octahydropyranopyridine.

11. The compound, or an enantiomer, diastereomer, racemate, solvate, hydrate, or pharmaceutically acceptable salt thereof according to claim 1, the G is absent (direct bond), H, halo, cyano, hydroxy, amino, nitro, ether (—O—), thioether (—S—), sulfinyl (—SO—), sulfonyl(—SO₂—), sulfonylamido(—SO₂NR⁴—), aminosulfonyl(—NR⁴SO₂—), carbonyl(—(CO)—), amido(—(CO)NR⁴—), reverse amido(—NR⁴(CO)—), ester (—(CO)O—), substituted or unsubstituted mono- or bicyclic C₃₋₈ cycloalkyl, substituted or unsubstituted mono- or bicyclic C₃-8 heterocycloalkyl, substituted or unsubstituted mono- or bicyclic C₆₋₁₀ aryl and substituted or unsubstituted mono- or bicyclic C₅₋₈ heteroaryl, wherein the mono- or bicyclic C₃₋₈ heterocycloalkyl and mono- or bicyclic C₅₋₈ heteroaryl comprises one or more heteroatoms selected from the group consisting of N, O and S.

12. The compound, or an enantiomer, diastereomer, racemate, solvate, hydrate, or pharmaceutically acceptable salt thereof according to claim 1, R¹ is absent, H, halo, cyano, hydroxy, amino, N(R⁵)₂, OR⁵, substituted or unsubstituted C₁₋₄ alkyl, C₃₋₈ cycloalkyl, C₁₋₄ alkylhydroxy, C₁₋₄ alkenylhydroxy, C₁₋₄ alkynylhydroxy, C₁₋₄ alkylamine, C₁₋₄ alkenylamine, C₁₋₄ alkynylamine, substituted or unsubstituted mono- or bicyclic C₃₋₈ heterocycloalkyl and substituted or unsubstituted mono- or bicyclic C₅₋₈ heteroaryl, phosphate, substituted or unsubstituted C₁₋₃ alkyl phosphate, wherein the mono- or bicyclic C₃₋₈ heterocycloalkyl and mono- or bicyclic C₅₋₈ heteroaryl comprises one or more heteroatoms selected from the group consisting of N, O and S.

13. The compound, or an enantiomer, diastereomer, racemate, solvate, hydrate, or pharmaceutically acceptable salt thereof according to claim 1, wherein the compound is selected from any one of the compounds 1 to 376:
1. (R)-2-((6-(4-chlorophenyl)-2-(pyridin-3-yl)pyrimidin-4-yl)amino)butan-1-ol
2. (S)-2-((6-(4-chlorophenyl)-2-(pyridin-3-yl)pyrimidin-4-yl)amino)propan-1-ol
3. (S)-2-((4-(4-chlorophenyl)-6-(pyridin-3-yl)pyrimidin-2-yl)amino)propan-1-ol
4. 2-((6-(4-chlorophenyl)-2-(pyridin-3-yl)pyrimidin-4-yl)amino)-2-methylpropan-1-ol
5. 2-((4-(4-chlorophenyl)-6-(pyridin-3-yl)pyrimidin-2-yl)amino)-2-methylpropan-1-ol
6. 2-((6-(4-chlorophenyl)-2-(pyridin-3-yl)pyrimidin-4-yl)amino)ethan-1-ol
7. 3-((6-(4-chlorophenyl)-2-(pyridin-3-yl)pyrimidin-4-yl)amino)propan-1-ol
8. (S)-1-((6-(4-chlorophenyl)-2-(pyridin-3-yl)pyrimidin-4-yl)amino)propan-2-ol
9. (R)-1-((6-(4-chlorophenyl)-2-(pyridin-3-yl)pyrimidin-4-yl)amino)propan-2-ol
10. 3-((6-(4-chlorophenyl)-2-(pyridin-3-yl)pyrimidin-4-yl)amino)propane-1,2-diol
11. (R)-2-((6-(4-chlorophenyl)-2-(pyridin-3-yl)pyrimidin-4-yl)amino)propan-1-ol
12. 2-((6-(4-chlorophenyl)-2-(pyridin-3-yl)pyrimidin-4-yl)amino)-3-methylbutan-1-ol
13. (S)-2-((6-(4-chlorophenyl)-2-(pyridin-3-yl)pyrimidin-4-yl)amino)-3-methylbutan-1-ol
14. (R)-2-((6-(4-chlorophenyl)-2-(pyridin-3-yl)pyrimidin-4-yl)amino)-3-methylbutan-1-ol
15. 2-((6-(4-chlorophenyl)-2-(pyridin-3-yl)pyrimidin-4-yl)amino)butan-1-ol
16. 2-((6-(4-chlorophenyl)-2-(pyridin-3-yl)pyrimidin-4-yl)amino)propane-1,3-diol
17. (R)-2-((6-(4-chlorophenyl)-2-(pyridin-3-yl)pyrimidin-4-yl)amino)-1-phenylethan-1-ol
18. (S)-2-((6-(4-chlorophenyl)-2-(pyridin-3-yl)pyrimidin-4-yl)amino)-1-phenylethan-1-ol
19. 6-(4-chlorophenyl)-2-(pyridin-3-yl)-N-((tetrahydro-2H-pyran-4-yl)methyl)pyrimidin-4-amine
20. N¹-(6-(4-chlorophenyl)-2-(pyridin-3-yl)pyrimidin-4-yl)-N³,N³-dimethylpropane-1,3-diamine
21. 6-(4-chlorophenyl)-N-ethyl-2-(pyridin-3-yl)pyrimidin-4-amine
22. 6-(4-chlorophenyl)-N-propyl-2-(pyridin-3-yl)pyrimidin-4-amine
23. N-butyl-6-(4-chlorophenyl)-2-(pyridin-3-yl)pyrimidin-4-amine
24. 1-((6-(4-chlorophenyl)-2-(pyridin-3-yl)pyrimidin-4-yl)amino)butan-2-ol
25. 6-(4-chlorophenyl)-N-(cyclopropylmethyl)-2-(pyridin-3-yl)pyrimidin-4-amine
26. 6-(4-chlorophenyl)-N-cyclopentyl-2-(pyridin-3-yl)pyrimidin-4-amine
27. 4-(4-chlorophenyl)-6-(4-methylpiperidin-1-yl)-2-(pyridin-3-yl)pyrimidine
28. N-(tert-butyl)-6-(4-chlorophenyl)-2-(pyridin-3-yl)pyrimidin-4-amine
29. (1R,2R)-2-((6-(4-chlorophenyl)-2-(pyridin-3-yl)pyrimidin-4-yl)amino)cyclopentan-1-ol
30. (1S,2R)-2-((6-(4-chlorophenyl)-2-(pyridin-3-yl)pyrimidin-4-yl)amino)cyclopentan-1-ol
31. 6-(4-chlorophenyl)-N-(pyridin-2-ylmethyl)-2-(pyridin-3-yl)pyrimidin-4-amine
32. 6-(4-chlorophenyl)-2-(pyridin-3-yl)-N-(pyridin-3-ylmethyl)pyrimidin-4-amine
33. 6-(4-chlorophenyl)-2-(pyridin-3-yl)-N-(pyridin-4-ylmethyl)pyrimidin-4-amine
34. trans-4-((6-(4-chlorophenyl)-2-(pyridin-3-yl)pyrimidin-4-yl)amino)cyclohexan-1-ol
35. trans-2-((6-(4-chlorophenyl)-2-(pyridin-3-yl)pyrimidin-4-yl)amino)cyclohexan-1-ol
36. (1-(6-(4-chlorophenyl)-2-(pyridin-3-yl)pyrimidin-4-yl)piperidin-2-yl)methanol
37. 2-(1-(6-(4-chlorophenyl)-2-(pyridin-3-yl)pyrimidin-4-yl)piperidin-2-yl)ethan-1-ol
38. (R)-1-(6-(4-chlorophenyl)-2-(pyridin-3-yl)pyrimidin-4-yl)piperidin-3-ol
39. 1-(6-(4-chlorophenyl)-2-(pyridin-3-yl)pyrimidin-4-yl)piperidin-3-ol
40. (1-(6-(4-chlorophenyl)-2-(pyridin-3-yl)pyrimidin-4-yl)piperidin-3-yl)methanol
41. 1-(6-(4-chlorophenyl)-2-(pyridin-3-yl)pyrimidin-4-yl)piperidin-4-ol
42. (1-(6-(4-chlorophenyl)-2-(pyridin-3-yl)pyrimidin-4-yl)piperidin-4-yl)methanol
43. 2-(1-(6-(4-chlorophenyl)-2-(pyridin-3-yl)pyrimidin-4-yl)piperidin-4-yl)ethan-1-ol
44. 3-(1-(6-(4-chlorophenyl)-2-(pyridin-3-yl)pyrimidin-4-yl)piperidin-4-yl)propan-1-ol
45. 4-(4-chlorophenyl)-6-(4-methoxypiperidin-1-yl)-2-(pyridin-3-yl)pyrimidine
46. 4-(4-chlorophenyl)-6-(piperidin-1-yl)-2-(pyridin-3-yl)pyrimidine
47. 4-(4-chlorophenyl)-6-(2-methylpiperidin-1-yl)-2-(pyridin-3-yl)pyrimidine
48. 4-(4-chlorophenyl)-6-(3-methylpiperidin-1-yl)-2-(pyridin-3-yl)pyrimidine
49. 4-(4-chlorophenyl)-6-(2,6-dimethylpiperidin-1-yl)-2-(pyridin-3-yl)pyrimidine
50. 4-(4-chlorophenyl)-6-(3,5-dimethylpiperidin-1-yl)-2-(pyridin-3-yl)pyrimidine
51. 4-(4-chlorophenyl)-6-(3,3-difluoropiperidin-1-yl)-2-(pyridin-3-yl)pyrimidine
52. 4-(4-chlorophenyl)-2-(pyridin-3-yl)-6-(3-(trifluoromethyl)piperidin-1-yl)pyrimidine
53. 4-(4-chlorophenyl)-6-(3-ethylpiperidin-1-yl)-2-(pyridin-3-yl)pyrimidine
54. 6-(4-chlorophenyl)-N-(piperidin-4-yl)-2-(pyridin-3-yl)pyrimidin-4-amine
55. 6-(4-chlorophenyl)-N-(piperidin-3-ylmethyl)-2-(pyridin-3-yl)pyrimidin-4-amine
56. 6-(4-chlorophenyl)-N-(piperidin-4-ylmethyl)-2-(pyridin-3-yl)pyrimidin-4-amine
57. 6-(4-chlorophenyl)-N-(1-methylpiperidin-4-yl)-2-(pyridin-3-yl)pyrimidin-4-amine
58. 6-(4-chlorophenyl)-N-(2-(piperidin-4-yl)ethyl)-2-(pyridin-3-yl)pyrimidin-4-amine
59. (1-(6-(4-chlorophenyl)-2-(pyridin-3-yl)pyrimidin-4-yl)piperidin-2-yl)methanamine
60. (R)-1-(6-(4-chlorophenyl)-2-(pyridin-3-yl)pyrimidin-4-yl)piperidin-3-amine
61. (S)-1-(6-(4-chlorophenyl)-2-(pyridin-3-yl)pyrimidin-4-yl)piperidin-3-amine
62. (1-(6-(4-chlorophenyl)-2-(pyridin-3-yl)pyrimidin-4-yl)piperidin-3-yl)methanamine
63. (S)-(1-(6-(4-chlorophenyl)-2-(pyridin-3-yl)pyrimidin-4-yl)piperidin-3-yl)methanamine
64. 1-(6-(4-chlorophenyl)-2-(pyridin-3-yl)pyrimidin-4-yl)piperidin-4-amine
65. (1-(6-(4-chlorophenyl)-2-(pyridin-3-yl)pyrimidin-4-yl)piperidin-4-yl)methanamine
66. (1R,2S)-2-((6-(4-chlorophenyl)-2-(pyridin-3-yl)pyrimidin-4-yl)amino)cyclopentan-1-ol
67. (1S,2S)-2-((6-(4-chlorophenyl)-2-(pyridin-3-yl)pyrimidin-4-yl)amino)cyclopentan-1-ol
68. trans-2-((6-(4-chlorophenyl)-2-(pyridin-3-yl)pyrimidin-4-yl)amino)cyclopentan-1-ol
69. (1R,2R)-2-((6-(4-chlorophenyl)-2-(pyridin-3-yl)pyrimidin-4-yl)amino)cyclohexan-1-ol
70. cis-4-(6-(4-chlorophenyl)-2-(pyridin-3-yl)pyrimidin-4-yl)-2,6-dimethylmorpholine
71. 4-(6-(4-chlorophenyl)-2-(pyridin-3-yl)pyrimidin-4-yl)morpholine
72. 6-(4-chlorophenyl)-N-(morpholin-2-ylmethyl)-2-(pyridin-3-yl)pyrimidin-4-amine
73. 4-(1-(6-(4-chlorophenyl)-2-(pyridin-3-yl)pyrimidin-4-yl)pyrrolidin-3-yl)morpholine
74. 4-(6-(4-chlorophenyl)-2-(pyridin-3-yl)pyrimidin-4-yl)thiomorpholine
75. 6-(4-chlorophenyl)-N-(3-morpholinopropyl)-2-(pyridin-3-yl)pyrimidin-4-amine
76. (R)-4-(4-chlorophenyl)-6-(2-methylpiperazin-1-yl)-2-(pyridin-3-yl)pyrimidine
77. (R)-(4-(6-(4-chlorophenyl)-2-(pyridin-3-yl)pyrimidin-4-yl)-3-methylpiperazin-1-yl)(phenyl)methanone
78. methyl (R)-4-(6-(4-chlorophenyl)-2-(pyridin-3-yl)pyrimidin-4-yl)piperazine-2-carboxylate
79. (R)-(4-(6-(4-chlorophenyl)-2-(pyridin-3-yl)pyrimidin-4-yl)piperazin-2-yl)methanol
80. 4-(4-chlorophenyl)-6-(4-(2,3-dichlorophenyl)piperazin-1-yl)-2-(pyridin-3-yl)pyrimidine
81. 4-(4-chlorophenyl)-6-(4-(2,5-dimethoxybenzyl)piperazin-1-yl)-2-(pyridin-3-yl)pyrimidine
82. 2-(4-(6-(4-chlorophenyl)-2-(pyridin-3-yl)pyrimidin-4-yl)piperazin-1-yl)ethan-1-ol
83. 4-(4-chlorophenyl)-6-(4-(2-methoxyphenyl)piperazin-1-yl)-2-(pyridin-3-yl)pyrimidine
84. 4-(4-chlorophenyl)-6-(4-(2-ethoxyphenyl)piperazin-1-yl)-2-(pyridin-3-yl)pyrimidine
85. 4-(4-chlorophenyl)-6-(4-(2-fluorophenyl)piperazin-1-yl)-2-(pyridin-3-yl)pyrimidine
86. (4-(6-(4-chlorophenyl)-2-(pyridin-3-yl)pyrimidin-4-yl)piperazin-1-yl)(furan-2-yl)methanone
87. 4-(4-chlorophenyl)-6-(4-phenethylpiperazin-1-yl)-2-(pyridin-3-yl)pyrimidine
88. 6-(4-chlorophenyl)-N-(2-(piperazin-1-yl)ethyl)-2-(pyridin-3-yl)pyrimidin-4-amine
89. 4-(4-chlorophenyl)-6-(4-(pyridin-2-yl)piperazin-1-yl)-2-(pyridin-3-yl)pyrimidine
90. 4-(4-chlorophenyl)-2-(pyridin-3-yl)-6-(4-(pyrimidin-2-yl)piperazin-1-yl)pyrimidine
91. 4-(2-(4-(6-(4-chlorophenyl)-2-(pyridin-3-yl)pyrimidin-4-yl)piperazin-1-yl)ethyl)morpholine
92. 4-(4-chlorophenyl)-6-(4-(4-methylpiperazin-1-yl)piperidin-1-yl)-2-(pyridin-3-yl)pyrimidine
93. trans-4-(4-chlorophenyl)-6-(4-cinnamylpiperazin-1-yl)-2-(pyridin-3-yl)pyrimidine
94. 4-(6-(4-chlorophenyl)-2-(pyridin-3-yl)pyrimidin-4-yl)piperazin-2-one
95. 4-(4-chlorophenyl)-6-(4-phenylpiperazin-1-yl)-2-(pyridin-3-yl)pyrimidine
96. 4-(4-chlorophenyl)-6-(4-propylpiperazin-1-yl)-2-(pyridin-3-yl)pyrimidine
97. 4-(4-(benzo[d][1,3]dioxol-5-ylmethyl)piperazin-1-yl)-6-(4-chlorophenyl)-2-(pyridin-3-yl)pyrimidine
98. (S)-(4-(6-(4-chlorophenyl)-2-(pyridin-3-yl)pyrimidin-4-yl)piperazin-2-yl)methanol
99. 4-(4-chlorophenyl)-6-(4-(4-fluorophenyl)piperazin-1-yl)-2-(pyridin-3-yl)pyrimidine
100. 6-(4-chlorophenyl)-N-(1,2,2,6,6-pentamethylpiperidin-4-yl)-2-(pyridin-3-yl)pyrimidin-4-amine
101. 6-(4-chlorophenyl)-N-(piperidin-3-yl)-2-(pyridin-3-yl)pyrimidin-4-amine
102. 4-(4-chlorophenyl)-6-(piperazin-1-yl)-2-(pyridin-3-yl)pyrimidine
103. Trans-4-(4-chlorophenyl)-6-(2,5-dimethylpiperazin-1-yl)-2-(pyridin-3-yl)pyrimidine
104. Cis-4-(4-chlorophenyl)-6-(3,5-dimethylpiperazin-1-yl)-2-(pyridin-3-yl)pyrimidine
105. 4-(4-chlorophenyl)-6-(4-methylpiperazin-1-yl)-2-(pyridin-3-yl)pyrimidine
106. 4-(4-chlorophenyl)-6-(4-ethylpiperazin-1-yl)-2-(pyridin-3-yl)pyrimidine
107. 4-(4-chlorophenyl)-6-(4-(methylsulfonyl)piperazin-1-yl)-2-(pyridin-3-yl)pyrimidine
108. 1-(4-(6-(4-chlorophenyl)-2-(pyridin-3-yl)pyrimidin-4-yl)piperazin-1-yl)ethan-1-one
109. 4-(4-chlorophenyl)-6-(3-ethylpiperazin-1-yl)-2-(pyridin-3-yl)pyrimidine
110. ethyl 4-(6-(4-chlorophenyl)-2-(pyridin-3-yl)pyrimidin-4-yl)piperazine-1-carboxylate
111. 4-(6-(4-chlorophenyl)-2-(pyridin-3-yl)pyrimidin-4-yl)piperazine-2-carboxylic acid
112. methyl 1-(6-(4-chlorophenyl)-2-(pyridin-3-yl)pyrimidin-4-yl)piperazine-2-carboxylate
113. (S)-4-(4-chlorophenyl)-6-(2-phenylpiperazin-1-yl)-2-(pyridin-3-yl)pyrimidine
114. 4-(4-chlorophenyl)-2-(pyridin-3-yl)-6-(4-(o-tolyl)piperazin-1-yl)pyrimidine
115. 4-(4-chlorophenyl)-2-(pyridin-3-yl)-6-(4-(p-tolyl)piperazin-1-yl)pyrimidine
116. 4-(4-chlorophenyl)-2-(pyridin-3-yl)-6-(4-(m-tolyl)piperazin-1-yl)pyrimidine
117. 4-(4-chlorophenyl)-2-(pyridin-3-yl)-6-(4-(3-(trifluoromethyl)phenyl)piperazin-1-yl)pyrimidine
118. 4-(4-chlorophenyl)-6-(4-(2,3-dimethylphenyl)piperazin-1-yl)-2-(pyridin-3-yl)pyrimidine
119. 4-(4-chlorophenyl)-6-(4-(3,4-dichlorophenyl)piperazin-1-yl)-2-(pyridin-3-yl)pyrimidine
120. 4-(4-chlorophenyl)-6-(4-(4-methoxyphenyl)piperazin-1-yl)-2-(pyridin-3-yl)pyrimidine
121. 4-(4-chlorophenyl)-6-(4-(4-nitrophenyl)piperazin-1-yl)-2-(pyridin-3-yl)pyrimidine
122. 4-(4-chlorophenyl)-6-(3-(4-methylpiperazin-1-yl)pyrrolidin-1-yl)-2-(pyridin-3-yl)pyrimidine
123. 4-(4-benzhydrylpiperazin-1-yl)-6-(4-chlorophenyl)-2-(pyridin-3-yl)pyrimidine
124. 4-(4-chlorophenyl)-6-(4-((4-chlorophenyl)(phenyl)methyl)piperazin-1-yl)-2-(pyridin-3-yl)pyrimidine
125. 1′-(6-(4-chlorophenyl)-2-(pyridin-3-yl)pyrimidin-4-yl)spiro[indene-1,4′-piperidine]
126. 6-(4-chlorophenyl)-2-(pyridin-3-yl)-N-(pyrrolidin-3-yl)pyrimidin-4-amine
127. (R)-6-(4-chlorophenyl)-2-(pyridin-3-yl)-N-(pyrrolidin-3-yl)pyrimidin-4-amine
128. (R)-6-(4-chlorophenyl)-2-(pyridin-3-yl)-N-(pyrrolidin-3-ylmethyl)pyrimidin-4-amine
129. 6-(4-chlorophenyl)-2-(pyridin-3-yl)-N-(2-(pyrrolidin-1-yl)ethyl)pyrimidin-4-amine
130. 6-(4-chlorophenyl)-2-(pyridin-3-yl)-N-(3-(pyrrolidin-1-yl)propyl)pyrimidin-4-amine
131. 6-(4-chlorophenyl)-N-(2-(1-methylpyrrolidin-2-yl)ethyl)-2-(pyridin-3-yl)pyrimidin-4-amine
132. N-(1-benzylpyrrolidin-3-yl)-6-(4-chlorophenyl)-2-(pyridin-3-yl)pyrimidin-4-amine
133. (3R,4S)-4-((6-(4-chlorophenyl)-2-(pyridin-3-yl)pyrimidin-4-yl)amino)pyrrolidin-3-ol
134. (3S,4R)-4-((6-(4-chlorophenyl)-2-(pyridin-3-yl)pyrimidin-4-yl)amino)pyrrolidin-3-ol
135. 4-(4-chlorophenyl)-2-(pyridin-3-yl)-6-(pyrrolidin-1-yl)pyrimidine
136. 4-(4-chlorophenyl)-6-(2-methylpyrrolidin-1-yl)-2-(pyridin-3-yl)pyrimidine
137. (S)-1-(6-(4-chlorophenyl)-2-(pyridin-3-yl)pyrimidin-4-yl)pyrrolidin-3-ol
138. (1-(6-(4-chlorophenyl)-2-(pyridin-3-yl)pyrimidin-4-yl)pyrrolidin-3-yl)methanol
139. (R)-4-(4-chlorophenyl)-6-(3-fluoropyrrolidin-1-yl)-2-(pyridin-3-yl)pyrimidine
140. 1-(6-(4-chlorophenyl)-2-(pyridin-3-yl)pyrimidin-4-yl)pyrrolidin-3-amine
141. 1-(6-(4-chlorophenyl)-2-(pyridin-3-yl)pyrimidin-4-yl)-N-methylpyrrolidin-3-amine
142. methyl (6-(4-chlorophenyl)-2-(pyridin-3-yl)pyrimidin-4-yl)prolinate
143. N-(1-(6-(4-chlorophenyl)-2-(pyridin-3-yl)pyrimidin-4-yl)pyrrolidin-3-yl)acetamide
144. (2R,3R)-3-((6-(4-chlorophenyl)-2-(pyridin-3-yl)pyrimidin-4-yl)amino)butan-2-ol
145. 3-((6-(4-chlorophenyl)-2-(pyridin-3-yl)pyrimidin-4-yl)amino)butan-2-ol
146. 1-(4-((6-(4-chlorophenyl)-2-(pyridin-3-yl)pyrimidin-4-yl)amino)piperidin-1-yl)ethan-1-one
147. (R)-(1-(6-(4-chlorophenyl)-2-(pyridin-3-yl)pyrimidin-4-yl)piperidin-3-yl)methanol
148. (S)-(1-(6-(4-chlorophenyl)-2-(pyridin-3-yl)pyrimidin-4-yl)piperidin-3-yl)methanol
149. 6-(4-chlorophenyl)-N-(2-(piperidin-1-yl)ethyl)-2-(pyridin-3-yl)pyrimidin-4-amine
150. 1-(6-(4-chlorophenyl)-2-(pyridin-3-yl)pyrimidin-4-yl)piperidine-4-carbonitrile
151. 1-(6-(4-chlorophenyl)-2-(pyridin-3-yl)pyrimidin-4-yl)-4-(3-(trifluoromethyl)phenyl)piperidin-4-ol
152. 4-(4-chlorophenyl)-2-(pyridin-3-yl)-6-(4-(pyrrolidin-1-yl)piperidin-1-yl)pyrimidine
153. 4-(4-chlorophenyl)-1-(6-(4-chlorophenyl)-2-(pyridin-3-yl)pyrimidin-4-yl)piperidin-4-ol
154. 1-(4-(((6-(4-chlorophenyl)-2-(pyridin-3-yl)pyrimidin-4-yl)amino)methyl)piperidin-1-yl)ethan-1-one
155. 1-(1-(6-(4-chlorophenyl)-2-(pyridin-3-yl)pyrimidin-4-yl)-4-phenylpiperidin-4-yl)ethan-1-one
156. 4-(1-(6-(4-chlorophenyl)-2-(pyridin-3-yl)pyrimidin-4-yl)piperidin-4-yl)morpholine
157. 4-(4-chlorophenyl)-6-(4-(3,5-dichlorophenyl)piperidin-1-yl)-2-(pyridin-3-yl)pyrimidine
158. 6-(4-chlorophenyl)-N-((1-cyclohexylpiperidin-3-yl)methyl)-2-(pyridin-3-yl)pyrimidin-4-amine
159. N-((1-benzylpiperidin-4-yl)methyl)-6-(4-chlorophenyl)-2-(pyridin-3-yl)pyrimidin-4-amine
160. ethyl 3-(1-(6-(4-chlorophenyl)-2-(pyridin-3-yl)pyrimidin-4-yl)piperidin-4-yl)-3-oxopropanoate
161. ethyl 2-(1-(6-(4-chlorophenyl)-2-(pyridin-3-yl)pyrimidin-4-yl)piperidin-4-yl)acetate
162. (1S,3R)-3-((6-(4-chlorophenyl)-2-(pyridin-3-yl)pyrimidin-4-yl)amino)cyclopentan-1-ol
163. (S)-1-(6-(4-chlorophenyl)-2-(pyridin-3-yl)pyrimidin-4-yl)piperidin-3-ol
164. 1-(6-(4-chlorophenyl)-2-(pyridin-3-yl)pyrimidin-4-yl)-N,N-dimethylpyrrolidin-3-amine
165. 2-(1-(6-(4-chlorophenyl)-2-(pyridin-3-yl)pyrimidin-4-yl)pyrrolidin-2-yl)-N,N-dimethylethan-1-amine
166. 1-(6-(4-chlorophenyl)-2-(pyridin-3-yl)pyrimidin-4-yl)piperidin-4-one
167. 6-(4-chlorophenyl)-N-methyl-N-(piperidin-4-yl)-2-(pyridin-3-yl)pyrimidin-4-amine
168. 6-(4-chlorophenyl)-N-(2-(1-methylpiperidin-2-yl)ethyl)-2-(pyridin-3-yl)pyrimidin-4-amine
169. 6-(4-chlorophenyl)-N-(1-(1-methylpiperidin-4-yl)ethyl)-2-(pyridin-3-yl)pyrimidin-4-amine
170. 6-(4-chlorophenyl)-N-((1-(2-methoxyethyl)piperidin-4-yl)methyl)-2-(pyridin-3-yl)pyrimidin-4-amine
171. methyl 2-(4-((6-(4-chlorophenyl)-2-(pyridin-3-yl)pyrimidin-4-yl)amino)piperidin-1-yl)acetate
172. 1-(1-(6-(4-chlorophenyl)-2-(pyridin-3-yl)pyrimidin-4-yl)piperidin-4-yl)ethyl 2,2,2-trifluoroacetate
173. 6-(4-chlorophenyl)-N-(1-methylpiperidin-3-yl)-2-(pyridin-3-yl)pyrimidin-4-amine
174. (1S,2R)-2-((6-(4-chlorophenyl)-2-(1-methyl-1H-pyrazol-4-yl)pyrimidin-4-yl)amino)cyclopentan-1-ol
175. (R)-1-(6-(4-chlorophenyl)-2-(1-methyl-1H-pyrazol-4-yl)pyrimidin-4-yl)piperidin-3-ol
176. 1-(6-(4-chlorophenyl)-2-(1-methyl-1H-pyrazol-4-yl)pyrimidin-4-yl)piperidin-4-ol
177. (1-(6-(4-chlorophenyl)-2-(1-methyl-1H-pyrazol-4-yl)pyrimidin-4-yl)piperidin-4-yl)methanol
178. 2-(1-(6-(4-chlorophenyl)-2-(1-methyl-1H-pyrazol-4-yl)pyrimidin-4-yl)piperidin-4-yl)ethan-1-ol
179. 3-(1-(6-(4-chlorophenyl)-2-(1-methyl-1H-pyrazol-4-yl)pyrimidin-4-yl)piperidin-4-yl)propan-1-ol
180. 4-(4-chlorophenyl)-2-(1-methyl-1H-pyrazol-4-yl)-6-(4-methylpiperidin-1-yl)pyrimidine
181. 4-(4-chlorophenyl)-2-(1-methyl-1H-pyrazol-4-yl)-6-(4-methylpiperazin-1-yl)pyrimidine
182. 2-(4-(6-(4-chlorophenyl)-2-(1-methyl-1H-pyrazol-4-yl)pyrimidin-4-yl)piperazin-1-yl)ethan-1-ol
183. (S)-1-(6-(4-chlorophenyl)-2-(1-methyl-1H-pyrazol-4-yl)pyrimidin-4-yl)pyrrolidin-3-ol
184. 1-(6-(4-chlorophenyl)-2-(1-methyl-1H-pyrazol-4-yl)pyrimidin-4-yl)piperidine-4-carbonitrile
185. (R)-(1-(6-(4-chlorophenyl)-2-(1-methyl-1H-pyrazol-4-yl)pyrimidin-4-yl)piperidin-3-yl)methanol
186. (R)-1-(6-(4-chlorophenyl)-2-(1-methyl-1H-pyrazol-4-yl)pyrimidin-4-yl)pyrrolidin-3-ol
187. (1S,3R)-3-((6-(4-chlorophenyl)-2-(1-methyl-1H-pyrazol-4-yl)pyrimidin-4-yl)amino)cyclopentan-1-ol
188. (R)-2-((6-(4-chlorophenyl)-2-(1-methyl-1H-pyrazol-4-yl)pyrimidin-4-yl)amino)butan-1-ol
189. Trans-4-((6-(4-chlorophenyl)-2-(1-methyl-1H-pyrazol-4-yl)pyrimidin-4-yl)amino)cyclohexan-1-ol
190. 7-(6-(4-chlorophenyl)-2-(pyridin-3-yl)pyrimidin-4-yl)octahydro-2H-pyrano[2,3-c]pyridine
191. 7-(6-(4-chlorophenyl)-2-(pyridin-3-yl)pyrimidin-4-yl)octahydro-2H-pyrano[2,3-c]pyridin-4-ol
192. (2R,3R)-3-((6-(4-chlorophenyl)-2-(pyridin-3-yl)pyrimidin-4-yl)amino)pentan-2-ol
193. 6-(4-chlorophenyl)-N-((1-methylpiperidin-4-yl)methyl)-2-(pyridin-3-yl)pyrimidin-4-amine
194. (R)-1-(6-(4-chlorophenyl)-2-(pyridin-3-yl)pyrimidin-4-yl)pyrrolidin-3-ol
195. (S)-6-(4-chlorophenyl)-N-(2-(methoxymethyl)pyrrolidin-1-yl)-2-(pyridin-3-yl)pyrimidin-4-amine
196. (S)-4-(4-chlorophenyl)-6-(3-fluoropyrrolidin-1-yl)-2-(pyridin-3-yl)pyrimidine
197. 4-(4-chlorophenyl)-2-(pyridin-3-yl)-6-(2-(trifluoromethyl)pyrrolidin-1-yl)pyrimidine
198. 4-(4-chlorophenyl)-6-(3,3-difluoropyrrolidin-1-yl)-2-(pyridin-3-yl)pyrimidine
199. 4-(1-(6-(4-chlorophenyl)-2-(pyridin-3-yl)pyrimidin-4-yl)pyrrolidin-3-yl)morpholine
200. 5-(((6-(4-chlorophenyl)-2-(pyridin-3-yl)pyrimidin-4-yl)amino)methyl)pyrrolidin-2-one
201. Trans-6-(4-chlorophenyl)-2-(pyridin-3-yl)-N-(4-(pyrrolidin-1-yl)tetrahydrofuran-3-yl)pyrimidin-4-amine
202. 6-(4-chlorophenyl)-N-((3S,4S)-4-methoxy-1-methylpyrrolidin-3-yl)-2-(pyridin-3-yl)pyrimidin-4-amine
203. (R)-6-(4-chlorophenyl)-N-(piperidin-3-yl)-2-(pyridin-3-yl)pyrimidin-4-amine
204. 6-(4-chlorophenyl)-N-(piperidin-3-yl)-2-(pyridin-3-yl)pyrimidin-4-amine
205. 6-(4-chlorophenyl)-N-((3R,4R)-3-fluoropiperidin-4-yl)-2-(pyridin-3-yl)pyrimidin-4-amine
206. (S)-6-(4-chlorophenyl)-2-(pyridin-3-yl)-N-(pyrrolidin-3-ylmethyl)pyrimidin-4-amine
207. methyl (2R,4R)-4-((6-(4-chlorophenyl)-2-(pyridin-3-yl)pyrimidin-4-yl)amino)pyrrolidine-2-carboxylate
208. (2R,4S)-4-((6-(4-chlorophenyl)-2-(pyridin-3-yl)pyrimidin-4-yl)amino)pyrrolidine-2-carboxylic acid
209. Trans-4-((6-(4-chlorophenyl)-2-(pyridin-3-yl)pyrimidin-4-yl)amino)-1-isopropylpyrrolidin-3-ol
210. (R)-4-(3-(chloromethyl)pyrrolidin-1-yl)-6-(4-chlorophenyl)-2-(pyridin-3-yl)pyrimidine
211. (S)-4-(3-(chloromethyl)pyrrolidin-1-yl)-6-(4-chlorophenyl)-2-(pyridin-3-yl)pyrimidine
212. 1-(6-(4-chlorophenyl)-2-(pyridin-3-yl)pyrimidin-4-yl)pyrrolidine-3-carbonitrile
213. (R)-1-((6-(4-chlorophenyl)-2-(pyridin-3-yl)pyrimidin-4-yl)amino)butan-2-ol
214. (1R,3S)-3-((6-(4-chlorophenyl)-2-(pyridin-3-yl)pyrimidin-4-yl)amino)cyclopentan-1-ol
215. Cis-(4-((6-(4-chlorophenyl)-2-(pyridin-3-yl)pyrimidin-4-yl)amino)cyclohexyl)methanol
216. Cis-4-((6-(4-chlorophenyl)-2-(pyridin-3-yl)pyrimidin-4-yl)amino)cyclohexan-1-ol
217. Trans-4-((6-(4-chlorophenyl)-2-(pyridin-3-yl)pyrimidin-4-yl)amino)cyclohexan-1-ol
218. 4-(4-chlorophenyl)-6-(4-(2-methoxyethyl)piperazin-1-yl)-2-(pyridin-3-yl)pyrimidine
219. (3S,4R)-1-(6-(4-chlorophenyl)-2-(pyridin-3-yl)pyrimidin-4-yl)-3-fluoropiperidin-4-ol
220. (3R,4S)-1-(6-(4-chlorophenyl)-2-(pyridin-3-yl)pyrimidin-4-yl)-3-fluoropiperidin-4-ol
221. (3R,4R)-1-(6-(4-chlorophenyl)-2-(pyridin-3-yl)pyrimidin-4-yl)-3-fluoropiperidin-4-ol
222. (3S,4S)-1-(6-(4-chlorophenyl)-2-(pyridin-3-yl)pyrimidin-4-yl)-3-fluoropiperidin-4-ol
223. 1-(4-(6-(4-chlorophenyl)-2-(pyridin-3-yl)pyrimidin-4-yl)piperazin-1-yl)-2-hydroxyethan-1-one
224. 2-(4-(6-(4-chlorophenyl)-2-(pyridin-3-yl)pyrimidin-4-yl)piperazin-1-yl)propan-1-ol
225. N-(1-(6-(4-chlorophenyl)-2-(pyridin-3-yl)pyrimidin-4-yl)piperidin-4-yl)-2-methoxyacetamide
227. (1-(2-(pyridin-3-yl)-6-(4-(trifluoromethoxy)phenyl)pyrimidin-4-yl)piperidin-4-yl)methanol
228. (1-(6-(4-methoxyphenyl)-2-(pyridin-3-yl)pyrimidin-4-yl)piperidin-4-yl)methanol
229. (1-(2-(pyridin-3-yl)-6-(p-tolyl)pyrimidin-4-yl)piperidin-4-yl)methanol
230. (3R,4R)-1-(6-(4-chlorophenyl)-2-(pyridin-3-yl)pyrimidin-4-yl)pyrrolidine-3,4-diol
231. (3R,4S)-1-(6-(4-chlorophenyl)-2-(pyridin-3-yl)pyrimidin-4-yl)pyrrolidine-3,4-diol
232. 1-(3-(6-(4-chlorophenyl)-2-(pyridin-3-yl)pyrimidin-4-yl)tetrahydropyrimidin-1 (2H)-yl)ethan-1-one
233. 1-(6-(4-chlorophenyl)-2-(pyridin-3-yl)pyrimidin-4-yl)-4-(hydroxymethyl)piperidin-3-ol
233. 1-(6-(4-chlorophenyl)-2-(pyridin-3-yl)pyrimidin-4-yl)-4-(hydroxymethyl)piperidin-3-ol
234. (3R,4R)-1-(6-(4-chlorophenyl)-2-(pyridin-3-yl)pyrimidin-4-yl)-4-fluoropyrrolidin-3-ol
235. (3R,4S)-1-(6-(4-chlorophenyl)-2-(pyridin-3-yl)pyrimidin-4-yl)-4-fluoropyrrolidin-3-ol
236. 1-(6-(4-chlorophenyl)-2-(pyridin-3-yl)pyrimidin-4-yl)-4-(hydroxymethyl)pyrrolidin-3-ol
237. N-(1-(6-(4-chlorophenyl)-2-(pyridin-3-yl)pyrimidin-4-yl)piperidin-4-yl)-2-hydroxypropanamide
238. N-(1-(6-(4-chlorophenyl)-2-(pyridin-3-yl)pyrimidin-4-yl)piperidin-4-yl)-2-hydroxyacetamide
239. 2-((4-(6-(4-chlorophenyl)-2-(pyridin-3-yl)pyrimidin-4-yl)piperazin-1-yl)sulfonyl)ethan-1-ol
240. (S)-(1-(6-(4-chlorophenyl)-2-(pyridin-3-yl)pyrimidin-4-yl)pyrrolidin-3-yl)methanol
241. N-((3R,4R)-1-(6-(4-chlorophenyl)-2-(pyridin-3-yl)pyrimidin-4-yl)-4-hydroxypyrrolidin-3-yl)acetamide
242. (3R,4R)-4-acetamido-1-(6-(4-chlorophenyl)-2-(pyridin-3-yl)pyrimidin-4-yl)pyrrolidin-3-yl acetate
243. N-((3R,4S)-1-(6-(4-chlorophenyl)-2-(pyridin-3-yl)pyrimidin-4-yl)-4-hydroxypyrrolidin-3-yl)acetamide
244. N-((3S,4R)-1-(6-(4-chlorophenyl)-2-(pyridin-3-yl)pyrimidin-4-yl)-4-hydroxypyrrolidin-3-yl)acetamide
245. N-((3S,4S)-1-(6-(4-chlorophenyl)-2-(pyridin-3-yl)pyrimidin-4-yl)-4-hydroxypyrrolidin-3-yl)acetamide
246. (1-(6-(4-chloro-3-fluorophenyl)-2-(pyridin-3-yl)pyrimidin-4-yl)piperidin-4-yl)methanol
247. (3S,4R)-1-(6-(4-chlorophenyl)-2-(pyridin-3-yl)pyrimidin-4-yl)-4-(hydroxymethyl)piperidin-3-ol
248. (3S,4R)-4-(hydroxymethyl)-1-(2-(pyridin-3-yl)-6-(4-(trifluoromethyl)phenyl)pyrimidin-4-yl)piperidin-3-ol
249. ((3S,4R)-1-(6-(4-chlorophenyl)-2-(pyridin-3-yl)pyrimidin-4-yl)-3-fluoropiperidin-4-yl)methanol
250. ((3S,4R)-3-fluoro-1-(2-(pyridin-3-yl)-6-(4-(trifluoromethyl)phenyl)pyrimidin-4-yl)piperidin-4-yl)methanol
251. ((3R,4S)-1-(6-(4-chlorophenyl)-2-(pyridin-3-yl)pyrimidin-4-yl)-3-fluoropiperidin-4-yl)methanol
252. ((3R,4S)-3-fluoro-1-(2-(pyridin-3-yl)-6-(4-(trifluoromethyl)phenyl)pyrimidin-4-yl)piperidin-4-yl)methanol
253. ((3R,4R)-1-(6-(4-chlorophenyl)-2-(pyridin-3-yl)pyrimidin-4-yl)-3-fluoropiperidin-4-yl)methanol
254. ((3R,4R)-3-fluoro-1-(2-(pyridin-3-yl)-6-(4-(trifluoromethyl)phenyl)pyrimidin-4-yl)piperidin-4-yl)methanol
255. (3S,4R)-1-(6-(4-chlorophenyl)-2-(pyridin-3-yl)pyrimidin-4-yl)-4-(hydroxymethyl)pyrrolidin-3-ol
256. (3S,4R)-4-(hydroxymethyl)-1-(2-(pyridin-3-yl)-6-(4-(trifluoromethyl)phenyl)pyrimidin-4-yl)pyrrolidin-3-ol
257. (3R,4R)-1-(6-(4-chlorophenyl)-2-(pyridin-3-yl)pyrimidin-4-yl)-4-(hydroxymethyl)pyrrolidin-3-ol
258. (3R,4R)-4-(hydroxymethyl)-1-(2-(pyridin-3-yl)-6-(4-(trifluoromethyl)phenyl)pyrimidin-4-yl)pyrrolidin-3-ol
259. (3R,4R)-1-(6-(4-chlorophenyl)-2-(pyridin-3-yl)pyrimidin-4-yl)-4-(hydroxymethyl)piperidin-3-ol
260. (3R,4R)-4-(hydroxymethyl)-1-(2-(pyridin-3-yl)-6-(4-(trifluoromethyl)phenyl)pyrimidin-4-yl)piperidin-3-ol
261. (3S,4S)-1-(6-(4-chlorophenyl)-2-(pyridin-3-yl)pyrimidin-4-yl)-4-(hydroxymethyl)piperidin-3-ol
262. (3S,4S)-4-(hydroxymethyl)-1-(2-(pyridin-3-yl)-6-(4-(trifluoromethyl)phenyl)pyrimidin-4-yl)piperidin-3-ol
263. (S)-1-(2-(4-methylpyridin-3-yl)-6-(4-(trifluoromethyl)phenyl)pyrimidin-4-yl)pyrrolidin-3-ol
264. (3R,4S)-1-(6-(4-chlorophenyl)-2-(pyridin-3-yl)pyrimidin-4-yl)-4-(hydroxymethyl)piperidin-3-ol
265. (3R,4S)-4-(hydroxymethyl)-1-(2-(pyridin-3-yl)-6-(4-(trifluoromethyl)phenyl)pyrimidin-4-yl)piperidin-3-ol
266. (3S,4S)-1-(6-(4-chlorophenyl)-2-(pyridin-3-yl)pyrimidin-4-yl)-4-(hydroxymethyl)pyrrolidin-3-ol
267. (3S,4S)-4-(hydroxymethyl)-1-(2-(pyridin-3-yl)-6-(4-(trifluoromethyl)phenyl)pyrimidin-4-yl)pyrrolidin-3-ol
268. (1-(6-(4-morpholinophenyl)-2-(pyridin-3-yl)pyrimidin-4-yl)piperidin-4-yl)methanol
269. (S)-1-(2-(2-methylpyridin-3-yl)-6-(4-(trifluoromethyl)phenyl)pyrimidin-4-yl)pyrrolidin-3-ol
270. (S)-3-(4-(3-hydroxypyrrolidin-1-yl)-6-(4-(trifluoromethyl)phenyl)pyrimidin-2-yl)pyridin-2-ol
271. 5-chloro-2-(6-(4-(hydroxymethyl)piperidin-1-yl)-2-(pyridin-3-yl)pyrimidin-4-yl)phenol
272. tert-butyl (S)-4-(6-(4-chlorophenyl)-2-(pyridin-3-yl)pyrimidin-4-yl)-2-(hydroxymethyl)piperazine-1-carboxylate
273. 2-chloro-5-(6-(4-(hydroxymethyl)piperidin-1-yl)-2-(pyridin-3-yl)pyrimidin-4-yl)phenol
274. N-(4-(6-(4-(hydroxymethyl)piperidin-1-yl)-2-(pyridin-3-yl)pyrimidin-4-yl)phenyl)methanesulfonamide
275. (1-(6-(4-(4-methylpiperazin-1-yl)phenyl)-2-(pyridin-3-yl)pyrimidin-4-yl)piperidin-4-yl)methanol
276. (1-(6-(3-fluoro-4-morpholinophenyl)-2-(pyridin-3-yl)pyrimidin-4-yl)piperidin-4-yl)methanol
277. (1-(2-(pyridin-3-yl)-6-(2,4,6-trifluorophenyl)pyrimidin-4-yl)piperidin-4-yl)methanol
278. (1-(2-(pyridin-3-yl)-6-(4-((tetrahydro-2H-pyran-2-yl)oxy)phenyl)pyrimidin-4-yl)piperidin-4-yl)methanol
279. (S)-(4-(2-(pyridin-3-yl)-6-(4-(trifluoromethyl)phenyl)pyrimidin-4-yl)morpholin-2-yl)methanol
280. (R)-(4-(2-(pyridin-3-yl)-6-(4-(trifluoromethyl)phenyl)pyrimidin-4-yl)morpholin-2-yl)methanol
281. ((3S,4S)-3-fluoro-1-(2-(pyridin-3-yl)-6-(4-(trifluoromethyl)phenyl)pyrimidin-4-yl)piperidin-4-yl)methanol
282. ((3S,4S)-1-(6-(4-chlorophenyl)-2-(pyridin-3-yl)pyrimidin-4-yl)-3-fluoropiperidin-4-yl)methanol
283. (3S,4S)-1-(2-(pyridin-3-yl)-6-(4-(trifluoromethyl)phenyl)pyrimidin-4-yl)pyrrolidine-3,4-diol
284. (3S,4S)-1-(6-(4-chlorophenyl)-2-(pyridin-3-yl)pyrimidin-4-yl)pyrrolidine-3,4-diol
285. 3-(4-(4-(hydroxymethyl)piperidin-1-yl)-6-(4-morpholinophenyl)pyrimidin-2-yl)pyridin-2-ol
286. (1-(2-(1-methyl-1H-pyrazol-4-yl)-6-(4-morpholinophenyl)pyrimidin-4-yl)piperidin-4-yl)methanol
287. (1-(6-(3-fluoro-4-morpholinophenyl)-2-(1-methyl-1H-pyrazol-4-yl)pyrimidin-4-yl)piperidin-4-yl)methanol
288. (1-(6-(1H-indazol-5-yl)-2-(pyridin-3-yl)pyrimidin-4-yl)piperidin-4-yl)methanol
289. (1-(6-(6-morpholinopyridin-3-yl)-2-(pyridin-3-yl)pyrimidin-4-yl)piperidin-4-yl)methanol
290. 5-(6-(4-(hydroxymethyl)piperidin-1-yl)-2-(pyridin-3-yl)pyrimidin-4-yl)indolin-2-one
291. 4-(4-(6-(4-(hydroxymethyl)piperidin-1-yl)-2-(pyridin-3-yl)pyrimidin-4-yl)phenyl)morpholin-3-one
292. 4-(6-(4-(hydroxymethyl)piperidin-1-yl)-2-(pyridin-3-yl)pyrimidin-4-yl)benzoic acid
293. 4 (1-(6-(1-methyl-1H-pyrazol-3-yl)-2-(pyridin-3-yl)pyrimidin-4-yl)piperidin-4-yl)methanol
294. (1-(6-(5-fluoropyridin-3-yl)-2-(pyridin-3-yl)pyrimidin-4-yl)piperidin-4-yl)methanol
295. (S)-3-(4-(3-hydroxypyrrolidin-1-yl)-6-(6-morpholinopyridin-3-yl)pyrimidin-2-yl)pyridin-2-ol
296. (S)-1-(6-(4-fluoro-3-nitrophenyl)-2-(pyridin-3-yl)pyrimidin-4-yl)pyrrolidin-3-ol
297. (S)-1-(6-(4-morpholino-3-nitrophenyl)-2-(pyridin-3-yl)pyrimidin-4-yl)pyrrolidin-3-ol
298. (S)-1-(6-(3-amino-4-morpholinophenyl)-2-(pyridin-3-yl)pyrimidin-4-yl)pyrrolidin-3-ol
299. (S)—N-(5-(6-(3-hydroxypyrrolidin-1-yl)-2-(pyridin-3-yl)pyrimidin-4-yl)-2-morpholinophenyl)acetamide
300. (S)-1-(6-(6-fluoropyridin-3-yl)-2-(pyridin-3-yl)pyrimidin-4-yl)pyrrolidin-3-ol
301. (S)-1-(6-(6-((2-(dimethylamino)ethyl)amino)pyridin-3-yl)-2-(pyridin-3-yl)pyrimidin-4-yl)pyrrolidin-3-ol
302. (3S)-1-(6-(6-(2,6-dimethylmorpholino)pyridin-3-yl)-2-(pyridin-3-yl)pyrimidin-4-yl)pyrrolidin-3-ol
303. 5-chloro-2-(6-(4-((2-hydroxyethyl)sulfonyl)piperazin-1-yl)-2-(pyridin-3-yl)pyrimidin-4-yl)phenol
304. (S)-3-(4-(4-chloro-2-hydroxyphenyl)-6-(3-hydroxypyrrolidin-1-yl)pyrimidin-2-yl)pyridin-2-ol
305. (S)-1-(6-(4-aminophenyl)-2-(pyridin-3-yl)pyrimidin-4-yl)pyrrolidin-3-ol
306. 4-(4-chlorophenyl)-2-(pyridin-3-yl)-6-(4-(vinylsulfonyl)piperazin-1-yl)pyrimidine
307. (1-(6-(2,4-dichlorophenyl)-2-(pyridin-3-yl)pyrimidin-4-yl)piperidin-4-yl)methanol
308. (S)-(4-(2-(pyridin-3-yl)-6-(4-(trifluoromethyl)phenyl)pyrimidin-4-yl)piperazin-2-yl)methanol
309. (R)-(4-(2-(pyridin-3-yl)-6-(4-(trifluoromethyl)phenyl)pyrimidin-4-yl)piperazin-2-yl)methanol
310. (R)-1-(2-(pyridin-3-yl)-6-(4-(trifluoromethyl)phenyl)pyrimidin-4-yl)pyrrolidine-3-carboxylic acid
311. (R)-1-(6-(4-chlorophenyl)-2-(pyridin-3-yl)pyrimidin-4-yl)pyrrolidine-3-carboxylic acid
312. (R)-1-(2-(1-methyl-1H-pyrazol-4-yl)-6-(4-(trifluoromethyl)phenyl)pyrimidin-4-yl)pyrrolidine-3-carboxylic acid
313. (R)-1-(6-(4-chlorophenyl)-2-(1-methyl-1H-pyrazol-4-yl)pyrimidin-4-yl)pyrrolidine-3-carboxylic acid
314. (R)-2-(2-(pyridin-3-yl)-6-(4-(trifluoromethyl)phenyl)pyrimidin-4-yl)isoxazolidin-4-ol
315. (S)-1-(6-(6-morpholinopyridin-3-yl)-2-(pyridin-3-yl)pyrimidin-4-yl)pyrrolidin-3-ol
316. (S)-1-(6-(4-chloro-2-hydroxyphenyl)-2-(pyridin-3-yl)pyrimidin-4-yl)pyrrolidin-3-ol
317. (S)-3-(6-(3-hydroxypyrrolidin-1-yl)-2-(pyridin-3-yl)pyrimidin-4-yl)pyridin-2-ol
318. (1-(6-(6-fluoropyridin-3-yl)-2-(pyridin-3-yl)pyrimidin-4-yl)piperidin-4-yl)methanol
319. 1-(6-(4-chlorophenyl)-2-(pyridin-3-yl)pyrimidin-4-yl)azetidin-3-ol
320. (1-(6-(4-chlorophenyl)-2-(pyridin-3-yl)pyrimidin-4-yl)azetidin-3-yl)methanol
321. 4-(4-chlorophenyl)-2-(1-methyl-1H-pyrazol-4-yl)-6-(4-(methylsulfonyl)piperazin-1-yl)pyrimidine
322. (S)-1-(6-(4-chlorophenyl)-2-(3-hydroxyphenyl)pyrimidin-4-yl)pyrrolidin-3-ol formate
322. (S)-1-(6-(4-chlorophenyl)-2-(3-hydroxyphenyl)pyrimidin-4-yl)pyrrolidin-3-ol
323. (S)-1-(6-(4-chlorophenyl)-2-(5-fluoropyridin-3-yl)pyrimidin-4-yl)pyrrolidin-3-ol
324. (S)-1-(6-(4-chlorophenyl)-2-(pyridin-4-yl)pyrimidin-4-yl)pyrrolidin-3-ol
325. 4-(4-chlorophenyl)-2-(5-fluoropyridin-3-yl)-6-(4-(methylsulfonyl)piperazin-1-yl)pyrimidine
326. 4-(4-chlorophenyl)-6-(4-(methylsulfonyl)piperidin-1-yl)-2-(pyridin-3-yl)pyrimidine
327. (S)-1-(2-(5-fluoropyridin-3-yl)-6-(4-(trifluoromethyl)phenyl)pyrimidin-4-yl)pyrrolidin-3-ol
328. 4-(4-chlorophenyl)-6-(4-(cyclopropylsulfonyl)piperazin-1-yl)-2-(pyridin-3-yl)pyrimidine
329. (S)-1-(6-(4-chlorophenyl)-2-(pyridazin-4-yl)pyrimidin-4-yl)pyrrolidin-3-ol
330. 1-(6-(4-chlorophenyl)-2-(pyridin-3-yl)pyrimidin-4-yl)azepan-4-ol
331. 2-(6-(4-chlorophenyl)-2-(pyridin-3-yl)pyrimidin-4-yl)-5-(methylsulfonyl)-2,5-diazabicyclo[2.2.1]heptane
332. (S)-1-(6-(4-chlorophenyl)-2-(2-methyl-2H-tetrazol-5-yl)pyrimidin-4-yl)pyrrolidin-3-ol
333. (S)-1-(6-(4-chlorophenyl)-2-(2-methyl-2H-tetrazol-5-yl)pyrimidin-4-yl)pyrrolidin-3-ol
334. 4-(4-chlorophenyl)-2-(2-methyl-2H-tetrazol-5-yl)-6-(4-(methylsulfonyl)piperazin-1-yl)pyrimidine
335. 4-(4-chlorophenyl)-6-(4-((2-fluoroethyl)sulfonyl)piperazin-1-yl)-2-(pyridin-3-yl)pyrimidine
336. (S)-1-(6-(4-chlorophenyl)-2-(isoxazol-4-yl)pyrimidin-4-yl)pyrrolidin-3-ol
337. (1-(6-(4-chlorophenyl)-2-(pyridin-3-yl)pyrimidin-4-yl)-4-(methylsulfonyl)piperidin-4-yl)methanol
338. 1-(6-(4-chlorophenyl)-2-(pyridin-3-yl)pyrimidin-4-yl)azepan-3-ol
339. 4-(4-chlorophenyl)-6-(4-((difluoromethyl)sulfonyl)piperazin-1-yl)-2-(pyridin-3-yl)pyrimidine
340. (S)-1-(2-(pyridin-3-yl)-6-(4-(trifluoromethyl)phenyl)pyrimidin-4-yl)pyrrolidin-3-ol
341. 4-(4-(methylsulfonyl)piperazin-1-yl)-2-(pyridin-3-yl)-6-(4-(trifluoromethyl)phenyl)pyrimidine
342. (S)-1-(6-(4-chlorophenyl)-2-(5,6-difluoropyridin-3-yl)pyrimidin-4-yl)pyrrolidin-3-ol
343. (3S,4R)-1-(2-(pyridin-3-yl)-6-(4-(trifluoromethyl)phenyl)pyrimidin-4-yl)pyrrolidine-3,4-diol
344. (S)-1-(6-(4-chlorophenyl)-[2,5′-bipyrimidin]-4-yl)pyrrolidin-3-ol
345. (S)-1-(6-(4-chlorophenyl)-2-(6-fluoropyridin-3-yl)pyrimidin-4-yl)pyrrolidin-3-ol
346. (S)-1-(6-(4-chlorophenyl)-2-(2-fluoropyridin-3-yl)pyrimidin-4-yl)pyrrolidin-3-ol
347. (S)-1-(6-(4-chlorophenyl)-2-(pyridin-2-yl)pyrimidin-4-yl)pyrrolidin-3-ol
348. 2-((4-(6-(4-chlorophenyl)-2-(1-methyl-1H-pyrazol-4-yl)pyrimidin-4-yl)piperazin-1-yl)sulfonyl)ethanol
349. 2-((4-(2-(pyridin-3-yl)-6-(4-(trifluoromethyl)phenyl)pyrimidin-4-yl)piperazin-1-yl)sulfonyl)ethan-1-ol
350. (S)-1-(2-(isothiazol-4-yl)-6-(4-(trifluoromethyl)phenyl)pyrimidin-4-yl)pyrrolidin-3-ol
351. (4-(methylsulfonyl)-1-(2-(pyridin-3-yl)-6-(4-(trifluoromethyl)phenyl)pyrimidin-4-yl)piperidin-4-yl)methanol
352. 1-(4-(6-(4-chlorophenyl)-2-(pyridin-3-yl)pyrimidin-4-yl)piperazin-1-yl)-3-hydroxypropan-1-one
353. 2-((4-(2-(1-methyl-1H-pyrazol-4-yl)-6-(4-(trifluoromethyl)phenyl)pyrimidin-4-yl)piperazin-1-yl)sulfonyl)ethanol
354. 1-(6-(4-chlorophenyl)-2-(pyridin-3-yl)pyrimidin-4-yl)-3-(dimethylamino)piperidin-4-ol
355. 1-(6-(4-chlorophenyl)-2-(pyridin-3-yl)pyrimidin-4-yl)-5-(dimethylamino)piperidin-3-ol
356. (1-(6-(4-chlorophenyl)-2-(1-methyl-1H-pyrazol-4-yl)pyrimidin-4-yl)-4-(methylsulfonyl)piperidin-4-yl)methanol
357. (1-(2-(1-methyl-1H-pyrazol-4-yl)-6-(4-(trifluoromethyl)phenyl)pyrimidin-4-yl)-4-(methylsulfonyl)piperidin-4-yl)methanol
358. (1-(6-(4-chlorophenyl)-2-(pyridin-3-yl)pyrimidin-4-yl)-3-(dimethylamino)piperidin-4-yl)methanol
359. 2-((4-(6-(4-chlorophenyl)-2-(pyridin-3-yl)pyrimidin-4-yl)-3-methylpiperazin-1-yl)sulfonyl)ethan-1-ol
360. 2-((1-(6-(4-chlorophenyl)-2-(pyridin-3-yl)pyrimidin-4-yl)piperidin-4-yl)amino)ethanol
361. 1-(4-(6-(4-chlorophenyl)-2-(pyridin-3-yl)pyrimidin-4-yl)piperazin-1-yl)-4-hydroxybutan-1-one
362. 3-((4-(6-(4-chlorophenyl)-2-(pyridin-3-yl)pyrimidin-4-yl)piperazin-1-yl)sulfonyl)propan-1-ol
363. 1-(4-(6-(4-chlorophenyl)-2-(pyridin-3-yl)pyrimidin-4-yl)piperazin-1-yl)-3,4-dihydroxybutan-1-one
364. 1-(4-(6-(4-chlorophenyl)-2-(pyridin-3-yl)pyrimidin-4-yl)piperazin-1-yl)-2,3-dihydroxybutan-1-one
365. 4-(6-(4-chlorophenyl)-2-(pyridin-3-yl)pyrimidin-4-yl)-6-methylpiperazin-2-one
366. (S)-3-(4-(3-hydroxypyrrolidin-1-yl)-6-(6-morpholinopyridin-3-yl)pyrimidin-2-yl)pyridin-2-ol
367. (S)-4-(4-(6-(3-hydroxypyrrolidin-1-yl)-2-(pyridin-3-yl)pyrimidin-4-yl)phenyl)morpholin-3-one
368. 2-((4-(6-(4-chlorophenyl)-2-(isothiazol-4-yl)pyrimidin-4-yl)piperazin-1-yl)sulfonyl)ethanol
369. 3-((4-(6-(4-chlorophenyl)-2-(pyridin-3-yl)pyrimidin-4-yl)piperazin-1-yl)sulfonyl)propane-1,2-diol
370. 2-((4-(2-(isothiazol-4-yl)-6-(4-(trifluoromethyl)phenyl)pyrimidin-4-yl)piperazin-1-yl)sulfonyl)ethanol
371. (S)-2-(4-(6-(4-chlorophenyl)-2-(pyridin-3-yl)pyrimidin-4-yl)piperazin-2-yl)ethanol
372. (S)-4-(5-(6-(3-hydroxypyrrolidin-1-yl)-2-(pyridin-3-yl)pyrimidin-4-yl)pyridin-2-yl)morpholin-3-one
373. (S)-3-(4-(3-fluoro-4-morpholinophenyl)-6-(3-hydroxypyrrolidin-1-yl)pyrimidin-2-yl)pyridin-2-ol
373. (S)-3-(4-(3-fluoro-4-morpholinophenyl)-6-(3-hydroxypyrrolidin-1-yl)pyrimidin-2-yl)pyridin-2-ol
374. (S)-1-(6-(4-((2-(dimethylamino)ethyl)amino)phenyl)-2-(pyridin-3-yl)pyrimidin-4-yl)pyrrolidin-3-ol
375. (S)-1-(6-(4-(2-(dimethylamino)ethoxy)phenyl)-2-(pyridin-3-yl)pyrimidin-4-yl)pyrrolidin-3-ol, and
376. (S)-1-(6-(4-((2-hydroxyethyl)amino)phenyl)-2-(pyridin-3-yl)pyrimidin-4-yl)pyrrolidin-3-ol.

14. A pharmaceutical composition comprising the compound of formula (I) according to claim 1, or an enantiomer, diastereomer, racemate, solvate, hydrate, or pharmaceutically acceptable salt thereof and a pharmaceutically acceptable carrier.

15-20. (canceled)

21. A method of modulating AhR activity in a subject comprising administering a therapeutically effective amount of the compound of formula (I) according to claim 1.

22. A method of preventing or treating a disease or condition mediated by aryl hydrocarbon receptor (AhR) in a subject comprising administering a therapeutically effective amount of the compound of formula (I) according to claim 1.

23. The method according to claim 22, wherein the disease or condition mediated by aryl hydrocarbon receptor (AhR) is cancer, cancerous conditions, tumor, fibrotic disorders, or conditions with dysregulated immune responses or other disorders associated with aberrant AhR signaling.

24. The method according to claim 23, wherein the cancer is selected from a group consisting of a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, and a chronic myeloblastic leukemia.

25. The method according to claim 23, wherein the fibrotic disorder is selected from a group consisting of hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, scleroderma, morphea, keloids, hypertrophic scarring, naevi, diabetic retinopathy, proliferative vitroretinopathy and sarcoidosis.

26. The method according to claim 23, wherein the condition with dysregulated immune responses is selected from a group consisting of sepsis, multiple organ failure, inflammatory disorders of the kidney, chronic intestinal inflammations, pancreatitis, peritonitis, inflammatory skin disorders and inflammatory eye disorders, rheumatoid diseases, systemic lupus erythematosus and multiple sclerosis.

27. A method of inhibiting proliferation, tissue invasion, metastasis and angiogenesis of cancer cells in a subject having a cancer, a cancerous condition, or a tumor comprising administering a therapeutically effective amount of the compound of formula (I) according to claim 1.
